(19) United States
(12) Patent Application Publication
(10) Pub. No.: US 2024/0366602 A1
(43) Pub. Date: Nov. 7, 2024

(54) COMBINATION THERAPY OF APELIN RECEPTOR AGONIST AND GLP-1 RECEPTOR AGONIST FOR TREATING A DISEASE OR CONDITION ASSOCIATED WITH WEIGHT GAIN

(71) Applicant: BioAge Labs, Inc., Richmond, CA (US)

(72) Inventors: Kristen Patricia FORTNEY, Derry, NH (US); Eric Kim MORGEN, Toronto (CA); Paul David RUBIN, Henderson, NV (US); Yong PAN, Albany, CA (US); Yan WANG, Fremont, CA (US); Peng Khun LEONG, San Francisco, CA (US); Justin REBO, Derry, NH (US)

(21) Appl. No.: 18/754,784

(22) Filed: Jun. 26, 2024

Related U.S. Application Data
(63) Continuation of application No. PCT/US2024/010197, filed on Jan. 3, 2024.
(60) Provisional application No. 63/478,331, filed on Jan. 3, 2023.
(60) Provisional application No. 63/514,113, filed on Jul. 17, 2023.
(60) Provisional application No. 63/517,587, filed on Aug. 3, 2023.
(60) Provisional application No. 63/520,332, filed on Aug. 17, 2023.
(60) Provisional application No. 63/520,904, filed on Aug. 21, 2023.
(60) Provisional application No. 63/580,336, filed on Sep. 1, 2023.

(51) Int. Cl. A61K 31/506; A61K 38/22; A61K 38/26; A61P 3/04

(52) U.S. Cl. CPC A61K 31/506; A61K 38/22; A61K 38/26; A61P 3/04

(57) ABSTRACT

This disclosure provides methods for treating a condition or disease associated with weight gain that include coadministration of an apelin receptor agonist and a GLP-1 receptor agonist. Methods for increasing total weight loss in the subject relative to weight loss that would be caused by administration of a pre-determined amount of a GLP-1 receptor agonist alone are also provided. In some embodiments, the method is for preserving, or maintaining muscle mass, muscle function, and/or muscle strength in a subject undergoing weight loss therapy.

### 1. CROSS REFERENCE TO RELATED APPLICATIONS

This application is a bypass continuation of International Application No. PCT/US2024/010197, filed Jan. 3, 2024, which claims benefit of and priority to U.S. Provisional Application No. 63/478,331, filed Jan. 3, 2023, U.S. Provisional Application No. 63/514,113, filed Jul. 17, 2023, U.S. Provisional Application No. 63/517,587, filed Aug. 3, 2023, U.S. Provisional Application No. 63/520,332, filed Aug. 17, 2023, U.S. Provisional Application No. 63/520,904, filed Aug. 21, 2023, and U.S. Provisional Application No. 63/580,336, filed Sep. 1, 2023, the disclosures of which are incorporated herein by reference in their entireties.

### 2. BACKGROUND

Obesity is a globally increasing health problem associated with various diseases, particularly cardiovascular disease (CVD), type 2 diabetes, obstructive sleep apnea, certain types of cancer, and osteoarthritis. As a result, obesity has been found to reduce life expectancy. The rise in obesity drives an increase in diabetes, and approximately 90% of people with type 2 diabetes may be classified as obese. There are 246 million people worldwide with diabetes, and by 2025 it is estimated that 380 million will have diabetes.

Glucagon-like peptide-1 (GLP-1) receptor agonists are glucose-lowering drugs that induce clinically significant reductions in body weight. However, GLP-1 receptor agonists not only reduce fat mass, but have also been shown to reduce lean body mass and skeletal muscle.

Methods are presently needed to implement treatment with GLP-1 receptor agonists while preventing muscle loss and inducing preservation of muscle function in patients having a condition or disease associated with weight gain, including patients who are undergoing weight loss treatments.

### 3. SUMMARY

This disclosure provides methods for treating a condition or disorder associated with weight gain by co-administration of an apelin receptor agonist and a glucagon-like-peptide-1 (GLP-1) receptor agonist.

Although weight loss therapies provide a treatment of weight-gain induced comorbidities, such as obesity-associated comorbidities, weight loss therapies can have an impact on body composition. Body composition includes free mass (FM), fat free mass (FFM), lean body mass (LBM), skeletal muscle mass, bone mineral content, and total body water (TBW). Free mass is a mass of all adipose tissue, FFM is a total body mass minus total fat mass, LBM includes organs, skin, bones, total body water, and muscle mass minus total fat mass, skeletal muscle mass includes lean body mass minus connective tissue, skin, and other organs, and TBW is the summation of intra- and extra-cellular water. A GLP-1 receptor agonist (GLP-1RA) used to induce weight loss in a subject in need of weight loss therapy, can also induce loss of LBM and/or skeletal muscle associated with weight loss induced by the GLP-1RA. Such loss in LBM and/or skeletal muscle associated with weight loss can make these patients susceptible to muscle atrophies, sarcopenia, and frailty. In some embodiments, a subject who is overweight and recommended for weight loss therapy is already vulnerable to an increased risk of conditions such as diabetes, insulin resistance physical frailty, sarcopenia, and muscle atrophy. Subjects considered overweight include patients with a body mass index (BMI) of 25 or greater.

The present inventors discovered that co-administration of an apelin receptor agonist with a GLP-1 receptor agonist can induce or increase total weight loss (e.g., fat mass loss) but also preserve muscle function and muscle mass (e.g., lean muscle), and thus prevent loss of skeletal muscle and lean body mass that follows treatment with a GLP-1 receptor agonist. The present inventors discovered that the combination therapy can lead to increased total weight loss, reduction of fat mass percentage, increase in lean mass percentage, and/or improvement in body composition (higher lean mass/fat mass ratio) relative to that caused by administration of a pre-determined amount of a GLP-1 receptor agonist alone.

Agonists of the apelin receptor were tested in combination with various GLP-1 receptor agonists in mouse models of obesity. The apelin receptor agonists tested included BGE-105 and BAL-1480. BGE-105 has the structure shown below:

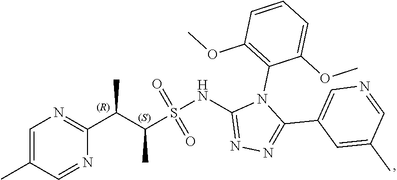

or a pharmaceutically acceptable salt thereof.

The GLP-1 receptor agonists tested in combinations with the apelin receptor agonist included semaglutide and tirzepatide.

Accordingly, a first aspect of the present disclosure is a method for treating a disease or condition associated with weight gain, including co-administering to a subject in need thereof: an effective dose of an apelin receptor agonist or a pharmaceutically acceptable salt thereof, and an effective dose of a GLP-1 receptor agonist or a pharmaceutically acceptable salt thereof. In some embodiments, the subject is obese. In some embodiments, the apelin receptor agonist is BGE-105, or a pharmaceutically acceptable salt thereof.

Aspects of this disclosure include a method of increasing total weight loss caused by administration of a pre-determined amount of a GLP-1 receptor agonist to a subject in need thereof. In some embodiments, the method includes co-administering to a subject in need thereof an effective dose of an apelin receptor agonist and an effective dose of a GLP-1 receptor agonist, to increase total weight loss in the subject. The increase in total weight loss in the subject can be relative to weight loss that would be caused by administration of a pre-determined amount of a GLP-1 receptor agonist alone.

The present disclosure also provides a method for inducing weight loss with maintenance of muscle mass and/or muscle strength (e.g., lean muscle mass) in a subject in need thereof (e.g., a subject undergoing weight loss therapy). The method can include co-administering to the subject in need thereof an effective dose of an apelin receptor agonist or a pharmaceutically acceptable salt thereof, and an effective amount of a GLP-1 receptor agonist, or a pharmaceutically acceptable salt thereof to maintain lean muscle mass while inducing fat and weight loss in the subject.

The present disclosure also provides a method for treating or preventing further muscle mass decrease caused by administration of a GLP-1 receptor agonist in a subject in need thereof. The method can include adding an effective dose of an apelin receptor agonist to the GLP-1 receptor agonist treatment regimen of a subject in need thereof to treat or prevent lean muscle mass decrease in the subject after administration of the GLP-1 receptor agonist.

The inventors discovered that co-administering of the apelin receptor agonist in conjunction with the GLP-1 receptor agonist according to the methods of this disclosure stimulates muscle mass preservation or an increase in muscle mass in the subject. In some embodiments, the subject exhibits loss of fat mass after the co-administration of the apelin receptor agonist, while at the same time maintaining lean muscle mass and/or improving the ratio of lean muscle to fat mass, e.g., relative to baseline values prior to the co-administration.

In some embodiments of the methods, the apelin receptor agonist is of formula (I) or (II), or a pharmaceutically acceptable salt thereof, as described herein. In some embodiments, the apelin receptor agonist is BGE-105, or a pharmaceutically acceptable salt thereof.

In some embodiments of the methods of this disclosure, the subject is an obese human and/or has, or is identified as having, or susceptible to or at risk of having, one or more of diabetes mellitus, insulin insensitivity, cardiovascular disease, cardiorenal disease, neurologic disease, obesity, is obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, or nighttime hypoglycemia. In some embodiments, the diabetes is type 1 diabetes, type 2 diabetes, or gestational diabetes.

### 4. BRIEF DESCRIPTION OF THE DRAWINGS

These and other features, aspects, and advantages of the present invention will become better understood with regard to the following description, and accompanying drawings, where:

FIG. 1 shows fat body mass (FBM) measurements of aged diet-induced obese (DIO) mice as measured using Echo-MRI for the various treatment groups in Example 1.

FIG. 2 shows lean body mass (LBM) as a percentage of total body weight (BW) of aged diet-induced obese (DIO) mice as measured using Echo-MRI for the various treatment groups in Example 1.

FIGS. 3A-3B show assessment of muscle function using grid hang tests of aged DIO mice for the various treatment groups of Example 1. FIG. 3A shows analysis using One-Way ANOVA with Tukey's multiple comparisons test, whereas FIG. 3B shows analysis using One-Way ANOVA without multiple comparisons test (Fisher's LSD)).

FIGS. 4A-4B show measurements of perigonadal fat weight (FIG. 4A) and quadriceps weight as a percentage of total body weight (FIG. 4B) for aged DIO mice treatment groups in Example 1.

FIG. 5 shows measurements of plasma neutrophil gelatinase-associated lipocalin (NGAL) level for the DIO-aged mice treatment groups in Example 1.

FIGS. 6A-6B show lean body mass and body weight measurements of female vs male aged DIO mice of Example 2.

FIGS. 7A-7D show measurements of body weight (FIG. 7A), fat mass (FIG. 7C), lean mass (FIG. 7D), and fed glucose (FIG. 7B) for both male and female mice groups used for randomization in Example 3.

FIGS. 8A-8B show daily water intake measured for all treatment groups every 3 days throughout the duration of the study of Example 2.

FIGS. 9A-9B show daily food intake measured for all treatment groups every 3 days throughout the duration of the study of Example 2.

FIGS. 10A-10B show cumulative water intake measured for all treatment groups every 3 days throughout the duration of the study of Example 2.

FIGS. 11A-11B show cumulative food intake measured for all treatment groups every 3 days throughout the duration of the study of Example 2.

FIGS. 12A-12C show body weight (BW) loss of treatment groups Example 2 with tirzepatide at three different dosages (3 nmol/kg (FIG. 12A), 10 nmol/kg (FIG. 12B), or 30 nmol/kg (FIG. 12C)) alone or in combination with BGE-105 (at 0.275 g/L or 1.1 g/L).

FIG. 13 shows that BGE-105 treatment results in a significant, dose-dependent increase in overall weight loss compared to tirzepatide alone as measured by body weight loss (BWL) %. See Example 2.

FIGS. 14A-14C show body composition of mice treated in the various treatment groups of Example 2. FIG. 14A shows change of fat body mass over body weight (FBM/BW) %. FIG. 14B shows change of lean body mass over body weight (LBM/BW) %. FIG. 14C shows change of lean body mass over fat ratio (Lean/Fat Ratio).

FIG. 15 shows fed glucose loss % of mice treated in the various treatment groups of Example 2.

FIGS. 16A-16B illustrate overall weigh loss. BGE-105 in combination with tirzepatide significantly reduced body weight compared to BGE-105 or tirzepatide monotherapy in % change (FIG. 16A) and absolute body weight (g) (FIG. 16B). See Example 3.

FIG. 17 shows daily food consumption (g/gBW/day) of mice treatment groups of Example 3. Tirzepatide monotherapy or BGE-105 combination with tirzepatide treatment reduced food daily food consumption compared to the DIO control group of Example 3.

FIGS. 18A-18B illustrate percentage of lean mass and percentage fat mass assessed by echo-MRI. BGE-105 combination with tirzepatide treatment increased percentage of lean mass (FIG. 18A) and reduced percentage of fat mass (FIG. 18B) and restored the levels to that comparable to the lean control group at the end of the treatment (Day 21). See Example 3.

FIGS. 19A-19B illustrate absolute lean mass and absolute fat mass assessed by Echo-MRI. BGE-105 combination with tirzepatide treatment dramatically decreased absolute (g) of fat body mass (FIG. 19B). BGE-105 combination with tirzepatide treatment restored absolute fat mass (FIG. 19B) the level to that comparable to the lean control group at the end of the treatment (Day 21). See Example 3.

FIG. 20 illustrates lean/fat ratio in mice treatment groups. BGE-105 with tirzepatide treated mice had increased lean/fat ratio as compared to tirzepatide alone. The mice treated with high dose of BGE-105 with tirzepatide combination showed comparable lean/fat ratio to the lean control group at the measurement on day 20.

FIG. 21 shows addition of high dose BGE-105 lowered the fed glucose levels achieved with tirzepatide. The data demonstrates that a combination therapy may benefit patients with insulin resistance.

FIGS. 22A-22C illustrate muscle function in mice treatment groups as assessed via grid hang tests. FIG. 22C shows an image of the grid hang test. FIG. 22A shows a graph of latency of fall (s), and FIG. 22B shows graph of body weight×latency of fall (g*s). The results show that addition of BGE-105 to tirzepatide restored muscle function to that of lean controls.

FIGS. 23A-23B shows that there were comparable effects on fat mass for BGE-105 in combination with tirzepatide in obese mice, as for bimagrumab (Example 4).

FIGS. 24A-24B shows that there were comparable effects on lean mass for BGE-105 in combination with tirzepatide in obese mice as for bimagrumab (Example 4).

FIGS. 25A-25B shows the monoclonal antibody bimagrumab in combination with tirzepatide provided a comparable lean/fat ratio (FIG. 25B) as BGE-105 (1.1 g/L) in combination with tirzepatide (FIG. 25A). The data demonstrate comparable effects of BGE-105 and bimagrumab on body composition when co-administered with tirzepatide.

FIGS. 26A-26B illustrate BGE-105 and tirzepatide combination reduced body weight and body weight percentage in adult mice (Example 5).

FIGS. 27A-27B show food and water consumption of adult mice in treatment groups of Example 5.

FIGS. 28A-28E show lean mass (FIG. 28A), fat mass (FIG. 28B), lean mass percentage (FIG. 28C), fat mass percentage (FIG. 28D), and lean/fat mass ratio (FIG. 28E) in treated adult mice of Example 5. BGE-105 and tirzepatide combination treatment showed significant reduction of absolute fat mass (FIG. 28B).

FIG. 29 shows blood glucose level in adult mice treatment groups of Example 5.

FIGS. 30A-31B illustrate that a BAL-1480 and tirzepatide combination treatment reduced body weight and body weight percentage.

FIGS. 31A-31B show food and water consumption of mice in treatment groups of Example 6.

FIG. 32 shows hydration ratio of mice in treatment groups of Example 6.

FIGS. 33A-33B show lean mass and lean mass percentage of mice in treatment groups. BAL-1480 and tirzepatide combination restored lean mass percentage to lean control level.

FIGS. 34A-34C illustrate fat mass, fat mass percentage and lean/fat ratio in mice treatment groups. BAL-1480 and tirzepatide combination restored fat mass (FIG. 34A), fat mass percentage (FIG. 34B), and lean/fat ratio (FIG. 34C) to lean control level.

FIG. 35 shows blood glucose level in mice treatment groups of Example 6.

FIG. 36 shows rectal temperature of treated mice at Day 15.

FIGS. 37A-37P illustrate fatty liver weight and fat tissue weights in tirzepatide treated mice. Shown are results of fatty liver (FIG. 37A), fatty liver percentage (FIG. 37B), inguinal fat (FIG. 37C), inguinal fat percentage (FIG. 37D), perigonadal fat (FIG. 37E), perigonadal fat percentage (FIG. 37F), brown fat (FIG. 37G), brown fat percentage (FIG. 37H), tibialis anterior (TA) muscle (FIG. 37I), TA percentage (FIG. 37J), quadricep (quad) muscle (FIG. 37K), quadricep muscle percentage (FIG. 37L), gastrocnemius (gastric) muscle (FIG. 37M), gastrocnemius muscle percentage (FIG. 37N), total muscle (FIG. 37O), and total muscle percentage (FIG. 37P).

FIGS. 38A-38B illustrate that a combination of BGE-105 and semaglutide reduced body weight and body weight percentage in a dose dependent fashion. See Example 7.

FIGS. 39A-39B show food and water consumption of mice in semaglutide treatment groups of Example 7.

FIGS. 40A-40B show lean mass and lean mass percentage in semaglutide treated mice. BGE-105 and semaglutide combination restored lean mass percentage to lean control level in a dose dependent fashion.

FIGS. 41A-41C illustrate fat mass, fat mass percentage and lean/fat ratio in semaglutide treated mice. BGE-105 and semaglutide combination restored fat mass (FIG. 41A), fat mass percentage (FIG. 41B), and lean/fat ratio (FIG. 41C) to lean control level in a dose dependent fashion.

FIG. 42 shows semaglutide treated blood glucose level in mice treatment groups.

FIG. 43 shows rectal temperature of semaglutide treated mice at Day 15.

FIGS. 44A-44P illustrate fatty liver weight and fat tissue weights in semaglutide treated mice. Shown are results of fatty liver (FIG. 44A), fatty liver percentage (FIG. 44B), inguinal fat (FIG. 44C), inguinal fat percentage (FIG. 44D), perigonadal fat (FIG. 44E), perigonadal fat percentage (FIG. 44F), brown fat (FIG. 44G), brown fat percentage (FIG. 44H), tibialis anterior (TA) muscle (FIG. 44I), TA percentage (FIG. 44J), quadricep (quad) muscle (FIG. 44K), quadricep muscle percentage (FIG. 44L), gastrocnemius (gastric) muscle (FIG. 44M), gastrocnemius muscle percentage (FIG. 44N), total muscle (FIG. 44O), and total muscle percentage (FIG. 44P). See Example 7.

FIG. 45 provides a study dosing outline for BGE-105 in a Phase 1 clinical study with single ascending dose (SAD) cohorts 1-3, or multiple doses (MD) cohorts 1A-1C of Example 8.

FIG. 46 provides a screening and pre-treatment outline of MD cohorts of Part B in Example 8.

FIG. 47 provides a treatment and follow up outline of MD cohorts of Part B in Example 8.

FIGS. 48A-48B shows Preliminary PK data from the 3 SAD cohorts demonstrating dose proportionality. The Cmax remained within the expected range and for the highest dose (240 mg/1440 mg) the AUClast was 1062 μg*hr/mL.

FIG. 49 shows that preliminary results for SAD cohorts 1-3 (study Part A of Example 8), where there is a 17% increase from baseline in HOMA-IR on the Day 3 Predose visit and 12% increase from baseline in HOMA-IR on the Day 4 visit for the Placebo group. In the BGE-105 treated patients (cohorts 1, 2, and 3) there is a decrease in the percent change from baseline in HOMA-IR for both visits with the largest decrease in the highest dosing group (cohort 3) indicating a positive effect on insulin sensitivity and an improvement in insulin resistance.

FIGS. 50A-50C show the effects of BGE-105 on rest-induced reduction in thigh circumference, Vastus Lateralis diameter % (thickness), Vastus Lateralis cross sectional area (CSA) %, muscle degeneration, and cumulative protein synthesis rate % (as measured from a biopsy). FIG. 50C shows thigh circumference of patients received placebo (Cohort 1A of MD study of Example 8) and BGE-105 treatment (Cohort 1B of MD study of Example 8) period is measured and presented as percent change from baseline over the course or after the 10-day bed rest duration. p=0.0004. Measurements are made 15 cm superior of the mid patella. BGE-105 significantly reduced muscle atrophy across multiple key endpoints, in healthy volunteers aged≥65 years. The higher rate of muscle protein synthesis in BGE-105 treated patients vs. placebo group provides a potential mechanistic basis for BGE-105's protective effect on muscle dimensions. The findings of the Phase 1b trial support investigation of BGE-105 as a treatment of a wide range of age-related syndromes driven by loss of muscle. These conditions include acute myopathies in hospitalized patients on mechanical ventilation, as well as chronic medical conditions. BGE-105 significantly prevented muscle atrophy across multiple endpoints: (circumference (p<0.001), Diameter (p<0.01), cross sectional area (p<0.05), muscle grade (progression) (p<0.005), and cumulative protein synthesis (p<0.005).

FIGS. 51A-51B shows effects of patients treated with BGE-105 on muscle diameter and cross-sectional area of the vastus lateralis via ultrasound (FIG. 51A) after 10-days of bed rest. The vastus lateralis diameter or vastus lateralis cross sectional area of patients treated with BGE-105 or placebo is presented as a mean percent change from baseline. p=0.0297 (FIG. 51B).

FIGS. 52A-52C shows fatty degeneration of the vastus lateralis via echo density as measured using an ultrasound muscle quality grading scale (FIGS. 52A-B). FIG. 52A is a diagram illustrating a grade of 1 in normal muscle (1), FIG. 52B illustrates a grade 2 of muscle containing some fatty streaks (2). Shown are muscle quality at baseline and after 10 days of bed rest observed in each of placebo and BGE-105 group (FIG. 52C) (p=0.0019).

FIG. 53 shows rate of muscle protein synthesis in the vastus lateralis, measured via microbiopsy in individual subjects in the BGE-105 treated group and the placebo group. Muscle protein synthesis is measured after 10 days bed rest and normalized fraction synthetic rate by subject is presented. p=0.0043.

FIG. 54 shows the step ratio of patients who wore a wearable activity device during Day 10 through Day 60 (post-bedrest time period) of the Phase 1b, MD, Part B clinical trial of Example 8. The ratio is calculated by dividing eat subjects later step counts by their baseline step count, which is the mean daily steps seen over the pre-bedrest period. Patients treated with BGE-105 showed an increase in physical activity by determining the number of steps taken by the patient using the wearable activity device as compared to the patients who received a placebo. The activity levels start off near the baseline and then increase for the BGE-105 treated group for several weeks before the curve converges again.

FIGS. 55A-55D show proteomic profiling analysis performed on serum collected from subjects of the phase 1B clinical trial of Example 8. 11 treated and 11 placebo subject's serum levels were profiled for their proteomics collected at day −1 (baseline), day 5 and day 11. FIG. 55A shows the number of proteins associated with frailty (functionality), walk speed, instrumental activities of daily living (IADL) (functional instrument), and grip strength that are changed in patients treated with BGE-105. IADL is a specific class of functional activities being measured. For frailty (functionality), 69 out of 992 proteins associated with frailty in patients of the phase 1b clinical trial were changed by treatment with BGE-105 (p<0.05). For walk speed, 35 out of 526 proteins associated with walk speed in patients of the phase 1b clinical trial were changed by treatment with BGE-105. For grip strength, 58 out of 379 proteins associated with grip strength were changed by treatment with BGE-105. FIG. 55B shows that BGE-105 shifted the serum proteome towards a healthier state, recapitulating the benefits of naturally high apelin levels in subjects treated with BGE-105. FIG. 55C shows changes in baseline energy expenditure in BGE-105 treated and placebo treated subjects using a SomaSignal test on the proteomic data. FIG. 55D shows changes in cardiorespiratory fitness (VO2) max and basal metabolic rate in BGE-105 treated and placebo treated subjects using a SomaSignal test on the proteomic data.

FIG. 56 shows BGE-105 preserves synthesis rate of structural proteins to maintain muscle mass.

FIG. 57, panels A-B, show BGE-105 treatment shift the proteome towards an estimated higher basal metabolic rate and V02 max by SomaSignal tests.

FIG. 58 shows BGE-105 improves post-bedrest recovery by wearable accelerometer.

FIGS. 59A-59B show BGE-105 shifted the serum proteome towards a healthier state, recapitulating benefits of naturally high apelin levels. FIG. 59A summarizes shifting of serum protein groups showing concurrent phenotype (grip strength group, walking speed) and future outcomes (strenuous activity, longevity, walking speed, physical function). FIG. 59B shows protein associations with future walk speed impairment in the human aging cohort. FIG. 59B shows proteins associated with preservation of walking speed after 10 days bed rest. FIG. 59B also shows protein associated with future walking speed impairment after 10 days bed rest.

FIGS. 60A-60B illustrates resting energy expenditure and cardiorespiratory fitness in subjects treated with BGE-105.

FIGS. 61A-61B illustrates correlation of exercise and proteomes in BGE-105 treated subjects. BGE-105 prevented increase of exercise negatively associated protein change from baseline (p=6.1E-05).

FIG. 62 shows that differentially regulated pathways suggest BGE-105 beneficial effects on key muscle and adipocyte processes.

FIG. 63 shows BGE-105 prevents bed rest-induced downregulation of muscle contractile proteins in fast and slow skeletal muscles. p values reflect change after 10 days of bedrest, compared to baseline. BGE-105 prevented reduction of troponin C (TNNC1), which is involved in calcium binding during skeletal muscle contraction (p=0.008); myosin heavy chain beta (MYH7), which provides structural support for the myosin motor (p=0.012); and calcium-ATPase type 2 in the sarco-/endoplasmic reticulum (SERCA2), which plays a critical role in contraction (p=0.028). p values reflect change after 10 days of bedrest, compared to baseline.

FIGS. 64A-64C show BGE-105 prevents bed rest-induced downregulation of mitochondrial biogenic regulator PGC-1α and all respiratory complexes. Shown are representative genes including PGC-1α (p=0.029), COMPLEX 1: NDUFA8 (p=0.011), COMPLEX II: SDHD (p=0.033), COMPLEX III: UQCRB (p=0.046), COMPLEXT IV: COX10 (p=0.049), COMPLEX V: ATP5PB (p=0.0088).

FIGS. 65A-65B show single-nuclei transcriptomics of BGE-105 preserved gene expression involved in glucose metabolism. FIG. 65A shows differential expression of representative genes (PGC-1α, p=0.029; EIF 4EBP1, p=0.009; PHKA1, p=0.015) in insulin signaling pathway (p=2.61E-03). FIG. 65B shows differential expression of representative genes (MLYCD, p=0.015; EEF2K, p=0.031; CD36, p=0.05) in AMPK signaling pathway (p=5.04E-03). p values reflect change after 10 days of bedrest, compared to baseline.

FIG. 66 shows BGE-105 decreased expression of genes involved in fat storage in muscle interstitial adipocytes. p values reflect change after 10 days of bedrest, compared to baseline, based on 727 adipocytes captured. BGE-105 prevented reduction of G0/G1 switch gene (GOS2), which inhibits lipolysis by directly binding to adipose triglyceride lipase (p=0.042); cyl-CoA:diacylglycerol acyltransferases 2 (DGAT2), which catalyzes the final step in triglyceride synthesis, leading to the storage of fats (p=0.22); and fatty acid binding protein 4 (FABP4), which is an intracellular lipid-binding protein that facilitates the transport of fatty acid (p=0.069).

FIGS. 67-89 show details and results of snRNAseq analyses of human muscle tissue samples from a Phase 1b clinical study of BGE-105 for treating muscle atrophy.

FIG. 67 illustrates streamlined sample preparation workflow including nuclei isolation and 10× Genomics single cell transcriptome gene expression technology used to assess tissue samples from clinical trial.

FIGS. 68A-68B show 11 cell types were identified, consistent with published muscle atlas. Two methods and two annotations were used. Top 20 variable genes within clusters were used as markers for cell type annotation.

FIG. 69 shows that there is consistency between the top 20 cell-type specific expressed genes and known cell type markers. Plot shows log 2 (fold change) in expression in a specific cell-type compared to rest for the top 20 cell-type specific expressed genes. Analysis was performed using all samples.

FIG. 70 shows that differentially expressed genes associated with BGE105 were identified for each cell type.

FIG. 71 shows that signaling pathways that control muscle loss and promote muscle growth were enriched in BGE105 treatment associated genes in fast skeletal muscle.

FIGS. 72A-72B show that treatment association of most of significant genes (padj<0.1) in muscle growth/loss related signaling pathways were in the anticipated direction.

FIGS. 73A-73C show that bulk expression level of VEGFA, PPRGC1A and COL1A1 were higher in treated group than that in placebo group on day 11.

FIGS. 74A-74B show that bulk expression level of TNNC1 and MYH7 were higher in treated compared to placebo on day 11.

FIG. 75 shows that for fast skeletal muscle: 10 groups of 5+ enriched pathways were identified.

FIG. 76 shows that for slow skeletal muscle: 13 groups of 5+ enriched pathways were identified.

FIGS. 77A-77B show cell type-specific patterns of differential gene expression associated with BGE-105 treatment in muscle biopsies were identified.

FIG. 78 shows that differentially regulated pathways suggest BGE-105 beneficial effects on key muscle and adipocyte processes.

FIG. 79 shows BGE-105 prevents bed rest-induced downregulation of muscle contractile proteins in fast and slow skeletal muscles.

FIG. 80 shows BGE-105 prevents bed rest-induced downregulation of mitochondrial biogenic regulator PGC-1α and all respiratory complexes.

FIG. 81 shows BGE-105 prevented detrimental expression level of genes involved in muscle metabolic processes.

FIG. 82 shows BGE-105 prevents bed rest-induced upregulation of genes involved in triglyceride storage and fatty acid metabolism, potential mechanism for promoting fat loss.

FIG. 83 shows APLNR was expressed in more Endothelial cells in the treated group.

FIG. 84 Cell differentiation trajectory and pseudotime inference. Pseudotime inference for cell differentiation is a computational approach to model dynamic changes and transitions between different cellular states. It allows for the distinction between early and late stages of a biological process. Pseudotime analysis can help uncover the chronological sequence of gene expression changes during a biological process, thereby allowing us to understand the underlying molecular mechanisms.

FIG. 85 shows a plot indicating cells were less differentiated with treatment for Fast/Slow skeletal muscle, macrophages, T/NK cells and muscle stem cell.

FIGS. 86-88 illustrate the results of secondary analyses. Evaluation of signal from protein synthesis rate analysis. Investigation of aging and muscle signatures from published research.

FIG. 86 shows BGE-105 resulted in relatively higher muscle protein synthesis in the vastus lateralis, measured via microbiopsy (p<0.005).

FIG. 87 shows the validation of muscle protein synthesis assay results in snRNA-seq analysis.

FIG. 88 shows that BGE-105 treatment shifts the transcriptome of fast/slow skeletal muscles towards a state linked to younger muscle.

FIG. 89 shows graph that indicate % of cells that have Mitochondria reads more than 5% within a sample. Y-axis is the percentage of cells that MT reads>5% within a sample (62 samples=21 patients*3 time points) compared to that in baseline. P. values of T-test are slightly different from the Slide 10 because the cells that have low qualities have been removed before calculating the proportion of cells that have more than 5% Mitochondrial reads for each patient (cells with less than 200 genes or less than 500 reads were removed, total remaining MT cells (>5%):6136).

### 5. DETAILED DESCRIPTION OF THE INVENTION

### 5.1. Methods of Treating a Condition or Disorder Associated with Weight Gain

The present disclosure provides a method of treating a subject for a condition associated with weight gain, using a combination of an apelin receptor agonist and a GLP-1 receptor agonist. In some embodiments, the GLP-1 receptor agonist is referred to as a GLP-1 analog. In some embodiments, the method is a method of weight loss in a subject in need thereof. In some embodiments, the method includes co-administering to a subject a therapeutically effective amount of an apelin receptor agonist (e.g., as described herein), and a therapeutically effective amount GLP-1 receptor agonist (e.g., as described herein).

A receptor agonist is a compound that binds to a receptor and elicits a response typical of the natural ligand. A full agonist may be defined as one that elicits a response of the same magnitude as the natural ligand.

The “condition associated with weight gain” (referred to interchangeably herein as an “weight gain-related muscle condition” or “fat gain-related muscle condition”) refers to a disease or condition associated with weight gain in a mammalian subject, such as obesity-associated comorbidities. In some embodiments, weight gain includes fat gain. In some embodiments, weight gain consists of fat gain.

Examples of conditions that can be targeted for treatment according to the methods of this disclosure include, but are not limited to, obesity, diabetes mellitus, insulin insensitivity, cardiovascular disease, cardiorenal disease, neurologic disease, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, nighttime hypoglycemia, or a combination of treatments including obesity and sarcopenia, diabetes mellitus and sarcopenia, insulin insensitivity and sarcopenia, cardiovascular disease and sarcopenia, cardiorenal disease and sarcopenia, neurologic disease and sarcopenia, obesity-linked gallbladder disease and sarcopenia, obesity-induced sleep apnea and sarcopenia, diabetes and sarcopenia, excessive appetite and sarcopenia, fatty liver disease and sarcopenia, non-alcoholic fatty liver disease (NASH) and sarcopenia, dyslipidemia and sarcopenia, metabolic syndrome and sarcopenia, insufficient satiety and sarcopenia, hyperinsulinemia and sarcopenia, nighttime hypoglycemia and sarcopenia, obesity and frailty, diabetes mellitus and frailty, insulin insensitivity and frailty, cardiovascular disease and frailty, cardiorenal disease and frailty, neurologic disease and frailty, obesity-linked gallbladder disease and frailty, obesity-induced sleep apnea and frailty, diabetes and frailty, excessive appetite and frailty, fatty liver disease and frailty, non-alcoholic fatty liver disease (NASH) and frailty, dyslipidemia and frailty, metabolic syndrome and frailty, insufficient satiety and frailty, hyperinsulinemia and frailty, or nighttime hypoglycemia and frailty.

In some embodiments, the weight gain associated condition is obesity. In some embodiments, the weight gain associated condition is excessive weight gain. In some embodiments, the weight gain associated condition is diabetes mellitus. In some embodiments, the weight gain associated condition is insulin insensitivity. In some embodiments, the weight gain associated condition is cardiovascular disease. In some embodiments, the weight gain associated condition is neurologic disease. In some embodiments, the condition is obesity-linked gallbladder disease. In some embodiments, the weight gain associated condition is obesity-induced sleep apnea. In some embodiments, the condition is diabetes. In some embodiments, the weight gain associated condition is excessive appetite. In some embodiments, the weight gain associated condition is fatty liver disease. In some embodiments, the weight gain associated condition is non-alcoholic fatty liver disease (NASH). In some embodiments, the weight gain associated condition is dyslipidemia. In some embodiments, the condition is metabolic syndrome. In some embodiments, the condition is insufficient satiety. In some embodiments, the weight gain associated condition is hyperinsulinemia. In some embodiments, the weight gain associated condition is nighttime hypoglycemia.

In another aspect, the present disclosure provides methods of inducing weight loss in a subject while preserving or maintaining muscle mass and/or muscle function, using a combination therapy of apelin receptor agonist and GLP-1 receptor agonist.

Aspects of the present disclosure include methods of using a combination of the apelin receptor agonist and GLP-1 receptor agonist include use as an adjunct to a reduced-calorie diet and/or increased physical activity for chronic weight management in overweight or obese subjects, e.g., adults with an initial body mass index (BMI) of: 30 kg/m² or greater (obesity) or BMI of 27 kg/m² or greater (overweight). In some embodiments, the subject to be treated is overweight and in the presence of at least one weight-related comorbid condition (e.g., hypertension, dyslipidemia, type 2 diabetes mellitus, obstructive sleep apnea or cardiovascular disease).

Aspects of the present disclosure include methods of using of the apelin receptor agonist in combination with a GLP-1 receptor agonist and/or another drug that reduces caloric intake, as an adjunct to a reduced-calorie diet and/or increased physical activity for chronic weight management in overweight or obese subjects (e.g., adults with an initial body mass index (BMI) of: 30 kg/m² or greater (obesity) or BMI of 27 kg/m² or greater (overweight). In some embodiments, the subject to be treated is overweight and in the presence of at least one weight-related comorbid condition (e.g., hypertension, dyslipidemia, type 2 diabetes mellitus, obstructive sleep apnea or cardiovascular disease).

A drug that reduces caloric intake is a drug that can regulate appetite to make a subject feel less hungry and/or feel full faster after eating less food, resulting in fewer calories and less food being consumed by the subject. In some embodiments, a drug that reduces caloric intake is an appetite suppressant (e.g., as described herein). In some embodiments, the drug that reduces caloric intake is a cannabinoid receptor 1 (CB1r or CANN6 or CNR1) antagonist (e.g., as described herein).

### 5.2. Methods of Increasing Weight Loss, or Inducing Weight Loss while Maintaining Muscle Mass or Muscle Strength

The present inventors demonstrated that co-administration of the apelin receptor agonist with the GLP-1 receptor agonist produced more weight loss, e.g., including more fat loss, than would have been expected from administration of GLP-1 receptor agonist alone. Accordingly, aspects of this disclosure include a method of increasing total weight loss caused by administration of a pre-determined amount of a GLP-1 receptor agonist to a subject in need thereof. In some embodiments, the method includes co-administering to a subject in need thereof an effective dose of an apelin receptor agonist and an effective dose of a GLP-1 receptor agonist, to increase total weight loss and/or fat loss in the subject. The increase in total weight loss or fat loss in the subject can be relative to weight loss that would be caused by administration of a pre-determined amount of a GLP-1 receptor agonist alone.

In some embodiments, the method includes adding an effective dose of an apelin receptor agonist to the GLP-1 receptor agonist treatment regimen of a subject in need thereof to increase total weight loss or fat loss caused by administration of a pre-determined amount of a GLP-1 receptor agonist to the subject.

In some embodiments, the increase in total weight loss is an increase of 5% or more over the weight loss that would be caused by, or expected for, administration of a pre-determined amount of a GLP-1 receptor agonist alone to the subject, such as an increase of 6% or more, 7% or more, 8% or more, 9% or more, 10% or more, 15% or more, 20% or more, 25% or more, 30% or more, 35% or more, 40% or more, 45% or more, or 50% or more in total weight loss.

In some embodiments, the increase in fat loss is an increase of 5% or more over the fat loss that would be caused by, or expected for, administration of a pre-determined amount of a GLP-1 receptor agonist alone to the subject, such as an increase of 6% or more, 7% or more, 8% or more, 9% or more, 10% or more, 15% or more, 20% or more, 25% or more, 30% or more, 35% or more, 40% or more, 45% or more, or 50% or more in fat loss.

Aspects of this disclosure include a method for inducing or increasing weight loss while maintaining and/or increasing muscle mass and/or muscle strength in a subject that has a condition or disease associated with weight gain. In some embodiments, the method is for maintenance of lean muscle mass. In some embodiments, the subject is undergoing weight loss therapy.

In various embodiments, an apelin receptor agonist (e.g., as described herein) is administered to the subject to maintain or increase muscle mass (lean muscle) and/or muscle strength in skeletal muscle of the subject.

The muscle mass and/or muscle strength of a subject can be monitored during treatment and compared to a baseline level assessed prior to dosing with the apelin receptor agonist and the GLP-1 receptor agonist. In some embodiments, the muscle mass (e.g., lean muscle) or muscle strength of a subject is at least maintained at or near baseline levels during treatment, e.g., within 10% of baseline levels. In some embodiments, the subject is one who has suffered from declining muscle mass and/or muscle strength over time, and administration of the apelin receptor agonist according to methods of this disclosure reverses and/or ameliorates the decline.

Fat mass levels and lean muscle mass levels in a subject can be assessed prior to administration of either of the compounds (e.g., in a subject naïve to treatment with a GLP-1 receptor agonist). Baseline levels of fat mass and lean muscle mass in the subject can be assessed immediately prior to co-administration. In some embodiments, the subject exhibits loss of fat mass relative to baseline level but not a loss of lean muscle mass relative to baseline level after the co-administration. In some embodiments, the subject exhibits loss of fat mass relative to baseline level, an increase in lean to fat mass ratio, and/or increase in lean mass percentage, relative to baseline level in the subject (e.g., a baseline level in a subject naïve to treatment with a GLP-1 receptor agonist) after the co-administration of the apelin receptor agonist and the GLP-1 receptor agonist.

In some embodiments, a decrease of fat mass (or fat % of body weight BW) relative to baseline level is a decrease of 10% or more, such as a decrease of 15% or more, 20% or more, 25% or more, 30% or more, 35% or more, 40% or more, 45% or more, or 50% or more. In some embodiments, a decrease of fat % of body weight (BW) relative to baseline level is a decrease of 10% or more, such as a decrease of 15% or more, 20% or more, 25% or more, 30% or more, 35% or more, 40% or more, 45% or more, or 50% or more.

In some embodiments, the lean mass is maintained at a level that is within 10% of to baseline level, such as within 5% of baseline level. In some embodiments, the increase in lean muscle % of body weight is an increase of 5% or more relative to baseline level, such as an increase of 10% or more, 15% or more, 20% or more, 25% or more, 30% or more, 35% or more, 40% or more, 45% or more, or 50% or more. In some embodiments, the increase in lean to fat ratio is an increase of 5% or more relative to baseline level, such as an increase of 10% or more, 15% or more, 20% or more, 25% or more, 30% or more, 35% or more, 40% or more, 45% or more, or 50% or more.

Aspects of this disclosure include methods of treating or preventing further muscle mass decrease caused by administration of a GLP-1 receptor agonist to a subject in need thereof. Thus, the method can include adding an effective dose of an apelin receptor agonist to the GLP-1 receptor agonist treatment regimen of a subject in need thereof. In some embodiments, the method treats or prevents lean muscle mass decrease in the subject after administration of the GLP-1 receptor agonist.

Fat mass levels and lean muscle mass levels in a subject undergoing GLP-1 receptor agonist therapy can be assessed prior to administration of the GLP-1 receptor agonist. A decrease in lean muscle mass caused by GLP-1 receptor agonist monotherapy over time can be assessed. Baseline levels of fat mass and lean muscle mass in the subject can be assessed immediately prior to administration of the apelin receptor agonist. Further decreases from baseline in lean muscle mass after administration of the apelin receptor agonist can be at least ameliorated and/or prevented using the methods of this disclosure. In some embodiments, the subject exhibits loss or decrease of fat mass relative to baseline level but not a loss of lean muscle mass relative to baseline level after the co-administration of the apelin receptor agonist. In some embodiments, the subject exhibits more fat mass loss relative to baseline level. In some embodiments, the subject exhibits an increase in lean to fat mass ratio relative to baseline level. In some embodiments, the subject exhibits an increase in lean mass percentage, relative to baseline level in the subject after the co-administration of the apelin receptor agonist.

In some embodiments, the subject exhibits an increased lean mass percentage, or increased lean/fat mass ratio after the co-administration, relative to a baseline level assessed before the co-administration.

In some embodiments, the subject exhibits a normal fed glucose level after the co-administration, e.g., within 20 days or less, such as 12 days or less, or 6 days or less of the co-administration, where baseline fed glucose levels were elevated above normal. A normal blood glucose level can be readily determined by the skilled artisan and can vary depending on, e.g., whether the patient has diabetes.

### 5.3. Apelin Receptor Agonists

Apelin is the endogenous peptide ligand for the apelin receptor (also referred to as APJ, or APLNR). The apelin receptor is a member of the rhodopsin-like G protein-coupled receptor (GPCR) family. The apelin/APJ system is distributed in diverse periphery organ tissues and can play various roles in the physiology and pathophysiology of many organs. The apelin/APJ system participates in various cell activities such as proliferation, migration, apoptosis or inflammation. An apelin receptor agonist is any compound capable of promoting or activating the apelin/APJ system directly or indirectly, competitively, or non-competitively. Agonistic activities of a compound toward apelin receptor may be determined by any suitable method in the art. For example, the agonist can be assessed using the natural agonist of apelin receptor (i.e. apelin) and its receptor for promotion of the function of the receptor.

In some embodiments, the apelin receptor agonist is a polypeptide, such as an apelin polypeptide, e.g., one of several active isoforms ranging from 36 to 12 amino acids in length, or a fragment or analog thereof. Exemplary polypeptides that can be apelin receptor agonists include, but are not limited to, apelin-36, apelin-17, apelin-13, [Pyr1] apelin-13, and metabolically stable apelin analogs described in International Publication No. WO2016102648.

In some embodiments, the apelin receptor agonist is a small molecule. The term “small molecule” refers to an organic molecule of a size comparable to those organic molecules generally used in pharmaceuticals. The term excludes biological macromolecules (e.g., proteins, nucleic acids, etc.). Preferred small organic molecules range in size up to 5000 Da, more preferably up to 2000 Da, and most preferably up to 1000 Da.

Exemplary apelin receptor agonists of interest include, but are not limited to, E339-3D6 (see, e.g., Iturrioz et al. (FASEB Journal, Volume 24, Issue 5, May 2010, Pages 1506-1517), ML233, BMS-986224, ANPA-0073, AMG986, and the like.

As further described below, in some embodiments of the methods of this disclosure, the apelin receptor agonist is a compound described in U.S. Pat. Nos. 9,573,936, 9,868,721, International Publication No. WO2016196771, U.S. Pat. No. 10,011,594, U.S. Pat. No. RE49,594 E (a reissue of U.S. Pat. No. 10,100,059) or Narayanan et al. (J. Med. Chem. 2021, 64, 3006-3025), the disclosures of which are herein incorporated by reference in their entirety.

As known by those skilled in the art, certain compounds of this disclosure may exist in one or more tautomeric forms. Because one chemical structure may only be used to represent one tautomeric form, it will be understood that for convenience, referral to a compound of a given structural formula includes tautomers of the structure represented by the structural formula.

In some embodiments, the apelin receptor agonist is a compound of formula (I) or (II):

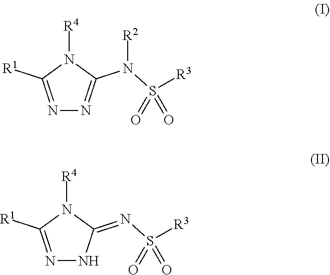

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

In some embodiments, the apelin receptor agonist is a compound of formula (I) or (II):

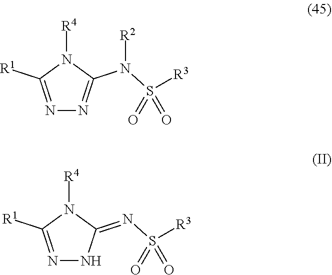

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆alkyl, —C₁-C₆haloalkyl, —C₁-C₆perhaloalkyl, —OH, —O—(C₁-C₆alkyl), —O—(C₁-C₆ haloalkyl), —O—(C₁-C₆perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, or S;
R² is selected from —H, or C₁-C₄ alkyl or is absent in the compounds of Formula II;
R³ is a group of formula —(CR^{3d}R^{3e})—(CR^{3f}R^{3g})-Q;
R^{3d} and R^{3e} are independently selected from —H, —F, —Cl, —CN, —C₁-C₆ alkyl, —C₁-C₆haloalkyl, —C₁-C₆ perhaloalkyl, —OH, —O—(C₁-C₆ alkyl), —O—(C₁-C₆haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆ alkyl)₂;
R^{3f} and R^{3g} are independently selected from —H, —F, —Cl, —CN, —C₁-C₆ alkyl, —C₁-C₆haloalkyl, —C₁-C₆ perhaloalkyl, —OH, —O—(C₁-C₆ alkyl), —O—(C₁-C₆haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆ alkyl)₂;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, or a heteroaryl group, and the Q heterocyclyl group may be substituted with 1 oxo substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, or S, or a monocyclic or bicyclic heterocyclyl group with 5 to 10 ring members containing 1, 2, 3, or 4 heteroatoms independently selected from N, O, or S, wherein the C₆-C₁₀ aryl group, the heteroaryl group, or the heterocyclyl group are unsubstituted or are substituted with 1, 2, or 3 R^{4a} substituents; and
R^{4a} in each instance is independently selected from —F, —Cl, —Br, —I, —CN, —C₁-C₆alkyl, —C₁-C₆haloalkyl, —C₁-C₆perhaloalkyl, —OH, —O—(C₁-C₆alkyl), —O—(C₁-C₆haloalkyl), —O—(C₁-C₆perhaloalkyl), —NH₂, —NH(C₁-C₆alkyl), —N(C₁-C₆alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), or —C(═O)N(C₁-C₆ alkyl)₂, and the heterocyclyl R⁴ group may be further substituted with 1 oxo substituent.

As noted above, apelin receptor agonist compounds of this disclosure may exist in multiple tautomeric forms. This is particularly true in compounds of Formula I where R² is H. These forms are illustrated below as Tautomer A and Tautomer B:

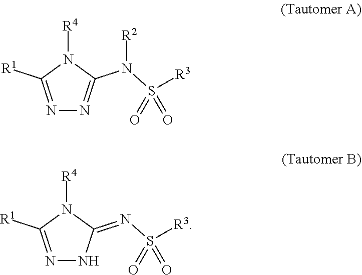

Apelin receptor agonist compounds of this disclosure are depicted structurally and generally named as compounds in the “Tautomer A” form. However, it is specifically contemplated and known that the compounds exist in “Tautomer B” form and thus compounds in “Tautomer B” form are expressly considered to be part of this disclosure. For this reason, the claims refer to compounds of Formula I and Formula II. Depending on the compound, some compounds may exist primarily in one form more than another. Also, depending on the compound and the energy required to convert one tautomer to the other, some compounds may exist as mixtures at room temperature whereas others may be isolated in one tautomeric form or the other.

In some embodiments of formula (I) and (II), R¹ is an unsubstituted pyridyl or is a pyridyl substituted with 1 or 2 R^{1a} substituents.

In some embodiments of formula (I) and (II), R^{1a} in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

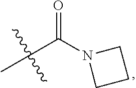 [SMILES: *C(=O)N1CCC1]

wherein the symbol [symbol] , when drawn across a bond, indicates the point of attachment to the rest of the molecule.

In some embodiments of formula (I) and (II), R¹ is selected from

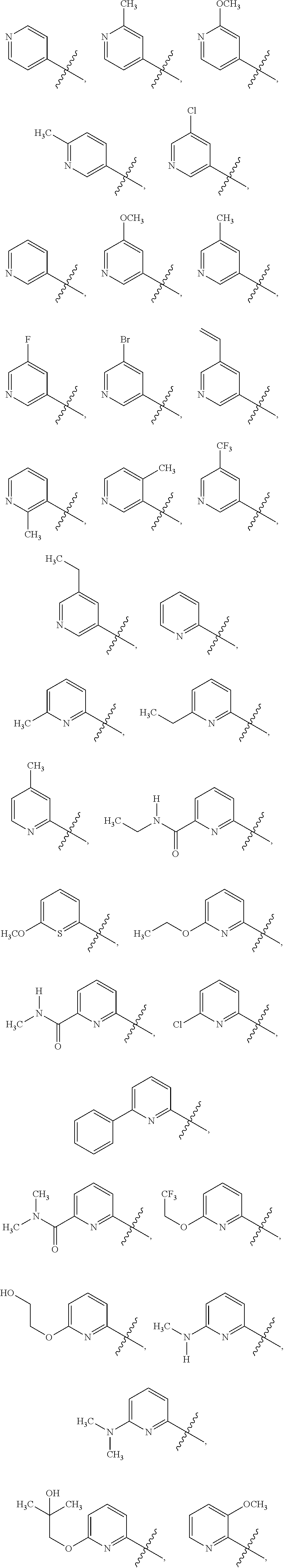

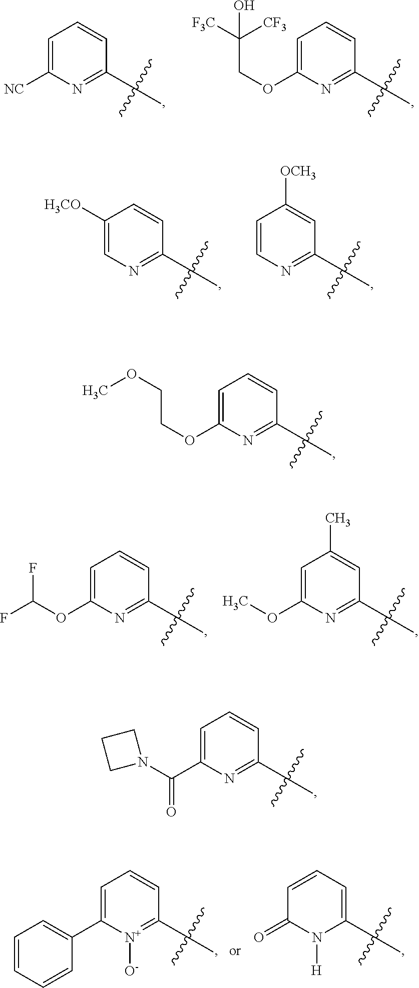

wherein the symbol [symbol] , when drawn across a bond, indicates the point of attachment to the rest of the molecule.

In some embodiments of formula (I) and (II), R¹ is selected from

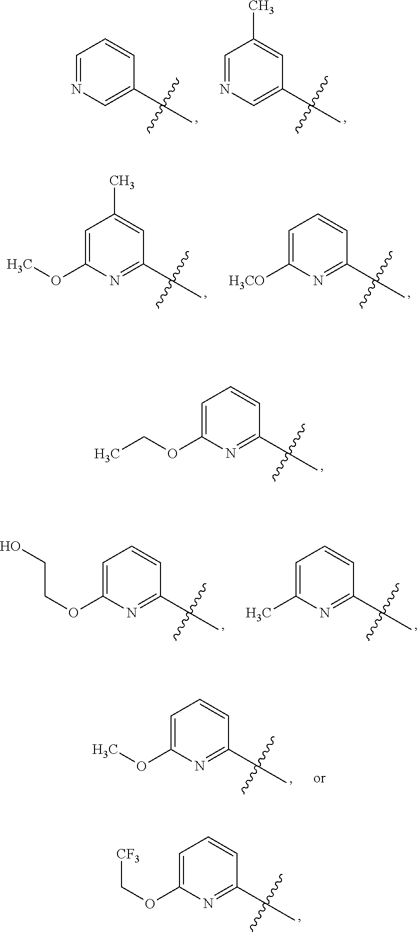

wherein the symbol [symbol] , when drawn across a bond, indicates the point of attachment to the rest of the molecule.

In some embodiments of formula (I) and (II), R² is —H.

In some embodiments of formula (I) and (II), R⁴ is a phenyl, pyridyl, pyrimidinyl, isoxazolyl, indolyl, naphthyl, or pyridinyl any of which may be unsubstituted or substituted with 1, 2, or 3 R^{4a} substituents. In some embodiments of formula (I) and (II), R⁴ is a phenyl substituted with 1 or 2 R^{4a} substituents. In some embodiments of formula (I) and (II), the 1 or 2 R^{4a} substituents are —O—(C₁-C₂ alkyl) groups.

In some embodiments of formula (I) and (II), R⁴ is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

In some embodiments of formula (I) and (II), R⁴ is selected from:

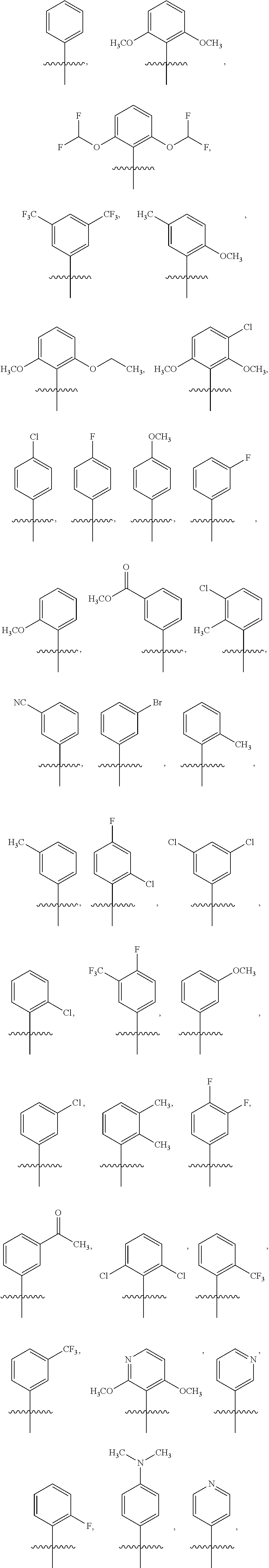

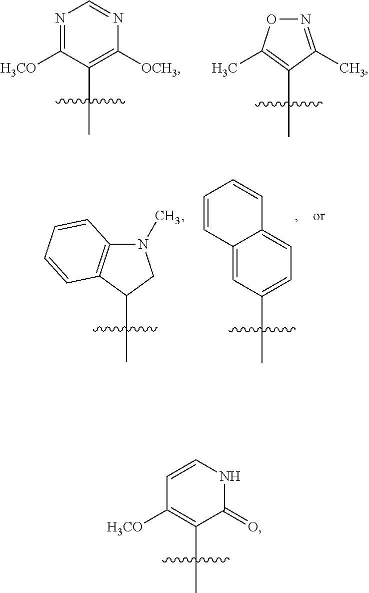

wherein the symbol [symbol] , when drawn across a bond, indicates the point of attachment to the rest of the molecule.

In some embodiments of formula (I) and (II), R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

In some embodiments of formula (I) and (II), Q is a monocyclic heteroaryl group with 5 or 6 ring members containing 1 or 2 heteroatoms selected from N, O, or S and Q is unsubstituted or is substituted with 1 or 2 R^{Q} substituents.

In some embodiments of formula (I) and (II), Q is selected from

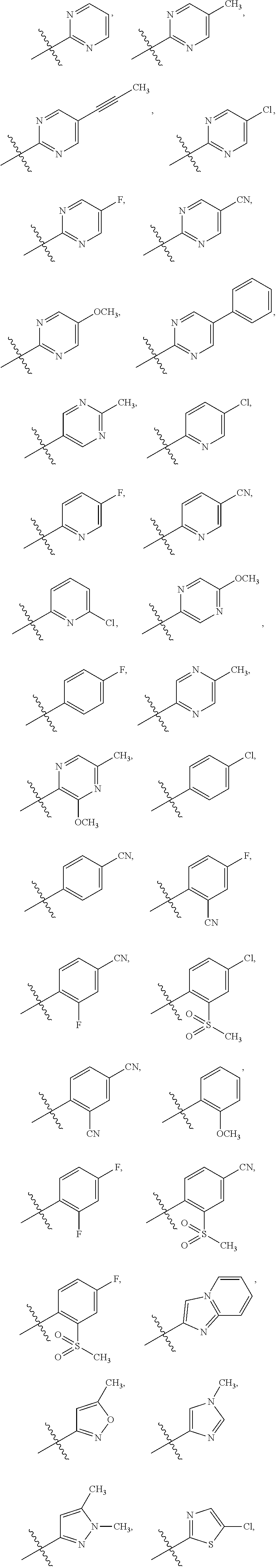

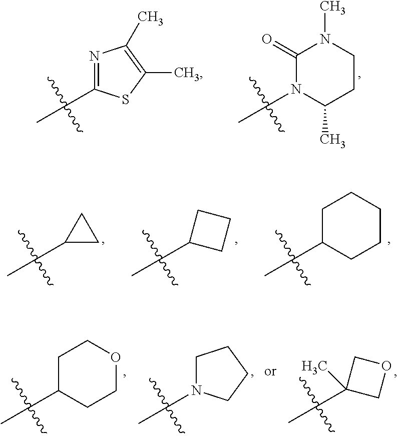

wherein the symbol [symbol] , when drawn across a bond, indicates the point of attachment to the rest of the molecule.

In some embodiments of formula (I) and (II), R³ is a group of formula-(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), R³ is a group of formula —(CR^{3d}R^{3e})—(CR^{3f}R^{3g})-Q.

In some embodiments of formula (I) and (II), R³ has one of the formula

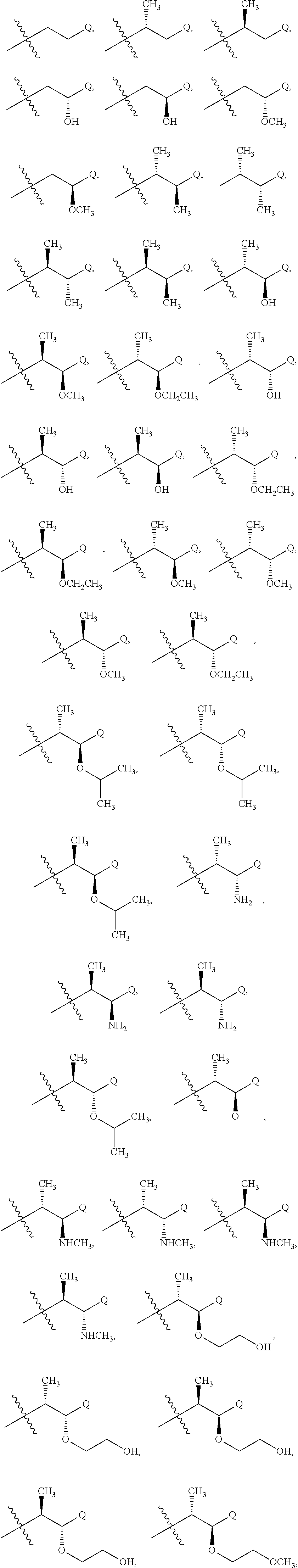

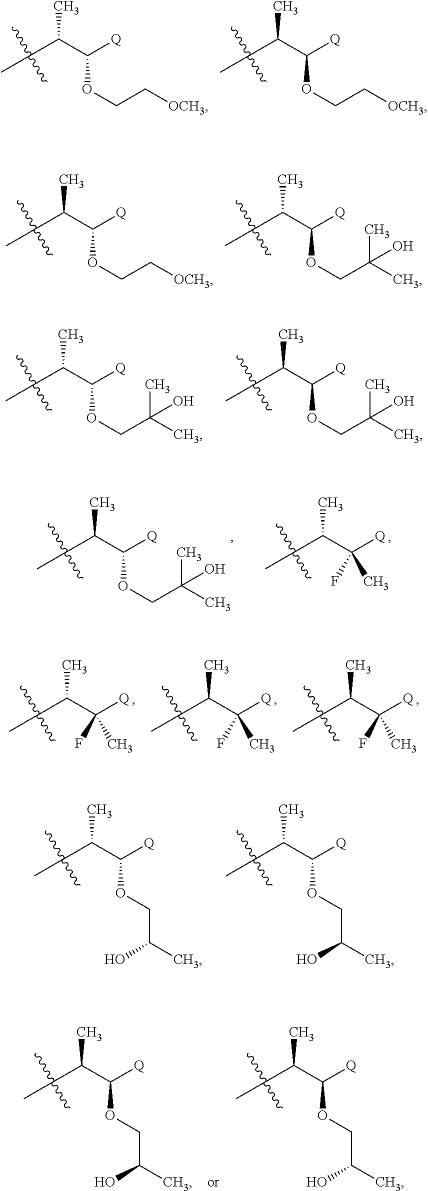

wherein the symbol [symbol] , when drawn across a bond, indicates the point of attachment to the rest of the molecule.

In some embodiments of formula (I) and (II), R³ has one of the formula

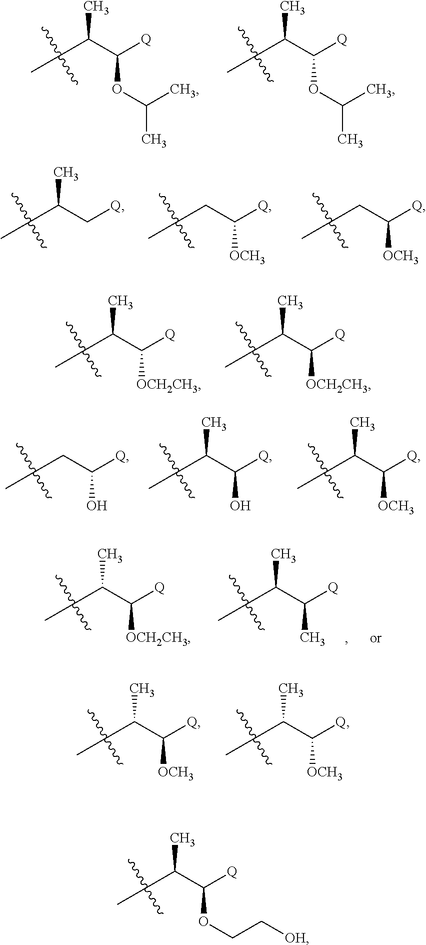

wherein the symbol [symbol] , when drawn across a bond, indicates the point of attachment to the rest of the molecule.

In particular embodiments of formula (I) and (II), the apelin receptor agonist is
(1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide;
(1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide;
(1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide;
(1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide;
(1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide;
(1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide;
(1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide;
(1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide;
(1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide;
(1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide; or
(1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide or the pharmaceutically acceptable salt thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propane sulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (JR,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is (JR,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is (JR,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide, or a pharmaceutically acceptable salt thereof.

In a particular embodiment of formula (I) and (II), the apelin receptor agonist is (IS,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide, or a pharmaceutically acceptable salt thereof.

In a particular embodiment of formula (I) and (II), the apelin receptor agonist is (IS,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide, or a pharmaceutically acceptable salt thereof.

In a particular embodiment of formula (I) and (II), the apelin receptor agonist is (JR,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide, or a pharmaceutically acceptable salt thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof.

In a particular embodiment of formula (I) and (II), the apelin receptor agonist is (IS,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide, or a pharmaceutically acceptable salt thereof.

In a particular embodiment of formula (I) and (II), the apelin receptor agonist is (JR,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide, or a pharmaceutically acceptable salt thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide, or a pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide, or a pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide, or a pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide, or a pharmaceutically acceptable salt thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof.

In a particular embodiment of formula (I) and (II), the apelin receptor agonist is (1R,2S)—N-(4-(2,6-difluorophenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is (1R,2S)—N-(4-(4,6-dimethoxy-5-pyrimidinyl)-5-(2-pyridinyl)-4H-1,2,4-triazol-3-yl)-1-methoxy-1-(5-methyl-2-pyrimidinyl)-2-propanesulfonamide, or a pharmaceutically acceptable salt thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide (BGE-105) or a pharmaceutically acceptable salt thereof.

In a particular embodiment of formula (I) and (II), the apelin receptor agonist is

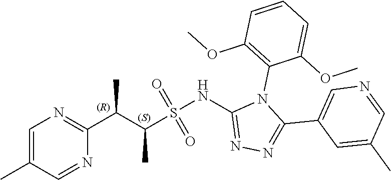

(BGE-105) or a pharmaceutically acceptable salt thereof.

U.S. Pat. Nos. 9,573,936, 9,868,721, 9,745,286, 9,656,997, 9,751,864, 9,656,998, 9,845,310, 10,058,550, 10,221,162, and 10,344,016, the disclosures of which are incorporated herein by reference in their entirety, describe apelin receptor agonists of formula (I) or (II), and methods of synthesizing such triazole agonists of the apelin receptor, including BGE-105. See e.g., Example 263.0 of U.S. Pat. No. 9,573,936.

In some embodiments, the apelin receptor agonist is a compound of Formula (XI)

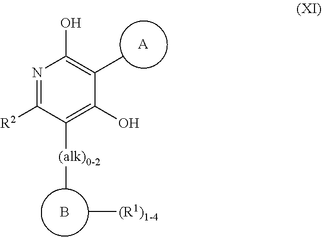

or a stereoisomer, a tautomer, or a pharmaceutically acceptable salt thereof, wherein:
alk is C₁₋₆ alkyl substituted with 0-5 R^{e};
ring A is independently selected from the group consisting of

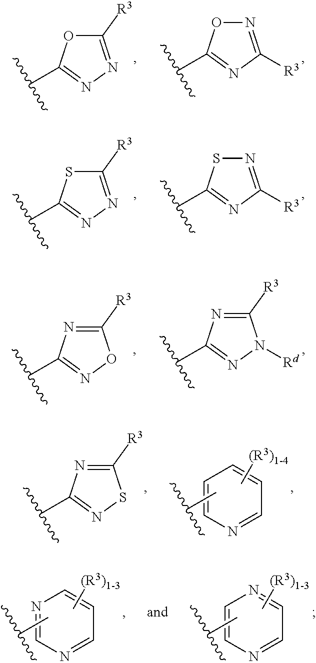

ring B is independently selected from the group consisting of:

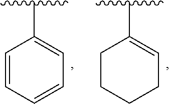

and 6-membered heteroaryl;
R¹ is independently selected from the group consisting of: H, halogen, NO₂, —(CH₂)ₙOR^{b}, (CH₂)ₙS(O)ₚR_{c}, —(CH₂)ₙC(═O)R^{b}, —(CH₂)ₙNR^{a}R^{a}, —(CH₂)ₙCN, —(CH₂)ₙC(═O)NR^{a}R^{a}, —(CH₂)ₙNR^{a}C(═O)R^{b}, —(CH₂)ₙNR^{a}C(═O)NR^{a}R^{a}, —(CH₂)ₙNR^{a}C(═O)OR^{b}, —(CH₂)ₙOC(═O)NR^{a}R^{a}, —(CH₂)ₙC(═O)OR^{b}, —(CH₂)ₙS(O)ₚNR^{a}R^{a}, —(CH₂)ₙNR^{a}S(O)ₚNR^{a}R^{a}, —(CH₂)ₙNR^{a}S(O)ₚR^{c}, C₁₋₄ alkyl substituted with 0-3 R^{e}, —(CH₂)ₙ—C₃₋₆ carbocyclyl substituted with 0-3 R^{e}, and —(CH₂)ₙ-heterocyclyl substituted with 0-3 R^{e};
R² is independently selected from the group consisting of: C₁₋₅ alkyl substituted with 0-3 R^{e}, C₁₋₅ alkenyl substituted with 0-3 R^{e}, and C₁₋₆ cycloalkyl substituted with 0-3 R^{e}; provided when R² is C₁₋₅ alkyl, the carbon atom except the one attached directly to the pyridine ring may be replaced by O, N, and S;
R³ is independently selected from the group consisting of:
(1) —(CR⁴R⁴)ᵣC(═O)OC₁₋₄ alkyl substituted with 0-5 R^{e},
(2) —(CR⁴R⁴)ᵣNR^{a}R^{a},
(3) —(CR⁴R⁴)ᵣC(═O)NR^{a}R^{a},
(4) —(CR⁴R⁴)ᵣNR^{a}C(═O)C₁₋₄alkyl substituted with 0-5 R^{e},
(5) —(CR⁴R⁴)ᵣNR^{a}C(═O)(CR⁴R⁴)ₙOC₁₋₄alkyl substituted with 0-5 R^{e},
(6) —(CR⁴R⁴)ᵣ—R⁵,
(7) —(CR⁴R⁴)ᵣ—OR⁵,
(8) —(CR⁴R⁴)ᵣNR^{a}C(═O)(CR⁴R⁴)ₙR⁵, and
(9) (CR⁴R⁴)ᵣC(═O)NR^{a}(CR⁴R⁴)ₙR⁵;
R⁴ is independently selected from the group consisting of: H, halogen, NR^{a}R^{a}, OC₁₋₄ alkyl, and C₁₋₄ alkyl; or R⁴ and R⁴ together with the carbon atom to which they are both attached form C₃₋₆ cycloalkyl substituted with 0-5 R^{e};
R⁵ is independently selected from the group consisting of: —(CH₂)ₙ—C₃₋₁₀ carbocycle and —(CH₂)ₙ-heterocycle, each substituted with 0-3 R⁶;
R⁶ is independently selected from: H, halogen, ═O, —(CH₂)ₙOR^{b}, (CH₂)ₙS(O)ₚR_{c}, —(CH₂)ₙC(═O)R^{b}, —(CH₂)ₙNR^{a}R^{a}, —(CH₂)ₙCN, —(CH₂)ₙC(═O)NR^{a}R^{a}, (CH₂)ₙNR^{a}C(═O)R^{b}, —(CH₂)ₙNR^{a}C(═O)NR^{a}R^{a}, —(CH₂)ₙNR^{a}C(═O)OR^{b}, —(CH₂)ₙOC(═O)NR^{a}R^{a}, —(CH₂)ₙC(═O)OR^{b}, —(CH₂)ₙS(O)ₚNR^{a}R^{a}, —(CH₂)ₙNR^{a}S(O)ₚNR^{a}R^{a}, —(CH₂)ₙNR^{a}S(O)_{P}R, C₁₋₅ alkyl substituted with 0-3 R^{e}, (CH₂)ₙ—C₃₋₆ carbocyclyl substituted with 0-3 R^{e}, and —(CH₂)ₙ-heterocyclyl substituted with 0-3 R^{e};
R^{a} is independently selected from the group consisting of: H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b} is independently selected from the group consisting of: H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c} is independently selected from the group consisting of: C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d} is independently selected from the group consisting of: H and C₁₋₄alkyl substituted with 0-5 R^{e};
R^{e} is independently selected from the group consisting of: C₁₋₆ alkyl substituted with 0-5 R_{f}, C₂₋₆ alkenyl, C₂₋₆ alkynyl, —(CH₂)ₙ—C₃₋₆ cycloalkyl, —(CH₂)ₙ—C₄₋₆ heterocyclyl, —(CH₂)ₙ-aryl, —(CH₂)ₙ-heteroaryl, F, Cl, Br, CN, NO₂, ═O, CO₂H, —(CH₂)ₙOR_{f}, S(O)ₚR^{f}, C(═O)NR^{f}R^{f}, NR^{f}C(═O)R^{f}, S(O)ₚNR^{f}R^{f}, NR^{f}S(O)ₚR^{f}, NR^{f}C(═O)OR^{f}, OC(═O)NR^{f}R^{f}, and —(CH₂)ₙNR^{f}R^{f};
R_{f} is independently selected from the group consisting of: H, F, Cl, Br, CN, OH, C₁₋₅alkyl (optimally substituted with halogen and OH), C₃₋₆ cycloalkyl, and phenyl, or R^{f} and R^{f} together with the nitrogen atom to which they are both attached form a heterocyclic ring optionally substituted with C₁₋₄alkyl;
n is independently selected from zero, 1, 2, and 3;
r is independently selected from zero, 1, 2, and 3; and
p is independently selected from zero, 1, and 2.

In some embodiments of the compound of Formula (XI), the compound is of Formula (XV):

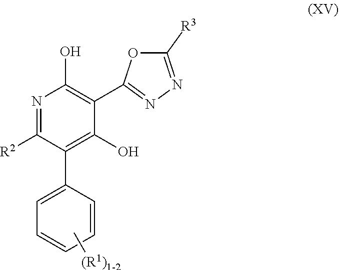

or a stereoisomer, a tautomer, or a pharmaceutically acceptable salt thereof, wherein:
R¹ is independently selected from the group consisting of: —CH₂OH, —OCH₃, —OCF₃, CH₃, CH₂CH₃, CH(CH₃)₂, and cyclopropyl;
R² is independently selected from the group consisting of: C₁₋₄ alkyl substituted with 0-3 R^{e}, C₂₋₄ alkenyl, C₁₋₆ cycloalkyl, and CH₂O(CH₂)₁₋₃CH₃;
R³ is independently selected from the group consisting of:
(1) —CH₂C(═O)OC₁₋₄ alkyl substituted with 0-3 R^{e},
(2) —CH₂NR^{a}R^{a},
(3) —CH₂C(═O)NR^{a}R^{a},
(4) —CH₂NHC(═O)C₁₋₄alkyl substituted with 0-3 R^{e},
(5) —CH₂NR^{a}C(═O)(CH₂)₀₋₂OC₁₋₄alkyl substituted with 0-3 R^{e},
(6) —CH₂—R⁵,
(7) —CH₂—OR⁵,
(8) —CH₂NR^{a}C(═O)(CH₂)₀₋₂R⁵, and
(9) —CH₂C(═O)NR^{a}(CH₂)₀₋₂R⁵;
R⁵ is independently selected from the group consisting of: aryl, C₃₋₆ cycloalkyl, and heterocycle, each substituted with 0-3 R⁶;
R⁶ is independently selected from the group consisting of: H, F, Cl, Br, —OR^{b}, ═O, —(CH₂)ₙC(═O)R^{b}, —(CH₂)ₙC(═O)OR^{b}, —(CH₂)NR^{a}R^{a}, CN, —(CH₂)ₙC(═O)NR^{a}R^{a}, —S(O)₂NH₂, C₁₋₄ alkyl substituted with 0-3 R^{e}, (CH₂)ₙ—C₃₋₆ carbocyclyl substituted with 0-3 R^{e}, and —(CH₂)ₙ-heterocyclyl substituted with 0-3 R^{e};
R^{a} is independently selected from the group consisting of: H, C₁₋₆ alkyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b} is independently selected from the group consisting of: H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{e} is independently selected from the group consisting of: C₁₋₆ alkyl (optionally substituted with F and Cl), OH, OCH₃, OCF₃, —(CH₂)ₙ—C₃₋₆ cycloalkyl, —(CH₂)ₙ—C₄₋₆ heterocyclyl, —(CH₂)ₙ-aryl, —(CH₂)ₙ-heteroaryl, F, Cl, Br, CN, NO₂, ═O, and CO₂H; and
n is independently selected from zero, 1, 2, and 3.

In some embodiments, the apelin receptor agonist is a compound having the structure:

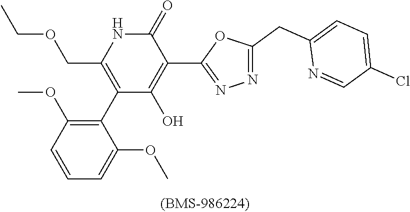 [SMILES: CCOCc1[nH]c(=O)c(-c2nnc(Cc3ccc(Cl)cn3)o2)c(O)c1-c1c(OC)cccc1OC]

or a stereoisomer, a tautomer, or a pharmaceutically acceptable salt thereof

In some embodiments, the apelin receptor agonist is a compound having the structure:

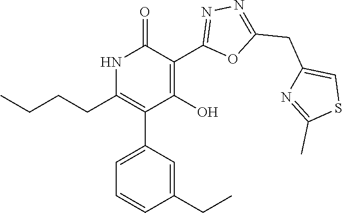 [SMILES: CCCCc1[nH]c(=O)c(-c2nnc(Cc3csc(C)n3)o2)c(O)c1-c1cccc(CC)c1]

or a stereoisomer, a tautomer, or a pharmaceutically acceptable salt thereof.

In some embodiments, the apelin receptor agonist is a pyrazole agonist as described in U.S. Pat. No. RE49,594 E (a reissue of U.S. Pat. No. 10,100,059) or by Narayanan et al. (J. Med. Chem. 2021, 64, 3006-3025). In some embodiments, the apelin receptor agonist is a compound of Formula (XXI):

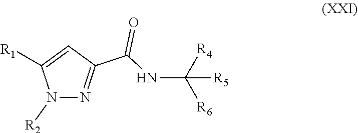

or a pharmaceutically acceptable salt thereof, a prodrug thereof, or a salt of a prodrug thereof, wherein
R₁ is represented by the formula:

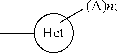

wherein

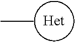

is a monocyclic aryl or heteroaryl group;
each A is independently fluoro substituted C₁-C₃ alkoxy or fluoro substituted C₁-C₃ alkyl;
n is 1, 2, 3, 4, or 5:
R₂ is C₃₋₈ alkyl, C₁₋₈ alkyl (C₃₋₈ cycloalkyl), C₃₋₈ cycloalkyl, heteroaryl, or substituted aryl;
R⁴ is adamantanyl aryl, C₁₋₈ alkyl, C₁₋₈ alkyl alcohol, C₁₋₈ alkylamino, C₁₋₈ alkyl amido, C₂₋₈ alkyl(aryl), C₁₋₈ alkyl (C₃₋₈ cycloalkyl), C₁₋₈ alkyl (C₃₋₈ cycloakyl)-CO₂R₇, C₁₋₈ alkyl guanidinyl, C₁₋₈ alkyl heteroaryl, C₁₋₈ alkyl tetrazol-5-one, C₂₋₄ alkyl heterocycloalkyl, C₁₋₈ alkyl thioether, C₁₋₈ alkyl thiol, C₂₋₈ alkenyl, C₂₋₈ alkenyl(aryl), C₂₋₈ alkenyl(heteroaryl), C₃₋₈ alkynyl, C₃₋₈ cycloalkyl, C₃₋₈ cycloalkyl-CO₂R₇, (CH₂)ₓNR₇R₈, (CH₂)ₓOR₇, (CH₂)ₓNR₉COR₇, (CH₂)ₓNR₉SO₂R₇, (CH₂)ₓNR₉CO₂R₇, (CH₂)ₓNHCOR₇, (CH₂)ₓNHSO₂R₇, (CH₂)ₓNHCO₂R₇, (CH₂)ₓCONR₇R₈, (CH₂)ₓCONR₇(CH₂)_{y}CO₂R₉, (CH₂)ₓCONR₇(CH₂)_{y}C₂CONR₇R₈, (CH₂)ₓCONR₇(CH₂)_{y}R₉((CH₂)ₓ(COR₇, (CH₂)ₓCO₂R₇, (CH₂)ₓSO₂NR₇(CH₂)_{y}R₉, CHR₇COR₉, CHR₇CONHCHR₈COR₉, CONR₇R₈, CONR₇(CH₂)ₓCO₂R₈, CONR₇CHR₈CO₂R₉, CO₂R₉, NHC₂R₇, or (CH₂)ₓSO₂NR₇R₈;
R₅ and R₆ each are independently is adamantanyl aryl, C₁₋₈ alkyl, C₁₋₈ alkyl alcohol, C₁₋₈ alkyl amino, C₁₋₈ alkyl amido, C₂₋₈ alkyl(aryl), C₁₋₈ alkyl (C₃₋₈ cycloalkyl), C₁₋₈ alkyl (C₃₋₈ cycloalkyl)-CO₂R₇, C₁₋₈ alkyl guanidinyl, C₁₋₈ alkyl heteroaryl, C₁₋₈ alkyl tetrazol-5-one, C₂₋₄ alkyl heterocycloalkyl, C₁₋₈ alkyl thioether, C₁₋₈ alkyl thiol. C₂₋₈ alkenyl, C₂₋₈ alkenyl(aryl), C₂₋₈ alkenyl(heteroaryl), (C₃₋₈ alkynl, C₃₋₈ cycloalkyl, C₃₋₈ cycloalkyl-CO₂R₇, (CH₂)ₓNR₇R₈, (CH₂)ₓOR₇, (CH₂)ₓNR₉COR₇, (CH₂)ₓNR₉SO₂R₇, (CH₂)ₓNR₉CO₂R₇, (CH₂)ₓNHCOR₇, (CH₂)ₓNHSO₂R₇, (CH₂)ₓNHCO₂R₇, (CH₂)ₓCONR₇R₈, (CH₂)ₓCONR₇(CH₂)_{y}CO₂R₉, (CH₂)ₓCONR₇(CH₂)_{y}CONR₇R₈, (CH₂)ₓCONR₇(CH₂)_{y}R₉, (CH)₂COR₇, (CH₂)ₓCO₂R₇, (CH₂)ₓSO₂NR₇(CH₂)_{y}R₉, CHR₇COR₉, CHR₇CONHCHR₈COR₉, CONR₇R₈, CONR₇(CH₂)ₓCO₂R₈, CONR₇CHR₈CO₂R₉, CO₂R₉, NHCO₂R₇, or (CH₂)ₓSO₂NR₇R₈; or
R₄ and R₅ together make a 4-8 member ring which may be substituted with one or more heteroatoms; or
R₄ and R₅ together make a 5-8 nitrogen containing member ring with one or more carbonyl groups;
wherein the group R₄ is substituted with one or more fluorine atoms:
R₆ is H;
R₇ and R₈ each are independently H, C₁₋₈ alkoxy, aryl, C₁₋₈ alkyl, C₁₋₈ alkyl alcohol, C₁₋₈ alkyl amino, C₁₋₈ alkyl amido, C₁₋₈ alkyl(aryl), C₁₋₈ alkyl (C₃₋₈ cycloalkyl), C₁₋₈ alkyl tetrazol-5-one, C₁₋₈ alkyl guanidinyl, C₁₋₈ alkyl heteroaryl, C₁₋₈ alkyl thioether, C₁₋₈ alkyl thiol, C₁₋₈ alkenyl, C₃₋₈ alkynyl, C₃₋₈ cycloalkyl, (CH₂)ₓCONHR₉, (CH₂)ₓCOR₉, (CH₂)ₓCO₂R₉, or heteroaryl; or
R⁷ and R₈ together make a 3-9 member ring which may contain one or more heteroatoms, wherein the ring is substituted with at least two fluorine atoms; or
R⁷ and R₈ together make a 5-8 nitrogen containing member ring with one or more carbonyl groups;
R₉ is aryl, C₁₋₈ alkoxy, C₁₋₈ alkyl, C₁₋₈ alkyl(aryl), C₃₋₈ cycloalkyl, H, heteroaryl, or hydroxyl;
each x is independently 0-8; and
each y is independently 1-8.

In some embodiments of Formula (XXI), the apelin receptor agonist is a compound of the structure:

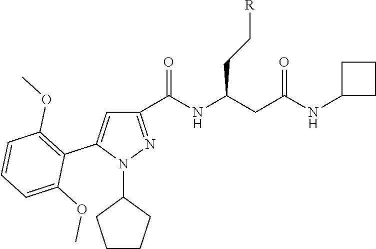

or a pharmaceutically acceptable salt thereof, wherein R is selected from:

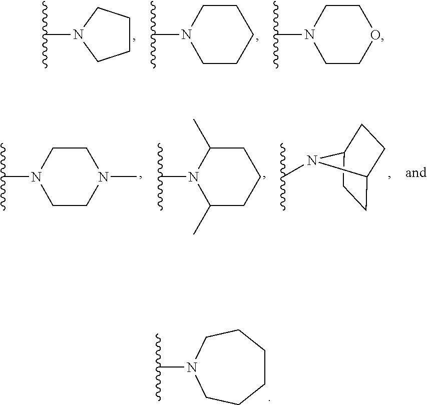

In some embodiments, the apelin receptor agonist is a compound of formula:

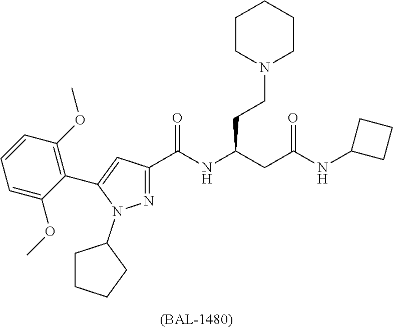 [SMILES: COc1cccc(OC)c1-c1cc(C(=O)N[C@@H](CCN2CCCCC2)CC(=O)NC2CCC2)nn1C1CCCC1]

or a pharmaceutically acceptable salt thereof. In some embodiments, the apelin receptor agonist is (S)—N-(1-(cyclobutylamino)-1-oxo-5-(piperidin-1-yl)pentan-3-yl)-5-(2,6-dimethoxyphenyl)-1-cyclopentyl-1H-pyrazole-3-carboxamide, or a pharmaceutically acceptable salt thereof, such as a hydrochloride salt of the compound.

### 5.4. Glucagon-Like Peptide-1 Receptor Agonists

Glucagon-like peptide 1 receptor (GLP-1R) belongs to Family B1 of the seven-transmembrane G protein-coupled receptors, and its natural agonist ligand is the peptide hormone glucagon-like peptide-1 (GLP-1). GLP-1 is a peptide hormone arising by its alternative enzymatic cleavage from proglucagon, the prohormone precursor for GLP-1, which is highly expressed in enteroendocrine cells of the intestine, the alpha cells of the endocrine pancreas (islets of Langerhans), and the colon. GLP-1 acts through a G protein-coupled cell surface receptor (GLP-1R) and enhances nutrient-induced insulin synthesis and release. GLP-1 stimulates insulin secretion (insulinotropic action) and cAMP formation. GLP-1(7-36) amide stimulates insulin release, lowers glucagon secretion, and inhibits gastric secretion and emptying. These gastrointestinal effects of GLP-1 are not found in vagotomized subjects, pointing to a centrally-mediated effect. GLP-1 binds with high affinity to isolated rat adipocytes, activating cAMP production and stimulating lipogenesis or lipolysis. GLP-1 stimulates glycogen synthesis, glucose oxidation, and lactate formation in rat skeletal muscle.

On activation, GLP-1 receptors couple to the α-subunit of G protein, with subsequent activation of adenylate cyclase and increase of cAMP levels, thereby potentiating glucose-stimulated insulin secretion. Therefore, GLP-1 is also an attractive therapeutic target to lower blood glucose and preserve the β-cells of the pancreas of diabetic patients or patients diagnosed with obesity. The capability of activating the human GLP-1 receptor may be determined in a medium containing membrane expressing the GLP-1 receptor, and/or in an assay with whole cells expressing the GLP-1 receptor. Alternatively, the response of the human GLP-1 receptor may be measured in a reporter gene assay.

A “GLP-1 receptor agonist” (GLP-1RA) is a compound which is capable of binding to the GLP-1 receptor (GLP-1R) and capable of activating it. In one embodiment the GLP-1 receptor is the human GLP-1 receptor. In some embodiments, the GLP-1RA is also capable of agonizing one or more additional receptors or functions. In some embodiments, the GLP-1RA is also an agonist of GIP receptor. In some embodiments, the GLP-1RA is also an agonist of glucagon receptor. In some embodiments, the GLP-1RA is also an agonist of GIP receptor and glucagon receptor.

Aspects of the present disclosure include a method of treating a condition or disease associated with weight gain by co-administering to a subject an effective dose of an apelin receptor agonist and a GLP-1 receptor agonist. In some embodiments, the GLP-1RA is administered orally. In some embodiments, the GLP-1RA is administered via injection, e.g., subcutaneously.

A variety of compounds which are agonists of GLP-1R can be used in the methods of this disclosure. In some embodiments, the GLP-1 receptor agonist (GLP-1RA) is a polypeptide or polypeptide analog. A GLP-1RA can be an incretin mimetic, or GLP-1 analog. In some embodiments, the GLP-1RA is a fusion protein, or fusion of a protein and peptide. In some embodiments, the GLP-1RA is a recombinant polypeptide. In some embodiments, the GLP-1RA is a synthetic polypeptide.

In some embodiments, the GLP-1RA is a fusion protein to agonize GLP1R for Type 2 diabetes.

In some embodiments, the GLP-1RA has additional agonist activity at one or more receptors or relevant biological targets. In some embodiments, the GLP-1RA is a dual agonist (also referred to as a twincretin). In some embodiments, the dual agonist is an agonist of GLP-1R and glucose-dependent insulinotropic peptide (GIP) receptor. Tirzepatide is an exemplary dual agonist.

In some embodiments, the GLP-1RA is an agonist of GLP-1R, and GIP receptor and/or glucagon receptor. In some embodiments, the GLP-1RA is an agonist of GLP-1R and glucagon receptor (GL R or GCGR). In some embodiments, the GLP-1RA is an agonist of GLP-1R and GIP receptor.

In some embodiments, the GLP-1RA is a peptide drug for diabetes and/or obesity that agonizes GLP-1 and GCGR.

In some embodiments, the GLP-TRA is a triple agonist (also referred to as a triple G agonist), e.g., an agonist of GLP-1R, GIP receptor and glucagon receptor. Retatrutide (LY3437943) is an exemplary triple G agonist. Other triple G agonists of interest include those described by Knerr et al. (Next generation GLP-1/GIP/glucagon triple agonists normalize body weight in obese mice, Mol. Metab. 2022 September; 63: 101533).

In some embodiments, the GLP-TRA is selected from: dulaglutide, exenatide, semaglutide, liraglutide, insulin degludec+liraglutide, insulin glargine+lixisenatide, tirzepatide, cagrilintide [INN]+semaglutide, albenatide [INN], cotadutide, CT-868, PF 06882961, efocipegtrutide, LY-3502970, NLY-001, pegapamodutide, pemvidutide, PF-07081532, retatrutide, RGT-075, TTP-273, vurolenatide, GZR-18, mazdutide, PB-119, AMG-133, dapiglutide, DD-01, DR-10627, ECC-5004, exenatide biobetter, GL-0034, GMA-105, HEC-88473, LY-3493269, NN-6177, NN-9847, NNC0519-0130, PB-1023, Peptides to Agonize GLP-1 and GCGR for Diabetes and Obesity, Peptides to Agonize GLP-1 and GCGR for Diabetes and Obesity, SCO—094, semaglutide, VK-2735, YH-25724, YN-012, and YN-015.

In some embodiments, the GLP-1RA is dulaglutide. Dulaglutide reduces fasting glucose concentrations and reduces postprandial glucose (PPG) concentrations in patients with type 2 diabetes mellitus through the agonism of the GLP-1 receptor. This drug primarily acts as an incretin mimetic hormone or analog of human glucagon-like peptide-1, which normally acts on the GLP-1 receptor. Dulaglutide activates the GLP-1 receptor found in pancreatic beta cells, increasing intracellular cyclic AMP (cAMP) in beta cells, leading to insulin release and subsequent reduction of blood glucose concentrations. Additionally, dulaglutide decreases glucagon secretion and slows gastric emptying.

In some embodiments, the GLP-1RA is exenatide. In some embodiments, the GLP-1RA is Byetta. Exenatide binds to the intact human Glucagon-like peptide-1 receptor (GLP-1R) in a similar way to the human peptide glucagon-like peptide-1 (GLP-1).

In some embodiments, the GLP-1RA is semaglutide. Semaglutide is a recombinant DNA produced polypeptide analogue of human glucagon-like peptide-1 (GLP-1) which is typically used in combination with diet and exercise in the therapy of type 2 diabetes, either alone or in combination with other antidiabetic agents. It is an agonist of glucagon-like peptide-1 receptors (GLP-1 AR) and used for the treatment of type 2 diabetes. semaglutide is a polypeptide that contains a linear sequence of 31 amino acids joined together by peptide linkages. It has a role as a hypoglycemic agent, a glucagon-like peptide-1 receptor agonist, an anti-obesity agent, a neuroprotective agent and an appetite depressant. It is a polypeptide and a lipopeptide.

In some embodiments, the GLP-1RA is liraglutide. Liraglutide is a lipopeptide that is an analogue of human GLP-1 in which the lysine residue at position 27 is replaced by arginine and a hexadecanoyl group attached to the remaining lysine via a glutamic acid spacer. Liraglutide is typically used as an adjunct to diet and exercise to improve glycemic control in adults with type 2 diabetes mellitus. It has a role as a glucagon-like peptide-1 receptor agonist and a neuroprotective agent. It is a lipopeptide and a polypeptide.

In some embodiments, the GLP-1RA is liraglutide. In certain embodiments, the method further comprises administering an additional therapeutic agent. In certain embodiments, the additional therapeutic agent is insulin degludec. Insulin degludec is typically used with a proper diet and exercise program to control high blood sugar in people with diabetes. The combination therapy of insulin degludec and liraglutide gives a robust glycemic control with a low risk for hypoglycemia and less weight gain or even weight loss.

In some embodiments, the GLP-1RA is lixisenatide. In some embodiments, the method further comprises administering GLP-1RA in combination with insulin glargine. In some embodiments, the insulin glargine in combination with lixisenatide is Soliqua 100/33. Insulin glargine and lixisenatide is a combination medicine that is typically used together with diet and exercise to improve blood sugar control in adults with type 2 diabetes. Insulin glargine is a long-acting insulin that starts to work several hours after injection and keeps working evenly for 24 hours. Lixisenatide is a drug that helps the pancreas produce insulin more efficiently.

In some embodiments, the GLP-1RA is tirzepatide. tirzepatide is a dual glucose-dependent insulinotropic polypeptide (GIP) and glucagon-like peptide-1 (GLP-1) receptor agonist (RA). Tirzepatide works by activating both the GLP-1 and GIP receptors in the body. This triggers the release of insulin from the pancreas that blocks glucagon, a hormone that increases blood sugar levels.

In some embodiments, the GLP-1RA is semaglutide.

In some embodiments, the GLP-1RA is albenatide.

In some embodiments, the GLP-1RA is albiglutide.

In some embodiments, the GLP-1RA is cotadutide. Cotadutide (MEDI0382), a dual GLP-1 and glucagon receptor agonist, is currently under development for type 2 diabetes and NASH.

In some embodiments, the GLP-1RA is CT-868. CT-868 is a dual GLP-1 and GIP receptor modulator that is optimized for improved tolerability at the GLP-1 receptor. The combined action of GLP-1 and GIP result in greater body weight loss and glucose control.

In some embodiments, the GLP-1RA is efocipegtrutide. Efocipegtrutide is a glucagon, gastric inhibitory polypeptide (GIP) and glucagon-like peptide 1 (GLP-1) receptors agonist. Efocipegtrutide shares sequence homology with glucagon, glucagon-like peptide 1 (GLP1) and gastric inhibitory polypeptide (GIP, glucose-dependent insulinotropic polypeptide, incretin hormone), where the gastric inhibitory peptide (GIP) and glucagon-like peptide-1 (GLP-1) triple full agonist is chemically conjugated with constant region of human immunoglobulin via non-peptidyl flexible linker.

In some embodiments, the GLP-1RA is NLY-001. NLY-001 is a microglia-targeted GLP-1RA. NLY-001 is a pegylated exendin-4 analogue of Glucagon Like Peptide-1 Receptor (GLP-1R) agonist.

In some embodiments, the GLP-1RA is pegapamodutide.

In some embodiments, the GLP-1RA is pemvidutide. Pemvidutide is a peptide-based GLP-1/glucagon dual receptor agonist developed for the treatment of obesity and non-alcoholic steatohepatitis (NASH). Pemvidutide has been shown to substantially decrease the amount of fat within the liver which could have beneficial effects on insulin resistance and cardiorenal risk, common problems in people with obesity. In clinical trials, pemvidutide demonstrated striking reductions in body weight, liver fat, serum lipids and markers of liver inflammation.

In some embodiments, the GLP-1RA is retatrutide. Retatrutide stimulates GIPR, GLP-1, and GLP-1 receptors

In some embodiments, the GLP-1RA is TTP-273.

In some embodiments, the GLP-1RA is vurolenatide. Vurolenatide is a GLP-1 receptor agonist that is administered via injection.

In some embodiments, the GLP-1RA is GZR-18. GZR-18 is an analog of glucagon-like peptide-1 (GLP-1). In vitro pharmacology and activity of GZR¹⁸ were previously characterized by a binding assay of GZR¹⁸ using human serum albumin (HSA), an activation assay in human GLP-1 receptor-expressing cell lines, and its effect on glucose-stimulated insulin secretion (GSIS) in primary mice islets.

In some embodiments, the GLP-1RA is mazdutide. Mazdutide (1B1362) is a glucagon-like peptide-1 (GLP-1) and glucagon receptor dual agonist. Mazdutide is a long-acting synthetic peptide related to mammalian oxyntomodulin (OXM), which uses a fatty acid side chain to prolong the duration of action and allow once-weekly administration. Mazdutide is thought to exert its biological effects by activating GLP-1 receptor and glucagon receptor in human beings, which is estimated to improve glucose tolerance and induce weight loss, mimicking the effects of endogenous oxyntomodulin.

In some embodiments, the GLP-1RA is PB-119. PB-119 is a pegylated human glucagon-like peptide-1 (GLP-1) receptor agonist.

In some embodiments, the GLP-1RA is AMG-133. AMG 133 is a bispecific glucose-dependent insulinotropic polypeptide receptor (GIPR) antagonist and glucagon-like peptide-1 (GLP-1) receptor agonist molecule. AMG 133 mimics the agonist effects of GLP-1 and antagonizes the effects of glucose-dependent insulinotropic polypeptide (GIP).

In some embodiments, the GLP-1RA is dapiglutide. Dapiglutide promotes significant intestinal growth, as indicated by significantly increased villus height as well as intestinal length. Dapiglutide reduces stool water losses, resulting in reduced plasma aldosterone. It has been shown that dapiglutide possesses specific and potent GLP-1R and GLP-2R agonist effects in rodents.

In some embodiments, the GLP-1RA is DD-01. DD-01 is a pegylated, long-acting, peptide based dual agonist of glucagon-like peptide 1 (GLP-1) receptor and glucagon receptor (GCGR).

In some embodiments, the GLP-1RA is DR-10627.

In some embodiments, the GLP-1RA is ECC-5004. ECC-5004 is an orally administered small-molecule GLP-1 RA.

In some embodiments, the GLP-1RA is exenatide biobetter.

In some embodiments, the GLP-1RA is GL-0034. GL0034 is a glucagon-like peptide-1 receptor (GLP-1R) agonist that has been shown to have glucose-lowering effects with increased insulin and C-peptide levels, reduced plasma glucagon levels, long-term reduction in HbA1C, and reduced body weight when tested in type 2 diabetic mice.

In some embodiments, the GLP-1RA is GMA-105. GMA-105 is a humanized anti-GLP-1R monoclonal antibody carrying a GLP-1 fragment.

In some embodiments, the GLP-1RA is HEC-88473. HEC88473 is a GLP-1/FGF21 dual agonist.

In some embodiments, the GLP-1RA is LY-3493269. LY-3493269 is a GIP/GLP coagonist peptide.

In some embodiments, the GLP-1RA is NN-6177. NN-6177 acts by targeting glucagon receptor (GCGR) and glucagon like peptide 1 receptor (GLP1R).

In some embodiments, the GLP-1RA is NN-9847.

In some embodiments, the GLP-1RA is NNC0519-0130.

In some embodiments, the GLP-1RA is PB-1023. PB-1023 is a recombinant GLP-1 analogue used to treat sarcopenia-related diseases.

In some embodiments, the GLP-1RA is SCO—094. SCO—094 is a dual agonist for GLP-1R and GIPR. Preclinical studies have shown that SCO—094 is more effective in improving diabetes and obesity than the GLP-1R mono-agonist.

In some embodiments, the GLP-1RA is semaglutide. semaglutide is a GLP-1 agonist and works by increasing insulin release, lowering the amount of glucagon released, delaying gastric emptying and reducing appetite.

In some embodiments, the GLP-1RA is VK-2735. VK-2735 is a dual agonist of the glucagon-like peptide 1 (GLP-1) and glucose-dependent insulinotropic polypeptide (GIP) receptors for the potential treatment of various metabolic disorders such as diabetes, obesity and NASH.

In some embodiments, the GLP-1RA is YH-25724. YH-25724 is a long-acting GLP-1/FGF21 dual agonist that lowers both non-alcoholic fatty liver disease activity score and fibrosis stage in a diet-induced obese mouse model of biopsy-confirmed non-alcoholic steatohepatitis.

In some embodiments, the GLP-1RA is YN-012.

In some embodiments, the GLP-1RA is and YN-015.

In some embodiments, the GLP-1 receptor agonist (GLP-1RA) is a small molecule agonist of the GLP-1 receptor. In some embodiments, the GLP-1RA is PF-07081532. PF-07081532 is an oral small molecule GLP-1 receptor agonist that is being developed for the treatment of Type 2 diabetes and obesity.

In some embodiments, the GLP-1RA is GSBR-1290, and orally delivered small molecule.

In some embodiments, the GLP-1RA is RGT-075. RGT-075 is an orally bioavailable, small-molecule GLP-1 RA.

In some embodiments, the GLP-1RA is orforglipron (LY-3502970). LY-3502970 is an orally active non-peptide agonist of glucagon-like peptide-1 (GLP-1) receptor. See Kawai et al., PNAS Nov. 11, 2020, 117 (47) 29959-29967.

In some embodiments, the GLP-1RA is danuglipron (PF 06882961). Danuglipron activates the canonical G protein signaling activity only in the Glucagon-like peptide-1 (GLP-1) receptor with Trp33^{ECD}. Danuglipron has been shown to potentiate glucose-stimulated insulin release and reduces food intake in monkeys.

GLP-1RA agents of interest which can be utilized in the methods of this disclosure include, but are not limited to, dapagliflozin+semaglutide, 4P-004, AP-026, BGM-0504, CT-996, DD-01, DR-10624, DR-10627, dulaglutide, ECC-5004, exenatide, exenatide biobetter, GL-0034, GLP-06, GMA-106, HB-1085, HDM-1002, HL-08, HZ-010, KN-056, liraglutide, MWN-101, NN-6177, NN-9847, NN-9904, PF-06954522, SAL-0112, SCO—094, TERN-601, XW-004, XW-014, YH-25724, YN-012, YN-015, and ZT-002.

Other GLP-1RA agents of interest, e.g., in clinical trials, which can be utilized in the methods of this disclosure include, but are not limited to, (semaglutide+GIP analogue), AZD-9550, CT-388, CT-868, danuglipron tromethamine, dapiglutide, E-2HSA, efinopegdutide, efocipegtrutide, exenatide SR, froniglutide, GMA-105, GSBR-1290, GXG-6, GZR-18, HEC-88473, HR-17031, HRS-7535, HRS-9531, HS-20004, HS-20094, JY-09, liraglutide biobetter, maridebart cafraglutide, MBX-1416, MDR-001, NLY-001, NN-9490, NNC0519-0130, PB-718, pegapamodutide, pemvidutide, semaglutide injection, TTP-273, and VK-2735.

Additional GLP-1RA agents of interest in clinical trials which can be utilized in the methods of this disclosure include, but are not limited to, (cagrilintide+semaglutide), retatrutide, (LAI-287+semaglutide), albenatide, avexitide acetate, Diabegone, ecnoglutide, efpeglenatide LA, GMA-102, liraglutide, mazdutide, NN-6535 (semaglutide), NN-9932 (semaglutide), orforglipron calcium, PB-119, SAL-015, survodutide, Uni-E4, and vurolenatide.

Further GLP-1RA agents of interest which can be utilized in the methods of this disclosure include, but are not limited to, (dorzagliatin+GLP-1), (exenatide+insulin aspart), ACT-1003, Adogel Sema, AER-601, AGM-212, BEBT-808, BZ-043B, C-2816, DAJC-1, DD-02, DR-10625, DR-10628, DS-004, DS-005, DS-006, DS-012, E-6, efpeglenatide+HM-12470, exenatide 2, exenatide LA, exenatide SR, Extendin-Fc, G-49, GB-7001, Gene Encoding GLP-1, GLP-1 Incretin Triagonist, GLP-1 Oral Preparation, GLP-1R Antagonist for Hypoglycemia, glucagon, Glucagon-Like Peptide-1+insulin human, GPCR-targeted Project 012, GPCR-targeted Project 013, GT-01123, HM-15275, HPG-5119, HSP-001, HSP-004, HSP-005, HSP012-C, Hydrogel Exenatide, I20-105S, I20-110, KP-405, LA-EX, liraglutide biobetter, liraglutide LA, MK-1462, MLX-7000, MWN-105, MWN-109, NLY-12, NPM-115, OGB-21502, OXM, P-11, PB-2301, PB-2309, RGT-028, RGT-274, RPC-8844, RT-104, SHX-022, SL-209, synthetic peptides to agonize GLP-1R and CCKBR for diabetes, TB-013, TB-222023, TB-592, TE-8105, THDBH-111, UDS-003, VTCG-15, XL-110, XL-310, XW-003+XW-015, XW-003+XW-017, Y-002, YGX-1, ZT-003, ZT-006, ZT-007, DA-1726, HDM-1005, (insulin degludec+liraglutide), DB-081, GW-002, HZCX-012, ID-110521156, THDB-0211, THDBH-110, THDBH-120, THDBH-121, UBT-251, ATBB-22, BEM-012, ClN-209, CIN-210, DD-03, exenatide+ND-017, exenatide+Synthetic Peptide 2, glucagon, Insulin-GLP1, MD-02, OGB-21501, P-01, PAT-201, PF-1807, and PT-3.

### 5.5. Additional Therapeutic Agents

The methods of the present disclosure comprise co-administering an effective amount of an apelin receptor agonist and an effective amount of a GLP-1 receptor agonist to the subject. In some embodiments, the methods of the present disclosure further comprise co-administering an effective amount of one or more additional therapeutic agents, i.e., pharmacologically active substances.

In some embodiments, the methods of the present disclosure include administration of an additional therapeutic agent. In certain embodiments, the additional therapeutic agent is an incretin receptor agonist. In certain embodiments, the additional therapeutic agent is amylin. In some embodiments, the additional therapeutic agent is cagrilintide. In some embodiments, the additional therapeutic agent is insulin degludec. In some embodiments, the additional therapeutic agent is insulin glargine.

In some embodiments, the additional therapeutic agent is a drug that reduces caloric intake that is not a GLP-1 receptor agonist. In some embodiments, the additional therapeutic agent is a compound that regulates appetite, e.g., an appetite suppressant.

In some embodiments, the additional therapeutic agent is a drug that reduces caloric intake selected from alpha amylase 2B (1,4-Alpha D-Glucan Glucanohydrolase 2B or Carcinoid Alpha Amylase or AMY2B or EC 3.2.1.1) inhibitor; gastric triacylglycerol lipase (Gastric Lipase or LIPF or EC 3.1.1.3) inhibitor; maltase glucoamylase (Alpha-1 4-Glucosidase or MGAM or EC 3.2.1.20) inhibitor; pancreatic alpha amylase (1,4 Alpha D Glucan Glucanohydrolase or AMY2A or EC 3.2.1.1) inhibitor; pancreatic triacylglycerol lipase (Pancreatic Lipase or Triacylglycerol Acylhydrolase or PNLIP or EC 3.1.1.3) inhibitor; and sucrase isomaltase intestinal (SI or EC 3.2.1.48 or EC 3.2.1.10) inhibitor. In some embodiments, the additional therapeutic agent is a drug that reduces caloric intake selected from TAS2R receptor agonist; bitter taste receptor agonist; Nutrient receptor agonist; Cannabinoid Receptor 1 (CB1 or CANN6 or CNR1) Antagonist; Alpha 1,6 Mannosyl Glycoprotein 2 Beta N Acetylglucosaminyltransferase (Beta 1,2 N Acetylglucosaminyltransferase II or Mannoside Acetylglucosaminyltransferase 2 or N Glycosyl Oligosaccharide Glycoprotein N Acetylglucosaminyltransferase II or GlcNAc-T II or MGAT2 or EC 2.4.1.143) Inhibitor; Glabridin analog, Distal jejunal-release dextrose; and Mucin-complexing polymer. Drugs that reduce caloric intake which can be utilized in the methods of this disclosure include, but are not limited to, EMP-16, APH-012, ARD-101, GLY-200, K-757+K-833, INV-202, S-309309, vutiglabridin, AMG-786, Amylin Agonist Long Acting, AZD-6234, CK-0045, ENT-03, ERX-1000, NO1820237, GUB-014295, CIN-109, Dacra QW II, nimacimab, RAY-1225, XEN-101, ZP-8396, and LY-3971297. In some embodiments, the additional therapeutic agent is a cannabinoid receptor 1 (CB1r or CANN6 or CNR1) antagonist, such as INV-202, or INV-300.

Obesity related agents that reduce energy or caloric intake which can be utilized in the methods of this disclosure include, but are not limited to, CRB-913, DBPR-211, PB-722, (efpeglenatide+HM-15136), ACE-167, AD-9308, AGEX-BAT1, AvR-2V10, AZ-12861903, AZ-13483342, AZD-3857, BEBT-809, BF-114, Cannabinoids, CKR-334, CLS-1, CNIO-PI3Ki, CV-08, CYTX-100, Era-107, ETBD-03, FM-801, Fusion Proteins to Activate GDF15 for Obesity, FZ-010, GCG-06, GMA-107, HM-15275, HTD-1804, HUM-234, 120-107, I2O-120, INHBE (Metabolic Disorders), ClN-110, KSB-10201, KY-19334, LR-19020, LR-19156, LY-3971297, M-43, MLX-0800, MLX-5000, MLX-7000, MNO—863, Monoclonal Antibody to Antagonize FSH Receptor for Obesity and Osteoporosis, MT-106, Myostatin antagonist, NM-136, NN-9056, NOVS-100, NPO—2237, OBE-2001, OLX-75016, orlistat, Peptide (PYY), Peptide to Antagonize MC3R for Obesity, Peptides for Non-Alcoholic Steatohepatitis and Obesity, Peptides to Agonize Oxytocin Receptor for Obesity, Peripheral CBT Blockers, PF-06645849, PL-8905, PL-9610, psilocybin, PSYLO-3002, PYY-1119, RB-014, Recombinant Protein to Agonize Leptin Receptor for Obesity, Rejuva, REMD-524, REP-003, RES-010, RES-020, RMD-1202, RP-1208, RSVI-301, RSVI-303, RT-210, SAL-0125, Gastrointestinal Hormones, SJT-4a, SJT-7a, sobetirome, SPN-007, SRK-439, TB-592, Tespria, TF-0062, TF-0103, ThermoStem, TLC-1235, VK-1430, XL-100, YH-34160, YN-103, YN-106, ZP-6590, ZYL-001, ADY-790011, ATC-601, AX-0601, BEBT-509, CBF-520, CYTA-002, DILOC-2, EB-012, ECN-0424, EMB-2, GM-60186, GPR75, GT-002, GUI-37, HLB-1007, HLB-1015, HMC-2073, and ICB-513.

Other drugs that reduce caloric intake which can be utilized in the methods of this disclosure include, but are not limited to, naltrexone-bupropion, phentermine-topiramate, benzphetamine, diethylpropion, phendimetrazine, phentermine, orlistat, and setmelanotide.

It is understood that the following description, e.g., of isomers, salts, and other forms, etc., can apply to any classes of compounds and drugs within the scope of the specification.

If any variable occurs more than one time in a chemical formula, its definition on each occurrence is independent of its definition at every other occurrence. If the chemical structure and chemical name conflict, the chemical structure is determinative of the identity of the compound. The compounds of this disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Certain compounds of this disclosure may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R⁴ is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

Unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed.

Various compounds of this disclosure contain one or more chiral centers, and can exist as racemic mixtures of enantiomers, mixtures of diastereomers or enantiomerically or optically pure compounds. This invention encompasses the use of stereoisomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents.

Compounds of the present disclosure include, but are not limited to, apelin receptor agonist compounds, GLP-1 receptor agonist compounds, and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. The term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

The compounds of this disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

The term “pharmaceutically acceptable salt” refers to a salt that is acceptable for administration to a subject. Examples of pharmaceutically acceptable salts include, but are not limited to: mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glycine salt, lysine salt, arginine salt, omithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt. The term “salt(s)” as used herein encompass hydrate salt(s).

Other examples of pharmaceutically salts include anions of the compounds of the present disclosure compounded with a suitable cation. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Furthermore, if the compounds of the present invention or salts thereof form hydrates or solvates, these are also included in the scope of the compounds of the present invention or salts thereof.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

### 5.6. Pharmaceutical Compositions

The pharmaceutical compositions can include the compound(s) or the pharmaceutically acceptable salt thereof, and at least one pharmaceutically acceptable excipient, carrier or diluent. In some such embodiments, the compound or the pharmaceutically acceptable salt thereof, according to any one of the embodiments is present in an amount effective for the treatment of a condition or disease (e.g., as described herein).

The apelin receptor agonist compounds and/or GLP-1RA compounds used in the methods described herein can be formulated in any appropriate pharmaceutical composition for administration by any suitable route of administration.

The GLP-1 receptor agonists or GLP-1 analogs used in the methods described herein can be formulated in any appropriate pharmaceutical composition for administration by any suitable route of administration. The pharmaceutical compositions can include the GLP-1 receptor agonists or analogues thereof or the pharmaceutically acceptable salt thereof, the tautomer thereof, the pharmaceutically acceptable salt of the tautomer, the stereoisomer of any of the foregoing, or the mixture thereof according to any one of the embodiments described herein and at least one pharmaceutically acceptable excipient, carrier or diluent. In some such embodiments, the GLP-1 receptor agonists or analogues thereof or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of a condition or disease (e.g., as described herein), for activating the GLP-1 receptor.

Suitable routes of administration for each of the apelin receptor agonists or GLP-1 receptor agonists include, but are not limited to, oral, topical, subcutaneous injection, and intravenous routes of administration. Suitable routes also include pulmonary administration, including by oral inhalation. In some embodiments, the route of administration is subcutaneous injection. The most suitable route may depend upon the condition and disorder of the recipient. The formulations may conveniently be presented in unit dosage form and may be prepared by any of the methods known in the art of pharmacy.

In some embodiments, the pharmaceutical composition is formulated for oral delivery whereas in other embodiments, the pharmaceutical composition is formulated for subcutaneous or intravenous delivery. In some embodiments, the pharmaceutical composition is formulated for oral administration once a day or QD, and in some such formulations is a tablet where the effective amount of the active ingredient ranges from 5 mg to 60 mg, from 6 mg to 58 mg, from 10 mg to 40 mg, from 15 mg to 30 mg, from 16 mg to 25 mg, or from 17 mg to 20 mg. In some such compositions, the amount of active ingredient is 17 mg.

All methods include the step of bringing into association an apelin agonist, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

All methods include the step of bringing into association an GLP-TRA, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein can be different for the apelin receptor agonist and the GLP-1 receptor agonist or the route of administration for the apelin receptor agonist and the GLP-1 receptor agonist is the same.

In some embodiments, the route of administration for the apelin receptor agonist is parenteral administration. In some embodiments, the route of administration for the apelin receptor agonist is intravenous administration (e.g., intravenous infusion). In some embodiments, the route of administration for the apelin receptor agonist is oral administration. In some embodiments, the route of administration for the apelin receptor agonist is constant intravenous infusion. In some embodiments, the route of administration for the apelin receptor agonist is subcutaneous injection.

In some embodiments, the route of administration for the GLP-1 receptor agonist is parenteral administration. In some embodiments, the route of administration for the GLP-1 receptor agonist is intravenous administration (e.g., intravenous infusion). In some embodiments, the route of administration for the GLP-1 receptor agonist is oral administration. In some embodiments, the route of administration for the GLP-1 receptor agonist is constant intravenous infusion. In some embodiments, the route of administration for the GLP-1 receptor agonist is subcutaneous injection.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain antioxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. The term “excipient” broadly refers to any component other than the active therapeutic ingredient(s). The excipient may be an inert substance, an inactive substance, and/or a not medicinally active substance.

Any suitable pharmaceutical excipient may be used, and one of ordinary skills in the art is capable of selecting suitable pharmaceutical excipients. The excipient may serve various purposes, e.g. as a carrier, vehicle, diluent, tablet aid, and/or to improve administration, and/or absorption of the active substance. Non-limiting examples of excipients are: Solvents, diluents, buffers, preservatives, tonicity regulating agents, chelating agents, and stabilizers.

Examples of formulations include liquid formulations, i.e. aqueous formulations comprising water. A liquid formulation may be a solution, or a suspension. An aqueous formulation typically comprises at least 50% w/w water, or at least 60%, 70%, 80%, or even at least 90% w/w of water.

Alternatively, a pharmaceutical composition may be a solid formulation, e.g. a freeze-dried or spray-dried composition, which may be used as is, or whereto the physician or the patient adds solvents, and/or diluents prior to use.

A pharmaceutical composition may comprise a buffer. A pharmaceutical composition may comprise a preservative. A pharmaceutical composition may comprise a chelating agent. A pharmaceutical composition may comprise a stabilizer. A pharmaceutical composition may comprise one or more surfactants. A pharmaceutical composition may comprise one or more protease inhibitors, e.g., when the active compound is a polypeptide.

A composition may be administered in several dosage forms, for example as a solution; a suspension; an emulsion; a microemulsion; multiple emulsions; an injection solution; an infusion solution.

Systemic or parenteral administration may be performed by subcutaneous, intramuscular, intraperitoneal, or intravenous injection by means of a syringe, optionally a pen-like syringe, or by means of an infusion pump.

### 5.7. Combination Pharmaceutical Products

Aspects of this disclosure include a pharmaceutical composition including a combination of an apelin receptor agonist and a GLP-1 receptor agonist in a single dosage form. In some embodiments, the pharmaceutical composition is formulated for oral administration, where the apelin receptor agonist and the GLP-1 receptor agonist are both suitable for oral administration. In some embodiments, the apelin receptor agonist and the GLP-1 receptor agonist are both small molecule drugs (e.g., as described herein).

In some embodiments, the pharmaceutical composition is formulated for subcutaneous administration.

Aspects of this disclosure include kits that include an apelin receptor agonist and a GLP-1 receptor agonist, e.g., each present in a unit dosage form.

### 5.8. Dosage Form

In some embodiments, an apelin receptor agonist or salt thereof is administered in a suspension. In other embodiments, an apelin receptor agonist or salt thereof is administered in a solution. In some embodiments, an apelin receptor agonist or salt thereof is administered in a solid dosage form. In some embodiments, the solid dosage form is a capsule. In some embodiments, the solid dosage form is a tablet. In specific embodiments, an apelin receptor agonist is in a crystalline or amorphous form. In some embodiments, an apelin receptor agonist is in amorphous form. In some embodiments, the apelin receptor agonist is an apelin receptor agonist.

In one aspect of the methods, the apelin receptor agonist, or the pharmaceutical composition including same, is administered intravenously, topically, orally, by inhalation, by infusion, by injection, intraperitoneally, intramuscularly, subcutaneously, intra-aurally, by intra-articular administration, by intra-mammary administration, by topical administration or by absorption through epithelial or mucocutaneous linings. In certain embodiments, the apelin receptor agonist, or the pharmaceutical composition including same, is administered via intravenous infusion.

In some embodiments, an GLP-1 receptor agonist or salt thereof is administered in a suspension. In other embodiments, an GLP-1 receptor agonist or salt thereof is administered in a solution. In some embodiments, an GLP-1 receptor agonist or salt thereof is administered in a solid dosage form. In particular embodiments, the solid dosage form is a capsule. In particular embodiments, the solid dosage form is a tablet. In specific embodiments, a GLP-1 receptor agonist is in a crystalline or amorphous form. In particular embodiments, a GLP-1 receptor agonist is in amorphous form.

In one aspect of the methods, the GLP-1 receptor agonist, or the pharmaceutical composition including same, is administered intravenously, topically, orally, by inhalation, by infusion, by injection, intraperitoneally, intramuscularly, subcutaneously, intra-aurally, by intra-articular administration, by intra-mammary administration, by topical administration or by absorption through epithelial or mucocutaneous linings. In certain embodiments, the GLP-1 receptor agonist, or the pharmaceutical composition including same, is administered via intravenous infusion. In certain embodiments, the GLP-1 receptor agonist, or the pharmaceutical composition including same, is administered via subcutaneous injection.

### 5.9. Apelin Receptor Agonist Dosing

In various embodiments, the dose of the apelin receptor agonist is at least 0.01 mg/kg, such as at least 0.5 mg/kg, or at least 1 mg/kg. In certain embodiments, the dose is 25 mg/kg to 1,000 mg/kg per day.

In some embodiments, the apelin receptor agonist is administered in a dose that is independent of patient weight or surface area (flat dose).

In various embodiments, the dose is 1-5000 mg. In various embodiments, the dose is 25-2000 mg. In some embodiments, the dose is at least 60 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 420 mg, at least 440 mg, at least 460 mg, at least 480 mg, at least 500 mg, at least 520 mg, at least 550 mg, at least 580 mg, at least 600 mg, at least 650 mg, at least 700 mg, at least 750 mg, at least 800 mg, at least 850 mg, at least 900 mg, at least 950 mg, at least 1000 mg, at least 1100 mg, at least 1200 mg, at least 1300 mg, at least 1400 mg, at least 1450 mg, or at least 1500 mg. In various embodiments, the dose is 25-2000 mg. In some embodiments, the dose is at least 200 mg. In various embodiments, the dose is 25-2000 mg. In some embodiments, the dose is at least 240 mg.

The apelin receptor agonist can be administered in a single dose or in multiple doses.

In some embodiments, the dose is administered daily.

In some embodiments, the dose is administered as a plurality of equally or unequally divided sub-doses.

In certain embodiments, the dose is administered continuously (e.g., IV infusion) for a period of time. In certain embodiments, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours). In certain embodiments, following the dose, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a dose and a 24 hour or 48-hour washout period, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a first dose and a 24 hour or 48-hour washout period, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours), followed by a second dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours).

In some embodiments, the apelin receptor agonist is administered orally, intravenously, intranasally, or intramuscularly. In some embodiments, the apelin receptor agonist is administered orally.

In some embodiments, the apelin receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the apelin receptor agonist is administered continuously for at least 10 minutes, at least 20 minutes, at least 30 minutes, at least 40 minutes, at least 50 minutes, at least 1 hour, at least 2 hours, at least 3 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 48 hours, at least 72 hours, at least 100 hours, at least 110 hours, at least 115 hours, at least 120 hours, or at least 125 hours.

### 5.10. GLP-1 Receptor Agonist Dosing

In various embodiments, the dose of an GLP-1 receptor agonist is GLP-1 receptor agonist is adjusted according to the patient's disease condition.

In various embodiments, the dose of the GLP-1 receptor agonist is at least 0.01 mg/kg, such as at least 0.5 mg/kg, or at least 1 mg/kg. In certain embodiments, the dose is 25 mg/kg to 1,000 mg/kg per day.

In some embodiments, the GLP-1 receptor agonist is administered in a dose that is independent of patient weight or surface area (flat dose).

In various embodiments, the dose is 0.01-5000 mg. In various embodiments, the dose is 0.05-5 mg. In various embodiments, the dose is at least 0.1 mg, at least 0.2 mg, at least 0.25 mg, at least 0.3 mg, at least 0.4 mg, at least 0.5 mg, at least 0.7 mg, at least 0.6 mg, at least 0.75 mg, at least 0.8 mg, at least 0.9 mg, at least 1 mg, at least 1.2 mg, at least 1.25 mg, at least 1.3 mg, at least 1.4 mg, at least 1.5 mg, at least 1.6 mg, at least 1.75 mg, at least 1.8 mg, at least 1.9 mg, at least 2 mg, at least 2.1 mg, at least 2.2 mg, at least 2.25 mg, at least 2.3 mg, at least 2.4 mg, at least 2.5 mg, at least 2.6 mg, at least 2.75 mg, at least 2.8 mg, at least 2.9 mg, at least 3 mg, at least 3.1 mg, at least 3.2 mg, at least 3.25 mg, at least 3.3 mg, at least 3.4 mg, at least 3.5 mg, at least 3.6 mg, at least 3.75 mg, at least 3.8 mg, at least 3.9 mg, at least 4 mg, at least 4.1 mg, at least 4.2 mg, at least 4.25 mg, at least 4.3 mg, at least 4.4 mg, at least 4.5 mg, at least 4.6 mg, at least 4.75 mg, at least 4.8 mg, 4.9 mg, at least 5 mg, at least 5.25 mg, at least 5.5 mg, at least 5.75 mg, at least 6 mg, at least 6.25 mg, at least 6.5 mg, at least 6.75 mg, at least 7 mg, at least 7.25 mg, at least 7.5 mg, at least 7.75 mg, at least 8 mg, at least 8.25 mg, at least 8.5 mg, at least 8.75 mg, at least 9 mg, at least 9.25 mg, at least 9.5 mg, at least 9.75 mg, or at least 10 mg. In various doses, the dose is at least 10.5 mg, at least 11 mg, at least 11.5 mg, at least 12 mg, at least 12.5 mg, at least 13 mg, at least 13.5 mg, at least 14 mg, at least 14.5 mg, at least 15 mg, at least 15.5 mg, at least 16 mg, at least 16.5 mg, at least 17 mg, at least 17.5 mg, at least 18 mg, at least 18.5 mg, at least 19 mg, at least 19.5 mg, or at least 20 mg. In various embodiments, the dose is 25-2000 mg. In some embodiments, the dose is at least 25 mg, at least 30 mg, at least 35 mg, at least 40 mg, at least 45 mg, at least 50 mg, at least 55 mg, at least 60 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 420 mg, at least 440 mg, at least 460 mg, at least 480 mg, at least 500 mg, at least 520 mg, at least 550 mg, at least 580 mg, at least 600 mg, at least 650 mg, at least 700 mg, at least 750 mg, at least 800 mg, at least 850 mg, at least 900 mg, at least 950 mg, at least 1000 mg, at least 1100 mg, at least 1200 mg, at least 1300 mg, at least 1400 mg, at least 1450 mg, or at least 1500 mg. In various embodiments, the dose is 25-2000 mg. In some embodiments, the dose is at least 200 mg. In various embodiments, the dose is 25-2000 mg. In some embodiments, the dose is at least 240 mg.

In various embodiments, the dose is 0.01-5000 mcg. In various embodiments, the dose is 0.05-5 mcg. In various embodiments, the dose is at least 0.1 mcg, at least 0.2 mcg, at least 0.25 mcg, at least 0.3 mcg, at least 0.4 mcg, at least 0.5 mcg, at least 0.7 mcg, at least 0.6 mcg, at least 0.75 mcg, at least 0.8 mcg, at least 0.9 mcg, at least 1 mcg, at least 1.2 mcg, at least 1.25 mcg, at least 1.3 mcg, at least 1.4 mcg, at least 1.5 mcg, at least 1.6 mcg, at least 1.75 mcg, at least 1.8 mcg, at least 1.9 mcg, at least 2 mcg, at least 2.1 mcg, at least 2.2 mcg, at least 2.25 mcg, at least 2.3 mcg, at least 2.4 mcg, at least 2.5 mcg, at least 2.6 mcg, at least 2.75 mcg, at least 2.8 mcg, at least 2.9 mcg, at least 3 mcg, at least 3.1 mcg, at least 3.2 mcg, at least 3.25 mcg, at least 3.3 mcg, at least 3.4 mcg, at least 3.5 mcg, at least 3.6 mcg, at least 3.75 mcg, at least 3.8 mcg, at least 3.9 mcg, at least 4 mcg, at least 4.1 mcg, at least 4.2 mcg, at least 4.25 mcg, at least 4.3 mcg, at least 4.4 mcg, at least 4.5 mcg, at least 4.6 mcg, at least 4.75 mcg, at least 4.8 mcg, 4.9 mcg, at least 5 mcg, at least 5.25 mcg, at least 5.5 mcg, at least 5.75 mcg, at least 6 mcg, at least 6.25 mcg, at least 6.5 mcg, at least 6.75 mcg, at least 7 mcg, at least 7.25 mcg, at least 7.5 mcg, at least 7.75 mcg, at least 8 mcg, at least 8.25 mcg, at least 8.5 mcg, at least 8.75 mcg, at least 9 mcg, at least 9.25 mcg, at least 9.5 mcg, at least 9.75 mcg, or at least 10 mcg. In various doses, the dose is at least 10.5 mcg, at least 11 mcg, at least 11.5 mcg, at least 12 mcg, at least 12.5 mcg, at least 13 mcg, at least 13.5 mcg, at least 14 mcg, at least 14.5 mcg, at least 15 mcg, at least 15.5 mcg, at least 16 mcg, at least 16.5 mcg, at least 17 mcg, at least 17.5 mcg, at least 18 mcg, at least 18.5 mcg, at least 19 mcg, at least 19.5 mcg, or at least 20 mcg. In various embodiments, the dose is 25-2000 mcg. In some embodiments, the dose is at least 25 mcg, at least 30 mcg, at least 35 mcg, at least 40 mcg, at least 45 mcg, at least 50 mcg, at least 55 mcg, at least 60 mcg, or at least 100 mcg.

The GLP-1 receptor agonist can be administered in a single dose or in multiple doses.

In some embodiments, the dose is administered daily. In some embodiments, the dose is administered once daily. In some embodiments, the dose is administered twice daily. In some embodiments, the dose is administered weekly. In some embodiments, the dose is administered monthly. In some embodiments, the dose is administered every 30 days. In some embodiments, the dose is administered weekly. In some embodiments, the dose is administered bimonthly. In some embodiments, the dose is administered once daily.

In some embodiments, the dose is administered as a plurality of equally or unequally divided sub-doses.

In some embodiments, the dose is administered as a single dose in the form of a pen. In some embodiments, the dose is administered at a single dose ranging from 0.5-6 mg once weekly. In some embodiments, the dose is administered at a single dose ranging from 0.75-4.5 mg once weekly.

In certain embodiments, the dose is administered continuously (e.g., IV infusion) for a period of time. In certain embodiments, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours). In certain embodiments, following the dose, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a dose and a 24 hour or 48-hour washout period, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a first dose and a 24 hour or 48-hour washout period, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours), followed by a second dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours).

In some embodiments, the GLP-1 receptor agonist is administered orally, intravenously, intranasally, or intramuscularly. In some embodiments, the GLP-1 receptor agonist is administered orally.

In some embodiments, the GLP-1 receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the GLP-1 receptor agonist is administered continuously for at least 10 minutes, at least 20 minutes, at least 30 minutes, at least 40 minutes, at least 50 minutes, at least 1 hour, at least 2 hours, at least 3 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 48 hours, at least 72 hours, at least 100 hours, at least 110 hours, at least 115 hours, at least 120 hours, or at least 125 hours.

In some embodiments, the GLP-1 receptor agonist is administered once weekly. In some embodiments, the GLP-1 receptor agonist is administered subcutaneously, once weekly. In some embodiments, the recommended starting dosage of the GLP-1 receptor agonist is 2.5 mg injected subcutaneously once weekly. In some embodiments, after an initial period (e.g., 4 weeks), dosage of the GLP-1 receptor is increased (e.g., in 2.5 mg increments to e.g., 5 mg) injected subcutaneously once weekly. In some embodiments, maintenance dosages of the GLP-1 receptor agonist of 5 mg, 10 mg, or 15 mg injected subcutaneously once weekly can be utilized. Treatment response and tolerability are considered when selecting a maintenance dosage. In some embodiments, the GLP-1 receptor agonist is tirzepatide or a pharmaceutically acceptable salt thereof.

### 5.11. Patients

In some embodiments of the methods of this disclosure, the subject is overweight or obese. In some embodiments, the subject has, is suspected of having, or is at risk of developing a metabolic disease. In some embodiments, the metabolic disease is weight gain or obesity. In some embodiments, the subject has, is suspected of having, or is at risk of developing weight gain. In some embodiments, the subject to be treated is overweight or obese and in the presence of at least one weight-related comorbid condition (e.g., hypertension, dyslipidemia, type 2 diabetes mellitus, obstructive sleep apnea or cardiovascular disease).

In some embodiments, the subject is obese. In some embodiments, the subject is overweight. In some embodiments, the subject has, is suspected of having, or is at risk of developing a disease or condition associated with obesity. In some embodiments, the subject has a BMI of 25 to <30 kg/m². In some embodiments, the subject has a BMI of 27 to <30 kg/m². In some embodiments, the subject has a BMI of 27 kg/m² or greater, which is overweight. In some embodiments, the subject has a BMI of 30 kg/m² or higher, which is obese. In some embodiments, the subject is Class 1 obese (BMI of 30 to <35), Class 2 obese (BMI of 35 to <40), or Class 3 three obese (BMI of 40 or higher). Body Mass Index (BMI) is calculated by: BMI=weight (kg)/[height (m)]².

In some embodiments, the subject can have, is suspected of having, is at risk of developing, or is diagnosed with diabetes type I, diabetes type II, diabetes type Illa, or a metabolic syndrome.

In some embodiments, the weight gain associated condition is obesity. In some embodiments, the weight gain associated condition is excessive weight gain. In some embodiments, the weight gain associated condition is diabetes mellitus. In some embodiments, the weight gain associated condition is insulin insensitivity. In some embodiments, the weight gain associated condition is cardiovascular disease. In some embodiments, the weight gain associated condition is neurologic disease. In some embodiments, the condition is obesity-linked gallbladder disease. In some embodiments, the weight gain associated condition is obesity-induced sleep apnea. In some embodiments, the condition is diabetes. In some embodiments, the weight gain associated condition is excessive appetite. In some embodiments, the weight gain associated condition is fatty liver disease. In some embodiments, the weight gain associated condition is non-alcoholic fatty liver disease (NASH). In some embodiments, the weight gain associated condition is dyslipidemia. In some embodiments, the condition is metabolic syndrome. In some embodiments, the condition is insufficient satiety. In some embodiments, the weight gain associated condition is hyperinsulinemia. In some embodiments, the weight gain associated condition is nighttime hypoglycemia.

It was previously demonstrated that aged mice (24-month-old) treated with BGE-105 exhibit a statistically significant increase in voluntary motor activity (p=0.00228) and a statistically significant improvement in grip strength (p=0.04) as compared to age-matched controls, indicating improved physical health and increased muscle strength. It was also previously demonstrated that aged mice (18-month-old) first injected with a cardiotoxin and then treated with BGE-105 showed significantly higher levels of several mRNA transcripts which are indicative of muscle regeneration. It was also previously demonstrated that immortalized muscle precursor cells from human patients showed a dose-dependent relationship between cell growth and differentiation, and concentration of BGE-105. Lastly, immobilized aged mice (20-months-old) that were orally dosed with BGE-105 displayed significantly reduced muscle atrophy as compared to immobilized mice that were injected with the control vehicle.

Thus, an apelin receptor agonist can increase physical performance, counteract age-related frailty, and can reduce age-related muscle weakness.

Thus, an apelin receptor agonist can increase physical performance, counteract age-related frailty, prevent and can reduce age-related muscle weakness and treat muscle atrophy.

In some embodiments, the patient has, or is at risk of developing: sarcopenia, frailty, muscle weakness, reduction in risk of hip fracture, ICU associated muscle weakness, muscle atrophy, diaphragm disfunction, diaphragm atrophy, immobilization associated muscle weakness, immobility associated muscle weakness, recovery from muscle injury, or muscle wasting.

In some embodiments, the patient is on bedrest.

In some embodiments of the methods of this disclosure, the subject is human and has low muscle strength, low muscle force, low muscle mass, and/or low muscle volume due to disuse atrophy after immobilization.

In some embodiments, the patient is susceptible to, or at risk of having, sarcopenia. Sarcopenia is a condition characterized by loss of skeletal muscle mass and function. When this condition is associated with aging, it can also be referred to as age-related sarcopenia. Diagnosis of sarcopenia can be achieved via an assessment of low muscle mass plus the presence of low muscle function (low muscle strength/weakness or low physical performance) (see e.g., Cruz-Jentoft et al., (2010) Sarcopenia: European consensus on definition and diagnosis Report of the European Working Group on Sarcopenia in Older People. Age and Ageing; 39: 412-423; Muscaritoli et al., (2010) Consensus definition of sarcopenia, cachexia and pre-cachexia: joint document elaborated by Special Interest Groups (SIG) “cachexia-anorexia in chronic wasting diseases” and “nutrition in geriatrics”. Clin Nutr. April, 29(2):154-9; Fielding et al. (2011) Sarcopenia: An Undiagnosed Condition in Older Adults. Current Consensus Definition: Prevalence, Etiology, and Consequences. International Working Group on Sarcopenia. J Am Med Dir Assoc, 12: 249-256; and Studenski et al. (2014) The FNIH Sarcopenia Project: Rationale, study description, conference recommendations and final estimates. J Gerontol A Biol Sci Med Sci 69(5): 547-558).

Frailty is a geriatric condition characterized by an increased vulnerability to external stressors. It is strongly linked to adverse outcomes, including mortality, nursing home admission, and falls. In some embodiments, the patient is susceptible to, or at risk of having, a condition associated with one or more characteristic measures of frailty. In some embodiments, the subject is classified as frail. In some embodiments, the subject is classified as pre-frail, and is at a high risk or progression to being frail. Frailty can be diagnosed and/or characterized according to various indices of frailty that are composite measures of age-related changes indices of frailty, such as methods based on the Fried's frailty scale (see e.g., Fried, et al., Frailty in older adults: evidence for a phenotype. J Gerontol A Biol Sci Med Sci. 2001, 56: M146-M156) and/or the Mitnitski's Frailty Index (see e.g., Mitnitski et al., Frailty, fitness and late-life mortality in relation to chronological and biological age. BMC Geriatr. 2002, 2: 1-10).

In some embodiments, the patient is susceptible to, or at risk of having, muscle atrophy. Muscle atrophy refers to any wasting or loss of muscle tissue resulting from lack of use. Muscle atrophy can lead to muscle weakness and cause disability. In some embodiments, the patient is susceptible to, or at risk of having, immobilization-associated muscle weakness, which refers to any wasting or loss of muscle tissue resulting from immobilization, e.g., for medical reasons.

In some embodiments, the patient is susceptible to, or at risk of having, muscle weakness, also referred to as muscle fatigue, which refers to a condition characterized by the subject's inability to exert force with skeletal muscles. Muscle weakness often follows muscle atrophy.

Muscle atrophy can be measured using various endpoints, such as skeletal muscle protein fractional synthetic rate (FSR) in a liquid biopsy. Other measurements of muscle atrophy include diaphragm thickness, echo-density (e.g. of vastus lateralis), muscle circumference (of muscles such as the thigh/vastus lateralis), muscle cross-sectional area, and the like. Detection of muscle circumference can be measured using ultrasound. Ultrasound can be used to assess muscle atrophy, diaphragm dysfunction, predict extubating success or failure, quantify respiratory effort, and detect atrophy in, for example, mechanically ventilated subjects or subjects on bedrest.

In some embodiments, the patient is susceptible to, or at risk of having, a skeletal muscle condition. In some embodiments, the condition is not a cardiovascular condition. In some embodiments, the subject is not suffering from, or identified as having, a cardiovascular disease or condition. In some embodiments, the subject is not suffering from, or at risk of, a heart failure. In some embodiments, the subject is suffering from, or identified as having, a cardiovascular disease or condition. In some embodiments, the subject is suffering from, or at risk of, a heart failure.

In some embodiments the muscle condition is associated with the loss-of-function, decrease in the ability to regenerate, or heal after injury of skeletal muscle. In some embodiments the condition is associated with the loss-of-function of muscle stem cells.

In some embodiments, the patient is susceptible to, or at risk of having, insulin insensitivity associated with muscle atrophy. Type 2 diabetes mellitus can be associated with an accelerated muscle loss during aging, decreased muscle function, and increased disability.

### 5.11.1. Patient Age

In some embodiments of the method of treating a subject for a condition, the subject has, or is suspected of having, a condition associated with weight gain. In some embodiments of the method of inducing weight loss and preserving muscle function, the subject has, or is suspected of having, a condition associated with weight gain.

In some embodiments, the subject is human. The subject can be a human patient suffering from, or a risk of, an age-related muscle condition. In some embodiments, the patient is at least 30-years-old. In some embodiments, the patient is at least 40-years-old. In some embodiments, the patient is at least 50-years-old. In some embodiments, the patient is at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 40-50 years old, 50-60 years old, 60-70 years old, 70-80 years old, or 80-90 years old.

### 5.11.2. Assessment of Patients

A subject can be being susceptible of having a condition or disease, at risk of having a condition or disease, or having a condition or disease and identified as in need of treatment according to the methods of this disclosure, using a variety of different assessment methods.

For example, in some embodiments, the subject is susceptible of having, or at risk of having or developing, a muscle condition such as sarcopenia or frailty. In certain embodiments, the subject is at risk of developing sarcopenia or frailty due to weight loss therapy. In alternative embodiments, the subject has a muscle condition such as sarcopenia or frailty in addition to a condition or disease associated with weight gain (e.g., obesity). In certain embodiments, the subject that has a muscle condition such as sarcopenia or frailty in addition to a condition or disease associated with weight gain (e.g., such as obesity) has the muscle condition prior to undergoing weight loss therapy.

In some embodiments, the human subject has, is susceptible of having, is at risk of having, sarcopenia. In some embodiments, the human subject is identified as having sarcopenia. In some embodiments, the human subject is susceptible of having sarcopenia. In some embodiments, the human subject is at risk of having or developing sarcopenia. In some embodiments, the human subject is identified as having frailty. In some embodiments, the human subject is susceptible of having frailty. In some embodiments, the human subject is at risk of having or developing frailty.

In some embodiments, the patient has a BMI of at least 25. In some embodiments, the patient has a BMI of at least 30. In some embodiments, the patient has a BMI of at least 35, at least 40, at least 45, at least 50, at least 55, or at least 60. In some embodiments, the patient has a BMI of at 25 or more. In some embodiments, the patient has a BMI of 30 or more, 35 or more, 40 or more, 45 or more, 50 or more, 55 or more, or 60 or more. In some embodiments, a patient with a BMI of 25 or more is considered overweight. In certain embodiments, a patient with a BMI 25 or more is considered obese. In certain embodiments, a patient with a BMI 30 or more is considered obese.

A sarcopenia diagnosis can be determined or confirmed by the presence of low muscle quantity or quality. When low muscle strength or force, low muscle quantity/quality and low physical performance are all detected, sarcopenia is considered severe. In some embodiments, the patient has low muscle quantity or quality as compared to criteria representative of a healthy human subject, e.g., a subject of the same age or younger.

Low muscle mass can be assessed using appendicular lean body mass (ALBM). In some embodiments, low muscle mass is indicated by an ALBM adjusted for body mass index (BMI) of <0.789 kg for men or <0.512 kg for women, where ALBM can be measured by dual energy X-ray absorptiometry (DXA) or echoMRI. Additional muscle mass measurements include DEXA, total body potassium (TBK), MRI, total body electrical conductivity (TOBEC), and CT.

Low muscle mass can be assessed by the appendicular skeletal muscle index (ASMI). In some low muscle mass is indicated by an appendicular skeletal muscle index (ASMI) of less than 7.26 kg/m² for men, or less than 5.5 kg/m² for women, said ASMI being defined as appendicular skeletal muscle mass divided by the square of height, said ASMI being measured by dual energy X-ray absorptiometry (DXA).

Low muscle strength can include low grip strength, and be determined using a handgrip strength test. In some embodiments, low grip strength is assessed by measuring the amount of static force that the hand can squeeze around a handgrip dynamometer, e.g., as indicated by a value of less than 30 kg, such as less than 26 kg for men, or less than 20 kg for women, such as less than 16 kg, in the handgrip strength test.

In some embodiments, the human subject has, or is identified as having, low muscle strength. In some embodiments, the human subject has, or is identified as having, low muscle force. In some embodiments of the methods of this disclosure, the subject is human and has, or is identified as having or is at risk of having, one or more of low muscle strength, low muscle force, low muscle mass, low muscle volume. In some embodiments, the muscle is skeletal muscle. In some embodiments, the muscle is the diaphragm, tibialis anterior, tibialis posterior, gastrocnemius, sartorius, quadriceps femoris (rectus femoris, vastus intermedius, vastus lateralis, and vastus medialis), soleus, or extensor digitorum longus.

In some embodiments, the human subject has, is susceptible of having, is at risk of having, low lower limb muscle mass. In some embodiments, the human subject has, or is identified as having, low upper limb muscle mass. In some embodiments, the human subject has, or is identified as having, after undergoing weight loss therapy, low lower limb muscle mass. In some embodiments, the human subject has, or is identified as having, after undergoing weight loss therapy, low upper limb muscle mass.

In some embodiments, the human subject has, is susceptible or having, or is at risk of having, low muscle volume. In some embodiments, the muscle volume is skeletal muscle volume. In some embodiments, the muscle is a skeletal muscle. In some embodiments, the skeletal muscle is a diaphragm. In some embodiments, the muscle is diaphragm, tibialis anterior, tibialis posterior, gastrocnemius, sartorius, vastus intermedius, vastus lateralis, vastus medialis, soleus, or extensor digitorum longus. In some embodiments, the muscle is diaphragm, tibialis anterior, tibialis posterior, sartorius, soleus, or extensor digitorum longus. In some embodiments, the muscle is diaphragm muscle.

In some embodiments, the muscle volume is the muscle volume of one or more upper limb muscles selected from the group consisting of: shoulder abductors, shoulder adductors, elbow flexors, elbow extensors, wrist flexors, and wrist extensors.

In some embodiments, muscle mass is assessed after the dosing. In some embodiments, muscle mass is assessed at least one day after dosing. In some embodiments, the muscle mass is assessed at least one week after dosing. In some embodiments, the muscle mass is assessed at least one month after dosing.

In some embodiments, the subject has, susceptible of having, or is at risk of having, a muscle condition. In some embodiments, the muscle condition is a skeletal muscle condition. In some embodiments, the skeletal muscle expresses the apelin receptor and administration of the apelin receptor agonist activates the apelin/APJ system (APLNR gene) in the muscle tissue of the subject. The muscle of interest expresses the apelin receptor, and in some embodiments, the level of expression of the apelin receptor can be assessed or determined in a muscle tissue of the subject prior to and/or after treatment. In some embodiments, the subject has, or is identified as having, a low circulating level of apelin. Apelin circulating levels can be assessed in a biological sample obtained from the subject, e.g., using a quantitative assay (e.g., ELISA assay, or LC/MS) for determining the amount of an apelin peptide in a sample.

In some embodiments, the muscle condition is a diaphragmatic muscle condition. In some embodiments, the diaphragmatic muscle condition is diaphragm atrophy. In some embodiments, the diaphragmatic muscle condition is diaphragm dysfunction. Dysfunction of the diaphragm ranges from a partial loss of the ability to generate pressure (weakness) to a complete loss of diaphragmatic function (paralysis). Patients with bilateral diaphragmatic paralysis or severe diaphragmatic weakness are likely to have dyspnea or recurrent respiratory failure. They can have considerable dyspnea at rest, when supine, with exertion, or when immersed in water above their waist. Further, patients with bilateral diaphragmatic paralysis are at an increased risk for sleep fragmentation and hypoventilation during sleep.

In some embodiments of the methods of this disclosure, the subject is human and has, is identified as having, or is at risk of having, one or more of diabetes mellitus, insulin insensitivity, and cardiovascular disease.

Muscle atrophy can be measured using various endpoints, such as skeletal muscle protein fractional synthetic rate (FSR) in a liquid biopsy. Other measurements of muscle atrophy include muscle thickness, echo-density (e.g. of vastus lateralis), muscle circumference (of muscles such as the thigh/vastus lateralis), muscle cross-sectional area, and the like. Detection of muscle circumference can be measured using ultrasound.

In some embodiments, the patient is diagnosed as obese. In some embodiments, the patient is diagnosed with diabetes mellitus. In some embodiments, the patient is diagnosed with insulin insensitivity. In some embodiments, the patient is diagnosed with cardiovascular disease. In some embodiments, the patient is diagnosed with obesity-linked gallbladder disease. In some embodiments, the patient is diagnosed with is obesity-induced sleep apnea. In some embodiments, the patient is diagnosed with diabetes. In some embodiments, the patient is diagnosed with excessive appetite. In some embodiments, the patient is diagnosed with fatty liver disease. In some embodiments, the patient is diagnosed with non-alcoholic fatty liver disease (NASH). In some embodiments, the patient is diagnosed with dyslipidemia. In some embodiments, the patient is diagnosed with insufficient satiety. In some embodiments, the patient is diagnosed with hyperinsulinemia. In some embodiments, the patient is diagnosed with nighttime hypoglycemia.

In some embodiments, the patient is diagnosed as obese and as having sarcopenia. In some embodiments, the patient is diagnosed with diabetes mellitus and sarcopenia. In some embodiments, the patient is diagnosed with insulin insensitivity and sarcopenia. In some embodiments, the patient is diagnosed with cardiovascular disease and sarcopenia. In some embodiments, the patient is diagnosed with obesity-linked gallbladder disease and sarcopenia. In some embodiments, the patient is diagnosed with is obesity-induced sleep apnea and sarcopenia. In some embodiments, the patient is diagnosed with diabetes and sarcopenia. In some embodiments, the patient is diagnosed with excessive appetite and sarcopenia. In some embodiments, the patient is diagnosed with fatty liver disease and sarcopenia. In some embodiments, the patient is diagnosed with non-alcoholic fatty liver disease (NASH) and sarcopenia. In some embodiments, the patient is diagnosed with dyslipidemia and sarcopenia. In some embodiments, the patient is diagnosed with insufficient satiety and sarcopenia. In some embodiments, the patient is diagnosed with hyperinsulinemia and sarcopenia. In some embodiments, the patient is diagnosed with nighttime hypoglycemia and sarcopenia.

In some embodiments, the patient is diagnosed as obese and as having frailty. In some embodiments, the patient is diagnosed with diabetes mellitus and frailty. In some embodiments, the patient is diagnosed with insulin insensitivity and frailty. In some embodiments, the patient is diagnosed with cardiovascular disease and frailty. In some embodiments, the patient is diagnosed with obesity-linked gallbladder disease and frailty. In some embodiments, the patient is diagnosed with is obesity-induced sleep apnea and frailty. In some embodiments, the patient is diagnosed with diabetes and frailty. In some embodiments, the patient is diagnosed with excessive appetite and frailty. In some embodiments, the patient is diagnosed with fatty liver disease and frailty. In some embodiments, the patient is diagnosed with non-alcoholic fatty liver disease (NASH) and frailty. In some embodiments, the patient is diagnosed with dyslipidemia and frailty. In some embodiments, the patient is diagnosed with insufficient satiety and frailty. In some embodiments, the patient is diagnosed with hyperinsulinemia and frailty. In some embodiments, the patient is diagnosed with nighttime hypoglycemia and frailty.

### 5.12. Additional Embodiments

The followed numbered embodiments are also included within the present disclosure.
1. A method of inducing weight loss with maintenance of lean muscle mass in a subject in need of weight loss, the method comprising:
   co-administering to a subject in need thereof:
      an effective dose of an apelin receptor agonist; and
      an effective dose of a GLP-1 receptor agonist, to maintain lean muscle mass while inducing fat and weight loss in the subject.
2. A method of increasing total weight loss caused by administration of a pre-determined amount of a GLP-1 receptor agonist to a subject in need thereof, the method comprising:
   co-administering to a subject in need thereof:
      an effective dose of an apelin receptor agonist; and
      an effective dose of a GLP-1 receptor agonist,
   to increase total weight loss in the subject relative to weight loss caused by administration of a pre-determined amount of a GLP-1 receptor agonist alone.
3. A method of treating or preventing further muscle mass decrease caused by administration of a GLP-1 receptor agonist to a subject in need thereof, the method comprising:
   adding an effective dose of an apelin receptor agonist to the GLP-1 receptor agonist treatment regimen of a subject in need thereof,
   to treat or prevent further lean muscle mass decrease in the subject.
4. The method of any one of embodiments 1 to 3, wherein the co-administering stimulates muscle mass preservation, or stimulates an increase in muscle mass in the subject relative to a baseline level.
5. The method of any one of embodiments 1 to 4, wherein the subject exhibits after the co-administration:
   loss of fat but not lean muscle;
   increased lean mass percentage;
   increased lean/fat mass ratio; and/or
   reduced or normal fed glucose level,
   relative to a baseline level immediately before administration.
6. The method of any one of embodiments 1 to 6, wherein the subject is overweight or obese.
7. The method of any one of embodiments 1 to 6, wherein the subject has a disease or condition associated with weight gain.
8. The method of any one of embodiment 7, wherein the disease or condition associated with weight gain is selected from obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, and nighttime hypoglycemia.
9. The method of any one of embodiments 1 to 8, wherein the subject has a metabolic disorder.
10. The method of embodiment 9, wherein the subject has diabetic obesity.
11. The method of embodiment 9 or 10, wherein the subject has type 1 diabetes, type 2 diabetes, or gestational diabetes.
12. The method of any one of embodiments 1 to 11, wherein the subject has one or more of hypertension, dyslipidemia, obstructive sleep apnea, and cardiovascular disease.
13. The method of any one of embodiments 1 to 12, wherein the GLP-1 receptor agonist is selected from albiglutide, exenatide, liraglutide, lixisenatide, semaglutide, and tirzepatide.
14. The method of embodiment 14, wherein the GLP-1 receptor agonist is semaglutide.
15. The method of any one of embodiments 1 to 12, wherein the GLP-1 receptor agonist is a dual-acting GLP-1 receptor agonist, and glucose-dependent insulinotropic polypeptide (GIP) receptor agonist or glucagon receptor agonist.
16. The method of embodiment 15, wherein the GLP-1 receptor agonist is tirzepatide.
17. The method of any one of embodiments 1 to 12, wherein the GLP-1 receptor agonist is a triple-acting GLP-1 receptor agonist, GIP receptor agonist, and glucagon receptor agonist.
18. The method of embodiment 17, wherein the GLP-1 receptor agonist is retatrutide.
19. The method of any one of embodiments 1 to 12, wherein the GLP-1 receptor agonist is selected from albenatide, albiglutide, avexitide, cafraglutide, cotadutide, danuglipron, dapiglutide, diabegone, dulaglutide, ecnoglutide, efpeglenatide, efinopegdutide, efocipegtrutide, exenatide, exenatide biobetter, exenatide SR, froniglutide, liraglutide, liraglutide biobetter, lixisenatide, CT-868, efocipegtrutide, LY-3502970, maridebart, mazdutide, NLY-001, orforglipron, pegapamodutide, pemvidutide, retatrutide (LY-3437943), semaglutide, semaglutide injection, survodutide, vurolenatide, dapagliflozin+semaglutide, (cagrilintide+semaglutide), (LAI-287+semaglutide), (semaglutide+GIP analogue), 4P-004, AMG-133, AP-026, AZD-9550, BGM-0504, BMS-686117, Zn/BMS-686117 adduct, CT-388, CT-868, CT-996, DD-01, DR-10624, DR-10627, ECC-5004, E-2HSA, GL-0034, GLP-06, GMA-105, GMA-106, GMA-102, GSBR-1290, GXG-6, GZR-18, HEC-88473, HR-17031, HRS-7535, HRS-9531, HS-20004, HS-20094, HB-1085, HDM-1002, HL-08, HZ-010, JY-09, KN-056, LY-3493269, MBX-1416, MDR-001, MWN-101, NLY-001, NN-9490, NNC0519-0130, NN-6177, NN-9847, NN-9904, NN-6535 (semaglutide), NN-9932 (semaglutide), PF-06954522, PF-07081532, PF-06882961, PB-1023, PB-119, PB-718, RGT-075, SAL-015, SAL-0112, SCO—094, TERN-601, TTP-273, Uni-E4, VK-2735, YH-25724, XW-004, XW-014, YH-25724, YN-012, YN-015, ZT-002, and pharmaceutically acceptable salts thereof.
20. The method of any one of embodiments 1 to 12, wherein the GLP-1 receptor agonist is selected from (dorzagliatin+GLP-1), (exenatide+insulin aspart), ACT-1003, Adogel Sema, AER-601, AGM-212, BEBT-808, BZ-043B, C-2816, DAJC-1, DD-02, DR-10625, DR-10628, DS-004, DS-005, DS-006, DS-012, E-6, efpeglenatide+HM-12470, exenatide 2, exenatide LA, exenatide SR, Extendin-Fc, G-49, GB-7001, Gene Encoding GLP-1, GLP-1 Incretin Triagonist, GLP-1 Oral Preparation, GLP-1R Antagonist for Hypoglycemia, glucagon, Glucagon-Like Peptide-1+insulin human, GPCR-targeted Project 012, GPCR-targeted Project 013, GT-01123, HM-15275, HPG-5119, HSP-001, HSP-004, HSP-005, HSP012-C, Hydrogel Exenatide, I20-105S, I20-110, KP-405, LA-EX, liraglutide biobetter, liraglutide LA, MK-1462, MLX-7000, MWN-105, MWN-109, NLY-12, NPM-115, OGB-21502, OXM, P-11, PB-2301, PB-2309, RGT-028, RGT-274, RPC-8844, RT-104, SHX-022, SL-209, synthetic peptides to agonize GLP-1R and CCKBR for diabetes, TB-013, TB-222023, TB-592, TE-8105, THDBH-111, UDS-003, VTCG-15, XL-110, XL-310, XW-003+XW-015, XW-003+XW-017, Y-002, YGX-1, ZT-003, ZT-006, ZT-007, DA-1726, HDM-1005, (insulin degludec+liraglutide), DB-081, GW-002, HZCX-012, ID-110521156, THDB-0211, THDBH-110, THDBH-120, THDBH-121, UBT-251, ATBB-22, BEM-012, CIN-209, CIN-210, DD-03, exenatide+ND-017, exenatide+Synthetic Peptide 2, glucagon, Insulin-GLP1, MD-02, OGB-21501, P-01, PAT-201, PF-1807, PT-3, and pharmaceutically acceptable salts thereof.
21. The method of any one of embodiments 1 to 20, wherein the apelin receptor agonist is selected from BAL-1480, BMS-986224, ANPA-0073, apelin-13, [Pyr1]apelin-13, E339-3D6, (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, (S)—N-(1-(cyclobutylamino)-1-oxo-5-(piperidin-1-yl)pentan-3-yl)-5-(2,6-dimethoxyphenyl)-1-cyclopentyl-1H-pyrazole-3-carboxamide, and pharmaceutically acceptable salts thereof.
22. The method of any one of embodiments 1 to 20, wherein the apelin receptor agonist is of formula (I) or (II):

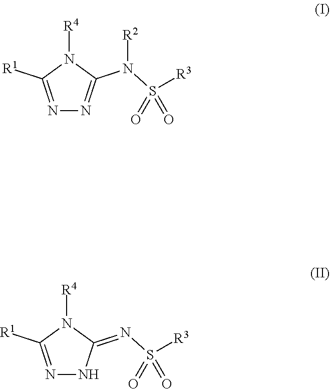

or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R³⁰ is not —H;
e) R¹ is not a 2-pyridyl group; or
f) R⁴ is substituted with two or more —O—(C₁-C₆ alkyl) groups.
23. The method of any one of embodiments 1 to 22, wherein the apelin receptor agonist is a compound of the structure

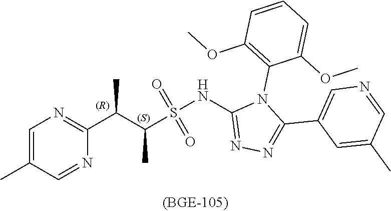

or a pharmaceutically acceptable salt thereof.
24. The method of embodiment 23, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.
25. The method of embodiment 23, wherein the apelin receptor agonist is a pharmaceutically acceptable salt of (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.
26. The method of any one of embodiments 1 to 20, wherein the apelin receptor agonist is of Formula (XXI):

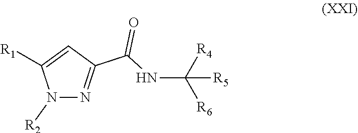

or a pharmaceutically acceptable salt thereof, wherein
   R₁ is represented by the formula:

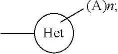

wherein

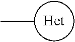

is a monocyclic aryl or heteroaryl group;
each A is independently fluoro substituted C₁-C₃ alkoxy or fluoro substituted C₁-C₃ alkyl;
n is 1, 2, 3, 4, or 5;
R₂ is C₃₋₈ alkyl, C₁₋₈ alkyl (C₃₋₈ cycloalkyl), C₃₋₈ cycloalkyl, heteroaryl or substituted aryl;
R₄ is adamantanyl, aryl, C₁₋₈ alkyl, C₁₋₈ alkyl alcohol, C₁₋₈ alkyl amino, C₁₋₈ alkyl amido, C₂₋₈ alkyl(aryl), C₁₋₈ alkyl (C₃₋₈ cycloalkyl), C₁₋₈ alkyl (C₃₋₈ cycloalkyl)-CO₂R₇, C₁₋₈ alkyl guanidinyl, C₁₋₈ alkyl heteroaryl, C₁₋₈ alkyl tetrazol-5-one, C₂₋₄ alkyl heterocycloalkyl, C₁₋₈ alkyl thioether, C₁₋₈ alkyl thiol, C₂₋₈ alkenyl, C₂₋₈ alkenyl(aryl), C₂₋₈alkenyl(heteroaryl), C₃₋₈ alkynyl, C₃₋₈ cycloalkyl, C₃₋₈ cycloalkyl-CO₂R₇, (CH₂)ₓNR₇R₈, (CH₂)ₓOR₇, (CH₂)ₓNR₉COR₇, (CH₂)ₓNR₉SO₂R₇, (CH₂)ₓNR₉CO₂R₇, (CH₂)ₓNHCOR₇, (CH₂)ₓNHSO₂R₇, (CH₂)ₓNHCO₂R₇, (CH₂)ₓCONR₇R₈, (CH₂)ₓCONR₇(CH₂)_{y}CO₂R₉, (CH₂)ₓCONR₇(CH₂)_{y}CONR₇R₈, (CH₂)ₓCONR₇(CH₂)_{y}R₉(CH₂)ₓCOR₇, (CH₂)ₓCO₂R₇, (CH₂)ₓSO₂NR₇(CH₂)_{y}R₉, CHR₇COR₉, CHR₇CONHCHR₈COR₉, CONR₇R₈, CONR₇(CH₂)ₓCO₂R₈, CONR₇CHR₈CO₂R₉, CO₂R₉, NHCO₂R₇, or (CH₂)ₓSO₂NR₇R₈;
R₅ and R₆ each are independently is adamantanyl, aryl, C₁₋₈ alkyl, Cis alkyl alcohol, C₁₋₈ alkyl amino, C₁₋₈ alkyl amido, C₂₋₈ alkyl(aryl), C₁₋₈ alkyl (C₃₋₈ cycloalkyl), C₁₋₈ alkyl (C₃₋₈ cycloalkyl)-CO₂R₇, C₁₋₈ alkyl guanidinyl, C₁₋₈ alkyl heteroaryl, C₁₋₈ alkyl tetrazol-5-one, C₂₋₄ alkyl heterocycloalkyl, C₁₋₈ alkyl thioether, C₁₋₈ alkyl thiol, C₂₋₈ alkenyl, C₂₋₈ alkenyl(aryl), C₂₋₈ alkenyl(heteroaryl), (C₃₋₈ alkynyl, C₃₋₈ cycloalkyl, C₃₋₈ cycloalkyl-CO₂R₇, (CH₂)ₓNR₇R₈, (CH₂)ₓOR₇, (CH₂)ₓNR₉COR₇, (CH₂)ₓNR₉SO₂R₇, (CH₂)ₓNR₉CO₂R₇, (CH₂)ₓNHCOR₇, (CH₂)ₓNHSO₂R₇, (CH₂)ₓNHCO₂R₇, (CH₂)ₓCONR₇R₈, (CH₂)ₓCONR₇(CH₂)_{y}CO₂R₉, (CH₂)ₓCONR₇(CH₂)_{y}CON₇R₈, (CH₂)ₓCONR₇(CH₂)_{y}R₉, (CH₂)ₓCOR₇, (CH₂)ₓCO₂R₇, (CH₂)ₓSO₂NR₇(CH₂)_{y}R₉, CHR₇COR₉, CHR₇CONHCHR₈COR₉, CONR₇R₈, CONR₇(CH₂)ₓCO₂R₈, CONR₇CHR₈CO₂R₉, CO₂R₉, NHCO₂R₇, or (CH₂)ₓSO₂NR₇R₈; or
R₄ and R₅ together make a 4-8 member ring which may be substituted with one or more heteroatoms; or
R₄ and R₅ together make a 5-8 nitrogen containing member ring with one or more carbonyl groups;
wherein the group R₄ is substituted with one or more fluorine atoms:
R₆ is H;
R₇ and R₈ each are independently H, C₁₋₈ alkoxy, aryl, C₁₋₈ alkyl, C₁₋₈ alkyl alcohol, C₁₋₈ alkyl amino, C₁₋₈ alkyl amido, C₁₋₈ alkyl(aryl), C₁₋₈ alkyl (C₃₋₈ cycloalkyl), C₁₋₈ alkyl tetrazol-5-one, C₁₋₈ alkyl guanidinyl, C₁₋₈ alkyl heteroaryl, C₁₋₈ alkyl thioether, C₁₋₈ alkyl thiol, C₁₋₈ alkenyl, C₃₋₈ alkynyl, C₃₋₈ cycloalkyl, (CH₂)ₓCONHR₉, (CH₂)ₓCOR₉, (CH₂)ₓCO₂R₉, or heteroaryl; or
R₇ and R₈ together make a 3-9 member ring which may contain one or more heteroatoms, wherein the ring is substituted with at least two fluorine atoms; or
R₇ and R₈ together make a 5-8 nitrogen containing member ring with one or more carbonyl groups;
R₉ is aryl, C₁₋₈ alkoxy, C₁₋₈ alkyl, C₁₋₈ alkyl(aryl), C₃₋₈ cycloalkyl, H, heteroaryl, or hydroxyl;
each x is independently 0-8; and
each y is independently 1-8.
27. The method of embodiment 26, wherein the apelin receptor agonist is a compound of the structure

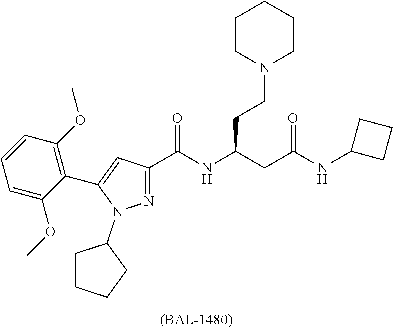 [SMILES: COc1cccc(OC)c1-c1cc(C(=O)N[C@@H](CCN2CCCCC2)CC(=O)NC2CCC2)nn1C1CCCC1]

or a pharmaceutically acceptable salt thereof.
28. The method of any one of embodiments 1 to 20, wherein the apelin receptor agonist is a compound of Formula (XV):

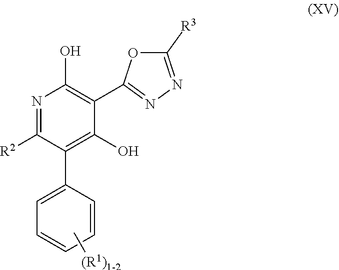

or a stereoisomer, a tautomer, or a pharmaceutically acceptable salt thereof, wherein:
   R¹ is independently selected from the group consisting of: —CH₂OH, —OCH₃, —OCF₃, CH₃, CH₂CH₃, CH(CH₃)₂, and cyclopropyl;
   R² is independently selected from the group consisting of: C₁₋₄ alkyl substituted with 0-3 R^{e}, C₂₋₄ alkenyl, C₁₋₆ cycloalkyl, and CH₂O(CH₂)₁₋₃CH₃;
   R³ is independently selected from the group consisting of
   (1) —CH₂C(═O)OC₁₋₄ alkyl substituted with 0-3 R^{e},
   (2) —CH₂NR^{a}R^{a},
   (3) —CH₂C(═O)NR^{a}R^{a},
   (4) —CH₂NHC(═O)C₁₋₄alkyl substituted with 0-3 R^{e},
   (5) —CH₂NR^{a}C(═O)(CH₂)₀₋₂OC₁₋₄alkyl substituted with 0-3 R^{e},
   (6) —CH₂—R⁵,
   (7) —CH₂—OR⁵,
   (8) —CH₂NR^{a}C(═O)(CH₂)₀₋₂R⁵, and
   (9) —CH₂C(═O)NR^{a}(CH₂)₀₋₂R⁵;
   R⁵ is independently selected from the group consisting of: aryl, C₃₋₆ cycloalkyl, and heterocycle, each substituted with 0-3 R⁶;
   R⁶ is independently selected from the group consisting of: H, F, Cl, Br, —OR^{b}, ═O, —(CH₂)ₙC(═O)R^{b}, —(CH₂)ₙC(═O)OR^{b}, —(CH₂)NR^{a}R^{a}, CN, —(CH₂)ₙC(═O)NR^{a}R^{a}, —S(O)₂NH₂, C₁₋₄ alkyl substituted with 0-3 R^{e}, (CH₂)ₙ—C₃₋₆ carbocyclyl substituted with 0-3 R^{e}, and —(CH₂)ₙ-heterocyclyl substituted with 0-3 R^{e};
   R^{a} is independently selected from the group consisting of: H, C₁₋₆ alkyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
   R^{b} is independently selected from the group consisting of: H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
   R^{e} is independently selected from the group consisting of: C₁₋₆ alkyl (optionally substituted with F and Cl), OH, OCH₃, OCF₃, —(CH₂)ₙ—C₃₋₆ cycloalkyl, —(CH₂)ₙ—C₄₋₆ heterocyclyl, —(CH₂)ₙ-aryl, —(CH₂)ₙ-heteroaryl, F, Cl, Br, CN, NO₂, ═O, and CO₂H; and n is independently selected from zero, 1, 2, and 3.
29. The method of embodiment 28, wherein the apelin receptor agonist is a compound having one of the following structures:

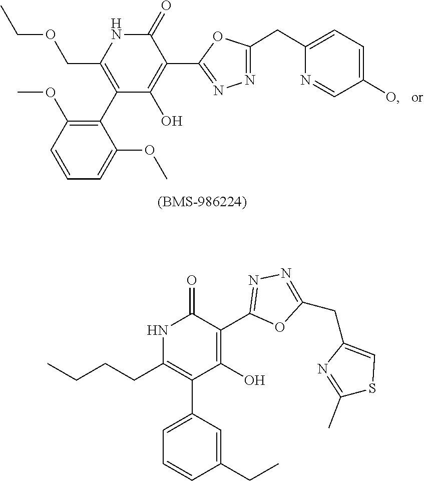

or a pharmaceutically acceptable salt thereof.
30. The method of any one of embodiments 1 to 29, wherein the method further comprises co-administering an additional therapeutic agent.
31. The method of embodiment 30, wherein the additional therapeutic agent is selected from insulin glargine, insulin degludec, cagrilintide, naltrexone-bupropion, phentermine-topiramate, benzphetamine, diethylpropion, phendimetrazine, phentermine, orlistat, and setmelanotide.
32. The method of any one of embodiments 1 to 31, wherein the GLP-1 receptor agonist is administered orally, intravenously, subcutaneously, intranasally, or intramuscularly.
33. The method of embodiment 32, wherein the GLP-1 receptor agonist is administered orally.
34. The method of embodiment 32 or 33, wherein the effective dose of the GLP-1 receptor agonist is administered daily.
35. The method of any one of embodiments 1 to 34, wherein the apelin receptor agonist is administered orally, intravenously, subcutaneously, intranasally, or intramuscularly.
36. The method of embodiment 35, wherein the apelin receptor agonist is administered orally.
37. The method of embodiment 35 or 36, wherein the effective dose of the apelin receptor agonist is administered daily.
38. The method of embodiment one of embodiments 1 to 33, further comprising, assessing lean muscle mass after the dosing.
39. A method for inducing weight loss that is adjunct to a reduced-calorie diet and increased physical activity for chronic weight management in subjects with an initial body mass index (BMI), the method comprising
co-administering to a subject in need thereof.
   an effective dose of an apelin receptor agonist; and
   an effective dose of a drug that reduces caloric intake.
wherein the subject (e.g., adult) is overweight or obese.
40. The method of embodiment 39, wherein the subject has a metabolic disorder.
41. The method of embodiment 40, wherein the subject has diabetic obesity.
42. The method of embodiment 40 or 41, wherein the subject has type 1 diabetes, type 2 diabetes, or gestational diabetes.
43. The method of any one of embodiments 39 to 42, wherein the subject has one or more of hypertension, dyslipidemia, obstructive sleep apnea, and cardiovascular disease.
44. The method of any one of embodiments 39 to 42, wherein the drug that reduces caloric intake is an appetite suppressor.
45. The method of any one of embodiments 39 to 44, wherein the drug that reduces caloric intake is selected from naltrexone-bupropion, phentermine-topiramate, benzphetamine, diethylpropion, phendimetrazine, phentermine, orlistat, setmelanotide, and pharmaceutically acceptable salts thereof.
46. The method of any one of embodiments 39 to 44, wherein the drug that reduces caloric intake is a GLP-1 receptor agonist.
47. The method of embodiment 46, wherein the GLP-1 receptor agonist is selected from albiglutide, exenatide, liraglutide, lixisenatide, semaglutide, and tirzepatide.
48. The method of embodiment 46, wherein the GLP-1 receptor agonist is selected from albenatide, albiglutide, avexitide, cafraglutide, cotadutide, danuglipron, dapiglutide, diabegone, dulaglutide, ecnoglutide, efpeglenatide, efinopegdutide, efocipegtrutide, exenatide, exenatide biobetter, exenatide SR, froniglutide, liraglutide, liraglutide biobetter, lixisenatide, CT-868, efocipegtrutide, LY-3502970, maridebart, mazdutide, NLY-001, orforglipron, pegapamodutide, pemvidutide, retatrutide (LY-3437943), semaglutide, semaglutide injection, survodutide, vurolenatide, dapagliflozin+semaglutide, (cagrilintide+semaglutide), (LAI-287+semaglutide), (semaglutide+GIP analogue), 4P-004, AMG-133, AP-026, AZD-9550, BGM-0504, BMS-686117, Zn/BMS-686117 adduct, CT-388, CT-868, CT-996, DD-01, DR-10624, DR-10627, ECC-5004, E-2HSA, GL-0034, GLP-06, GMA-105, GMA-106, GMA-102, GSBR-1290, GXG-6, GZR-18, HEC-88473, HR-17031, HRS-7535, HRS-9531, HS-20004, HS-20094, HB-1085, HDM-1002, HL-08, HZ-010, JY-09, KN-056, LY-3493269, MBX-1416, MDR-001, MWN-101, NLY-001, NN-9490, NNC0519-0130, NN-6177, NN-9847, NN-9904, NN-6535 (semaglutide), NN-9932 (semaglutide), PF-06954522, PF-07081532, PF-06882961, PB-1023, PB-119, PB-718, RGT-075, SAL-015, SAL-0112, SCO—094, TERN-601, TTP-273, Uni-E4, VK-2735, YH-25724, XW-004, XW-014, YH-25724, YN-012, YN-015, ZT-002, and pharmaceutically acceptable salts thereof.
49. The method of embodiment 46, wherein the GLP-1 receptor agonist is selected from (dorzagliatin+GLP-1), (exenatide+insulin aspart), ACT-1003, Adogel Sema, AER-601, AGM-212, BEBT-808, BZ-043B, C-2816, DAJC-1, DD-02, DR-10625, DR-10628, DS-004, DS-005, DS-006, DS-012, E-6, efpeglenatide+HM-12470, exenatide 2, exenatide LA, exenatide SR, Extendin-Fc, G-49, GB-7001, Gene Encoding GLP-1, GLP-1 Incretin Triagonist, GLP-1 Oral Preparation, GLP-1R Antagonist for Hypoglycemia, glucagon, Glucagon-Like Peptide-1+insulin human, GPCR-targeted Project 012, GPCR-targeted Project 013, GT-01123, HM-15275, HPG-5119, HSP-001, HSP-004, HSP-005, HSP012-C, Hydrogel Exenatide, I20-105S, I20-110, KP-405, LA-EX, liraglutide biobetter, liraglutide LA, MK-1462, MLX-7000, MWN-105, MWN-109, NLY-12, NPM-115, OGB-21502, OXM, P-11, PB-2301, PB-2309, RGT-028, RGT-274, RPC-8844, RT-104, SHX-022, SL-209, synthetic peptides to agonize GLP-1R and CCKBR for diabetes, TB-013, TB-222023, TB-592, TE-8105, THDBH-111, UDS-003, VTCG-15, XL-110, XL-310, XW-003+XW-015, XW-003+XW-017, Y-002, YGX-1, ZT-003, ZT-006, ZT-007, DA-1726, HDM-1005, (insulin degludec+liraglutide), DB-081, GW-002, HZCX-012, ID-110521156, THDB-0211, THDBH-110, THDBH-120, THDBH-121, UBT-251, ATBB-22, BEM-012, CIN-209, CIN-210, DD-03, exenatide+ND-017, exenatide+Synthetic Peptide 2, glucagon, Insulin-GLP1, MD-02, OGB-21501, P-01, PAT-201, PF-1807, PT-3, and pharmaceutically acceptable salts thereof.
50. The method of any one of embodiments 39 to 49, wherein the apelin receptor agonist is selected from BAL-1480, BMS-986224, ANPA-0073, apelin-13, [Pyr1]apelin-13, E339-3D6, (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, (S)—N-(1-(cyclobutylamino)-1-oxo-5-(piperidin-1-yl)pentan-3-yl)-5-(2,6-dimethoxyphenyl)-1-cyclopentyl-1H-pyrazole-3-carboxamide, and pharmaceutically acceptable salts thereof.
51. The method of any one of embodiments 39 to 49, wherein the apelin receptor agonist is a compound of the structure

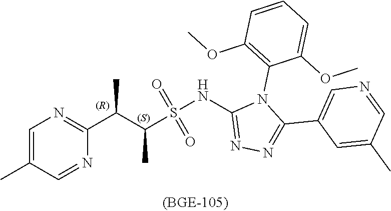

or a pharmaceutically acceptable salt thereof.
52. The method of any one of embodiments 39 to 49, wherein the apelin receptor agonist is a compound of the structure

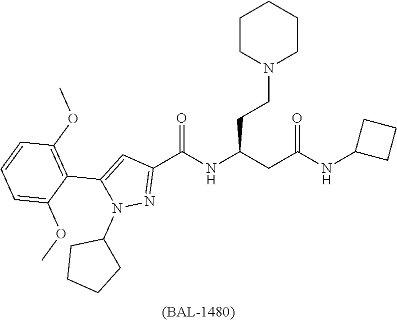 [SMILES: COc1cccc(OC)c1-c1cc(C(=O)N[C@@H](CCN2CCCCC2)CC(=O)NC2CCC2)nn1C1CCCC1]

or a pharmaceutically acceptable salt thereof.
53. The method of any one of embodiments 39 to 49, wherein the apelin receptor agonist is a compound having one of the following structures.

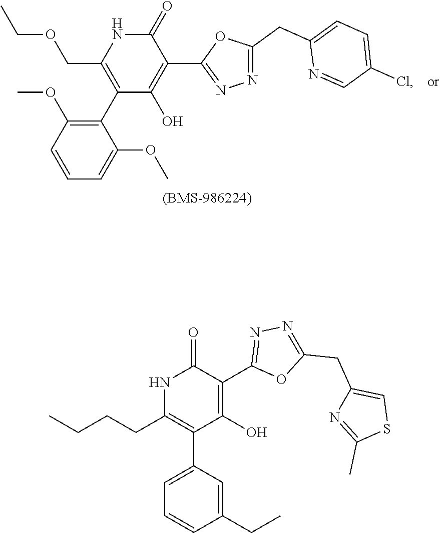 [SMILES: CCOCc1[nH]c(=O)c(-c2nnc(Cc3ccc(Cl)cn3)o2)c(O)c1-c1c(OC)cccc1OC ; CCCCc1[nH]c(=O)c(-c2nnc(Cc3csc(C)n3)o2)c(O)c1-c1cccc(CC)c1]

or a pharmaceutically acceptable salt thereof.

The followed series of numbered embodiments are also included within the present disclosure.
1. A method of treating a disease or condition associated with weight gain in a subject, the method comprising:
co-administering to a subject in need thereof:
   an effective dose of an apelin receptor agonist; and
   an effective dose of a glucagon-like peptide 1 receptor agonist (GLP-1RA) or analog thereof.
2. The method of embodiment 1, wherein the apelin receptor agonist is of formula (I) or (II):

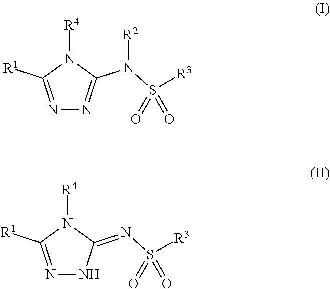

or a pharmaceutically acceptable salt thereof, a tautomer thereof, a pharmaceutically acceptable salt of the tautomer, a stereoisomer of any of the foregoing, or a mixture thereof, wherein:
   R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
   R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
   R² is selected from —H, and C₁-C₄ alkyl or is absent in the compounds of Formula II;
   R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
   R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
   R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
   Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
   R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
   R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
   R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
   further wherein:
   if R⁴ is an unsubstituted or substituted phenyl ring and R³ is a group of formula —(CR^{3b}═CR^{3c})-Q, then at least one of the following is true:
   a) R⁴ is substituted with at least one —O—(C₁-C₆ alkyl) group;
   b) Q is not an oxadiazole;
   c) R^{3b} is not —H;
   d) R^{3c} is not —H;
   e) R¹ is not a 2-pyridyl group; or
   f) R⁴ is substituted with two or more —O—(C₁-C₆ alkyl) groups.
3. The method of any one of embodiments 1 to 2, wherein the condition or disorder is obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, or nighttime hypoglycemia.
4. The method of embodiment 3, wherein the diabetes is type 1 diabetes, type 2 diabetes, or gestational diabetes.
5. The method of embodiment 4, wherein the subject exhibits lower fed glucose levels after treatment (e.g., within 20 days or less, such as 12 days or less, or 6 days or less of treatment).
6. The method of any one of embodiments 1 to 4, wherein the subject is human and at least 40-years-old.
7. The method of embodiment 6, wherein the subject is at least 50-years-old.
8. The method of embodiment 7, wherein the subject is at least 60-years-old.
9. The method of embodiment 8, wherein the subject is at least 65-years-old.
10. The method of embodiment 9, wherein the subject is at least 70-years-old or at least 75-years-old.
11. The method of embodiment 10, wherein the subject is at least 80-years-old.
12. The method of any one of embodiments 1 to 11, wherein the human subject has, or is at risk of having, low muscle strength or low muscle force.
13. The method of any one of embodiments 1 to 12, wherein the human subject has, or is at risk of having a muscle condition selected from: sarcopenia, frailty, muscle weakness, reduction in risk of hip fracture, ICU associated muscle weakness, muscle atrophy, diaphragm disfunction, diaphragm atrophy, immobilization associated muscle weakness, immobility associated muscle weakness, recovery from muscle injury, and muscle wasting.
14. The method of any one of embodiments 1 to 13, wherein the human subject has, or is or is at risk of having, low lower limb muscle mass.
15. The method of any one of embodiments 1 to 14, wherein the human subject has, or is at risk of having, low upper limb muscle mass.
16. The method of any one of embodiments 1 to 15, wherein the human subject has, or is at risk of having, low muscle volume.
17. The method of embodiment 16, wherein the muscle volume is skeletal muscle volume.
18. The method of embodiment 16, wherein the muscle is tibialis anterior, tibialis posterior, gastrocnemius, sartorius, vastus intermedius, vastus lateralis, vastus medialis, soleus, or extensor digitorum longus.
19. The method of any one of embodiments 1 to 18, wherein the apelin receptor agonist is administered orally, intravenously, subcutaneously, intranasally, or intramuscularly.
20. The method of any one of embodiments 1 to 19, wherein the effective dose of the apelin receptor agonist is administered daily.
21. The method of any one of embodiments 1 to 20, wherein the of the apelin receptor agonist is administered as a plurality of equally or unequally divided sub-doses.
22. The method of any one of embodiments 1 to 21, wherein the effective dose of the apelin receptor agonist is administered at varying dosing intervals.
23. The method of any one of embodiments 1 to 22, wherein the effective dose of the apelin receptor agonist is 200 mg.
24. The method of any one of embodiments 1 to 23, wherein the GLP-1RA or analog thereof is selected from: dulaglutide, exenatide, semaglutide, liraglutide, lixisenatide, tirzepatide, albenatide [INN], cotadutide, CT-868, PF 06882961, efocipegtrutide, LY-3502970, NLY-001, pegapamodutide, pemvidutide, PF-07081532, retatrutide, RGT-075, TTP-273, vurolenatide, GZR-18, mazdutide, PB-119, AMG-133, dapiglutide, DD-01, DR-10627, ECC-5004, exenatide biobetter, GL-0034, GMA-105, HEC-88473, LY-3493269, NN-6177, NN-9847, NNC0519-0130, PB-1023, SCO—094, VK-2735, YH-25724, YN-012, and YN-015.
25. The method of any one of embodiments 1 to 24 wherein the GLP-1RA or analog thereof is administered orally, intravenously, intranasally, or intramuscularly.
26. The method of any one of embodiments 1 to 25, wherein the effective dose of the GLP-1RA or analog thereof is administered daily.
27. The method of any one of embodiments 1 to 26, wherein the of the GLP-1RA or analog thereof is administered as a plurality of equally or unequally divided sub-doses.
28. The method of any one of embodiments 1 to 27, wherein the of the GLP-1RA or analog thereof is administered at varying dosing intervals.
29. The method of embodiment one of embodiments 1 to 28, further comprising, assessing muscle mass after the dosing.
30. The method of embodiment 29, wherein the muscle mass is assessed at least one day after dosing.
31. The method of embodiment 30, wherein the muscle mass is assessed at least one week after dosing.
32. The method of embodiment 30, wherein the muscle mass is assessed at least one month after dosing.
33. The method of any of embodiments 1-32, wherein the subject has a low circulating levels of apelin.
34. The method of any one of embodiments 1-33, wherein the subject has had surgery.
35. The method of any one of embodiments 1-34, wherein the subject has had weight loss surgery.
36. The method of any one of embodiments 1-35, further comprising co-administering an effective dose of an additional therapeutic agent.
37. The method of embodiment 36, wherein the additional therapeutic agent is an incretin receptor agonist.
38. The method of embodiment 36, wherein the additional therapeutic agent is selected from: cagrilintide [INN], insulin glargine, and insulin degludec.
39. A method of inducing weight loss in a subject in need thereof, the method comprising:
co-administering to a subject in need thereof:
an effective dose of an apelin receptor agonist; and
an effective dose of a glucagon-like peptide 1 (GLP-1) receptor agonist or analog thereof.
40. The method of embodiment 39, wherein the apelin receptor agonist is of formula (I) or (II):

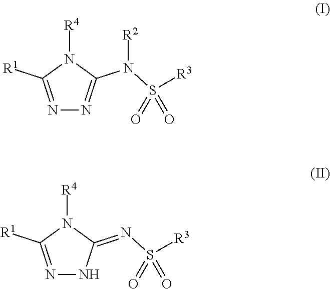

or a pharmaceutically acceptable salt thereof, a tautomer thereof, a pharmaceutically acceptable salt of the tautomer, a stereoisomer of any of the foregoing, or a mixture thereof, wherein:
   R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
   R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
   R² is selected from —H, and C₁-C₄ alkyl or is absent in the compounds of Formula II;
   R³ is selected from an unsubstituted C₁-C₁₀ alkyl, a C₁-C₁₀ alkyl substituted with 1, 2, or 3 R^{3a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
   R^{3a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
   R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
   R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
   Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
   R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo substituent;
   R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
   R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
   further wherein:
   if R⁴ is an unsubstituted or substituted phenyl ring and R³ is a group of formula —(CR^{3b}═CR^{3c})-Q, then at least one of the following is true:
   a) R⁴ is substituted with at least one —O—(C₁-C₆ alkyl) group;
   b) Q is not an oxadiazole;
   c) R^{3b} is not —H;
   d) R^{3c} is not —H;
   e) R¹ is not a 2-pyridyl group; or
   f) R⁴ is substituted with two or more —O—(C₁-C₆ alkyl) groups.
41. The method of any one of embodiments 39-40, wherein the subject is at least 60-years-old.
42. The method of embodiment 41, wherein the subject is at least 65-years-old.
43. The method of embodiment 42, wherein the subject is at least 70-years-old.
44. The method of embodiment 43, wherein the subject is at least 75-years-old.
45. The method of embodiment 44, wherein the subject is at least 80-years-old.
46. The method of any one of embodiments 39 to 45, wherein the human subject has, or is identified as having, low muscle strength.
47. The method of any one of embodiments 39 to 46, wherein the human subject has, or is identified as having, low muscle force.
48. The method of any one of embodiments 39 to 47, wherein the human subject has, or is identified as having, low lower limb muscle mass.
49. The method of any one of embodiments 39 to 48, wherein the human subject has, or is identified as having, low upper limb muscle mass.
50. The method of any one of embodiments 39 to 49, wherein the human subject has, or is identified as having, low muscle volume.
51. The method of embodiment 50, wherein the muscle volume is skeletal muscle volume.
52. The method of embodiment 51, wherein the muscle is the tibialis anterior, tibialis posterior, gastrocnemius, sartorius, vastus intermedius, vastus laterals, vastus medialis, soleus, or extensor digitorum longus.
53. The method of any one of embodiments 51 to 52, wherein the muscle is a skeletal muscle.
54. The method of any of embodiments 39 to 53, wherein the human subject has a low circulating apelin level.
55. The method of any one of embodiments 39 to 54, wherein the apelin receptor agonist is administered orally, intravenously, intranasally, or intramuscularly.
56. The method of any one of embodiments 39 to 55, wherein the dose is administered daily.
57. The method of any one of embodiments 39 to 56, wherein the dose is administered as a plurality of equally or unequally divided sub-doses.
58. The method of any one of embodiments 39 to 58, wherein the dose is administrated intravenously.
59. The method of any one of embodiments 39 to 58, wherein the GLP-1RA or analog thereof is selected from: dulaglutide, exenatide, semaglutide, liraglutide, liraglutide, lixisenatide, tirzepatide, semaglutide, albenatide [INN], cotadutide, CT-868, PF 06882961, efocipegtrutide, LY-3502970, NLY-001, pegapamodutide, pemvidutide, PF-07081532, retatrutide, RGT-075, TTP-273, vurolenatide, GZR-18, mazdutide, PB-119, AMG-133, dapiglutide, DD-01, DR-10627, ECC-5004, exenatide biobetter, GL-0034, GMA-105, HEC-88473, LY-3493269, NN-6177, NN-9847, NNC0519-0130, PB-1023, SCO—094, VK-2735, YH-25724, YN-012, and YN-015, preferably wherein the GLP-1RA or analog thereof is semaglutide, liraglutide, or tirzepatide.
60. The method of any one of embodiments 39 to 59, wherein the GLP-1RA is administered orally, subcutaneously, intravenously, intranasally, or intramuscularly.
61. The method of any one of embodiments 39 to 60, wherein the dose is administered daily.
62. The method of any one of embodiments 39 to 61, wherein the dose is administered as a plurality of equally or unequally divided sub-doses.
63. The method of any one of embodiments 39 to 62, wherein the dose is administered at varying dosing intervals.
64. The method of any one of embodiments 39 to 63, wherein R¹ is an unsubstituted pyridyl or is a pyridyl substituted with 1 or 2 R^{1a} substituents.
65. The method of any one of embodiments 39 to 64, wherein Ria in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

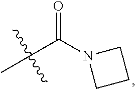 [SMILES: *C(=O)N1CCC1]

wherein the symbol [symbol] , when drawn across a bond, indicates the point of attachment to the rest of the molecule.
66. The method of any one of embodiments 38 to 62, wherein R¹ is selected from

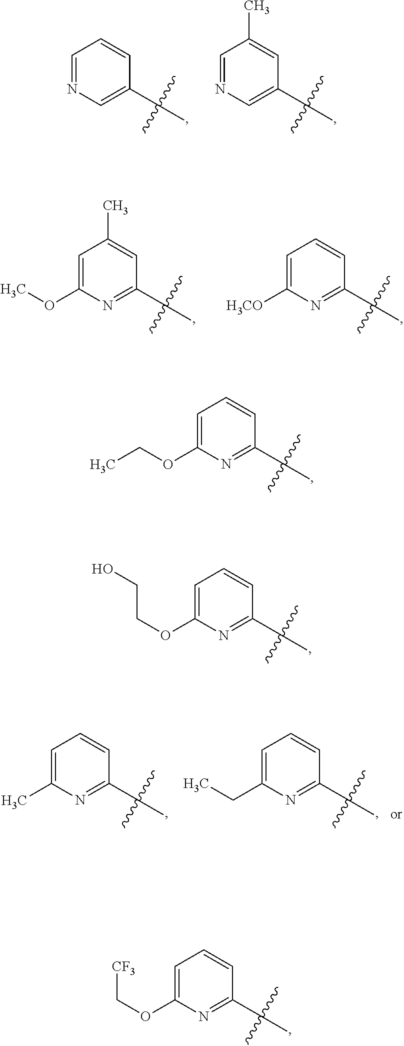

wherein the symbol [symbol] , when drawn across a bond, indicates the point of attachment to the rest of the molecule.
67. The method of any one of embodiments 39 to 66, wherein R² is —H.
68. The method of any one of embodiments 39 to 67, wherein R⁴ is a phenyl, pyridyl, pyrimidinyl, isoxazolyl, indolyl, naphthyl, or pyridinyl any of which may be unsubstituted or substituted with 1, 2, or 3 R^{4a} substituents.
69. The method of embodiment 68, wherein R⁴ is a phenyl substituted with 1 or 2 R^{4a} substituents.
70. The method of embodiment 69, wherein the 1 or 2 R^{4a} substituents are —O—(C₁-C₂ alkyl) groups.
71. The method of any one of embodiments 39 to 70, wherein R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.
72. The method of any one of embodiments 39 to 71, wherein R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR³R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.
73. The method of any one of embodiments 39 to 72, wherein Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.
74. The method of any one of embodiments 39 to 70, wherein Q is a monocyclic heteroaryl group with 5 or 6 ring members containing 1 or 2 heteroatoms selected from N, O, or S and Q is unsubstituted or is substituted with 1 or 2 R^{Q} substituents.
75. The method of any one of embodiments 39 to 74, wherein R³ is a group of formula —(CR^{3d}R^{3e})—(CR^{3f}R^{3g})-Q.
76. The method of any one of embodiments 39 to 75, wherein R³ has the formula

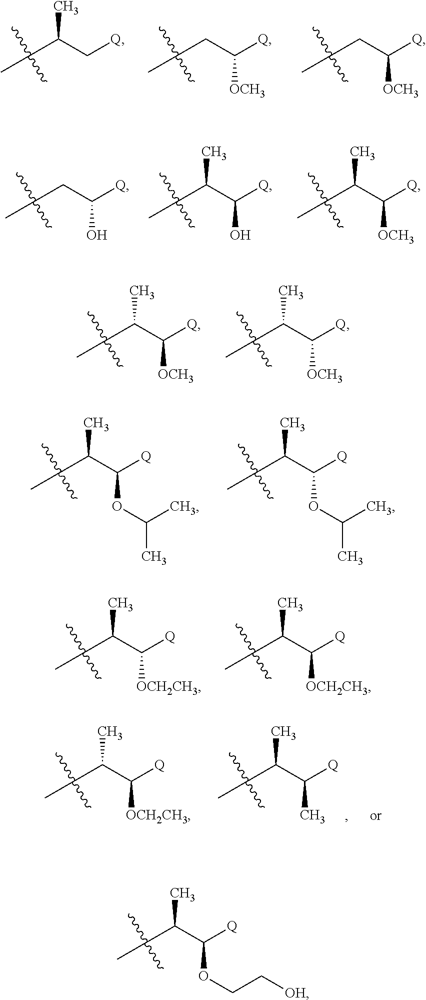

wherein the symbol [symbol] , when drawn across a bond, indicates the point of attachment to the rest of the molecule.
77. The method of any one of embodiments 1 to 76, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.
78. The method of embodiment 77, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.
79. The method of any one of embodiments 39 to 78, wherein the method further comprises co-administering an additional therapeutic agent.
80. The method of embodiment 79, wherein the additional therapeutic agent is an incretin receptor agonist.
81. The method of embodiment 79, wherein the additional therapeutic agent is selected from: insulin glargine, insulin degludec, and cagrilintide [INN].
82. The method of any one of embodiments 1 to 81, wherein the apelin receptor agonist is BGE-105 or a pharmaceutically acceptable salt thereof, and the glucagon-like peptide 1 receptor agonist (GLP-1RA) or analog thereof is semaglutide or a pharmaceutically acceptable salt thereof.
83. The method of any one of embodiments 1 to 81, wherein the apelin receptor agonist is BGE-105 or a pharmaceutically acceptable salt thereof, and the glucagon-like peptide 1 receptor agonist (GLP-1RA) or analog thereof is liraglutide or a pharmaceutically acceptable salt thereof.
84. The method of any one of embodiments 1 to 81, wherein the apelin receptor agonist is BGE-105 or a pharmaceutically acceptable salt thereof, and the glucagon-like peptide 1 receptor agonist (GLP-1RA) or analog thereof is tirzepatide or a pharmaceutically acceptable salt thereof.

### 5.13. Definitions

Unless defined otherwise, all technical and scientific terms used herein have the meaning commonly understood by a person skilled in the art to which this invention belongs.

The terms “individual,” “host,” and “subject” are used interchangeably, and refer to an animal to be treated, including but not limited to humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations. The term “patient” refers to a human subject.

The term “modulator” refers to a compound or composition that modulates the level of a target, or the activity or function of a target, which may be, but is not limited to, apelin receptor. In some embodiments, the modulator compound can agonize or activate the target, such as apelin receptor. An agonist or activator of a target can increase the level of activity or signaling associated with the target.

The terms “treating,” “treatment,” and grammatical variations thereof are used in the broadest sense understood in the clinical arts. Accordingly, the terms do not require cure or complete remission of disease, and the terms encompass obtaining any clinically desired pharmacologic and/or physiologic effect, including improvement in physiologic measures associated with “normal”, non-pathologic, aging. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a” “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.10% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remains operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that is generally safe, non-toxic and neither biologically nor otherwise undesirable, and include an excipient, diluent, carrier, and adjuvant that is acceptable for veterinary use as well as human pharmaceutical use. The phrase “pharmaceutically acceptable excipient” includes both one and more than one such excipient, diluent, carrier, and/or adjuvant.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an —OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group.

“Alkynyl” refers to an unsaturated branched or straight-chain hydrocarbon having at least one carbon-carbon triple bond derived by the removal of one hydrogen atom from a single carbon atom of a parent alkyne. Typical alkynyl groups include, but are not limited to, ethynyl; propynyl; butynyl, 2-pentynyl, 3-pentynyl, 2-hexynyl, 3-hexynyl and the like. In certain embodiments, an alkynyl group has 2 to 20 carbon atoms and in other embodiments, has 2 to 6 carbon atoms. An alkynyl group having 2 to 6 carbon atoms may be referred to as a —(C₂-C₆)alkynyl group.

“Alkoxy” refers to a radical —OR where R represents an alkyl group as defined herein. Representative examples include, but are not limited to, methoxy, ethoxy, propoxy, butoxy, cyclohexyloxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to the radical —C(O) or —C(═O) group.

“Carboxy” refers to the radical —C(O)OH.

“Cyano” refers to the radical —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, 117astric 117idi, cyclopentane, cyclohexane, cycloheptane, cyclooctane, and the like. Cycloalkyl groups may be described by the number of carbon atoms in the ring. For example a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₈)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

“Heterocyclyl” refers to a cyclic group that includes at least one saturated or unsaturated, but non-aromatic, cyclic ring. Heterocyclyl groups include at least one heteroatom as a ring member. Typical heteroatoms include O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring.

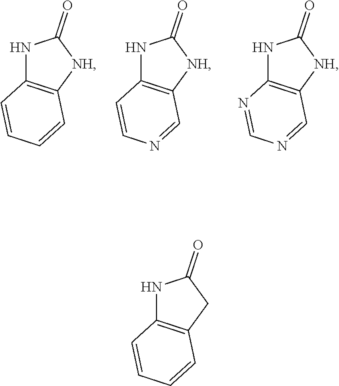 [SMILES: O=c1[nH]c2ccccc2[nH]1 ; O=c1[nH]c2ccncc2[nH]1 ; O=c1[nH]c2cncnc2[nH]1 ; O=C1Cc2ccccc2N1]

In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatoms are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Substituted heterocyclyl also includes ring systems substituted with one or more oxo (═O) or oxide (—O⁻) substituents, such as piperidinyl N-oxide, morpholinyl-N-oxide, 1-oxo-1-thiomorpholinyl, pyridinonyl, benzimidazolonyl, benzo[d]oxazol-2(3H)-onyl, 3,4-dihydroisoquinolin-1(2H)-onyl, indolin-onyl, 1H-imidazo[4,5-c]pyridin-2(3H)-onyl, 7H-purin-8(9H)-onyl, imidazolidin-2-onyl, 1H-imidazol-2(3H)-onyl, 1,1-dioxo-1-thiomorpholinyl, and the like.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2 In certain embodiments, the total number of S and O atoms in the aromatic heterocycle is not more than 1 Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 6. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### 6.1. Example 1: Pilot Study of the Effect of BGE-105 in Aged Diet-Induced Obese (DIO) Mice Treated with Semaglutide

### Pilot Study:

To assess the effects of BGE-105 on total weight loss, fat weight loss, and muscle loss/function in Diet-Induced Obese (DIO) mice treated concomitantly with the GLP-1 receptor agonist semaglutide.

Endpoints and measurements used to assess effects included:
Body Weight, Clinical Evaluation & Body Condition Score;
Group Food Intake, Water Intake;
Fed Glucose;
Body composition by Echo-MRI;
Baseline and endpoint grid hang tests; and
Endpoint tissue harvest.

### Methods:

25-month-old (aged) C57BL/6 female mice were used in this study. Diet for 25-month-old C57BL/6 mice included lean control D12450B (10 kcal % fat), or a diet induced-obesity (DIO) diet D12492 (60 kcal % fat) (Research Diets, Inc.). The study duration was 37 days. Treatment started on Day 0 and ended on Day 35-36.

Study Groups (n=9-11 per group) included the following:
Group 1: Lean Control mice
Group 2: Diet-induced obese (DIO) mice+Vehicle (VEH) (DIO control mice)
Group 3: DIO mice+BGE-105
Group 4: DIO mice+semaglutide
Group 5: DIO mice+semaglutide+BGE-105.

Semaglutide was purchased from MCE (Cat No. HY-114118/CS-0069903)

Mice from all Groups (1-5) were measured with fed glucose, and body weight and body composition for randomization. After randomization, mice were given treatment as shown below:
Group 1: Age-matched lean mice (Lean Control group) were given the following treatment: pH 8.5 drinking water+vehicle, 20 mM citrate buffer at pH 7.0, s.c., @ 4-5 PM every 3 days.
Group 2: Vehicle Control DIO mice (DIO mice+VEH group) were given pH 8.5 drinking water+vehicle, 20 mM citrate buffer at pH 7.0, s.c., @ 4-5 PM every 3 days.
Group 3: BGE-105 treated DIO mice (DIO mice+BGE-105) were given BGE-105 (1.1 mg/mL in drinking water, pH 8.5)+(vehicle, 20 mM citrate buffer at pH 7.0, s.c., @ 4-5 PM every 3 days).
Group 4: semaglutide treated DIO mice (DIO mice+semaglutide) were given semaglutide (100 nmol/kg, s.c., @ 4-5 PM every 3 days)+(pH 8.5 drinking water).
Group 5: semaglutide+BGE-105 treated DIO mice (DIO+semaglutide+BGE-105) were given a combination of semaglutide (100 nmol/kg, s.c., @ 4-5 PM every 3 days)+BGE-105 (1.1 mg/mL in drinking water, pH 8.5).

Body weight, group food intake, group water intake, body composition and grid hang tests were measured and monitored during the study.

On the day of necropsy, blood plasma and tissue samples were collected.

### Results:

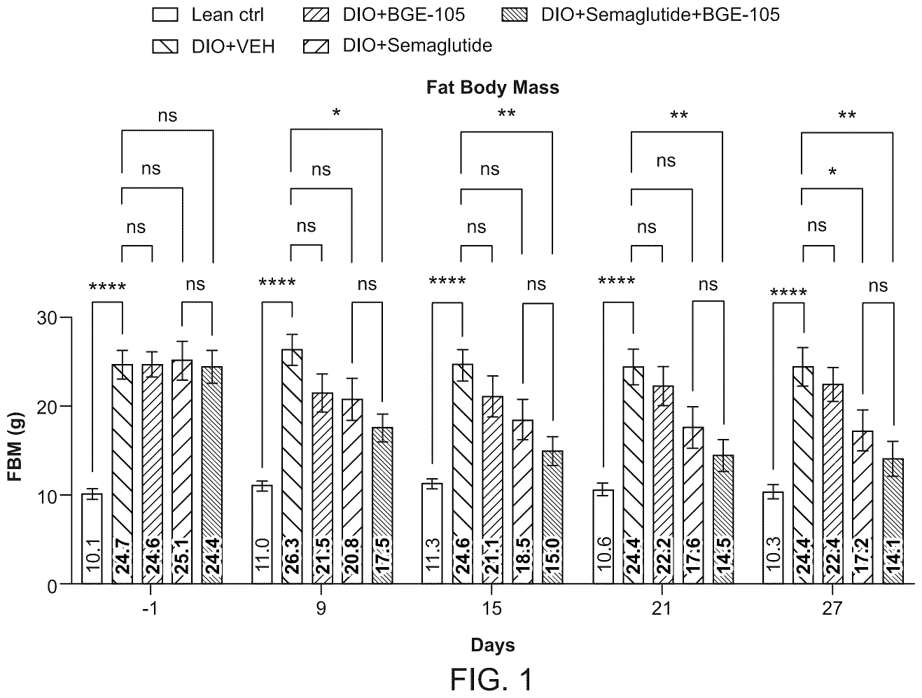

Fat body mass (FBM) was measured weekly using Echo-MRI. As shown in FIG. 1, treatment with the combination of BGE-105 and semaglutide (Group 5) reduced fat body mass (expressed as fat body mass (FBM)) to a greater extent than treatment with semaglutide alone (Group 4) in aged DIO mice. Data are expressed as mean±SEM and analyzed using Two-Way ANOVA with Tukey's multiple comparisons test.

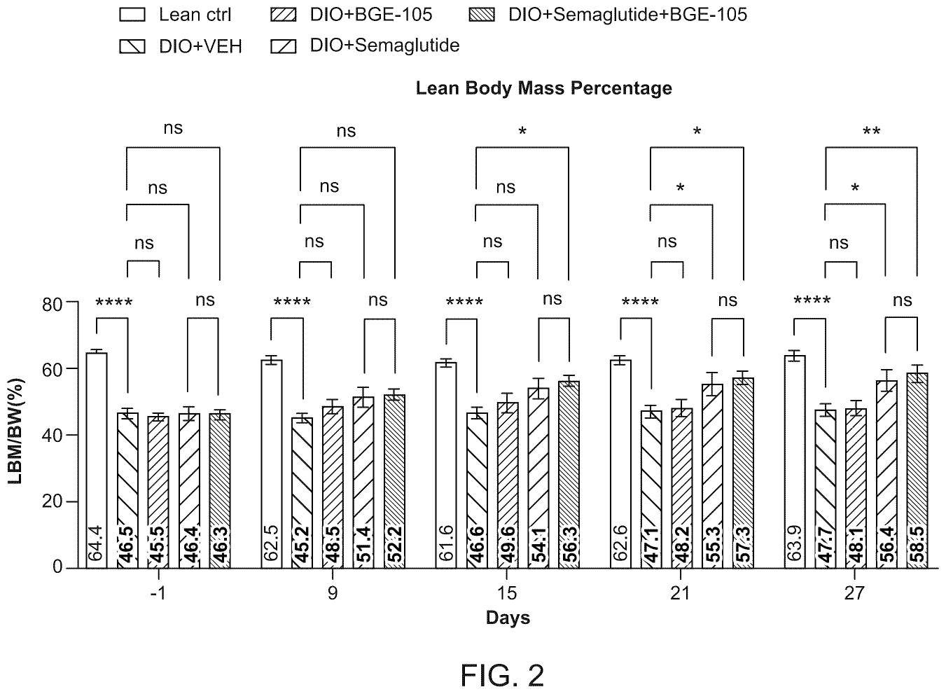

Lean body mass (LBM) was measured weekly using Echo-MRI. As shown in FIG. 2, treatment with the combination of semaglutide and BGE-105 (Group 5) increased lean body mass percentage (a percent of total body weight [BW]) to a greater extent than treatment with semaglutide alone (Group 4) in aged DIO mice. Data are expressed as mean SEM and analyzed using Two-Way ANOVA with Tukey's multiple comparisons test.

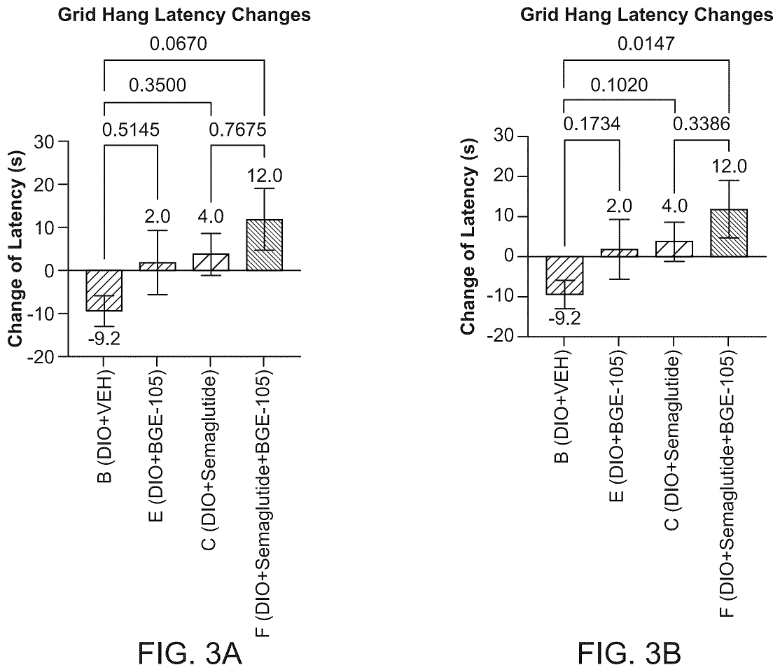

Muscle function was assessed using grid hang tests. As shown in FIGS. 3A-3B, treatment with semaglutide alone and with BGE-105 alone both increased muscle function as measured by an increase in latency before mice fell from the grid. Combination treatment with semaglutide and BGE-105 (Group 5) further improved muscle function as compared to treatment with either agent alone. Data are expressed as mean±SEM. FIG. 3A was analyzed using One-Way ANOVA with Tukey's multiple comparisons test, whereas FIG. 3B was analyzed using One-Way ANOVA without multiple comparisons test (Fisher's LSD)).

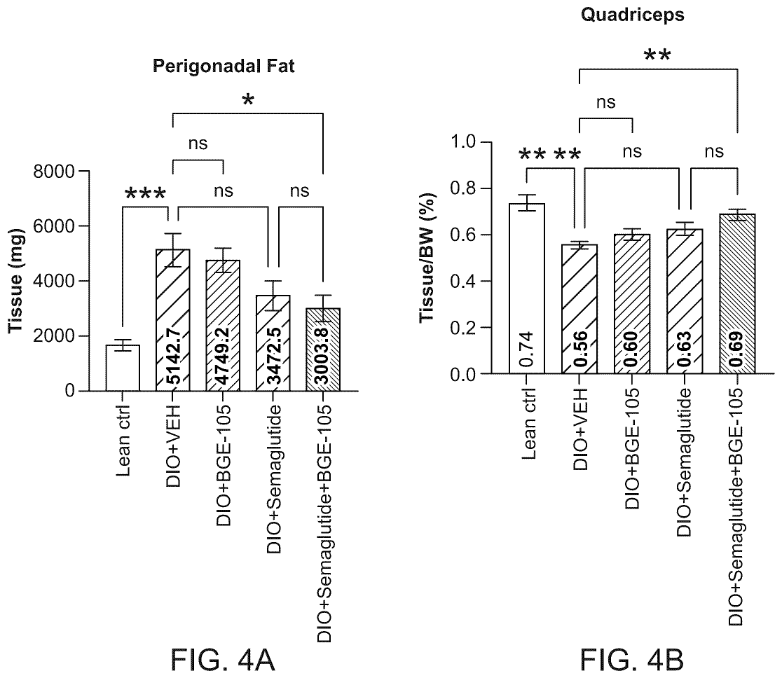

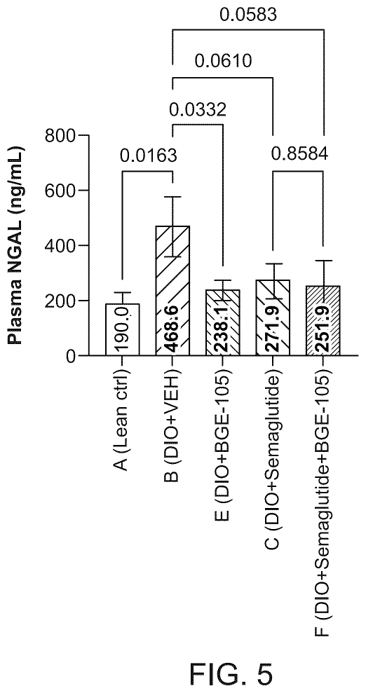

Weight of perigonadal fat (FIG. 4A) and quadriceps (FIG. 4B) were measured at the study endpoint. As shown in FIG. 4A, treatment with BGE-105 alone led to a statistically insignificant loss in perigonadal fat, as did treatment with semaglutide alone. Treatment with the combination of BGE-105 and semaglutide led to further loss of perigonadal fat, with the weight loss achieving statistical significance. As shown in FIG. 4B, treatment with BGE-105 alone and semaglutide alone increased quadricep weight as a percentage of total body weight, but the increases did not achieve statistical significance. Treatment with the combination of BGE-105 and semaglutide led to a statistically significant increase in quadriceps weight percentage as compared to DIO control mice. Data are expressed as mean±SEM and analyzed using One-Way ANOVA with Tukey's multiple comparisons test.

As shown in FIG. 5, the effect of obesity on renal function was assessed by evaluating urine neutrophil gelatinase-associated lipocalin (NGAL) level (ng/mL). Semaglutide-treated (Group 4), BGE-105-treated (Group 3), and combination of semaglutide+BGE-105-treated (Group 5) aged DIO mice had decreased plasma NGAL level at study endpoint as compared to the DIO control mice. Data are expressed as mean±SEM and analyzed using One-Way ANOVA without multiple comparisons test.

### 6.2. Example 2: Pilot Study of the Effect of BGE-105 in Aged Diet-Induced Obese (DIO) Mice Treated with Tirzepatide

### Pilot Study:

To assess the effects of BGE-105 on fat weight loss and muscle loss and muscle function in Diet-Induced Obese (DIO) mice treated with GLP-1 receptor agonist, tirzepatide.

### Study Parameters:

Two groups (lean mice group, DIO mice group) of aged female and male mice were used in this study. C57BL/6 aged female mice (lean n=10; DIO=56) were 21.3-months old at the time the study was initiated, and C57BL/6 male mice (lean n=10; DIO=39) were 17.2 months old at the time the study was initiated.

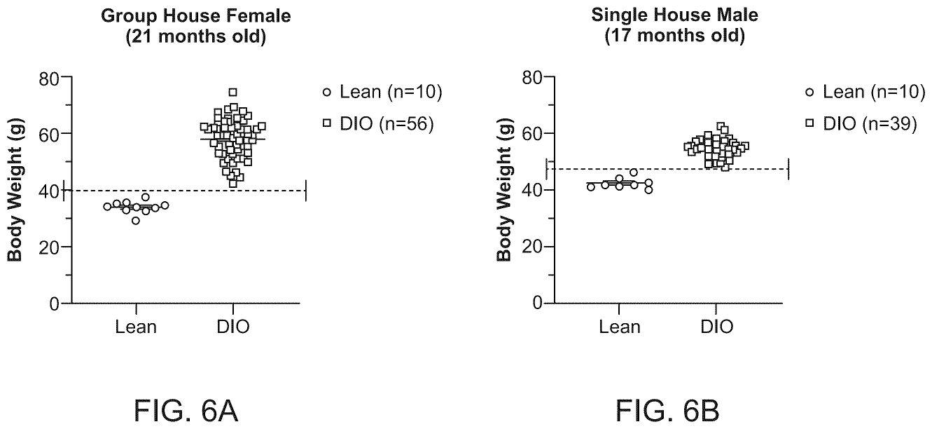

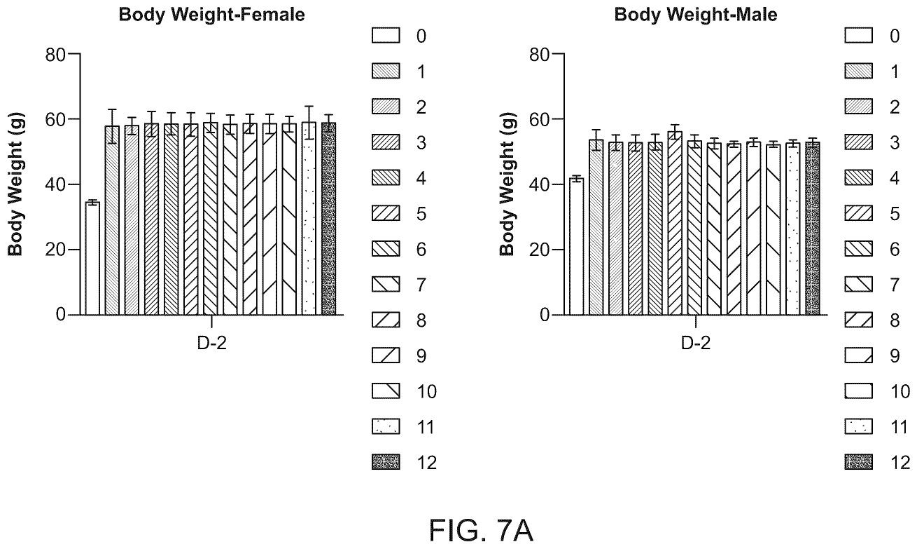

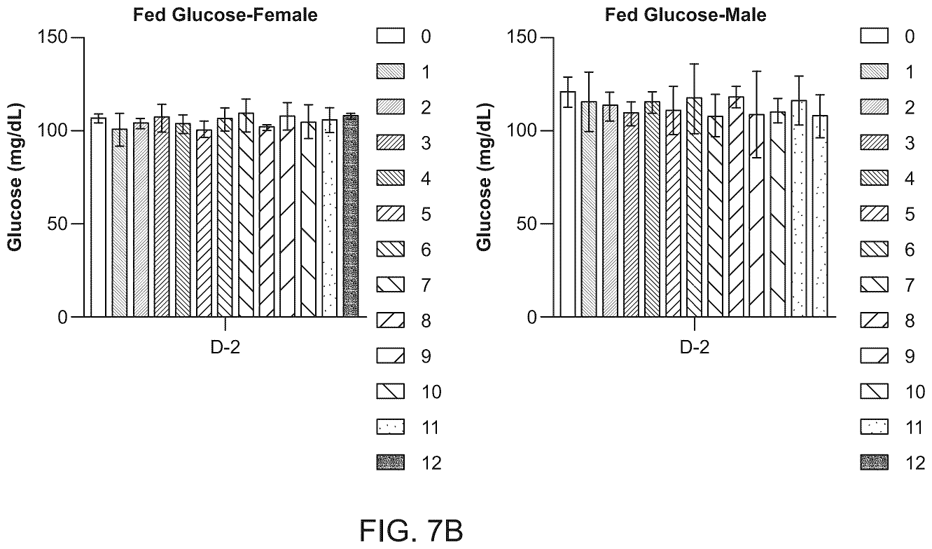

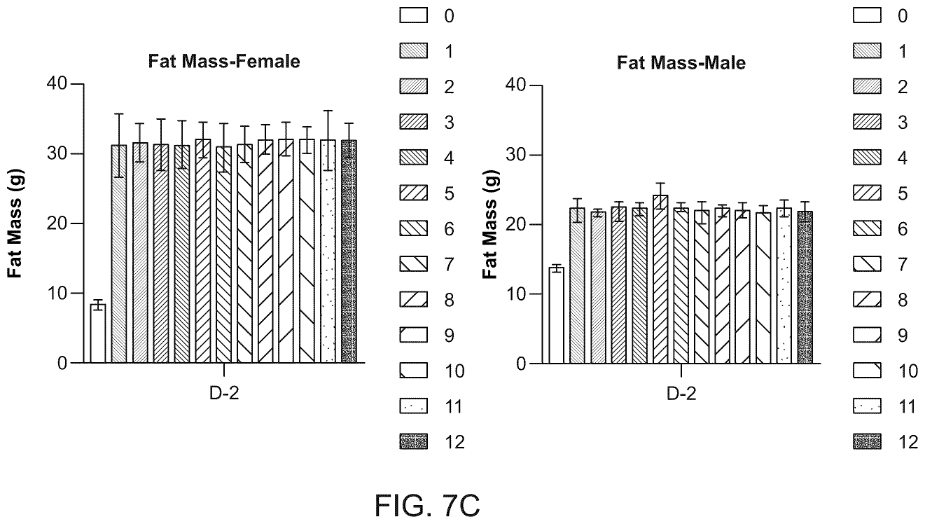

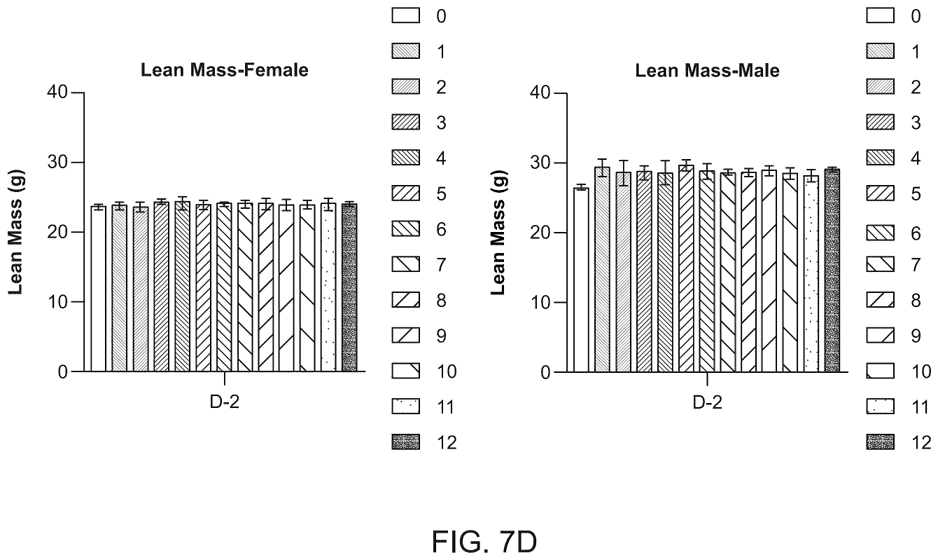

Prior to the start of any treatment (Day 0, “DO”), body weight for both female and male lean and DIO mice groups were measured. As shown in FIGS. 6A-6B, female mice in the lean group had a body weight ranging from 29-38 grams, while male mice in the lean group had a body weight ranging from 40-46 grams. All female and male DIO mice had a body weight greater than 50 grams. DIO female and male mice had a “DIO feed success rate” of 690 (female) and 51% (male), respectively. FIGS. 7A-7D show measurements of body weight, fed glucose, fat mass, and lean mass for both male and female mice groups used for randomization. Randomization occurred on Day 2 (“D-2”). The total duration of the study was 21 days, starting from Day 0.

After randomization, mice were given treatment per the following treatment groups:

TABLE 1
Treatment Groups and conditions

| | | Sample |
|---|---|---|
| | | size |
| Group # | Name/Treatment Condition | (n=) |
| 1 | DIO + VEH; (pH 8.5 drinking water + vehicle, 20 | 7 |
| | mM citrate buffer at pH 7.0, s.c., @ 4-5 PM every | |
| | 3 days) | |
| 2 | DIO + BGE-105 (0.275 g/L in drug water) | 7 |
| | monotherapy (low dose); (0.275 g/L in drug water, | |
| | pH 8.5) + (vehicle, 20 mM citrate buffer at pH 7.0, | |
| | s.c., @ 4-5 PM every 3 days) DIO mice | |
| 3 | DIO + BGE-105 (1.1 g/L in drug water) | 7 |
| | monotherapy (high dose); (1.1 g/L in drug water, | |
| | pH 8.5) + (vehicle, 20 mM citrate buffer at pH 7.0, | |
| | s.c., @ 4-5 PM every 3 days) DIO mice | |
| 4 | DIO + tirzepatide (3 nmol/kg); (3 nmol/kg, s.c., | 7 |
| | every 3 days) + (pH 8.5 drinking water) DIO mice | |
| 5 | DIO + tirzepatide (3 nmol/kg) + BGE-105 (0.275 | 5 |
| | g/L); (3 nmol/kg, s.c., every 3 days) + BGE-105 | |
| | (0.275 g/L in drug water, pH 8.5) DIO mice | |
| 6 | DIO + tirzepatide (3 nmol/kg) + BGE-105 (1.1 g/L); | 6 |
| | (3 nmol/kg, s.c., every 3 days) + BGE-105 (1.1 g/L | |
| | in drug water, pH 8.5) DIO mice | |
| 7 | DIO + tirzepatide (10 nmol/kg); (10 nmol/kg, s.c., | 6 |
| | every 3 days) + (pH 8.5 drinking water) DIO mice | |
| 8 | DIO + tirzepatide (10 nmol/kg) + BGE-105 (0.275 | 4 |
| | g/L); (10 nmol/kg, s.c., every 3 days) + BGE-105 | |
| | (0.275 g/L in drug water, pH 8.5) DIO mice | |
| 9 | DIO + tirzepatide (10 nmol/kg) + BGE-105 (1.1 | 5 |
| | g/L); (10 nmol/kg, s.c., every 3 days) + BGE-105 | |
| | (1.1 g/L in drug water, pH 8.5) DIO mice | |
| 10 | DIO + tirzepatide (30 nmol/kg); (30 nmol/kg, s.c., | 5 |
| | every 3 days) + (pH 8.5 drinking water) DIO mice | |
| 11 | DIO + tirzepatide (30 nmol/kg) + BGE-105 (0.275 | 5 |
| | g/L); (30 nmol/kg, s.c., every 3 days) + BGE-105 | |
| | (0.275 g/L in drug water, pH 8.5) DIO mice | |
| 12 | DIO + tirzepatide (30 nmol/kg) + BGE-105 (1.1 g/L); | 3 |
| | (30 nmol/kg, s.c., every 3 days) + BGE-105 (1.1 | |
| | g/L in drug water, pH 8.5) DIO mice | |

For BGE-105-treated Groups (Groups 2-3, 5-6, 8-9, and 11-12), BGE-105 was formulated and given in drinking water with 5 mM sucralose at pH 8.5. All arms were paired with s.c. vehicle and drinking water (5 mM sucralose at pH 8.5). For tirzepatide-treated groups, tirzepatide was given once every 3 days via s.c. injection. Groups were treated with vehicle, BGE-105, tirzepatide, or a combination of BGE-105 and tirzepatide every 3 days over the course of 21 days.

All groups were monitored and measured for body weight, water/food intake, fed glucose, and body composition. Data are expressed as mean±SEM and analyzed with 1-way or 2-way ANOVA without multiple comparisons.

### Measurement Parameters:

The following parameters were measured:

$$Fed Glucose Loss \% = ( Fed Glucose / Baseline Fed Glucose - 1 ) * 100$$

$$BW Loss \% = ( BW / Baseline BW - 1 ) * 100$$

$$Fat Mass Loss \% = ( Fat Mass / Baseline Fat Mass - 1 ) * 100$$

$$Lean Mass Loss \% = ( Lean Mass / Baseline Lean Mass - 1 ) * 100$$

$$FBM / BW Change = FBM / BW ( \% ) - Baseline FBM / BW ( \% )$$

$$LBM / BW Change = LBM / BW ( \% ) - Baseline LBM / BW ( \% )$$

### Results

### Water Intake

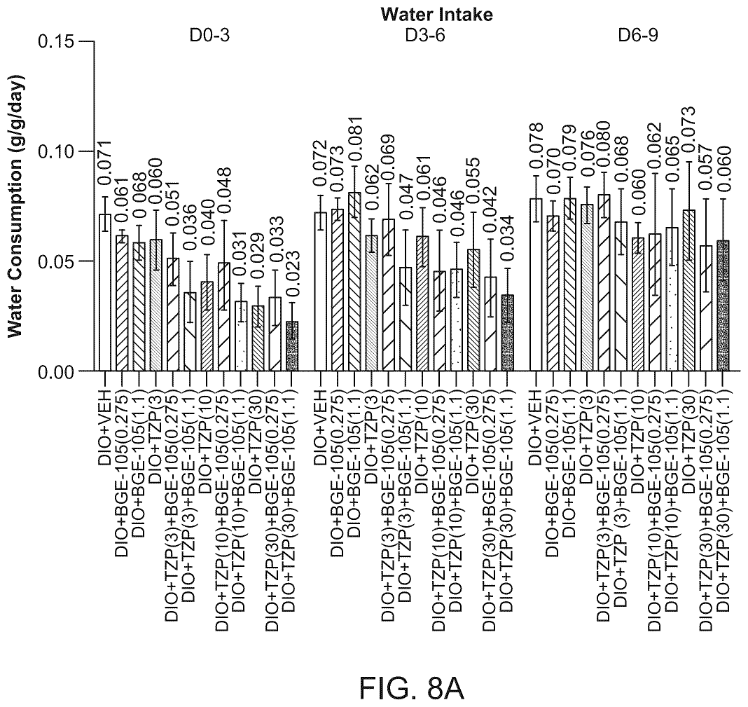

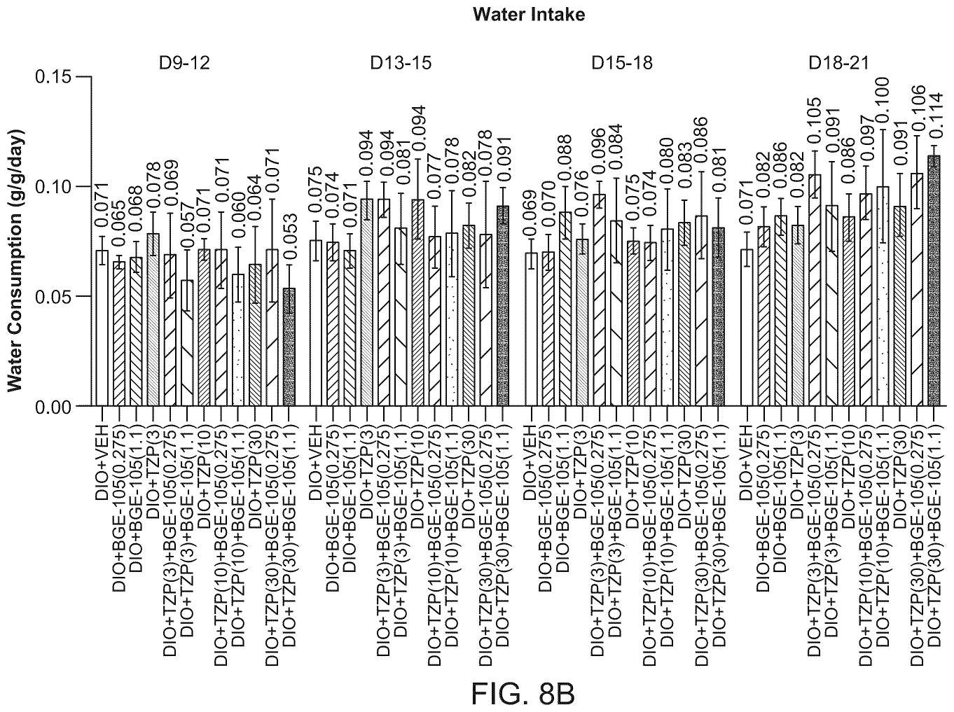

As shown in FIG. 8, daily water intake was monitored and measured for all groups (Groups 1-12) every 3 days throughout the duration of the study. The average amount of water consumption by aged DIO mice was calculated for each group (timepoints DO—3, D3-6, D6-9, D9-12, D13-15, D15-18, and D18-21) (D=“day”). As shown, groups treated with tirzepatide at higher titrations/higher dose (30 nmol/kg and 10 nmol/kg) had lower water intake as compared to groups treated with tirzepatide at lower titrations/lower dose (3 nmol/kg). Generally, animals had improved or maintained water intake after about 1 week.

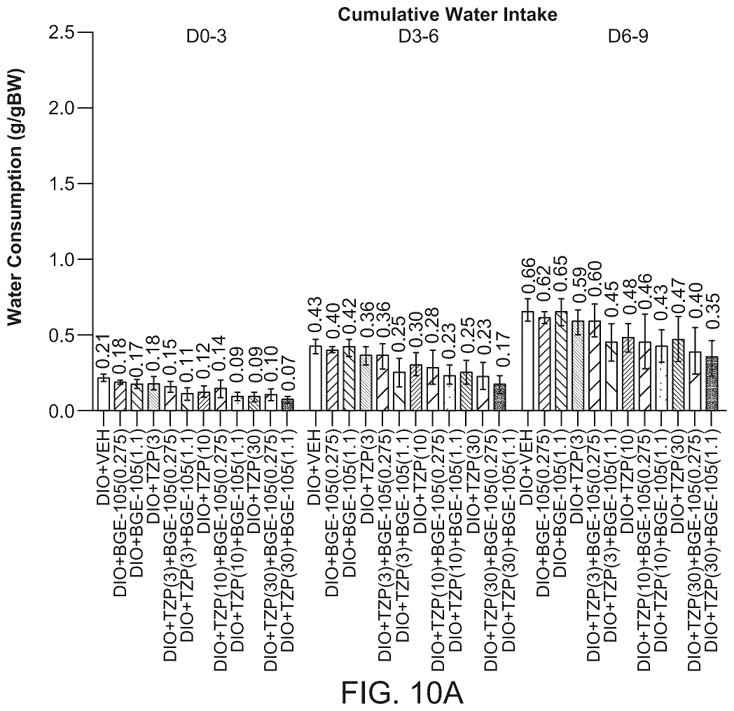

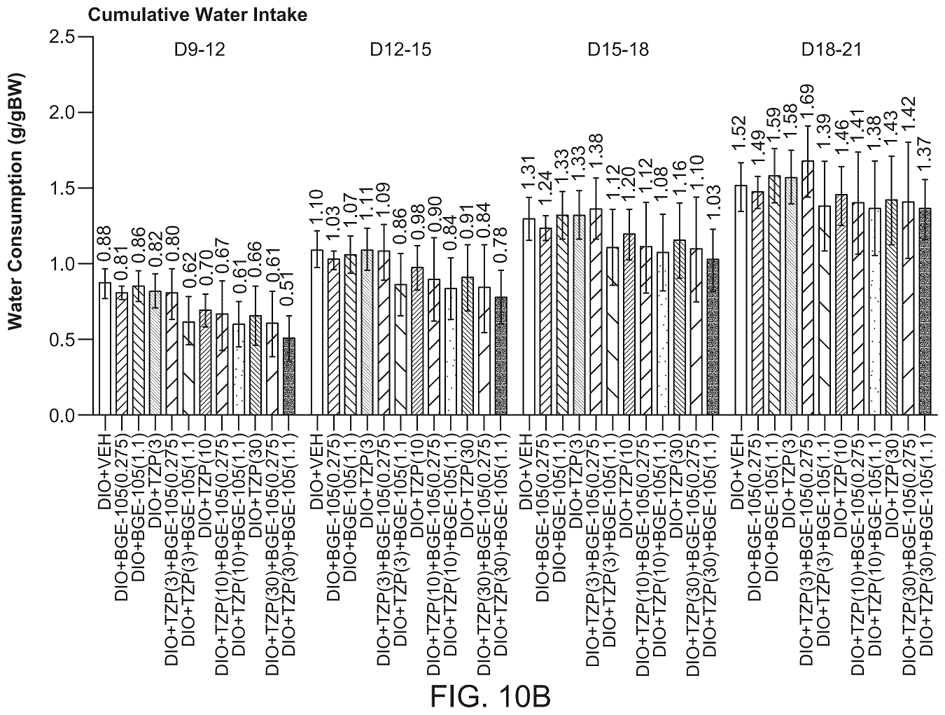

Cumulative water intake was also measured, as shown in FIG. 10, which graphs total amount of water, in grams, per gram of body weight (g/g/BW).

### Food Intake

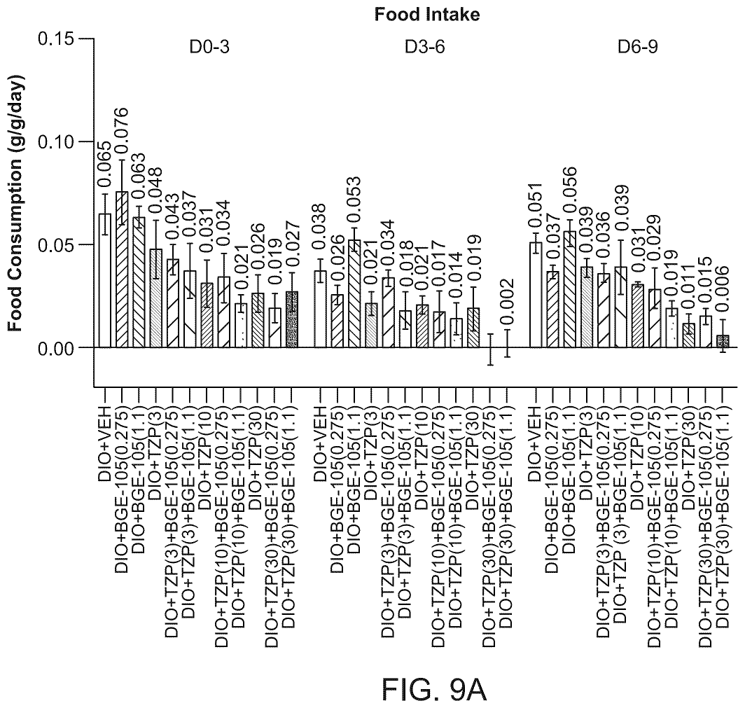

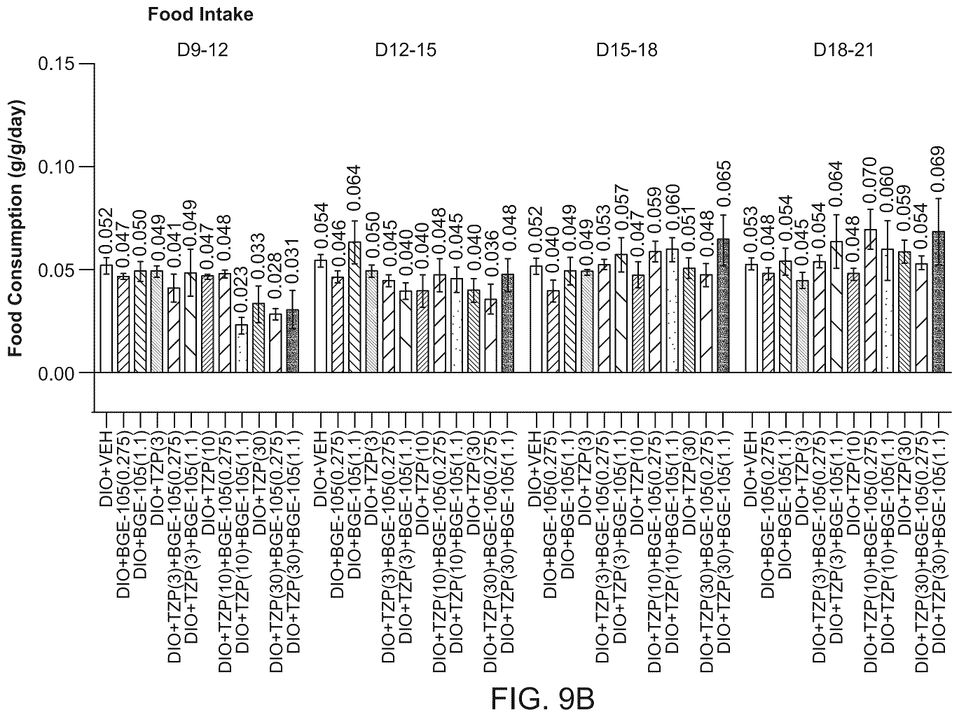

As shown in FIG. 9, food consumption was measured for all treatment groups every 3 days throughout the duration of the study. Due to drug effects, animals in tirzepatide or BGE-105 plus tirzepatide treated groups had lower food intake at the beginning of the study. However, after about 2 weeks, animals started to recover food intake and food intake was improved or maintained after about 12 days. Food intake on days D12-15 show that Group 3 (DIO+BGE-105 monotherapy; 1.1 g/L in drug water, high dose) mice had higher food intake than Group 4 (DIO+tirzepatide 3 nmol/kg)), Group 7 (DIO+tirzepatide 10 nmol/kg)), and Group 10 (DIO+tirzepatide 30 nmol/kg)).

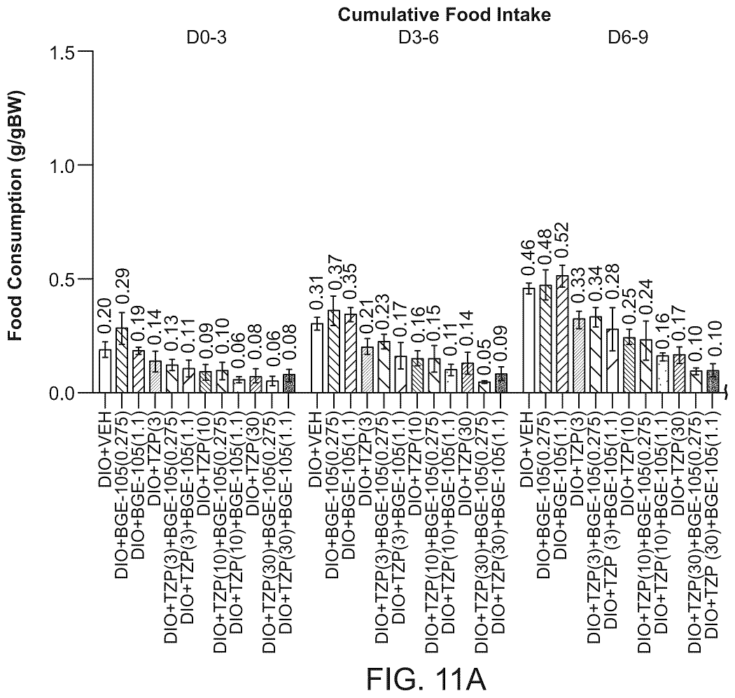

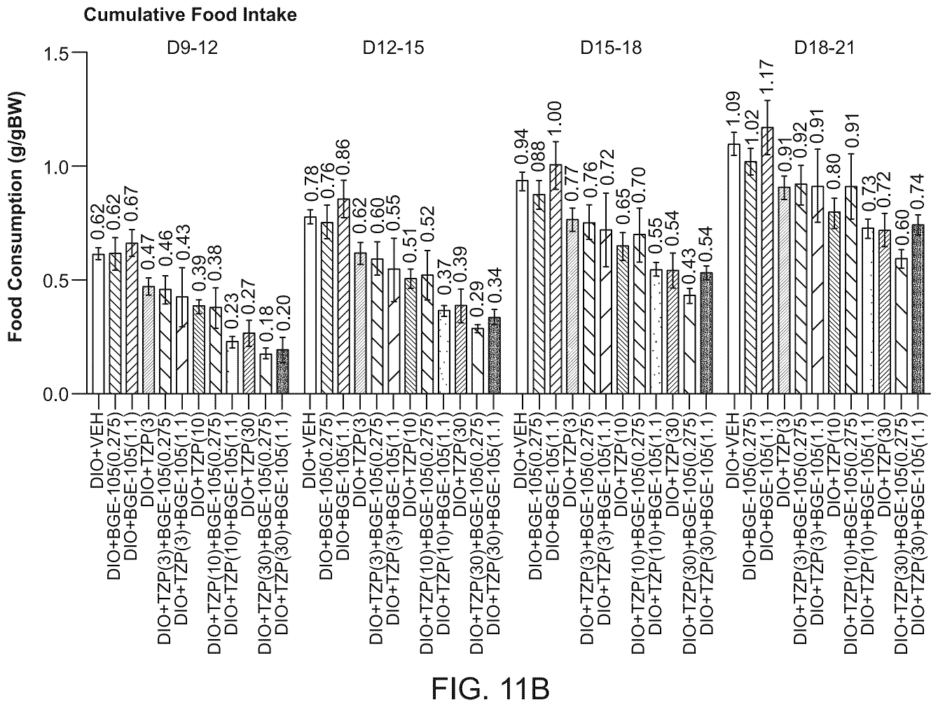

Cumulative food intake was also measured, as shown in FIG. 11 (total amount of food, in grams, per body weight in grams (g/g/BW).

### Body Weight (BW) Loss for Tirzepatide (TZP) Groups at Different Titrations

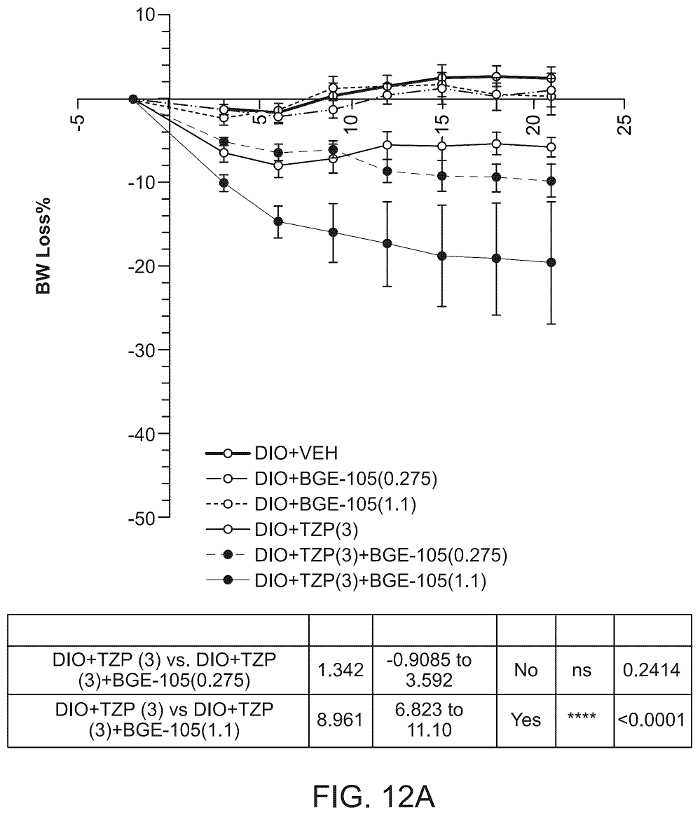

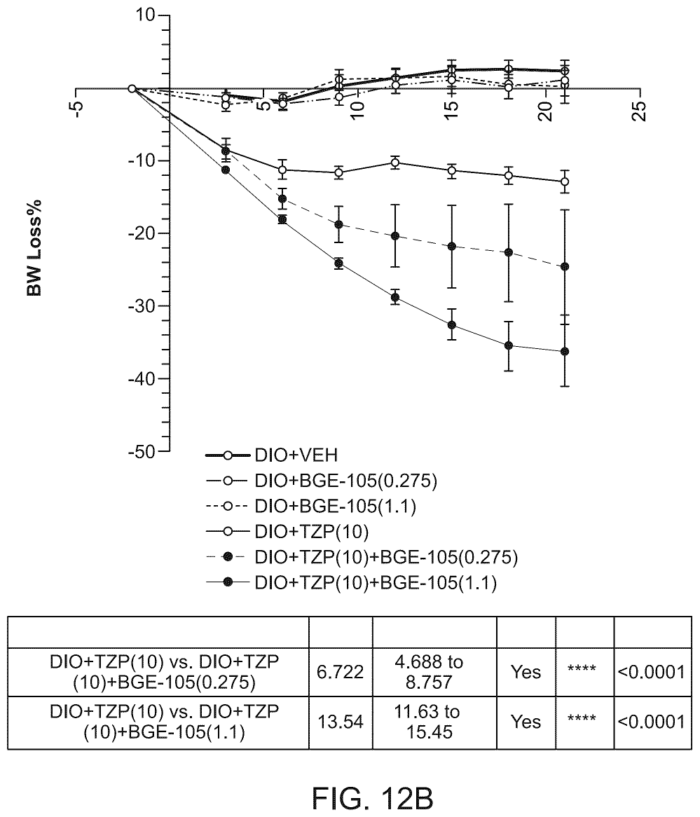

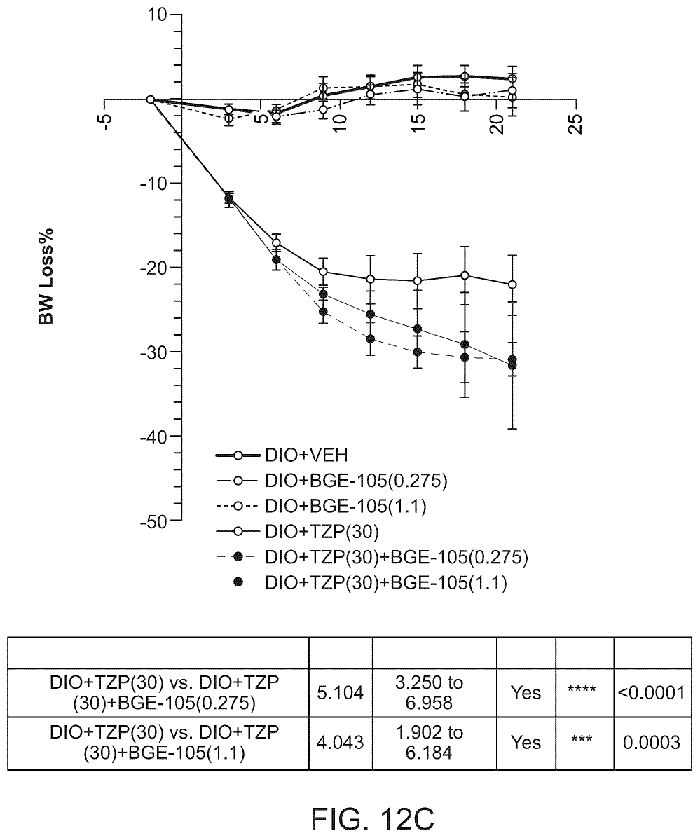

As shown in FIGS. 12A-12C, body weight loss, as a percentage of starting weight, was determined for the various treatment groups.

FIG. 12A shows data from groups treated with tirzepatide at 3 nmol/kg (“TZP(3)”), the lowest tirzepatide dose used in these experiments, and relevant controls. As shown in FIG. 12A, the % body weight loss of Group 2 (DIO+BGE-105 @ 0.275 g/L) (“BGE-105 (0.275)”) and Group 3 (DIO+BGE-105 @ 1.1 g/L) (“BGE-105 (1.1)”) was similar to the control group 1 (DIO+VEH). Group 4 (DIO+TZP 3), Group 5 (DIO+TZP(3)+BGE-105 (0.275)), and Group 6 (DIO+TZP(3)+BGE-105(1.1)) showed increased % body weight loss as compared to the control Group 1. Group 6, with the highest dose of BGE-105, resulted in significant increase in % body weight loss as compared to Group 4 (DIO+TZP (3)), that is, mice treated with tirzepatide alone (p<0.0001). This result was not expected. As shown in Example 2, apelin receptor agonist BGE-105 spared lean muscle from the weight loss caused by the GLP-1 receptor agonist, semaglutide; with weight loss caused by a GLP-1RA thereby limited to loss of fat, total weight loss was expected to be reduced as compared to that seen with treatment with the GLP-1RA, tirzepatide, alone.

FIG. 12B shows data from groups treated with tirzepatide at 10 nmol/kg (“TZP(10)”), and relevant controls. As shown in FIG. 12B, the % body weight loss of Group 2 (DIO+BGE-105 (0.275)) and Group 3 (DIO+BGE-105 (1.1)) is similar to the control group 1 (DIO+VEH). Group 7 (DIO+TZP (10)), Group 8 (DIO+TZP (10)+BGE-105 (0.275)), and Group 9 (DIO+TZP(10)+BGE-105 (1.1)) showed increased % body weight loss as compared to the control Group 1. There was a significant increase in % body weight loss in Group 8 (DIO+TZP (10)+BGE-105 (0.275)), and Group 9 (DIO+TZP(10)+BGE-105 (1.1)) as compared to Group 7 (DIO+TZP(10)) (p<0.0001). This result was not expected. As shown in Example 2, apelin receptor agonist BGE-105 spared lean muscle from the weight loss caused by the GLP-1 receptor agonist, semaglutide; with weight loss caused by a GLP-1RA thereby limited primarily to loss of fat, total weight loss was expected to be reduced as compared to that seen with treatment with the GLP-1RA, tirzepatide, alone.

FIG. 12C shows data from groups treated with tirzepatide at 30 nmol/kg (“TZP(30”) and relevant controls. As shown in FIG. 12C, the % body weight loss of Group 2 (DIO+BGE-105 (0.275)) and Group 3 (DIO+BGE-105 (1.1)) is similar to the control group 1 (DIO+VEH). Group 10 (DIO+TZP (30)), Group 11 (DIO+TZP (30)+BGE-105 (0.275), and Group 12 (DIO+TZP(30)+BGE-105 (1.1)) had increased % body weight loss as compared to the control Group 1. There was a significant increase of % body weight loss in Group 11 (DIO+TZP (30)+BGE-105 (0.275) (p<0.0001)), and Group 12 (DIO+TZP(30)+BGE-105 (1.1)) (p<0.0003) as compared to Group 10 (DIO+TZP(30)) treated mice alone at the highest dose (30 nmol/kg). This result was not expected. As shown in Example 2, apelin receptor agonist BGE-105 spared lean muscle from the weight loss caused by the GLP-1 receptor agonist, semaglutide; with weight loss caused by a GLP-1RA thereby limited primarily to loss of fat, total weight loss was expected to be reduced as compared to that seen with treatment with the GLP-1RA, tirzepatide, alone.

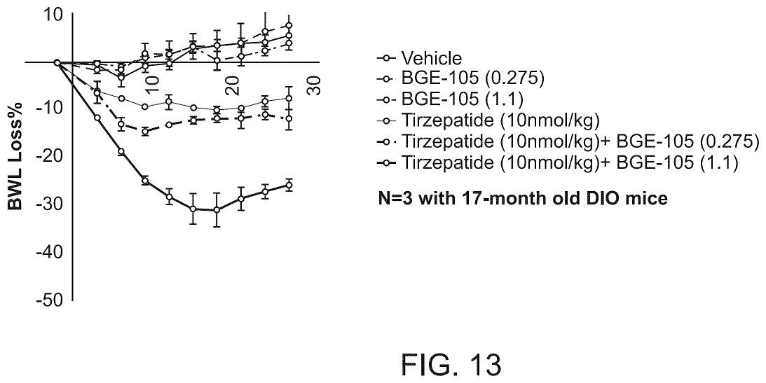

FIG. 13 shows data from the 17-month old mice in the TZP(10) arm of the study. 12 month old C57BI/6 mice are placed on DIO diet (60% fat diet) for 23 weeks during which time their weight increased from 30 g to 48-59 g. Drug treatment was then performed over a further 4 week period during which the DIO diet continued.

FIGS. 12A-12C and FIG. 13 show that BGE-105 treatment in combination with tirzepatide (TZP) results in a significant, dose-dependent, increase in overall weight loss as compared to treatment with tirzepatide alone. The lowest dose of tirzepatide (3nmol/kg) in combination with BGE-105 (Groups 5-6) resulted in a similar % of total body weight loss as did the highest dose of tirzepatide alone (Group 10). Therefore, improvement of total weight loss is shown with treatment of low doses of tirzepatide in combination with BGE-105.

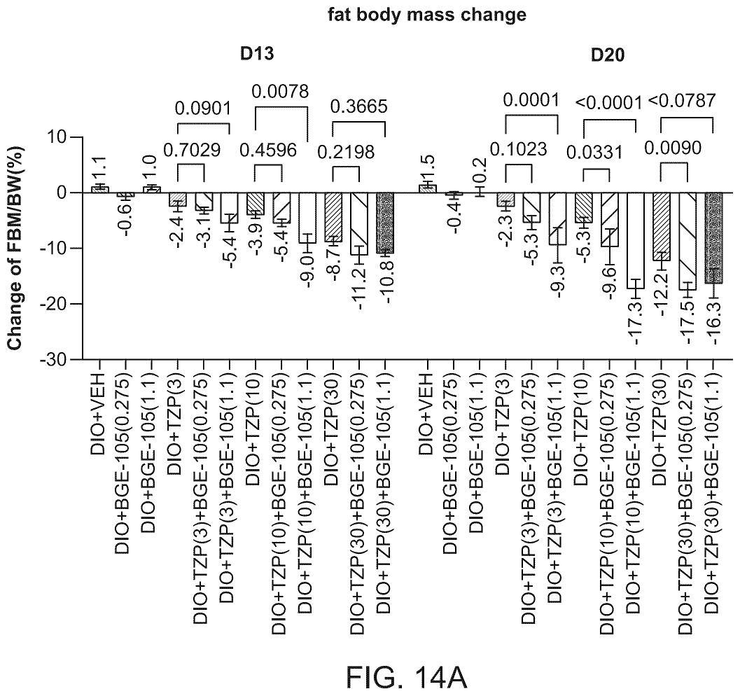

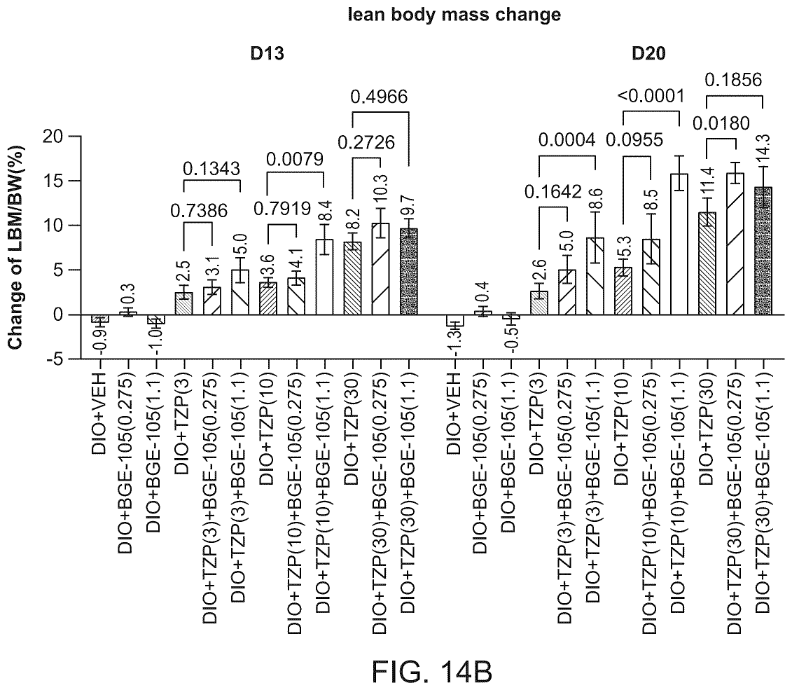

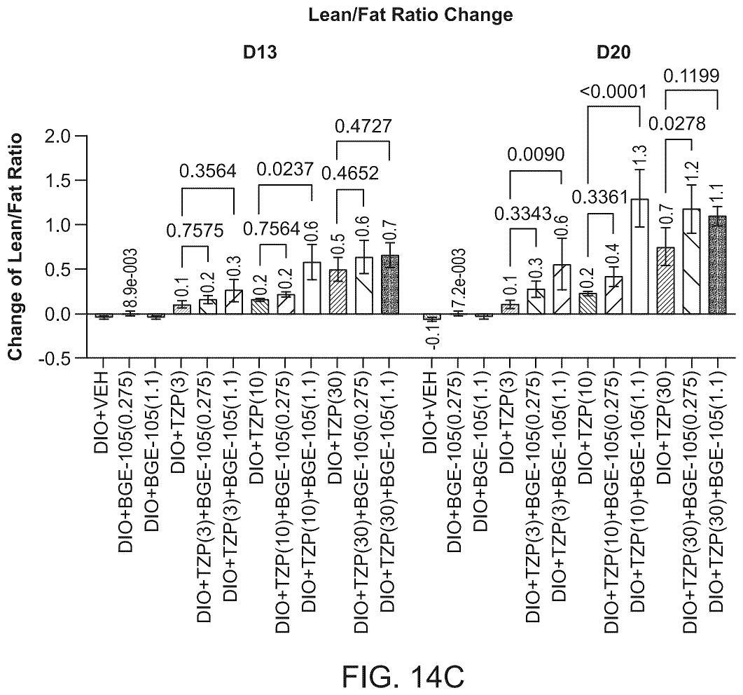

FIGS. 14A-14C show body composition of mice treated in the various treatment groups at days 13 or at day 20. In FIG. 14A, the Y axis charts change of fat body mass over body weight % (FBM/BW). In FIG. 14B, the Y axis shows change of lean body mass over body weight % (LBM/BW). In FIG. 14C, the Y axis shows change of lean body mass over fat ratio (Lean/Fat Ratio). As shown in FIG. 14A, on day 13 and 20, regardless at low dose (3nmol/kg), medium dose (10 nmol/kg), or high dose (30 nmol/kg) of tirzepatide in combination with BGE-105, there is a significant reduction of fat mass percentage as compared to groups treated with tirzepatide alone. This was unexpected. As shown in FIG. 14B, on Day 13 and Day 20 lean body mass percentage change was significantly improved in groups treated with BGE-105 and tirzepatide as compared to groups treated with tirzepatide alone. FIG. 14C shows a significant improvement of lean/fat ratio in groups treated with BGE-105 and tirzepatide as compared to groups treated with tirzepatide alone.

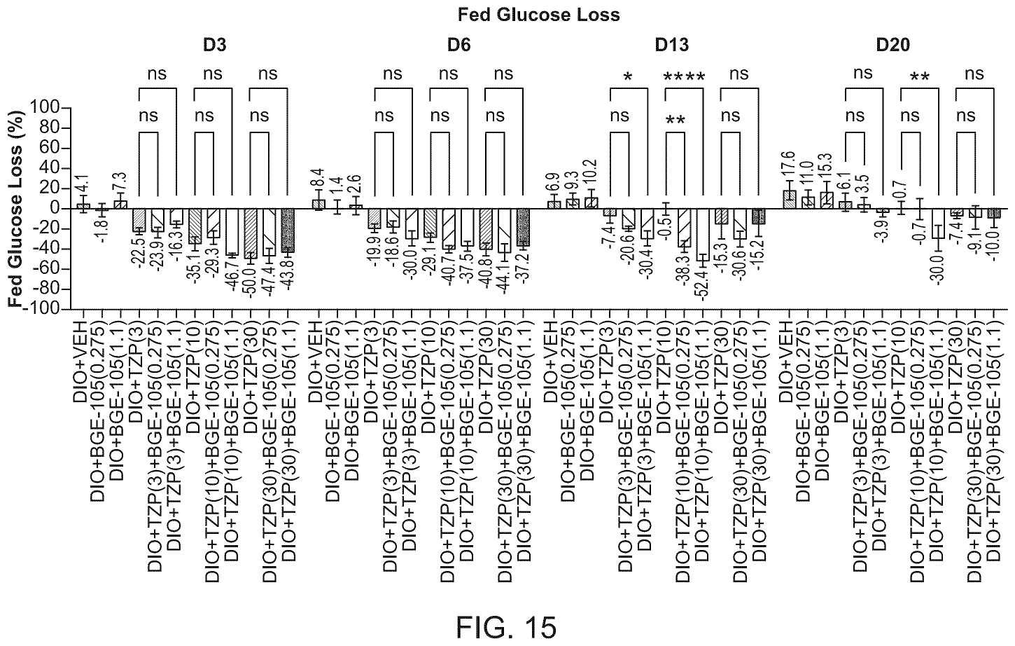

FIG. 15 shows fed glucose loss % of mice treated in the various treatment groups. On Days 13 and 20, there is an additive effect on glucose control in groups treated with tirzepatide and BGE-105, as compared to tirzepatide alone.

### Summary of Results

In comparison GLP-1 receptor agonist (“GLP-1”) monotherapy, we observed that the addition of BGE-105 to GLP-1 therapy with tirzepatide in DIO mice led to:
dose dependent increase in weight loss;
decrease in fat mass percentage;
increase in lean mass percentage;
improvement in body composition (higher lean mass/fat mass ratio); and
benefit over time.

### 6.3. Example 3: Effects of BGE-105 on Weight Loss or Muscle Loss/Function in Aged Diet-Induced Obese (DIO) Mice in Combination with Tirzepatide

A further study was performed to measure the effects of BGE-105 in Diet-Induced Obese (DIO) mice in combination with tirzepatide by adapting the methods described above.

### Study Objectives:

The objective of this study was to compare the effect of BGE-105+GLP-1 analog tirzepatide, using two different concentrations of BGE-105, on total body weight loss and muscle loss/function in DIO mice of 11.5 months in age.

### Methods:

6 week-old (young) C57BL/6 male mice were used at the beginning of this study. Diet for the mice included lean control D12450B, or DIO diet D12492 (60 kcal % fat diet) (Research Diets, Inc.) for 10 months before treatment (45-70 g). The age of the DIO mice at study initiation was 11.5 months, and the study duration was 3 weeks.

Study Groups included the following:
Group 1: Lean Control mice (n=9)
Group 2: DIO mice+vehicle (DIO+VEH) (n=8)
Group 3: DIO mice+BGE-105 (0.275 g/L) (n=6)
Group 4: DIO mice+BGE-105 (1.1 g/L) (n=8)
Group 5: DIO mice+tirzepatide (10 nmol/kg) (n=14)
Group 6: DIO mice+tirzepatide (10 nmol/kg)+BGE-105 (0.275 g/L) (n=14)
Group 7: DIO mice+tirzepatide (10 nmol/kg)+BGE-105 (1.1 g/L) (n=14)

Randomization: baseline body weight (BW), body composition, fed glucose and grid hang. Data were expressed as mean/−SEM, and analyzed using one-way or two-way ANOVA.

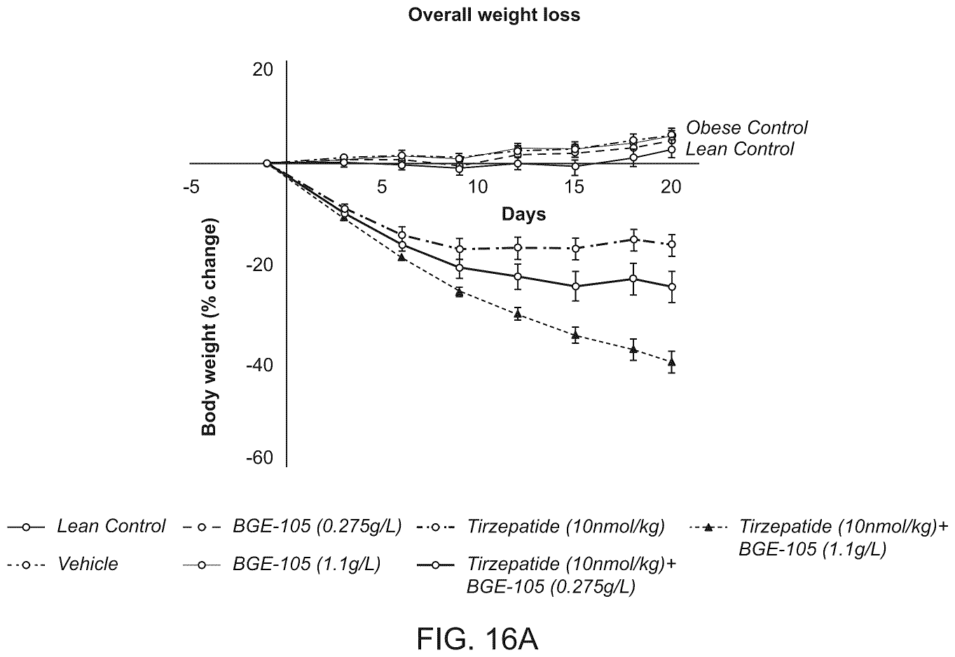

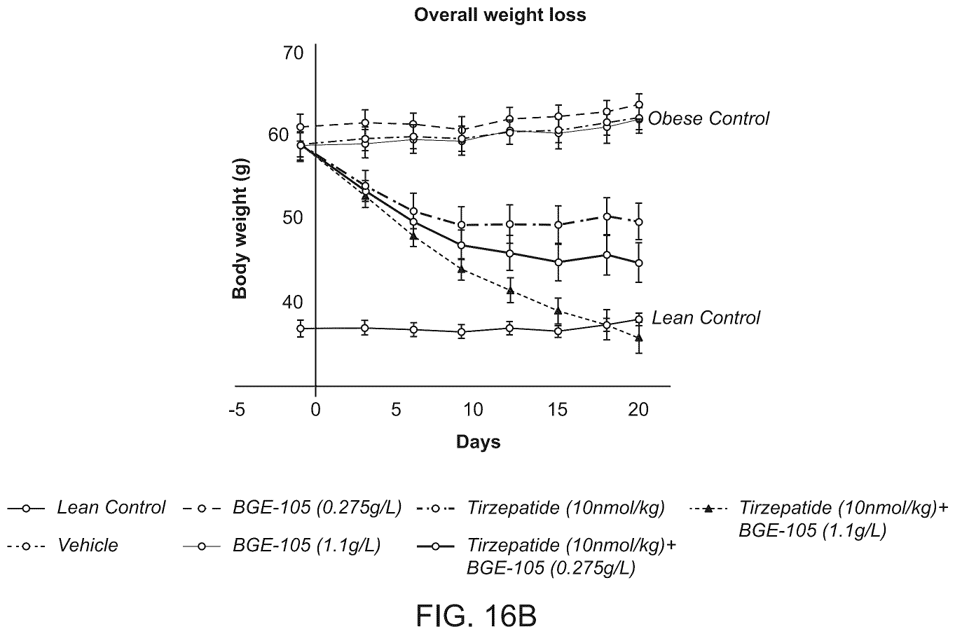

FIGS. 16A-16B show graphs of overall weight loss, including % change of body weight (FIG. 16A) and absolute body weight (g) over time of administration (FIG. 16B). BGE-105 increases overall weight loss with tirzepatide: approx. 40% overall weight loss, with restoration of body weight to that of lean control mice.

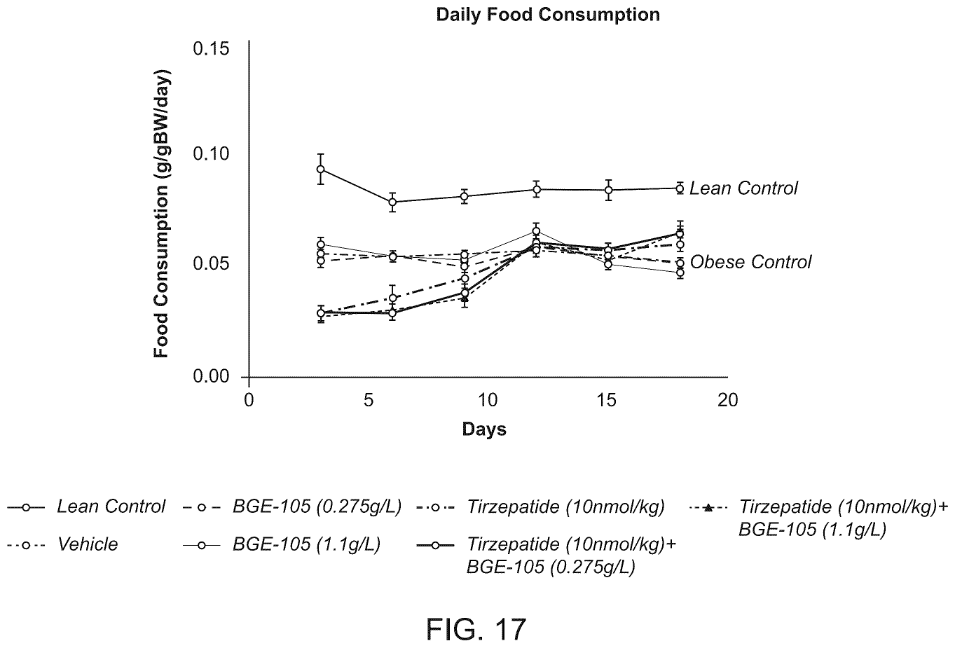

FIG. 17 shows daily food consumption (g/gBW/day) of mice treatment groups. Tirzepatide monotherapy or BGE-105 combination with tirzepatide treatment reduced food daily food consumption compared to the DIO control group.

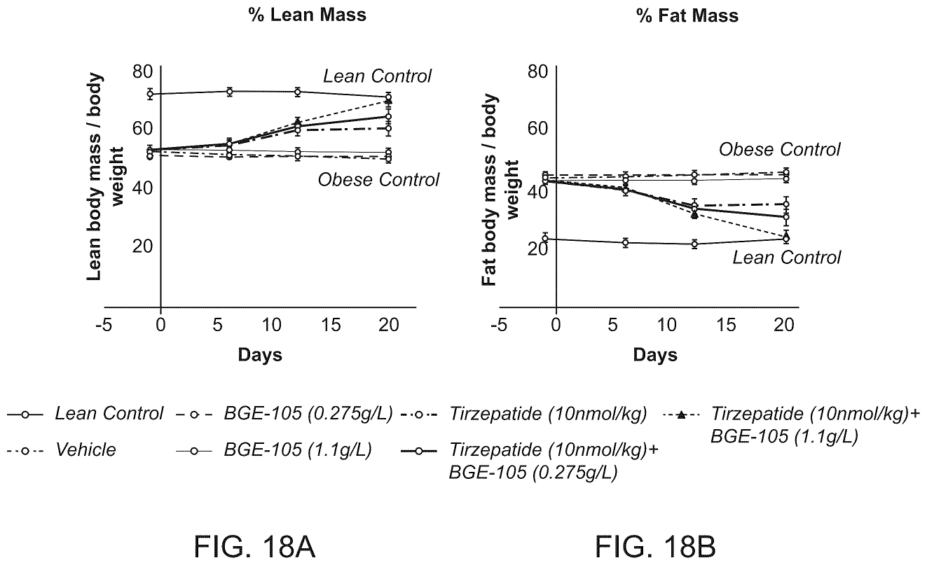

FIGS. 18A-18B illustrate percentage of lean mass and percentage fat mass assessed by Echo MRI. BGE-105 combination with tirzepatide treatment increased percentage of lean mass (FIG. 18A) and reduced percentage of fat mass (FIG. 18B) and restored the levels to that comparable to the lean control group at the end of the treatment (Day 21).

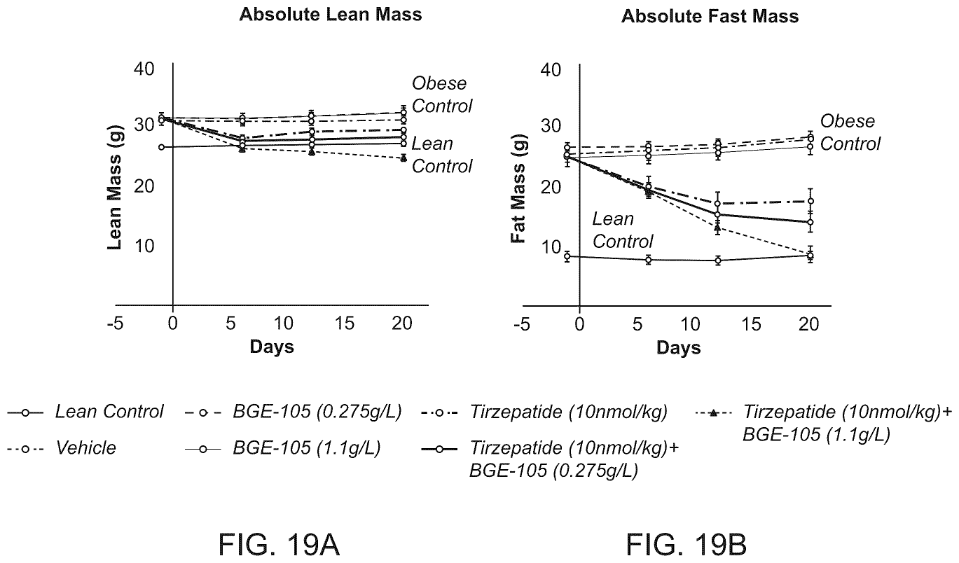

FIGS. 19A-19B illustrate absolute lean mass and absolute fat mass assessed by Echo MRI. BGE-105 combination with tirzepatide treatment dramatically decreased absolute (g) of fat body mass (FIG. 19B). BGE-105 combination with tirzepatide treatment restored absolute fat mass (FIG. 19B) level to that comparable to the lean control group at the end of the treatment (Day 21).

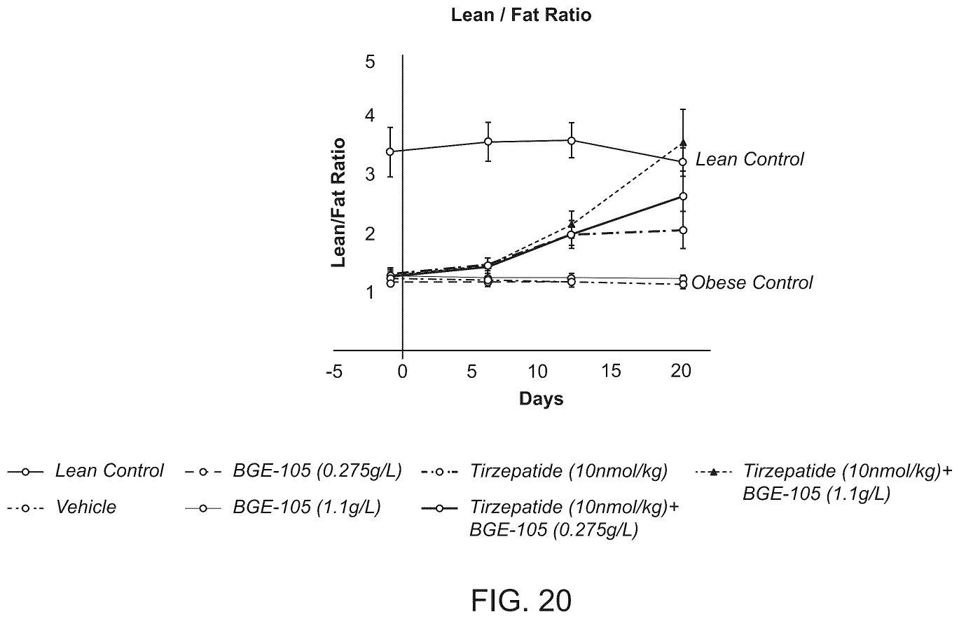

FIG. 20 illustrates lean/fat ratio in mice treatment groups. BGE-105 with tirzepatide had increased lean/fat ratio than tirzepatide alone. The high dose of BGE-105 with tirzepatide combination showed comparable lean/fat ratio to the lean control group at the measurement on day 20.

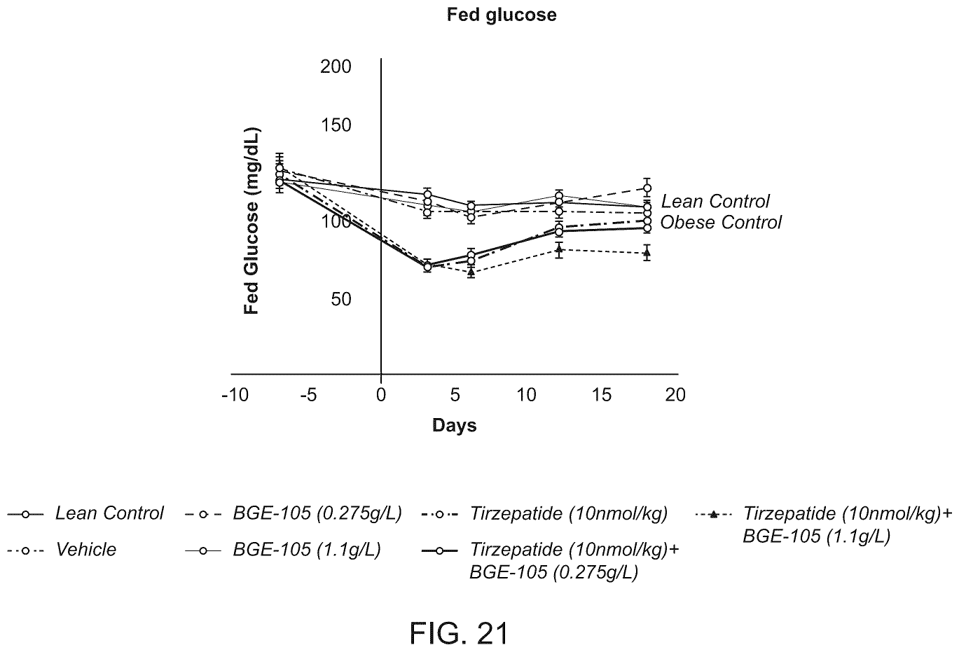

FIG. 21 shows addition of high dose BGE-105 lowered the fed glucose levels achieved with tirzepatide. The data demonstrate BGE-105 administered according to the methods of this disclosure may benefit patients with insulin resistance.

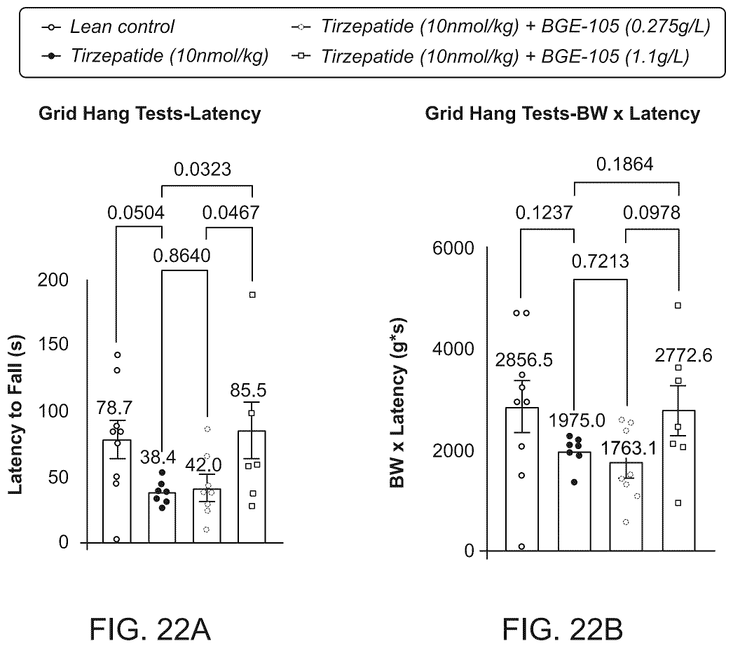

Muscle function of the mice treatment groups were assessed via grid hang tests (FIGS. 22A-22C). FIG. 22C shows an image of the grid hang test. FIG. 22A shows latency of fall (s), and FIG. 22B shows graph of body weight×latency of fall (g*s). The results of FIGS. 22A-B indicate that the combination of high dose BGE-105 and tirzepatide restored muscle function to that of lean controls.

### 6.4. Example 4: Comparison of the Effects of BGE-105 and Tirzepatide with Those of Bimagrumab and Tirzepatide Combination in Obese Mice

### Study Objective(s):

The effects of BGE-105+GLP-1 analog tirzepatide as assessed in Example 3 were compared with the effects of monoclonal antibody bimagrumab+GLP-1 analog tirzepatide, on total body weight loss and muscle loss/function in DIO mice.

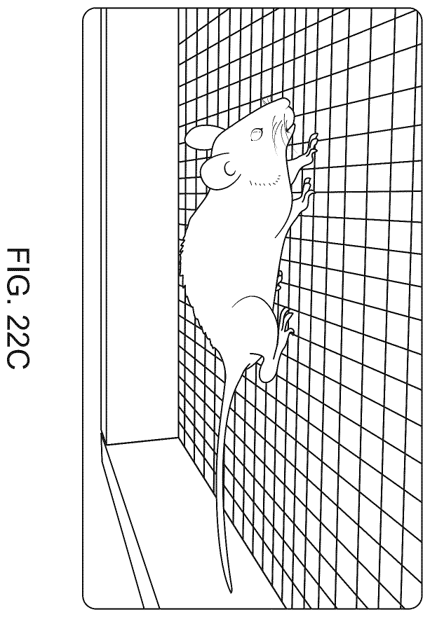

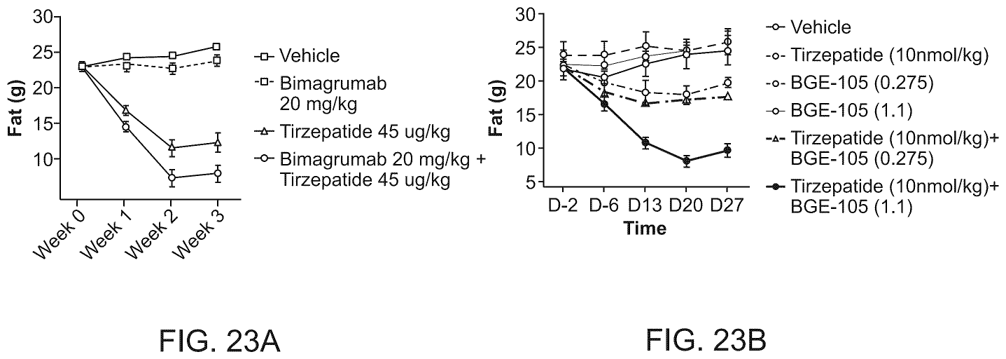

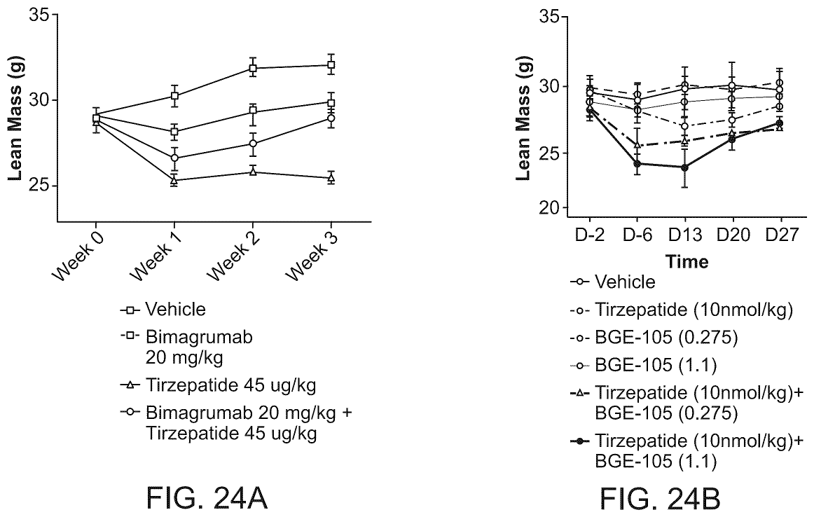

Bimagrumab is a monoclonal antibody that is an activin receptor type 2B (ACVR2B or EC 2.7.11.30) antagonist. The results of studies of the combination of murine bimagrumab with semaglutide or tirzepatide in DIO mouse have been reported (Versanis Obesity Week Presentation, Nov. 2, 2022). An exemplary bimagrumab experiment is summarized in Table 2, and data shown in FIG. 23A and FIG. 24A.

TABLE 2

| Treatment Group | Treatment Condition |
|---|---|
| Vehicle | (pH 8.5 drinking water + vehicle, 20 mM |
| | citrate buffer at pH 7.0, s.c., every 3 days) |
| Bimagrumab (20 mg/kg) | (20 mg/kg) |
| tirzepatide (45 μg/kg) | (45 μg/kg) |
| Bimagrumab (20 mg/kg) + | Bimagrumab (20 mg/kg) + tirzepatide |
| tirzepatide (45 μg/kg) | (45 μg/kg) |

A comparison of the data of Example 3 with the reported bimagrumab data indicates that BGE-105 provides comparable effects on body composition when co-administered with tirzepatide. FIGS. 23A-23B shows that BGE-105 in combination with tirzepatide provides comparable effects on loss of fat mass of BGE-105 as the combination of bimagrumab with tirzepatide in obese mice (FIG. 24A). FIGS. 24A-24B shows that BGE-105 in combination with tirzepatide provides comparable effects on retention of lean muscle mass as the combination of bimagrumab with tirzepatide in obese mice (FIG. 24A).

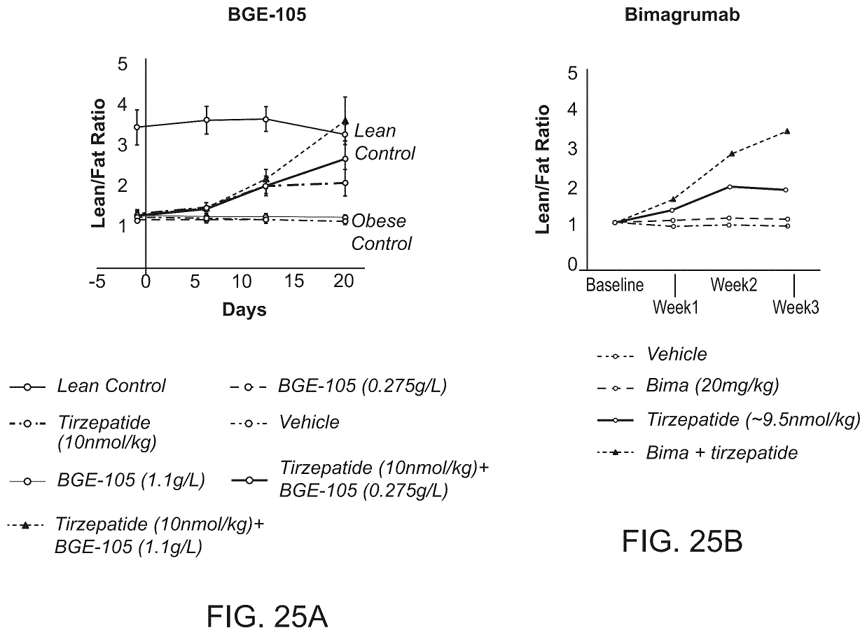

FIGS. 25A-25B shows the monoclonal antibody bimagrumab in combination with tirzepatide provided a comparable lean/fat ratio (FIG. 25B) as BGE-105 (1.1 g/L) in combination with tirzepatide (FIG. 25A). The data demonstrate comparable effects of BGE-105 and bimagrumab on body composition when co-administered with tirzepatide.

### 6.5. Example 5. Effects of Combination of BGE-105 and Tirzepatide in Obese Adult Mice

### Study Objective

To measure the effect of APJ agonist BGE-105 on weight loss, body composition, and blood glucose in younger DIO mice by GLP-1 receptor agonist tirzepatide.

### Methods

Measurements used and justifications include body weight, food intake, water intake, non-fasted blood glucose, body composition by Echo-MRI, and endpoint tissue harvest (blood, muscles, fat, etc.).

C57BL/6 obese male mice from Jax and housed at 6.5-7 months old were fed with a lean control diet D12450B (10 kcal % fat) and a diet-induced obesity (DIO) diet D12492 (60 kcal % fat) (Research Diets, Inc.).

Adult male DIO mice were acclimated to a single house cage for two weeks. Baseline blood samples (100 ul) were collected via tail nick. Body weight, morning non-fasted blood glucose, body composition grid hang tests were measured for randomization. After randomization, mice were given treatment as the following groups (All groups were paired with relative dosing s.c. vehicle or pH 8.5 drinking water with addition of 5 mM Sucralose to insure water consumption despite possible taste alterations from drug).

### Study Groups:

0. Lean ctrl, n=9
1. DIO+VEH, n=8
2. DIO+BGE-105 (0.275 g/L in drug water), n=9
3. DIO+BGE-105 (1.1 g/L in drug water), n=9
4. DIO+tirzepatide (10 nmol/kg), n=8
5. DIO+tirzepatide (10 nmol/kg)+BGE-105 (0.275 g/L), n=9
6. DIO+tirzepatide (10 nmol/kg)+BGE-105 (1.1 g/L), n=9

Tirzepatide (hydrochloride) was purchased from MCE (Cat No. HY—P1731B/CS-0107005, Lot No. 128902). Mw=4849.91, 10 nmol/kg=0.0485 mg/kg, 4 mL/kg, drug concentration: 0.012 mg/mL in s.c. vehicle, s.c. injection every 3 days. BGE-105: 0.275 mg/mL was prepared in drinking water with 5 mM Sucralose, pH 8.5. BGE-105: 1.1 mg/mL was prepared in drinking water with 5 mM Sucralose, pH 8.5.

Body weight, food intake, water intake, body composition and morning non-fasted blood glucose were measured during the entire study. After 15 days of treatment and measurement, mice were taken down to harvest tissues for further downstream analysis. On the day of necropsy, blood plasma and organ/tissue samples were collected. Tissue samples were weighed and processed for further tests. Terminal cardiac blood samples (Heparin plasma) are obtained.

Tissue: Half of the tissue samples are frozen down for molecular biology analysis and the other half are fixed in 10% neutral buffered formalin (or embedding in OTC) for histological analysis.

Fat: Inguinal fat, perigonadal fat, brown fat; Muscle: quadricep, tibialis anterior, gastroc, soleus and extensor digitorum longus (EDL); liver.

### Results

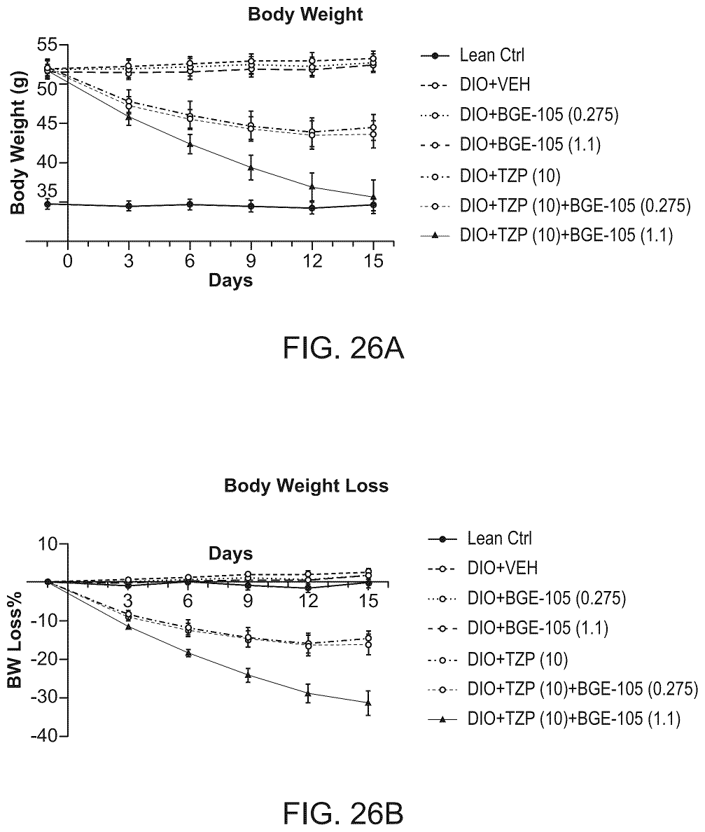

Body Weight: Combination of high dose BGE-105 and tirzepatide significantly decreased more body weight and body weight percentage in obese mice than tirzepatide monotherapy, and restored body weight to the similar level as lean control mice at Day 15. The results are shown in FIGS. 26A-276B and Tables 3-4 below.

TABLE 3
BGE-105 and Tirzepatide body weight in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGE-105 (0.275) | **** | <0.0001 |
| Lean Ctrl vs. DIO + TZP (10) + BGE-105 (1.1) | ns | 0.5574 |
| DIO + VEH vs. DIO + BGE-105 (0.275) | ns | 0.7554 |
| DIO + VEH vs. DIO + BGE-105 (1.1) | Ns | 0.6432 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. DIO + TZP (1O) + BGE-105 (1.1) | **** | <0.0001 |
| DIO + BGE-105 (0.275) vs. DIO + BGE-105 (1.1) | ns | 0.8757 |
| DIO + BGE-105 (0.275) vs. DIO + TZP (10) | **** | <0.0001 |
| DIO + BGE-105 (0.275) vs. DIO + TZP (10) + BGE-105 (0.275) | **** | <0.0001 |
| DIO + BGE-105 (0.275) vs. DIO + TZP (10) + BGE-105 (1.1) | **** | <0.0001 |
| DIO + BGE-105 (1.1) vs. D1O + TZP (10) | **** | <0.0001 |
| DIO + BGE-105 (1.1) vs. DIO + TZP (10) + BGE-105 (0.275) | **** | <0.0001 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.6129 |
| DIO + TZP (10) vs. DIO + TZP (10) + BGE-105 (1.1) | **** | <0.0001 |
| D1O + TZP (10) + BGE-105 (0.275) vs. DIO + TZP | **** | <0.0001 |
| (1O) + BGE-105 (1.1) | | |

TABLE 4
BGE-105 and tirzepatide body weight loss % in adult mice
Day 15 statistics

| | | Individual |
|---|---|---|
| Uncorrected Fisher's LSD | Summary | P Value |
| Lean Ctrl VS, DIO + VEH | ns | 0.1269 |
| Lean Ctrl VS, DIO + BGE-105 (0.275) | ns | 0.2918 |
| Lean Ctrl vs. DIO + BGE-105 (1.1) | ns | 0.2806 |
| Lean Ctrl VS, DIO + TZP (10) | **** | <0.0001 |
| Lean Ctrl VS, DIO + TZP (10) + BGE-105 (0.275) | **** | <0.0001 |
| Lean Ctrl vs. DIO + TZP (10) + BGE-105 (1.1) | **** | <0.0001 |
| DIO + VEH vs. DIO + BGE-105 (0.275) | ns | 0.6134 |
| DIO + VEH vs. DIO + BGE-105 (1.1) | ns | 0.6304 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. DIO + TZP (1O) + BGE-105 (1.1) | **** | <0.0001 |
| DIO + BGE-105 (0.275) vs. DIO + BGE-105 (1.1) | ns | 0.9802 |
| DIO + BGE-105 (0.275) vs. DIO + TZP (10) | **** | <0.0001 |
| DIO + BGE-105 (0.275) vs. DIO + TZP (1O) + BGE-105 (0.275) | **** | <0.0001 |
| DIO + BGE-105 (0.275) vs. D1O + TZP (1O) + BGE-105 (1.1) | **** | <0.0001 |
| DIO + BGE-105 (1.1) vs. DIO + TZP (10) | **** | <0.0001 |
| DIO + BGE-105 (1.1) vs. DIO + TZP (10) + BGE-105 (0.275) | **** | <0.0001 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.4323 |
| DIO + TZP (10) vs. DIO + TZP (10) + BGE-105 (1.1) | **** | <0.0001 |
| DIO + TZP (10) + BGE-105 (0.275) vs. DIO + TZP | **** | <0.0001 |
| (10) + BGE-105 (1.1) | | |

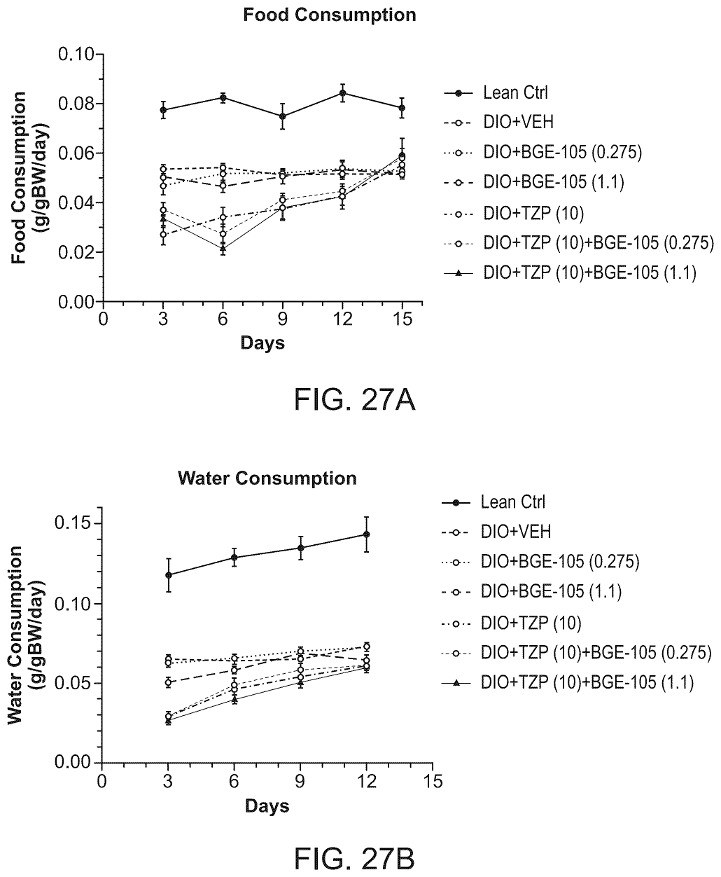

Daily Food Consumption: The food consumptions (g/gBW/day) of BGE-105 and tirzepatide combination groups were not significantly different from tirzepatide monotherapy. The results are shown in FIG. 27A and Table 5 below.

TABLE 5
BGE-105 and Tirzepatide food consumption in adult mice
Day 15 Statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGE-105 (0.275) | **** | <0.0001 |
| Lean Ctrl vs. DIO + TZP (10) + BGE-105 (1.1) | **** | <0.0001 |
| DIO + VEH vs. DIO + BGE-105 (0.275) | ns | 0.7951 |
| DIO + VEH vs. DIO + BGE-105 (1.1) | ns | 0.9884 |
| DIO + VEH vs. DIO + TZP (10) | ns | 0.4768 |
| DIO + VEH vs. DIO + TZP (10) + BGE-105 (0.275) | ns | 0.1913 |
| DIO + VEH vs. DIO + TZP (10) + BGE-105 (1.1) | ns | 0.1417 |
| DIO + BGE-105 (0.275) vs. DIO + BGE-105 (1.1) | ns | 0.8005 |
| DIO + BGE-105 (0.275) vs. DIO + TZP (10) | ns | 0.6365 |
| DIO + BGE-105 (0.275) vs. DIO + TZP (10) + BGE-105 (0.275) | ns | 0.2800 |
| DIO + BGE-105 (0.275) vs. DIO + TZP (10) + BGE-105 (1.1) | ns | 0.2120 |
| DIO + BGE-105 (1.1) vs. DIO + TZP (10) | ns | 0.4731 |
| DIO + BGE-105 (1.1) vs. DIO + TZP (10) + BGE-105 (0.275) | ns | 0.1829 |
| DIO + BGE-105 (1.1) vs. DIO + TZP (10) + BGE-105 (1.1) | ns | 0.1337 |
| DIO + TZP (10) vs. DIO + TZP (10) + BGE-105 (0.275) | ns | 0.5644 |
| DIO + TZP (10) vs. DIO + TZP (10) + BGE-105 (1.1) | ns | 0.4596 |
| DIO + TZP (10) + BGE-105 (0.275) vs. DIO + TZP (10) + | ns | 0.8662 |
| BGE-105 (1.1) | | |

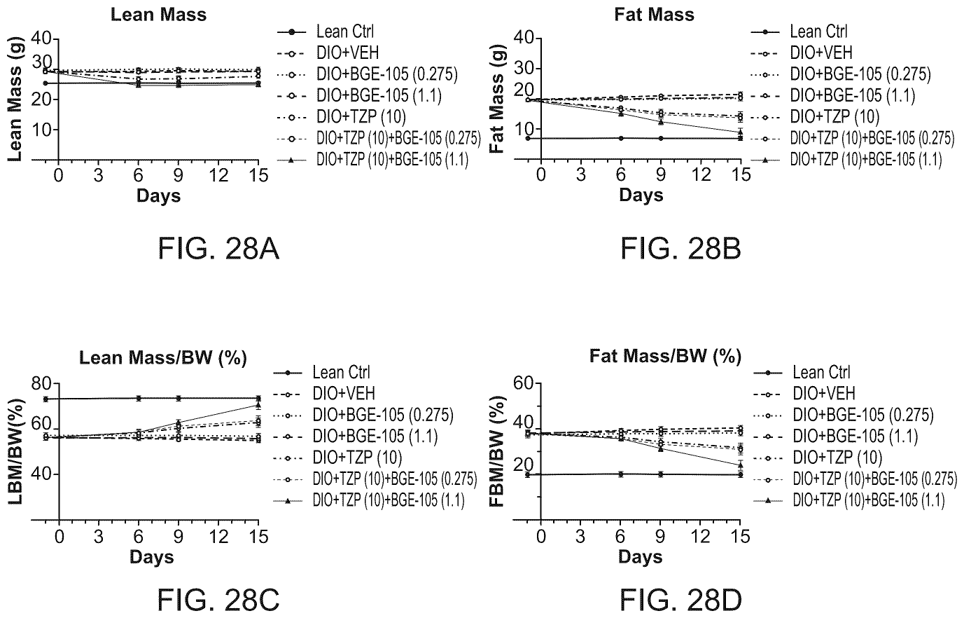

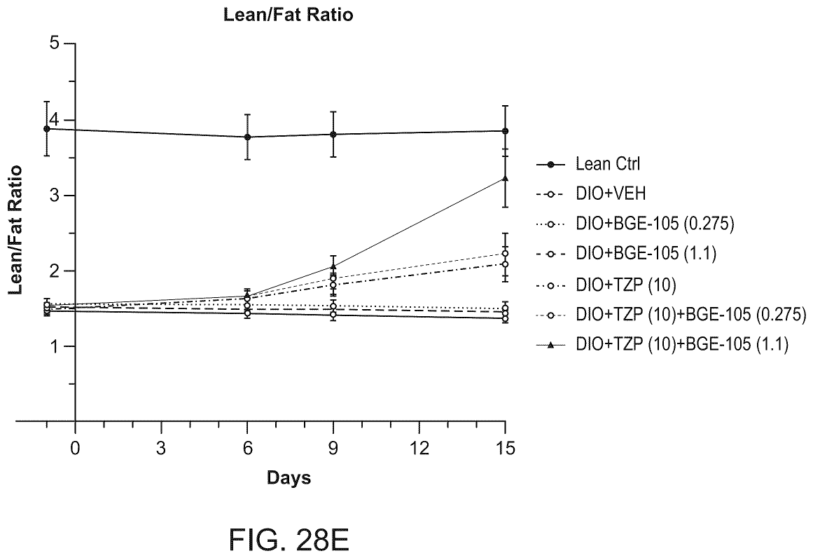

Water Consumption: The trends of water consumption in the BGE-105 and tirzepatide groups were similar to tirzepatide monotherapy at Day 12. The results are shown in FIG. 28B and Table 6 below.

TABLE 6
BGE-105 and tirzepatide water consumption in adult mice
Day 12 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGE-105 (0.275) | **** | <0.0001 |
| Lean Ctrl vs. DIO + TZP (10) + BGE-105 (1.1) | **** | <0.0001 |
| DIO + VEH vs. DIO + BGE-105 (0.275) | ns | 0.9343 |
| DIO + VEH vs. DIO + BGE-105 (1.1) | ns | 0.1€61 |
| DIO + VEH vs. DIO + TZP (10) | ns | 0.0707 |
| DIO + VEH vs. DIO + TZP (10) + BGE-105 (0.275) | ns | 0.0548 |
| DIO + VEH vs. DIO + TZP (10) + BGE-105 (1.1) | * | 0.0396 |
| DIO + BGE-105 (0.275) vs. DIO + BGE-105 (1.1) | ns | 0.1794 |
| DIO + BGE-105 (0.275) vs. DIO + TZP (10) | ns | 0.0754 |
| DIO + BGE-105 (0.275) vs. DIO + TZP (10) + BGE-105 (0.275) | ns | 0.0581 |
| DIO + BGE-105 (0.275) vs. DIO + TZP (10} + BGE-105 (1.1) | * | 0.0417 |
| DIO + BGE-105 (1.1) vs. DIO + TZP (10) | ns | 0.6319 |
| DIO + BGE-105 (1.1) vs. DIO + TZP (10} + BGE-105 (0.275) | ns | 0.5774 |
| DIO + BGE-105 (1.1) vs. DIO + TZP (10) + BGE-105 (1.1) | ns | 0.4836 |
| DIO + TZP (10) vs. DIO + TZP (10) + BGE-105 (0.275) | ns | 0.9509 |
| DIO + TZP (10) vs. DIO + TZP (10} + BGE-105 (1.1) | ns | 0.8409 |
| DIO + TZP (10) + BGE-105 (0.275) vs. DIO + TZP (10) + | ns | 0.8859 |
| BGE-105 (1.1) | | |

Absolute Lean Mass and Fat Mass: Addition of high dose of BGE-105 to Tirzepatide treatment showed significant reduction of absolute fat mass (FIG. 28B), which was more dramatic than the change of absolute lean mass (FIG. 28A). BGE-105 at 1.1 g/L and tirzepatide at 10 nmol/kg combination treatment reduced absolute fat mass (g) to a level close to the lean control group. The results are shown in FIGS. 28A-28B and Tables 7-8 below.

TABLE 7
BGE-105 and tirzepatide absolute lean mass in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | ** | 0.0070 |
| Lean Ctrl vs. DIO + TZP (10) + BGE-105 (0.275) | ** | 0.0098 |
| Lean Ctrl vs. DIO + TZP (10) + BGE-105 (1.1) | ns | 0.5218 |
| DIO + VEH vs. DIO + BGE-105 (0.275) | ns | 0.4608 |
| DIO + VEH vs. DIO + BGE-105 (1.1) | ns | 0.9944 |
| DIO + VEH vs. DIO + TZP (10) | ns | 0.0758 |
| DIO + VEH vs. DIO + TZP (10) + BGE-105 (0.275) | * | 0.0434 |
| DIO + VEH vs. DIO + TZP (10) + BGE-105 (1.1) | **** | <0.0001 |
| DIO + BGE-105 (0.275) vs. DIO + BGE-105 (1.1) | ns | 0.4515 |
| DIO + BGE-105 (0.275) vs. DIO + TZP (10) | * | 0.0107 |
| DIO + BGE-105 (0.275) vs. DIO + TZP (10) + BGE-105 (0.275) | ** | 0.0047 |
| DIO + BGE-105 (0.275) vs. DIO + TZP (10) + BGE-105 (1.1) | **** | <0.0001 |
| DIO + BGE-105 (1.1) vs. DIO + TZP (10) | ns | 0.0667 |
| DIO + BGE-105 (1.1) vs. DIO + TZP (10) + BGE-105 (0.275) | * | 0.0368 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.8451 |
| DIO + TZP (10) vs. DIO + TZP (10) + BGE-105 (1.1) | *** | 0.0010 |
| DIO + TZP (10) + BGE-105 (0.275) vs. DIO + TZP (10) + | ** | 0.0013 |
| BGE-105 (1.1) | | |

TABLE 8
BGE-105 and tirzepatide absolute fat mass in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGE-105 (0.275) | **** | <0.0001 |
| Lean Ctrl ys. DIO + TZP (10) + BGE-105 (1.1) | ns | 0.1339 |
| DIO + VEH vs. DIO + BGE-105 (0.275) | ns | 0.3183 |
| DIO + VEH vs. DIO + BGE-105 (1.1) | ns | 0.4578 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. DIO + TZP (10) + BGE-105 (1.1) | **** | <0.0001 |
| DIO + BGE-105 (0.275) vs. DIO + BGE-105 (1.1) | ns | 0.7919 |
| DIO + BGE-105 (0.275) vs. DIO + TZP (10) | **** | <0.0001 |
| DIO + BGE-105 (0.275) vs. DIO + TZP (10) + BGE-105 (0.275) | **** | <0.0001 |
| DIO + BGE-105 (0.275) vs. DIO + TZP (10) + 8GE-105 (1.1) | **** | <0.0001 |
| DIO + BGE-105 (1.1) vs. DIO + TZP (10) | **** | <0.0001 |
| DIO + BGE-105 (1.1) vs. DIO + TZP (10) + BGE-105 (0.275) | | <0.0001 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.6536 |
| DIO + TZP (10) vs. DIO + TZP (10) + BGE-105 (1.1) | **** | <0.0001 |
| DIO + TZP (10) + BGE-105 (0.275) vs. DIO + TZP (10) + | *** | 0.0002 |
| BGB-105 (1.1) | | |

Lean Mass Percentage: Addition of high dose BGE-105 to tirzepatide treatment showed significant improvement in lean mass percentage in a dose dependent manner. BGE-105 (1.1 mg/mL) and tirzepatide (10 nmol/kg) restored lean mass percentage to a level close to the lean control. The results are shown in FIG. 28C and Table 9 below.

TABLE 9
BGE-105 and Tirzepatide lean mass percentage in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGB-105 (0.275) | **** | <0.0001 |
| Lean Ctrl vs. DIO + TZP (10) + BGE-105 (1.1) | ns | 0.1185 |
| DIO + VEH vs. DIO + BGE-105 (0.275) | ns | 0.3067 |
| DIO + VEH vs. DIO + BGE-105 (1.1) | ns | 0.6223 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. 010 + TZP (10) + BGE-105 (1.1) | **** | <0.0001 |
| DIO + BGE-105 (0.275) vs. DIO + BGE-105 (1.1) | ns | 0.5845 |
| DIO + BGE-105 (0.275) vs. DIO + TZP (10) | ** | 0.0013 |
| DIO + BGE-105 (0.275) vs. DIO + TZP (10) + BGE-105 (0.275) | *** | 0.0001 |
| DIO + BGE-105 (0.275) vs. DIO + TZP (10) + BGE-105 (1.1) | **** | <0.0001 |
| DIO + BGE-105 (1.1) vs. DIO + TZP (10) | *** | 0.0002 |
| DIO + BGE-105 (1.1) vs. DIO + TZP (10) + BGE-105 (0.275) | **** | <0.0001 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.6117 |
| DIO + TZP (10) vs. DIO + TZP (10) + BGE-105 (1.1) | **** | <0.0001 |
| DIO + TZP (10) + BGE-105 (0.275) vs. DIO + TZP (10) + | *** | 0.0001 |
| BGE-105 (1.1) | | |

Fat Mass Percentage: Addition of high dose BGE-105 to tirzepatide treatment showed significant decrease in fat mass percentage. BGE-105 (1.1 mg/mL) and tirzepatide (10 nmol/kg) significantly reduced fat mass percentage to a level close to the lean control. The results are shown in FIG. 28D and Table 10 below.

TABLE 10
BGE-105 and tirzepatide fat mass percentage in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10) + BGE-105 (0.275) | **** | <0.0001 |
| Lean Ctrl vs. DIO + TZP (10) + BGE-105 (1.1) | * | 0.0349 |
| DIO + VEH vs. DIO + BGE-105 (0.275) | ns | 0.2655 |
| DIO + VEH vs. DIO + BGE-105 (1.1) | ns | 0.5159 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. DIO + TZP (10) + BGE-105 (1.1) | **** | <0.0001 |
| DIO + BGE-105 (0.275) vs. DIO + BGE-105 (1.1) | ns | 0.6317 |
| DIO + BGE-105 (0.275) vs. DIO + TZP (10) | *** | 0.0009 |
| DIO + BGE-105 (0.275) vs. DIO + TZP (10) + BGE-105 (0.275) | *** | 0.0002 |
| DIO + BGE-105 (0.275) vs. DIO + TZP (10) + BGE-105 (1.1) | **** | <0.0001 |
| DIO + BGE-105 (1.1) vs. DIO + TZP (10) | *** | 0.0002 |
| DIO + BGE-105 (1.1) vs. DIO + TZP (10) + BGE-105 (0.275) | **** | <0.0001 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | | 0.7120 |
| DIO + TZP (10) vs. DIO + TZP (10) + BGE-105 (1.1) | *** | 0.0001 |
| DIO + TZP (10) + BGE-105 (0.275) vs. DIO + TZP (10) + | ** | 0.0003 |
| BGE-105 (1.1) | | |

Lean/Fat Ratio: Addition of high dose BGE-105 to tirzepatide treatment showed significant increase in lean/fat ratio in a dose dependent manner. BGE-105 (1.1 mg/mL) and tirzepatide (10 nmol/kg) significantly restored lean/fat. The results are shown in FIG. 28E and Table 11 below.

TABLE 11
BGE-105 and tirzepatide lean/fat ratio in adult mice
Day 15 statistics

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (0.275) | **** | <0.0001 |
| Lean Ctrl vs. DIO + BGE-105 (1.1) | **** | <0.0001 |
| Lean Ctrl vs. DIO + TZP (10) | **** | <0.0001 |
| Lean Ctrl vs. DIO + TZP (10} + BGE-105 (0.275) | **** | <0.0001 |
| Lean Ctrl vs. DIO + TZP (10) + BGE-105 (1.1) | * | 0.0128 |
| DIO + VEH vs. DIO + BGE-105 (0.275) | ns | 0.5962 |
| DIO + VEH vs. DIO + BGE-105 (1.1) | ns | 0.8059 |
| DIO + VEH vs. DIO + TZP (10) | ** | 0.0068 |
| DIO + VEH vs. DIO + TZP (10} + BGE-105 (0.275) | ** | 0.0011 |
| DIO + VEH vs. DIO + TZP (10} + BGE-105 (1.1) | **** | <0.0001 |
| DIO + BGE-105 (0.275) vs. DIO + BGE-105 (1.1) | ns | 0.7695 |
| DIO + BGE-105 (0.275) vs. DIO + TZP (10) | * | 0.0235 |
| DIO + BGE-105 (0.275) vs. DIO + TZP (10} + BGE-105 (0.275) | ** | 0.0044 |
| DIO + BGE-105 (0.275) vs. DIO + TZP (10) + BGE-105 (1.1) | **** | <0.0001 |
| DIO + BGE-105 (1.1) vs. DIO + TZP (10) | * | 0.0110 |
| DIO + BGE-105 (1.1) vs. DIO + TZP (10) + BGE-105 (0.275) | ** | 0.0018 |
| DIO + BGE-105 (1.1) vs. DIO + TZP (10) + BGE-105 (1.1) | **** | <0.0001 |
| DIO + TZP (10) vs. DIO + TZP (10) + BGE-105 (0.275) | ns | 0.6121 |
| DIO + TZP (10) vs. DIO + TZP (10) + BGE-105 (1.1) | **** | <0.0001 |
| DIO + TZP (10) + BGE-105 (0.275) vs. DIO + TZP (10) + | **** | <0.0001 |
| BGE-105 (1.1) | | |

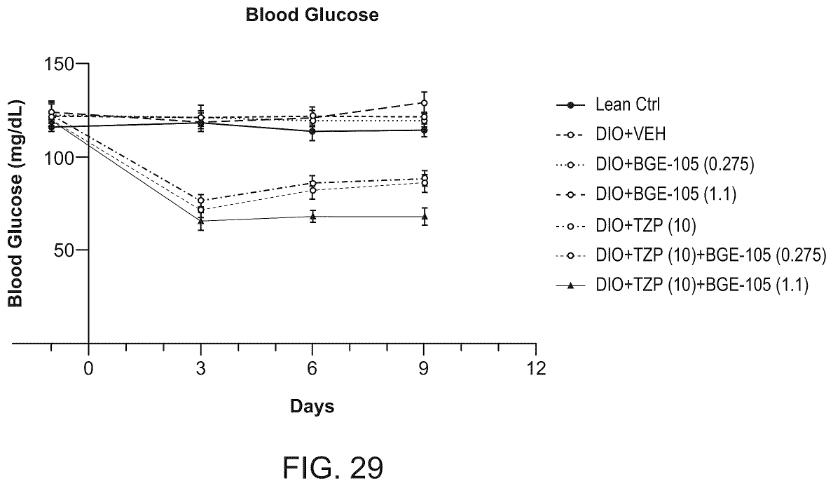

Morning non-fasted blood glucose: On day 9, blood glucose in the tirzepatide+BGE-105 (1.1) group was significantly lower than that in the tirzepatide monotherapy group. The results are shown in FIG. 29 and Table 12 below.

TABLE 12
BGE-105 and tirzepatide blood glucose in adult mice
Day 9 statistics

| | | Individual |
|---|---|---|
| Uncorrected Fisher's LSD | Summary | P Value |
| Lean Ctrl VS, DIO + VEH | ns | 0.3221 |
| Lean Ctrl VS, DIO + BGE-105 (0.275) | ns | 0.4293 |
| Lean Ctrl VS, DIO + BGE-105 (1.1) | * | 0.0352 |
| Lean Ctrl VS, DIO + TZP (10) | *** | 0.0004 |
| Lean Ctrl VS, DIO + TZP (10) + BGE-105 (0.275) | **** | <0.0001 |
| Lean Ctrl VS, DIO + TZP (10) + BGE-105 (1.1) | **** | <0.0001 |
| DIO + VEH vs. DIO + BGE-105 (0.275) | ns | 0.8229 |
| DIO + VEH vs. DIO + BGE-105 (1.1) | ns | 0.2885 |
| DIO + VEH vs. DIO + TZP (10) | **** | <0.0001 |
| DIO + VEH vs. DIO + TZP (10) + BGE-105 (0.275) | **** | <0.0001 |
| DIO + VEH vs. DIO + TZP (10) + BGE-105 (1.1) | **** | <0.0001 |
| DIO + BGE-105 (0.275) vs. DIO + BGE-105 (1.1) | ns | 0.1856 |
| DIO + BGE-105 (0.275) vs. DIO + TZP (10) | **** | <0.0001 |
| DIO + BGE-105 (0.275) vs. DIO + TZP (10) + BGE-105 (0.275) | **** | <0.0001 |
| DIO + BGE-105 (0.275) vs. DIO + TZP (10) + BGE-105 (1.1) | **** | <0.0001 |
| DIO + BGE-105 (1.1) vs. DIO + TZP (10) | **** | <0.0001 |
| DIO + BGE-105 (1.1) vs. DIO + TZP (10)tBGE-105 (0.275) | **** | <0.0001 |
| DIO + BGE-105 (1.1) vs. DIO + TZP (10) + BGE-105 (1.1) | **** | <0.0001 |
| DIO + TZP(10) vs. DIO + TZP (10) + BGE-105 (0.275) | ns | 0.7222 |
| DIO + TZP(10) vs. DIO + TZP (10) + BGE-105 (1.1) | ** | 0.0045 |
| DIO + TZP(10) + BGE-105 (0.275) vs. DIO + TZP (10) + | * | 0.0102 |
| BGE-105 (1.1) | | |

### 6.6. Example 6: Effects of Combination of BAL-1480 and Tirzepatide in Obese Mice

### Study Objective

To measure the effect of another APJ agonist, BAL-1480, compound 13 as described by Narayanan et al. in J. Med. Chem. 2021, 64, 3006-3025, on weight loss or body composition in Diet-Induced Obese Mice by GLP-1 receptor agonist tirzepatide.

### Methods

Measurement used and justifications include: body weight, food intake, water intake, non-fasted blood glucose, and body composition by Echo-MRI.

C57BL/6 obese male mice from Jax and housed to 9-months old were fed with a lean control diet D12450B (10 kcal % fat) and a diet-induced obesity (DIO) diet D12492 (60 kcal % fat) (Research Diets, Inc.).

Male DIO mice were acclimated to a single house cage for two weeks. Body weight, morning non-fasted blood glucose, and body composition were measured for randomization. Target DIO weight range at randomization 51.4-67.5 g. After randomization, mice were given treatment as the following groups (All groups were paired with relative dosing s.c. vehicle (20 mM citrate buffer at pH 7.0, 4 mL/kg) or drinking water with addition of 5 mM Sucralose (5 mM Sucralose, pH 3) to insure water consumption despite possible taste alterations from drug).

Study groups
0. Lean ctrl, n=7
1. DIO+VEH (vehicle), n=7
2. DIO+BAL-1480 (0.25 g/L in drug water), n=7
3. DIO+BAL-1480 (1 g/L in drug water), n=7
4. DIO+tirzepatide (10 nmol/kg), n=7
5. DIO+tirzepatide (10 nmol/kg)+BAL-1480 (0.25 g/L in drug water), n=7
6. DIO+tirzepatide (10 nmol/kg)+BAL-1480 (1 g/L in drug water), n=7

(0) Lean control: age matched lean mice control (5 mM sucralose water+vehicle, s.c., every 3 days); (1) DIO+VEH: diet-induced obese mice treated with vehicle control (5 mM sucralose water, pH 3+vehicle, s.c., every 3 days); (2) DIO+BAL-1480 (0.25 g/L in drug water): diet-induced obese mice treated with BAL-1480 at 0.25 g/L; (3) DIO+BAL-1480 (1 g/L in drug water): diet-induced obese mice treated with BAL-1480 at 1 g/L; (4) DIO+tirzepatide (10 nmol/kg): diet-induced obese mice treated with tirzepatide at 10 nmol/kg, s.c., every 3 days+5 mM sucralose water, pH 3; (5) DIO+tirzepatide (10 nmol/kg)+BAL-1480 (0.25 g/L in drug water): diet-induced obese mice treated with tirzepatide at 10 nmol/kg in combination with BAL-1480 at 0.25 g/L; (6) DIO+tirzepatide (10 nmol/kg)+BAL-1480 (1 g/L in drug water): diet-induced obese mice treated with tirzepatide at 10 nmol/kg in combination with BAL-1480 at 1 g/L.

Tirzepatide (hydrochloride) was purchased from MCE (Cat No. HY—P1731B/CS-0107005). Mw=4849.91, 10 nmol/kg=0.0485 mg/kg, 4 mL/kg, drug concentration: 0.012 mg/mL in s.c. vehicle, every 3 days. BAL-1480 0.25 g/L or 1 g/L was prepared in drinking water with 5 mM Sucralose, pH 3.

Body weight, food intake, water intake, body composition and morning non-fasted blood glucose were measured during the entire study. After 20 days of treatment and measurement, mice were taken down to harvest tissues for further downstream analysis. On the day of necropsy, blood plasma and organ/tissue samples were collected. Tissue samples were weighed and processed for further tests. Terminal cardiac blood samples (Heparin plasma) were obtained.

Tissue: Half of the tissue samples are frozen down for molecular biology analysis and the other half are fixed in 10% neutral buffered formalin (or embedding in OTC) for histological analysis.

Fat: Inguinal fat, perigonadal fat, brown fat; Muscle: quadricep, tibialis anterior, gastrocnemius; liver

Data were expressed as mean±SEM and statistically analyzed by 1-way or 2-way ANOVA

### Results

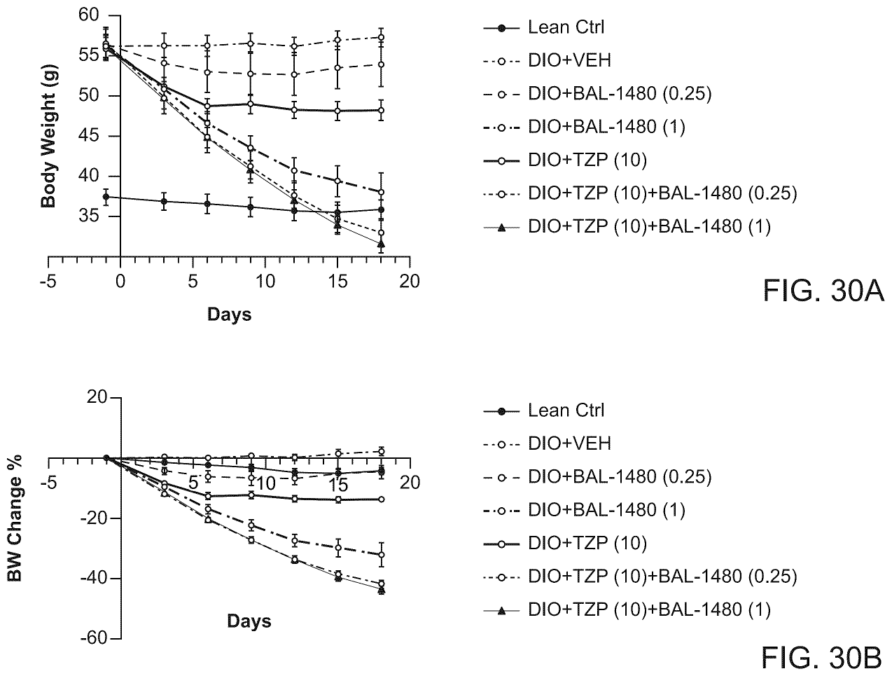

Body Weight: BAL-1480, an APJ agonist, showed a dose-dependent effect on weight loss in obese mice. Combination of BAL-1480 and tirzepatide significantly decreased more body weight in obese mice than tirzepatide monotherapy, and restored it to the similar level as lean control mice after two weeks of treatment. It is noted that the effect of the BAL-1480 and tirzepatide combination was independent of BAL-1480 dosage. The results are shown in FIGS. 30A-30B and Tables 13-14 below. FIGS. 30A-30B show absolute body weight and body weight percentage change in mice treatment groups at Day 18.

TABLE 13
BAL-1480 showed a dose-dependent effect on weight loss in obese mice.

| | | Individual |
|---|---|---|
| Uncorrected Fisher's LSD Day 18. | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | ns | 0.3308 |
| Lean Ctrl vs. DIO + TZP (10) | **** | <0.0001 |
| Lean Ctrl vs. DIO + TZP (10) + BAL-1480 (0.25) | ns | 0.2232 |
| Lean Ctrl vs. DIO + TZP (10) + BAL-1480 (1) | ns | 0.0709 |
| DIO + VEH vs. DIO + BAL-1480 (0.25) | ns | 0.1467 |
| DIO + VEH vs. DIO + BAL-1480 (1) | **** | <0.0001 |
| DIO + VEH vs. DIO + TZP (10) | *** | 0.0001 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | * | 0.0147 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | **** | <0.0001 |
| DIO + BAL-1480 (1) vs. DIO + TZP (10) + BAL-1480 (0.25) | * | 0.0290 |
| DIO + BAL-1480 (1) vs. DIO + TZP (10) + BAL-1480 (1) | ** | 0.0057 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.5544 |
| BAL-1480 (1) | | |

TABLE 14
BA-1480 showed a dose-dependent effect on
body weight change (%) in obese mice.

| | | Individual |
|---|---|---|
| Uncorrected Fisher's LSD Day 18 | Summary | P Value |
| Lean Ctrl vs. DIO + VEH | *** | 0.0008 |
| Lean Ctrl vs. DIO + BAL-1480 (0.25) | ns | 0.8556 |
| Lean Ctrl vs. DIO + BAL-1480 (1) | **** | <0.0001 |
| Lean Ctrl vs. DIO + TZP (10) | **** | <0.0001 |
| Lean Ctrl vs. DIO + TZP (10) + BAL-1480 (0.25) | **** | <0.0001 |
| Lean Ctrl vs. DIO + TZP (10) + BAL-1480 (1) | **** | <0.0001 |
| DIO + VEH vs. DIO + BAL-1480 (0.25) | *** | 0.0004 |
| DIO + VEH vs. DIO + BAL-1480 (1) | **** | <0.0001 |
| DIO + VEH vs. DIO + TZP (10) | **** | <0.0001 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | **** | <0.0001 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | **** | <0.0001 |
| DIO + BAL-1480 (1) vs. DIO + TZP (10) + BAL-1480 (0.25) | **** | <0.0001 |
| DIO + BAL-1480 (1) vs. DIO + TZP (10) + BAL-1480 (1) | **** | <0.0001 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.3435 |
| BAL-1480 (1) | | |

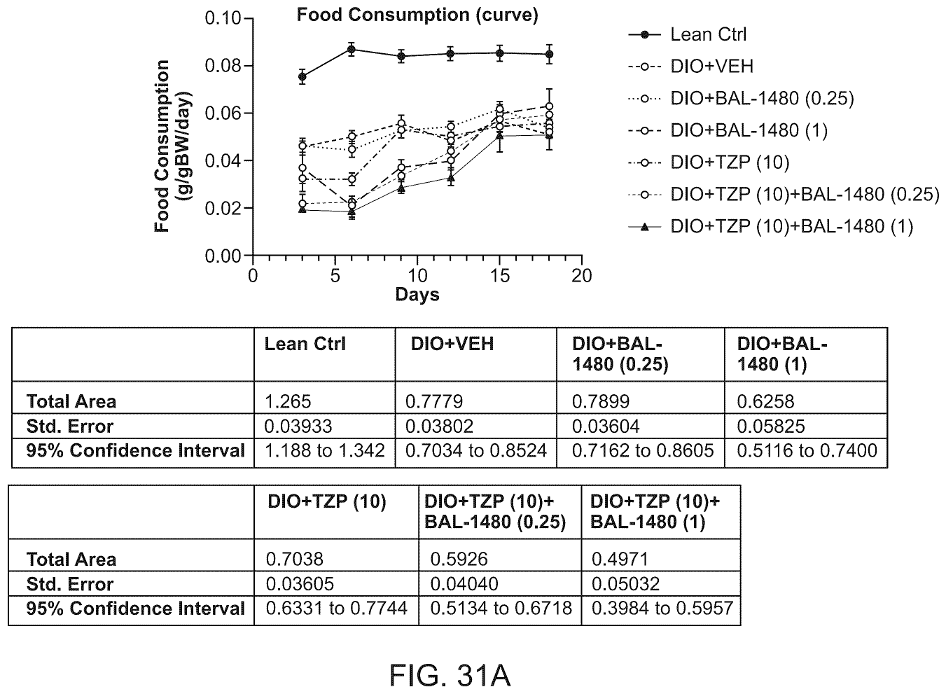

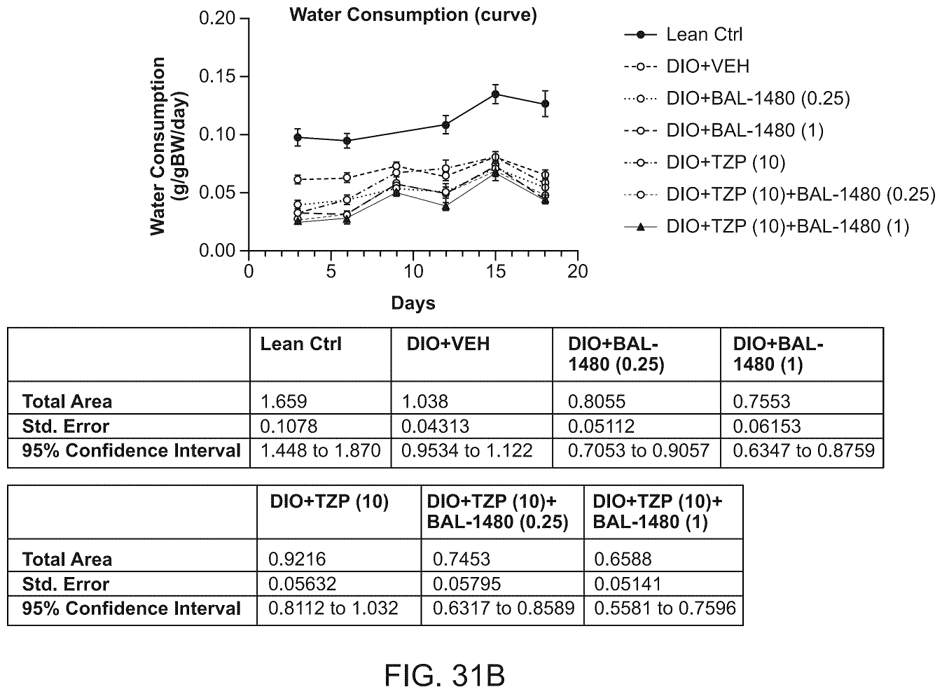

Daily Food Consumption: The food consumptions (g/gBW/day) of BAL-1480 and tirzepatide combination groups (DIO+TZP (10)+BAL-1480 (0.25); DIO+TZP (10)+BAL-1480 (1)) showed significantly less food intake than the tirzepatide monotherapy group by comparing the entire curve up to Day 18. tirzepatide combination with BAL-1480 significantly reduced food intake in a dose dependent fashion (p<0.005). The results are shown in FIG. 31A and Table 15 below. FIG. 31A shows daily food consumptions in mice treatment groups.

TABLE 15
Daily food consumptions (g/gBW/day) in treated mice

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | **** | <0.0001 |
| Lean Ctrl vs. DIO + TZP (10) | **** | <0.0001 |
| Lean Ctrl vs. DIO + TZP (10) + BAL-1480 (0.25) | **** | <0.0001 |
| Lean Ctrl vs. DIO + TZP (10) + BAL-1480 (1) | **** | <0.0001 |
| DIO + VEH vs. DIO + BAL-1480 (0.25) | ns | 0.6676 |
| DIO + VEH vs. DIO + BAL-1480 (1) | **** | <0.0001 |
| DIO + VEH vs. DIO + TZP (10) | * | 0.0188 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | ** | 0.0056 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | ns | 0.1065 |
| DIO + BAL-1480 (1) vs. DIO + TZP (10) + BAL-1480 (0.25) | ns | 0.0983 |
| DIO + BAL-1480 (1) vs. DIO + TZP (10) + BAL-1480 (1) | **** | <0.0001 |
| DIO + TZP (10) vs. DIO + TZP (10) + BAL-1480 (0.25) | ** | 0.0012 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ** | 0.0041 |
| BAL-1480 (1) | | |

Water Consumption: The water consumption in the BAL-1480 groups was significantly lower than the tirzepatide group by comparing the entire curve up to 18 days. The effect of tirzepatide combination with BAL-1480 (DIO+TZP (10)+BAL-1480 (0.25); DIO+TZP (10)+BAL-1480 (1)) on reducing water consumption is dose independent. The results are shown in FIG. 31B and Table 16 below. FIG. 31B shows daily water consumption in mice treatment groups.

TABLE 16
Daily water consumption (g/gBW/day) in treated mice

| | | Individual |
|---|---|---|
| Uncorrected Fisher's LSD | Summary | P Value |
| DIO + VEH vs. DIO + BAL-1480 (0.25) | **** | <0.0001 |
| DIO + VEH vs. DIO + BAL-1480 (1) | **** | <0.0001 |
| DIO + VEH vs. DIO + TZP (10) | *** | 0.0001 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | ns | 0.0801 |
| DIO + BAL-1480 (0.25) vs. DIO + TZP (10) | ** | 0.0094 |
| DIO + BAL-1480 (0.25) vs. DIO + TZP (10) + BAL-1480 (0.25) | * | 0.0391 |
| DIO + BAL-1480 (0.25) vs. DIO + TZP (10) + BAL-1480 (1) | **** | <0.0001 |
| DIO + BAL-1480 (1) vs. DIO + TZP (10) | **** | <0.0001 |
| DIO + BAL-1480 (1) vs. DIO + TZP (10) + BAL-1480 (0.25) | ns | 0.7513 |
| DIO + BAL-1480 (1) vs. DIO + TZP (10) + BAL-1480 (1) | * | 0.0158 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | * | 0.0356 |
| BAL-1480 (1) | | |

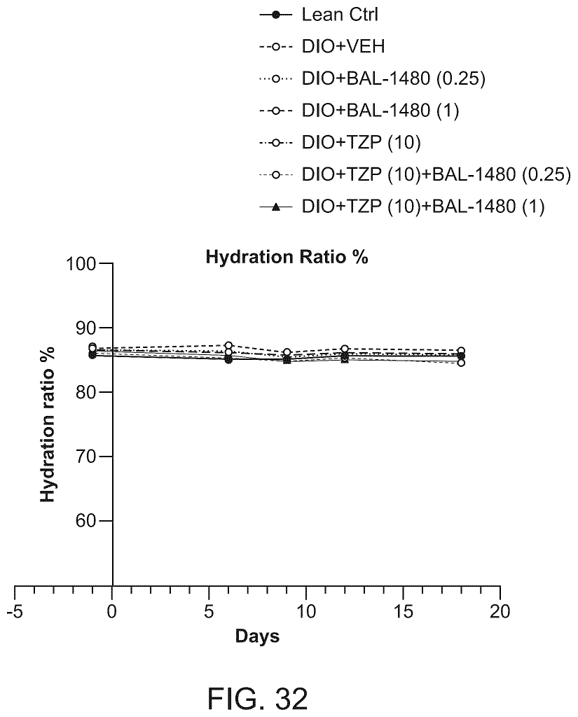

Hydration Ratio (%): For normal animals, the hydration ratio (Total Water−Free Water)/Lean is typically within a few percent of 80%. All of the groups had hydration ratios within the normal range. FIG. 32 shows hydration ratio in treatment groups.

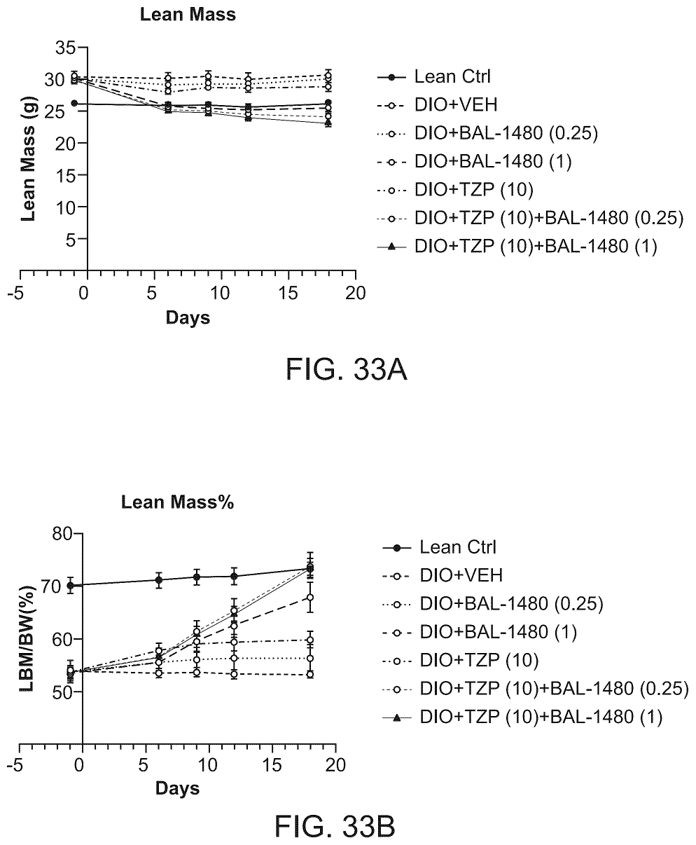

Absolute Lean Mass and Fat Mass: BAL-1480 monotherapy at 1 g/L and both combination groups (DIO+TZP (10)+BAL-1480 (0.25); DIO+TZP (10)+BAL-1480 (1)) showed significant reduction of absolute fat mass in comparison with monotherapy of tirzepatide, which was more dramatic than the change of absolute lean mass. The effects of tirzepatide and BAL-1480 on lean mass and lean mass percentage are dose independent. FIGS. 33A-33B and Table 17 show absolute lean mass and lean mass percentage.

TABLE 17
Absolute lean mass in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | ns | 0.3828 |
| Lean Ctrl vs. DIO + TZP (10) | ** | 0.0022 |
| Lean Ctrl vs. DIO + TZP (10) + BAL-1480 (0.25) | * | 0.0129 |
| Lean Ctrl vs. DIO + TZP (10) + BAL-1480 (1) | *** | 0.0002 |
| DIO + VEH vs. DIO + BAL-1480 (0.25) | ns | 0.5131 |
| DIO + VEH vs. DIO + BAL-1480 (1) | **** | <0.0001 |
| DIO + VEH vs. DIO + TZP (10) | * | 0.0329 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | ns | 0.1373 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | **** | <0.0001 |
| DIO + BAL-1480 (1) vs. DIO + TZP (10) + BAL-1480 (0.25) | ns | 0.1039 |
| DIO + BAL-1480 (1) vs. DIO + TZP (10) + BAL-1480 (1) | ** | 0.0047 |
| DIO + TZP (10) vs. DIO + TZP (10) + BAL-1480 (0.25) | **** | <0.0001 |
| DIO + TZP (10) vs. DIO + TZP (10) + BAL-1480 (1) | ***** | <0.0001 |
| DIO + TZP (10) + BAL-1480 (0.25) vs. DIO + TZP (10) + | ns | 0.2230 |
| BAL-1480 (1) | | |

Lean Mass Percentage: BAL-1480 monotherapy at 1 g/L and both combination groups (i.e., BAL-1480 at 0.25 g/L and Tirzepatide; BAL-1480 at 1 g/L and tirzepatide) in showed significant increase of lean mass percentage in comparison with monotherapy of Tirzepatide. The combination groups were able to restore the lean mass percentage to the similar level of lean control mice. It is noted that the effect of BAL-1480 is dosage independent in the combination groups. The results are shown FIG. 33B and Table 18 below.

TABLE 18
Lean mass percentage in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | * | 0.0220 |
| Lean Ctrl vs. DIO + TZP (10) | **** | <0.0001 |
| Lean Ctrl vs. DIO + TZP (10) + BAL-1480 (0.25) | ns | 0.8811 |
| Lean Ctrl vs. DIO + TZP (10) + BAL-1480 (1) | ns | 0.9312 |
| DIO + VEH vs. DIO + BAL-1480 (0.25) | ns | 0.2109 |
| DIO + VEH vs. DIO + BAL-1480 (1) | **** | <0.0001 |
| DIO + VEH vs. DIO + TZP (10) | ** | 0.0078 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | ns | 0.1537 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | ** | 0.0011 |
| DIO + BAL-1480 (1) vs. DIO + TZP (10) + BAL-1480 (0.25) | * | 0.0148 |
| DIO + BAL-1480 (1) vs. DIO + TZP (10) + BAL-1480 (1) | * | 0.0274 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.8135 |
| BAL-1480 (1) | | |

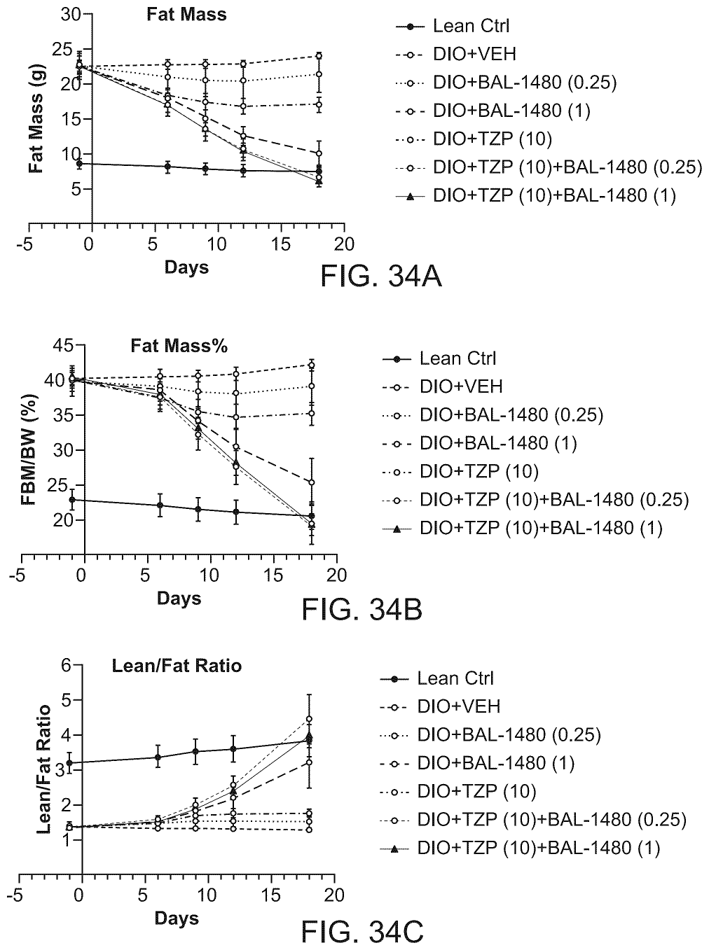

FIG. 34A and the Table 19 below show BAL-1480 monotherapy at 1 g/L and both combination groups showed significant reduction of absolute fat mass in comparison with monotherapy of tirzepatide. The effect of BAL-1480 and tirzepatide combinations on reducing absolute fat mass is dose independent of BAL-1480.

TABLE 19
Absolute fat mass in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | ns | 0.1903 |
| Lean Ctrl vs. DIO + TZP (10) | **** | <0.0001 |
| Lean Ctrl vs. DIO + TZP (10) + BAL-1480 (0.25) | ns | 0.6580 |
| Lean Ctrl vs. DIO + TZP (10) + BAL-1480 (1) | ns | 0.5003 |
| DIO + VEH vs. DIO + BAL-1480 (0.25) | ns | 0.1732 |
| DIO + VEH vs. DIO + BAL-1480 (1) | **** | <0.0001 |
| DIO + VEH vs. DIO + TZP (10) | *** | 0.0004 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | * | 0.0248 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | *** | 0.0004 |
| DIO + BAL-1480 (1) vs. DIO + TZP (10) + BAL-1480 (0.25) | ns | 0.0804 |
| DIO + BAL-1480 (1) vs. DIO + TZP (10) + BAL-1480 (1) | * | 0.0480 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.8168 |
| BAL-1480 (1) | | |

Fat Mass Percentage: BAL-1480 monotherapy at 1 g/L and both combination groups showed significant decrease of fat mass percentage in comparison with monotherapy of tirzepatide. The combination groups were able to restore the fat mass percentage to the similar level of lean control mice. The effect of BAL-1480 and tirzepatide combinations on reducing fat mass percentage is dose independent of BAL-1480. The results are shown in FIG. 34B and the Table 20 below.

TABLE 20
Fat mass percentage in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | ns | 0.0761 |
| Lean Ctrl vs. DIO + TZP (10) | **** | <0.0001 |
| Lean Ctrl vs. DIO + TZP (10) + BAL-1480 (0.25) | ns | 0.6395 |
| Lean Ctrl vs. DIO + TZP (10) + BAL-1480 (1) | ns | 0.6397 |
| DIO + VEH vs. DIO + BAL-1480 (0.25) | ns | 0.2504 |
| DIO + VEH vs. DIO + BAL-1480 (1) | **** | <0.0001 |
| DIO + VEH vs. DIO + TZP (10) | ** | 0.0097 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | ns | 0.1464 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | *** | 0.0003 |
| DIO + BAL-1480 (1) vs. DIO + TZP (10) + BAL-1480 (0.25) | * | 0.0254 |
| DIO + BAL-1480 (1) vs. DIO + TZP (10) + BAL-1480 (1) | * | 0.0254 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.9999 |
| BAL-1480 (1) | | |

Lean/Fat Ratio: BAL-1480 monotherapy at 1 g/L and both combination groups showed significant increase of lean/fat ratio in comparison with monotherapy of tirzepatide. The combination groups were able to restore the lean/fat ratio to the similar level of lean control mice. The effect of BAL-1480 and tirzepatide combinations on lean/fat ratio is dose independent of BAL-1480. The results are shown in FIG. 34C and Table 21 below.

TABLE 21
Lean/fat ratio in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BAL-1480 (0.25) | **** | <0.0001 |
| Lean Ctrl vs. DIO + BAL-1480 (1) | ns | 0.0987 |
| Lean Ctrl vs. DIO + TZP (10) | **** | <0.0001 |
| Lean Ctrl vs. DIO + TZP (10) + BAL-1480 (0.25) | ns | 0.1065 |
| Lean Ctrl vs. DIO + TZP (10) + BAL-1480 (1) | ns | 0.7426 |
| DIO + VEH vs. DIO + BAL-1480 (0.25) | ns | 0.5206 |
| DIO + VEH vs. DIO + BAL-1480 (1) | **** | <0.0001 |
| DIO + VEH vs. DIO + TZP (10) | ns | 0.2089 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | ns | 0.5379 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | *** | 0.0001 |
| DIO + BAL-1480 (1) vs. DIO + TZP (10) + BAL-1480 (0.25) | ** | 0.0012 |
| DIO + BAL-1480 (1) vs. DIO + TZP (10) + BAL-1480 (1) | * | 0.0482 |
| DIO + TZP (10) vs. DIO + TZP (10) + BAL-1480 (0.25) | **** | <0.0001 |
| DIO + TZP (10) vs. DIO + TZP (10) + BAL-1480 (1) | **** | <0.0001 |
| DIO + TZP (10) + BAL-1480 (0.25) vs. DIO + TZP (10) + | ns | 0.1977 |
| BAL-1480 (1) | | |

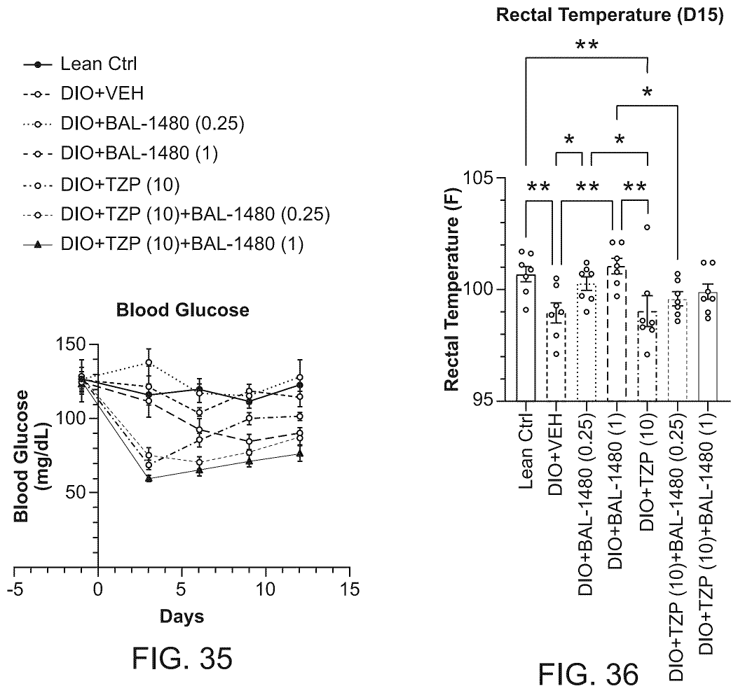

Morning non-fasted blood glucose: blood glucose in the BAL-1480 and tirzepatide combination groups were significantly lower than that in the Tirzepatide monotherapy group. The results are shown in FIG. 35 and Table 22 below.

TABLE 22
Blood glucose in treated mice

| | | Individual |
|---|---|---|
| Uncorrected Fisher's LSD | Summary | P Value |
| Lean Ctrl vs. DIO + VEH | ns | 0.5899 |
| Lean Ctrl vs. DIO + BAL-1480 (0.25) | ns | 0.2275 |
| Lean Ctrl vs. DIO + BAL-1480 (1) | **** | <0.0001 |
| Lean Ctrl vs. DIO + TZP (10) | **** | <0.0001 |
| Lean Ctrl vs. DIO + TZP (10) + BAL-1480 (0.25) | **** | <0.0001 |
| Lean Ctrl vs. DIO + TZP (10) + BAL-1480 (1) | **** | <0.0001 |
| DIO + VEH vs. DIO + BAL-1480 (0.25) | ns | 0.0815 |
| DIO + VEH vs. DIO + BAL-1480 (1) | *** | 0.0002 |
| DIO + VEH vs. DIO + TZP (10) | **** | <0.0001 |
| DIO + VEH vs. DIO + TZP (10) + BAL-1480 (0.25) | **** | <0.0001 |
| DIO + VEH vs. DIO + TZP (10) + BAL-1480 (1) | **** | <0.0001 |
| DIO + BAL-1480 (0.25) vs. DIO + BAL-1480 (1) | **** | <0.0001 |
| DIO + BAL-1480 (0.25) vs. DIO + TZP (10) | **** | <0.0001 |
| DIO + BAL-1480 (0.25) vs. DIO + TZP (10) + BAL-1480 (0.25) | **** | <0.0001 |
| DIO + BAL-1480 (0.25) vs. DIO + TZP (10) + BAL-1480 (1) | **** | <0.0001 |
| DIO + BAL-1480 (1) vs. DIO + TZP (10) | ns | 0.3590 |
| DIO + BAL-1480 (1) vs. DIO + TZP (10) + BAL-1480 (0.25) | ** | 0.0037 |
| DIO + BAL-1480 (1) vs. DIO + TZP (10) + BAL-1480 (1) | **** | <0.0001 |
| DIO + TZP (10) vs. DIO + TZP (10) + BAL-1480 (0.25) | * | 0.0448 |
| DIO + TZP (10) vs. DIO + TZP (10) + BAL-1480 (1) | *** | 0.0002 |
| DIO + TZP (10) + BAL-1480 (0.25) vs. DIO + TZP (10) + | ns | 0.0750 |
| BAL-1480 (1) | | |

Rectal Temperature: The rectal temperature was measured in the afternoon on day 15. Monotherapy of BAL-1480 showed a dose-dependent increase of body temperature than the VEH group. The results are shown in FIG. 36. *=zp<0.05; **=p<0.01.

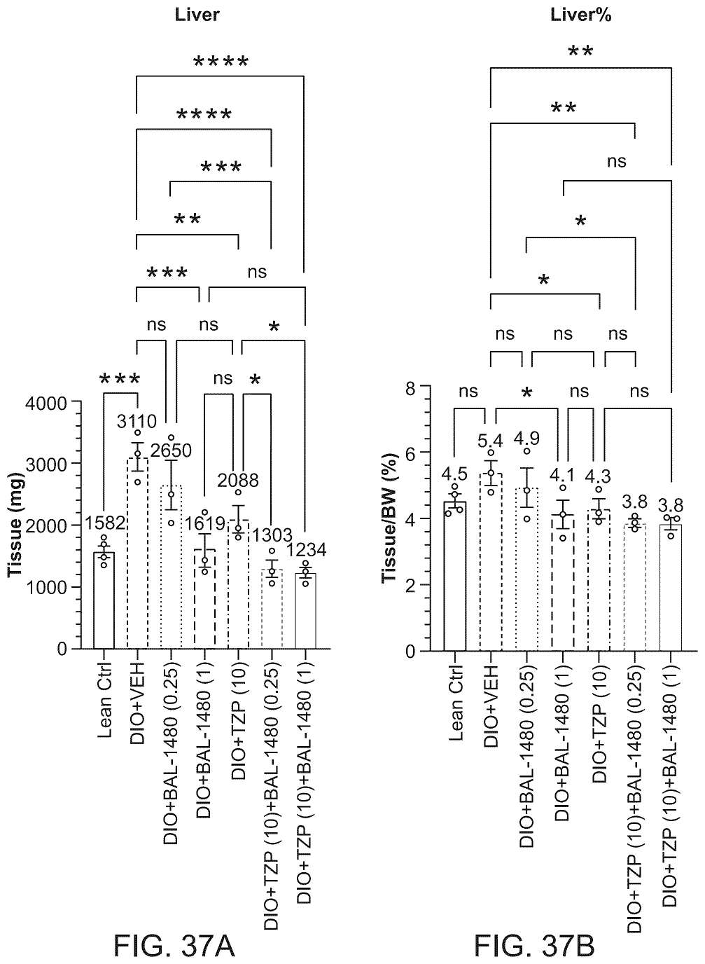

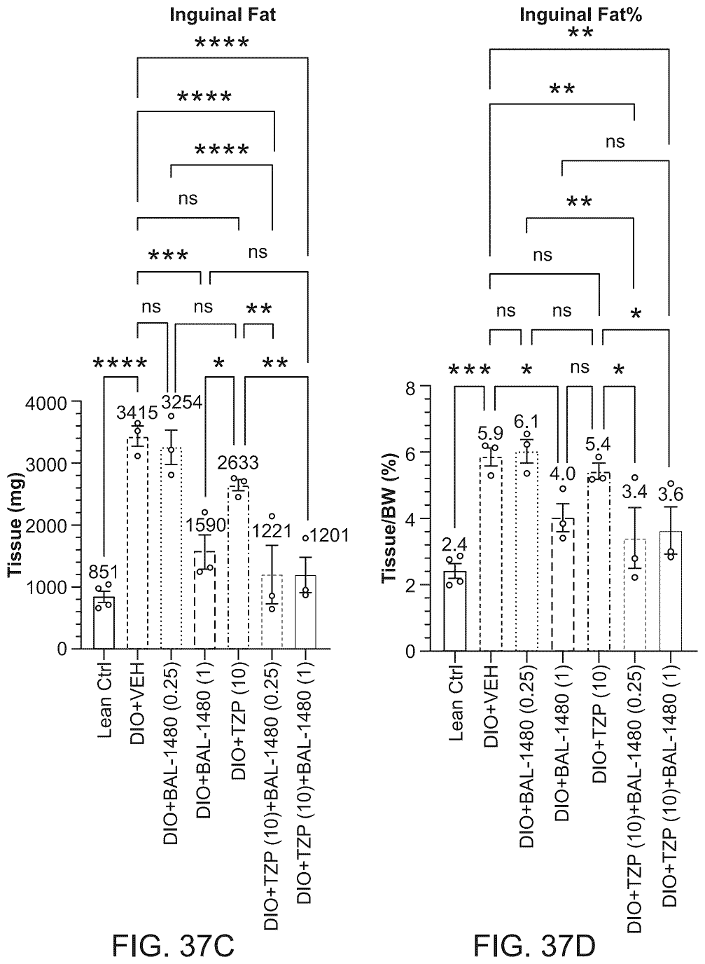

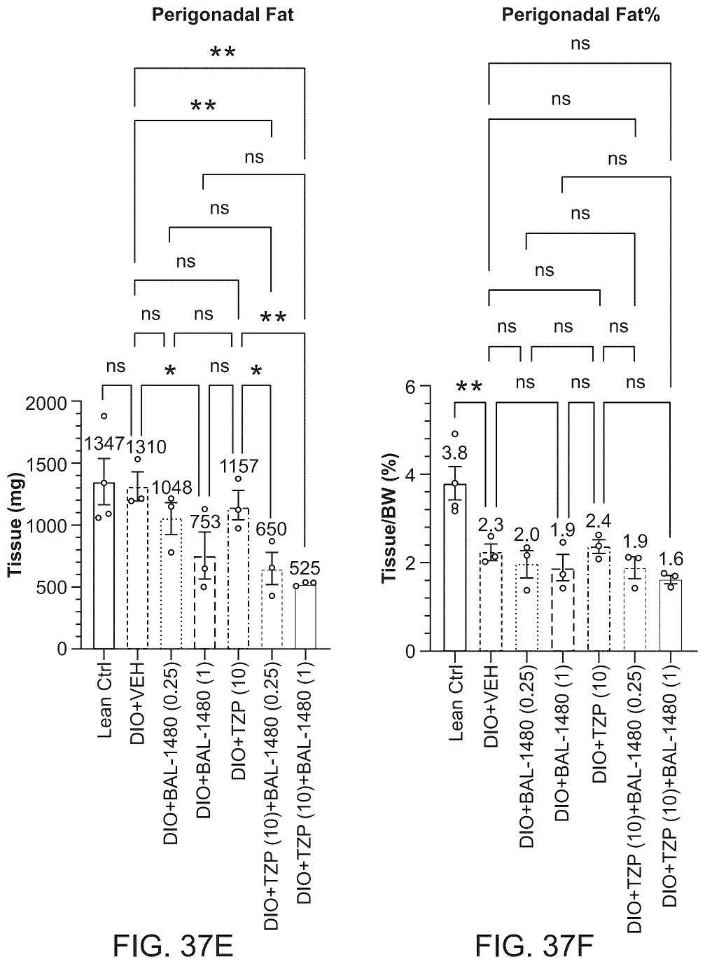

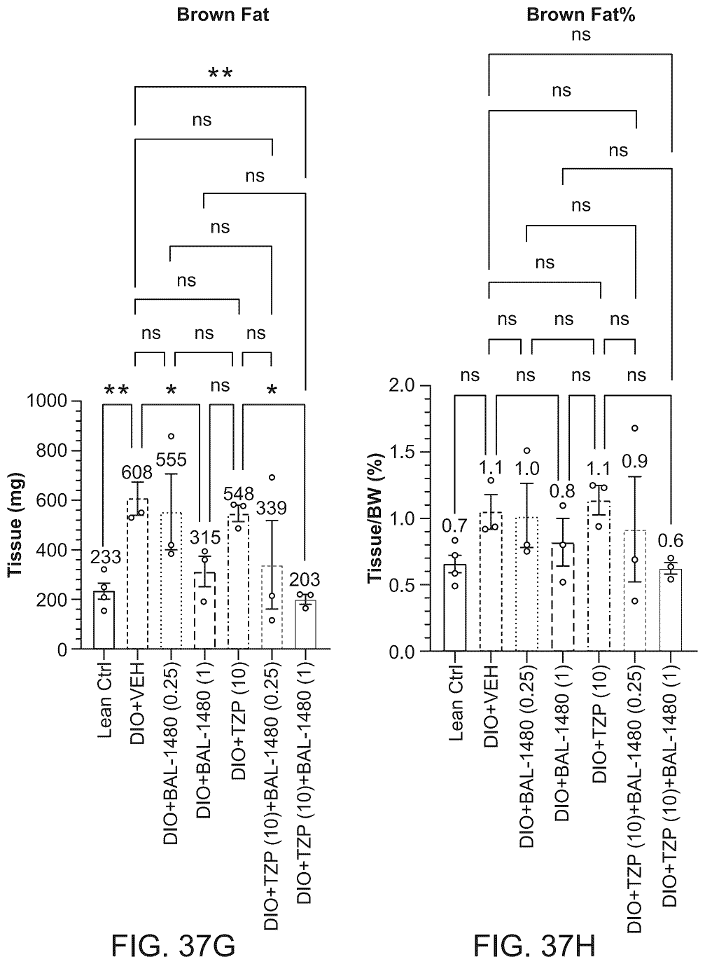

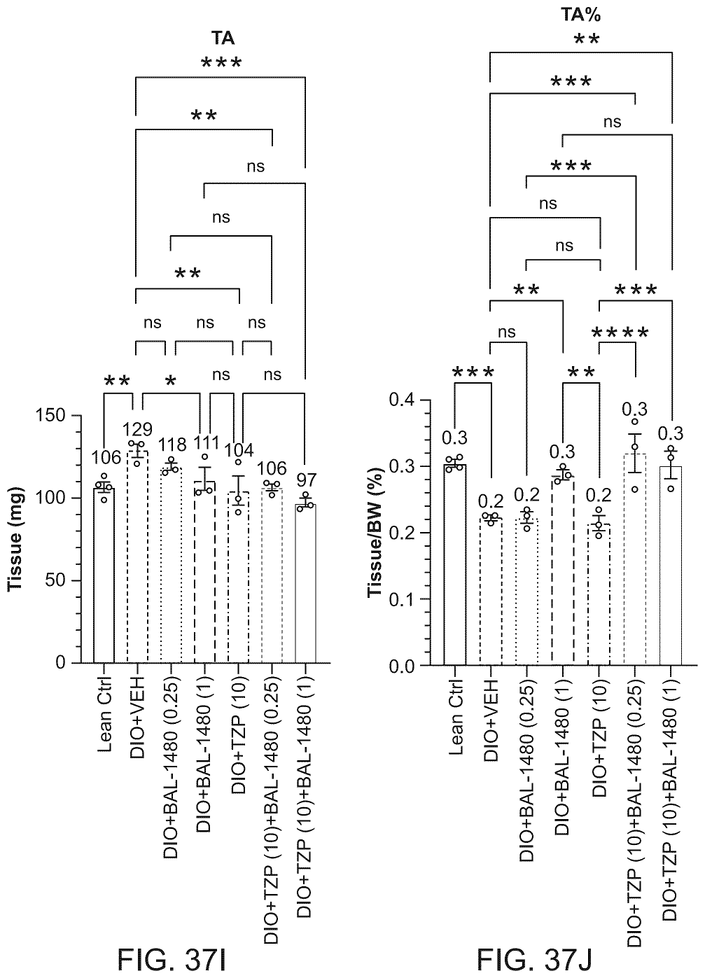

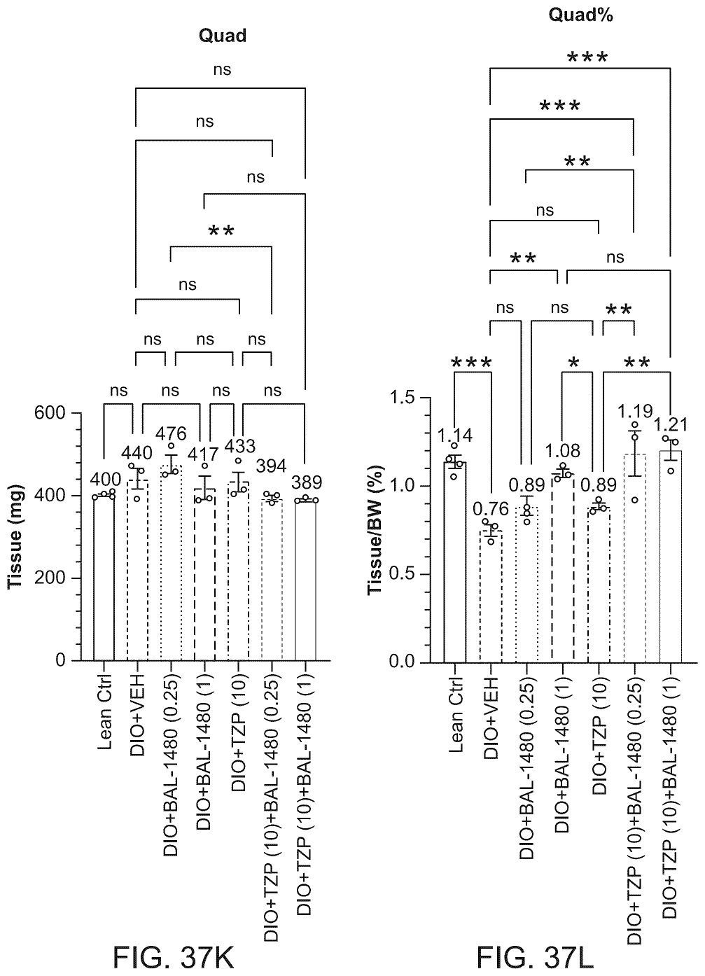

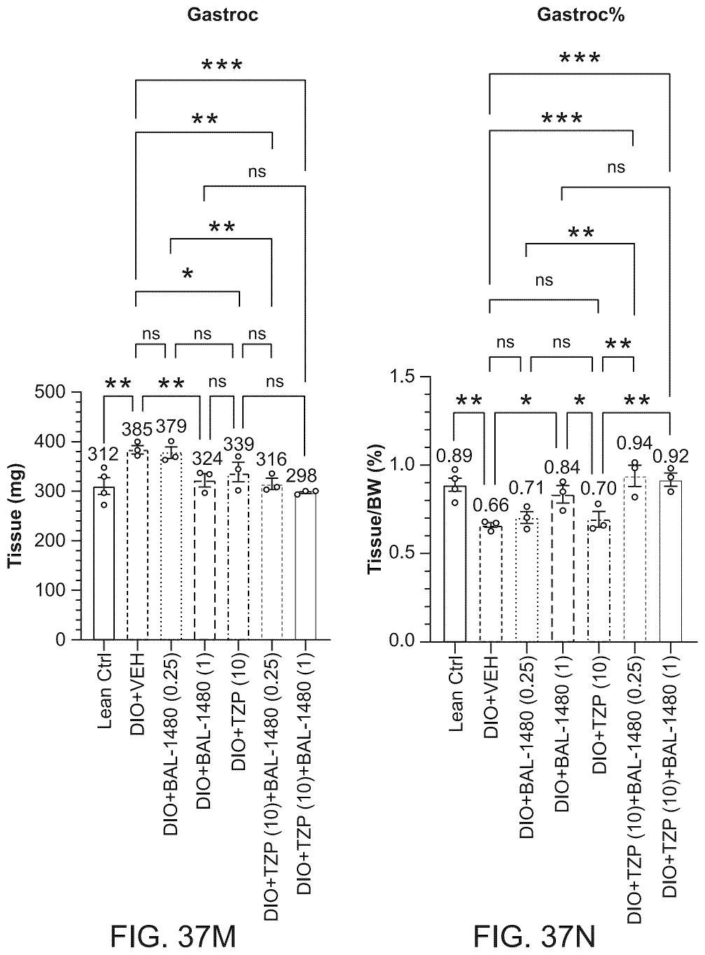

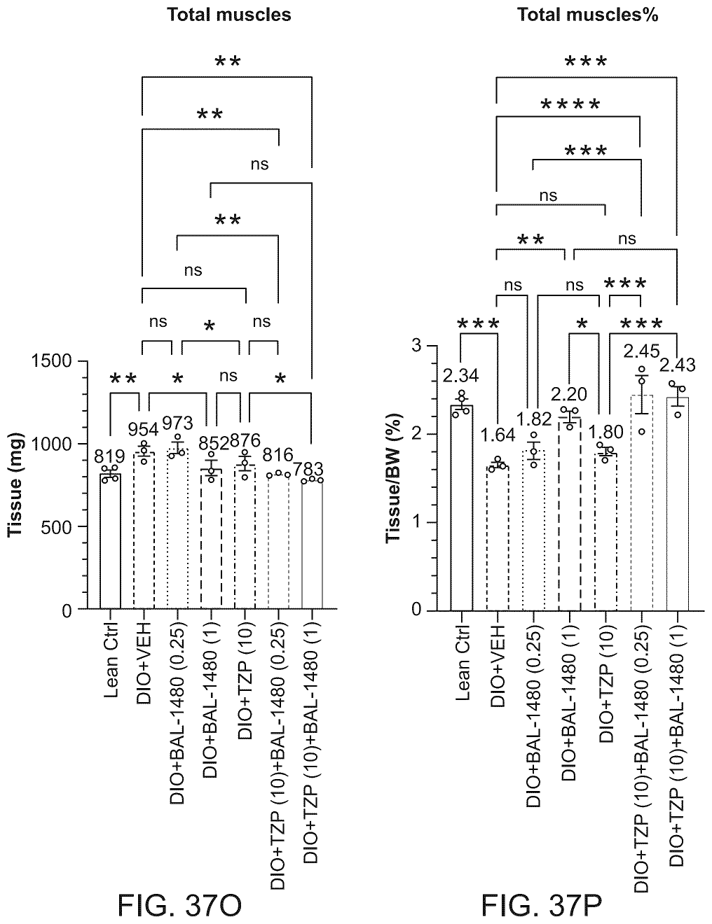

Terminal liver, fat and muscle harvest: Half of the mice (n=3/DIO groups) were taken-down two days after last dose of tirzepatide to harvest the liver, fat and muscle for further test. BAL-1480 at 1 g/L and combination groups reduced the fatty liver weight and fat tissue weights in comparison to tirzepatide monotherapy and dramatically increased the muscle to body weight percentages to the similar level as lean control mice. The results are shown in FIGS. 37A-37P. Quad: quadricep; TA: tibialis anterior; Gastroc: gastrocnemius. Total muscle is the sum of TA, Quad and Gastroc. *=p<0.05; **=p<0.01; ***=p<0.001; ****=p<0.000¹.

In summary, combination of BAL-1480 and tirzepatide is effective in decreasing body weight, restoring body weight to lean control level, and treating obesity.

### 6.7. Example 7: Effects of Combination of BGE-105 and Semaglutide in Obese Mice

### Study Objective

The study objective is to measure the effect of BGE-105 on weight loss or body composition in DIO Mice by GLP-1 receptor agonist semaglutide.

### Methods

Measurement used and justifications include: body weight, food intake, water intake, non-fasted blood glucose, and body composition by Echo-MRI.

C57BL/6 obese male mice from Jax and housed to 9-months old were fed with a lean control diet D12450B (10 kcal % fat) and a diet-induced obesity (DIO) diet D12492 (60 kcal % fat) (Research Diets, Inc.). Target DIO weight range at randomization: 50-62 g.

Male DIO mice were acclimated to a single house cage for two weeks. Body weight, morning non-fasted glucose, and body composition were measured for randomization. After randomization, mice were given treatment as the following groups (All groups were paired with relative dosing s.c. vehicle (20 mM citrate buffer at pH 7.0, 4 mL/kg) or pH 8.5 drinking water with addition of 5 mM sucralose (5 mM sucralose, pH 8.5) to insure water consumption despite possible taste alterations from drug).

Study groups
0. Lean control, n=7
1. DIO+VEH (vehicle control), n=8
2. DIO+BGE-105 (1.1 g/L in drug water), n=8
3. DIO+Semaglutide (30 nmol/kg), n=8
4. DIO+Semaglutide (30 nmol/kg)+BGE-105 (1.1 g/L in drug water), n=8

(0) Lean control: aged matched lean mice (5 mM sucralose water+vehicle, s.c., every 3 days); (1) DIO+VEH: diet-induced obese mice treated with vehicle control (5 mM sucralose water, pH 8.5+vehicle, s.c., every 3 days); (2) DIO+BGE-105 (1.1 g/L in drug water, 5 mM sucralose water, pH 8.5+vehicle, s.c., every 3 days): diet-induced obese mice treated with BGE-105 at 1.1 g/L; (3) DIO+semaglutide (30 nmol/kg): diet-induced obese mice treated with semaglutide at 30 nmol/kg; (4) DIO+semaglutide (30 nmol/kg)+BGE-105 (1.1 g/L in drug water): diet-induced obese mice treated with semaglutide at 30 nmol/kg in combination with BGE-105 at 1.1 g/L.

Semaglutide was purchased from MCE (Cat No. HY-114118/CS-0069903). MW=4113.64, 30 nmol/kg=0.123 mg/kg, 4 mL/kg, drug concentration: 0.031 mg/mL in s.c. vehicle, s.c. injection every 3 days. BGE-105: 1.1 mg/mL was prepared in drinking water with 5 mM sucralose, pH 8.5.

Body weight, food intake, water intake, body composition and morning non-fasted glucose were measured during the entire study. After 19 days of treatment and measurement, mice were taken down to harvest tissues for further downstream analysis. On the day of necropsy, blood plasma and organ/tissue samples were collected. Tissue samples were weighed and processed for further tests. Terminal cardiac blood samples (Heparin plasma) are obtained.

Tissue: Half of the tissue samples are frozen down for molecular biology analysis and the other half are fixed in 10% neutral buffered formalin (or embedding in OTC) for histological analysis.

Fat: Inguinal fat, perigonadal fat, brown fat; Muscle: quadricep, tibialis anterior, gastrocnemius; liver.

Data were expressed as mean±SEM and statistically analyzed by 1-way or 2-way ANOVA.

### Results

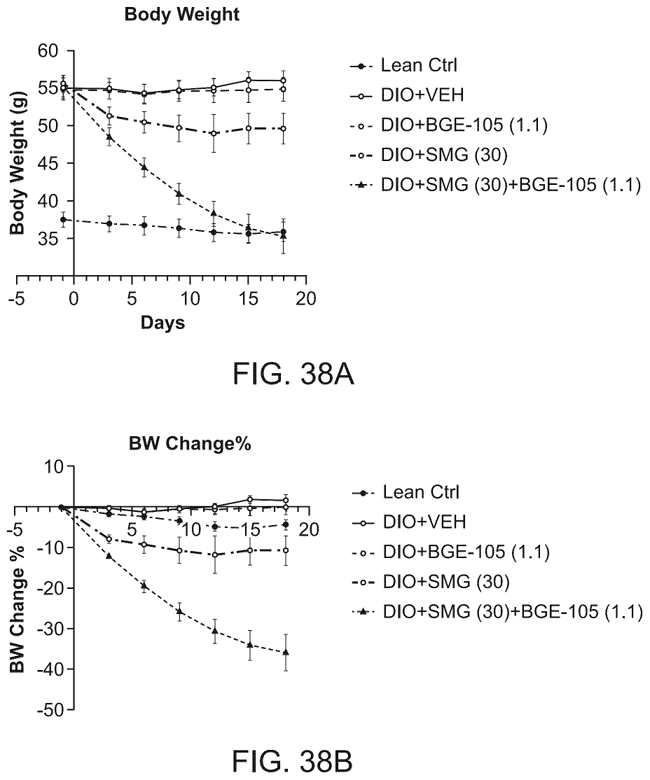

Body Weight: Combination of BGE-105 and semaglutide significantly decreased more body weight and body weight percentage in obese mice than semaglutide monotherapy, and restored it to the similar level as lean control mice at Day 18. The results are shown in FIGS. 38A-38B and Tables 23-24 below.

TABLE 23
Body weight in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | ns | 0.7726 |
| (30) + BGE-105 (1.1) | | |
| DIO + VEH vs. DIO + BGE-105 (1.1) | ns | 0.5833 |
| DIO + VEH vs. DIO + SMG (30) | ** | 0.0025 |
| DIO + VEH vs. DIO + SMG | **** | <0.0001 |
| (30) + BGE-105 (1.1) | | |
| DIO + BGE-105 (1.1) vs. | * | 0.0130 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

TABLE 24
Body weight change % in treated mice

| | | Individual |
|---|---|---|
| Uncorrected Fisher's LSD Day 18 | Summary | P Value |
| Lean Ctrl vs. DIO + VEH | * | 0.0342 |
| Lean Ctrl vs. DIO + BGE-105 (1.1) | ns | 0.1389 |
| Lean Ctrl vs. DIO + SMG (30) | * | 0.0314 |
| Lean Ctrl vs. DIO + SMG | **** | <0.0001 |
| (30) + BGE-105 (1.1) | | |
| DIO + VEH vs. DIO + BGE-105 (1.1) | ns | 0.5048 |
| DIO + VEH vs. DIO + SMG (30) | **** | <0.0001 |
| DIO + VEH vs. DIO + SMG | **** | <0.0001 |
| (30) + BGE-105 (1.1) | | |
| DIO + BGE-105 (1.1) vs. | *** | 0.0002 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

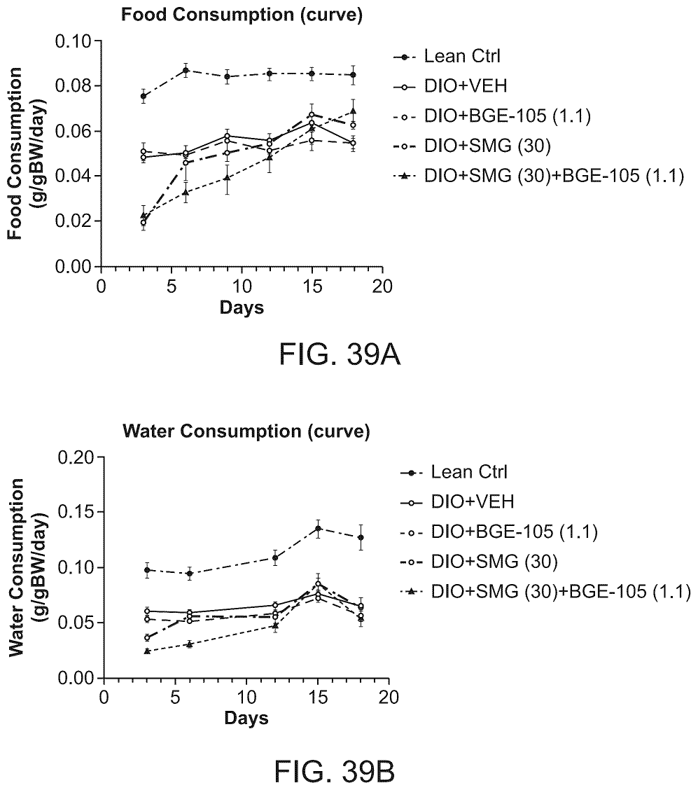

Daily Food Consumption: The food consumptions (g/gBW/day) of BGE-105 and semaglutide combination group was not significantly different from semaglutide monotherapy by comparing the entire curve up to Day 18. The results are shown in FIG. 39A and Table 25 below.

TABLE 25
Food consumption in treated mice

| | | Individual |
|---|---|---|
| Uncorrected Fisher's LSD | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | **** | <0.0001 |
| (30) + BGE-105 (1.1) | | |
| DIO + VEH vs. DIO + BGE-105 (1.1) | ns | 0.3711 |
| DIO + VEH vs. DIO + SMG (30) | * | 0.0363 |
| DIO + VEH vs. DIO + SMG | **** | <0.0001 |
| (30) + BGE-105 (1.1) | | |
| DIO + BGE-105 (1.1) vs. | ns | 0.2271 |
| DIO + SMG (30) | | |
| DIO + BGE-105 (1.1) vs. DIO + SMG | ** | 0.0019 |
| (30) + BGE-105 (1.1) | | |
| DIO + SMG (30) vs. DIO + SMG | ns | 0.0543 |
| (30) + BGE-105 (1.1) | | |

Water Consumption: The water consumption in the BGE-105 and Semaglutide combination group was significantly lower than the Semaglutide monotherapy group on day 6, then picked up from day 12 without significant difference from Semaglutide monotherapy group from day 12 to 18 (p=<0.0001). The results are shown in FIG. 39B and Table 26 below.

TABLE 26
Water consumption in semaglutide treated mice

| | | Individual |
|---|---|---|
| Uncorrected Fisher's LSD | Summary | P Value |
| Day 3 | | |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG (30) + BGE-105 (1.1) | **** | <0.0001 |
| DIO + VEH vs. DIO + BGE-105 (1.1) | ns | 0.3568 |
| DIO + VEH vs. DIO + SMG (30) | ** | 0.0023 |
| DIO + VEH vs. DIO + SMG (30) + BGE-105 (1.1) | **** | <0.0001 |
| DIO + BGE-105 (1.1) vs. DIO + SMG (30) | * | 0.0310 |
| DIO + BGE-105 (1.1) vs. DIO + SMG (30) + BGE-105 (1.1) | *** | 0.0003 |
| DIO + SMG (30) vs. DIO + SMG (30) + BGE-105 (1.1) | ns | 0.1198 |
| Day 6 | | |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG (30) + BGE-105 (1.1) | **** | <0.0001 |
| DIO + VEH vs. DIO + BGE-105 (1.1) | ns | 0.3426 |
| DIO + VEH vs. DIO + SMG (30) | ns | 0.6932 |
| DIO + VEH vs. DIO + SMG (30) + BGE-105 (1.1) | *** | 0.0003 |
| DIO + BGE-105 (1.1) vs. DIO + SMG (30) | ns | 0.5786 |
| DIO + BGE-105 (1.1) vs. DIO + SMG (30) + BGE-105 (1.1) | ** | 0.0069 |
| DIO + SMG (30) vs. DIO + SMG (30) + BGE-105 (1.1) | ** | 0.0012 |
| Day 12 | | |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG (30) + BGE-105 (1.1) | **** | <0.0001 |
| DIO + VEH vs. DIO + BGE-105 (1.1) | ns | 0.3123 |
| DIO + VEH vs. DIO + SMG (30) | ns | 0.1668 |
| DIO + VEH vs. DIO + SMG (30) + BGE-105 (1.1) | * | 0.0165 |
| DIO + BGE-105 (1.1) vs. DIO + SMG (30) | ns | 0.7080 |
| DIO + BGE-105 (1.1) vs. DIO + SMG (30) + BGE-105 (1.1) | ns | 0.1607 |
| DIO + SMG (30) vs. DIO + SMG (30) + BGE-105 (1.1) | ns | 0.3028 |
| Day 15 | | |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG (30) + BGE-105 (1.1) | **** | <0.0001 |
| DIO + VEH vs. DIO + BGE-105 (1.1) | ns | 0.6139 |
| DIO + VEH vs. DIO + SMG (30) | ns | 0.2277 |
| DIO + VEH vs. DIO + SMG (30) + BGE-105 (1.1) | ns | 0.2335 |
| DIO + BGE-105 (1.1) vs. DIO + SMG (30) | ns | 0.0879 |
| DIO + BGE-105 (1.1) vs. DIO + SMG (30) + BGE-105 (1.1) | ns | 0.0908 |
| DIO + SMG (30) vs. DIO + SMG (30) + BGE-105 (1.1) | ns | 0.9879 |
| Day 18 | | |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG (30) + BGE-105 (1.1) | **** | <0.0001 |
| DIO + VEH vs. DIO + BGE-105 (1.1) | ns | 0.2905 |
| DIO + VEH vs. DIO + SMG (30) | ns | 0.8273 |
| DIO + VEH vs. DIO + SMG (30) + BGE-105 (1.1) | ns | 0.1515 |
| DIO + BGE-105 (1.1) vs. DIO + SMG (30) | ns | 0.4011 |
| DIO + BGE-105 (1.1) vs. DIO + SMG (30) + BGE-105 (1.1) | ns | 0.7042 |
| DIO + SMG (30) vs. DIO + SMG (30) + BGE-105 (1.1) | ns | 0.2233 |

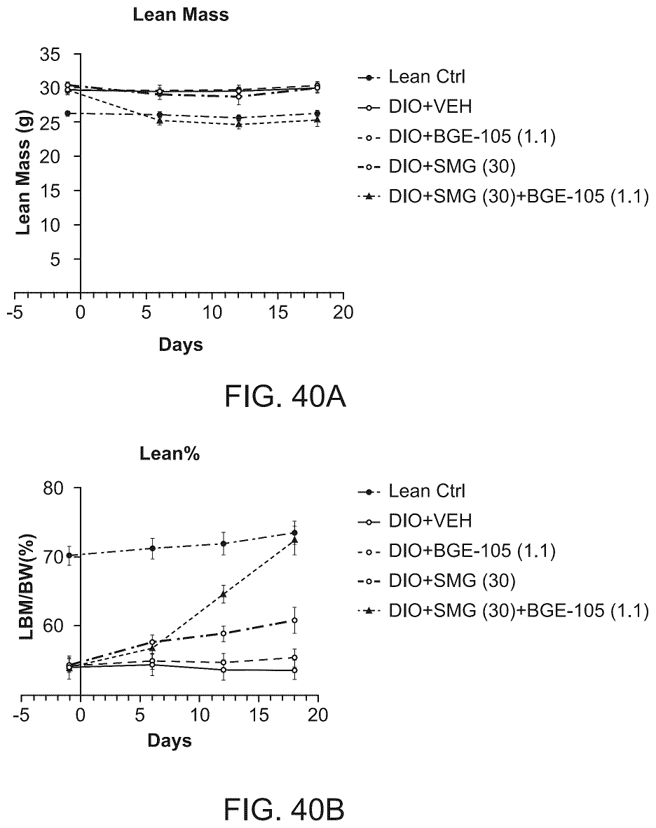

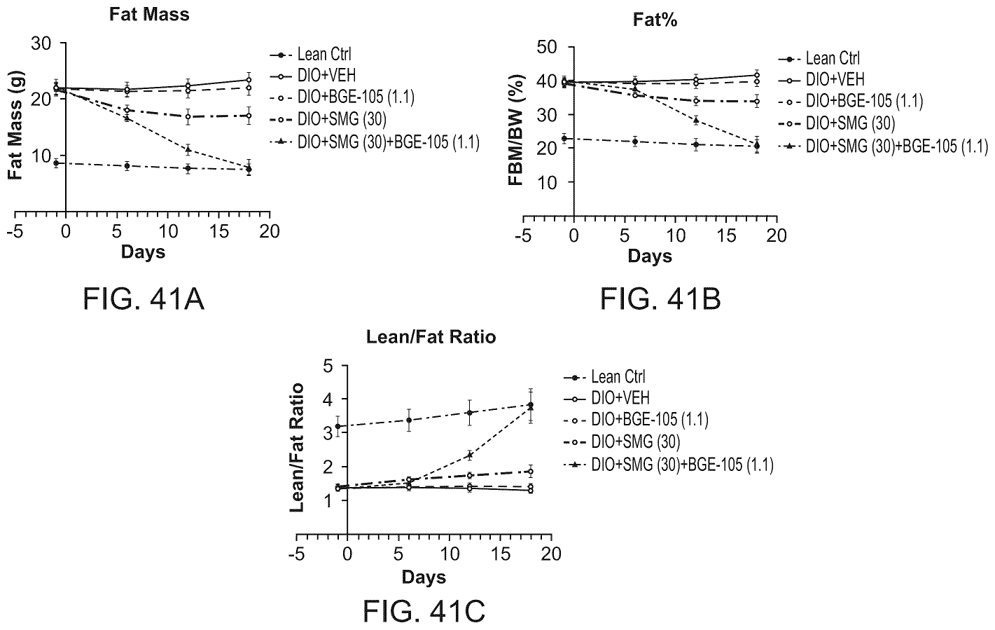

Absolute Lean Mass and Fat Mass: Addition of BGE-105 to semaglutide treatment showed dramatic reduction of absolute fat mass by about 10 g (FIG. 41A), which was more dramatic than the change of absolute lean mass by about 5 g (FIG. 40A). FIGS. 40A-40B and Tables 27-28 below show lean mass and lean mass percentage.

TABLE 27
Lean mass in semaglutide treated mice

| | | Individual |
|---|---|---|
| Uncorrected Fisher's LSD Day 18 | Summary | P Value |
| Lean Ctrl vs. DIO + VEH | *** | 0.0002 |
| Lean Ctrl vs. DIO + BGE-105 (1.1) | **** | <0.0001 |
| Lean Ctrl vs. DIO + SMG (30) | *** | 0.0002 |
| Lean Ctrl vs. DIO + SMG | ns | 0.2969 |
| (30) + BGE-105 (1.1) | | |
| DIO + VEH vs. DIO + BGE-105 (1.1) | ns | 0.7350 |
| DIO + VEH vs. DIO + SMG (30) | ns | 0.9894 |
| DIO + VEH vs. DIO + SMG | **** | <0.0001 |
| (30) + BGE-105 (1.1) | | |
| DIO + BGE-105 (1.1) vs. | ns | 0.7450 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

TABLE 28
Lean mass percentage in semaglutide treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | ns | 0.5788 |
| (30) + BGE-105 (1.1) | | |
| DIO + VEH vs. DIO + BGE-105 (1.1) | ns | 0.3608 |
| DIO + VEH vs. DIO + SMG (30) | *** | 0.0002 |
| DIO + VEH vs. DIO + SMG | **** | <0.0001 |
| (30) + BGE-105 (1.1) | | |
| DIO + BGE-105 (1.1) vs. | ** | 0.0051 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

Lean Mass Percentage: Addition of BGE-105 to semaglutide treatment showed improvement in lean mass percentage and was able to restore the lean mass percentage to the similar level of lean control mice. The results are shown in FIG. 40B and Table 28.

FIG. 41A and Table 29 below show addition of BGE-105 to Semaglutide treatment showed dramatic reduction of absolute fat mass.

TABLE 29
Fat mass in Semaglutide treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | ns | 0.8427 |
| (30) + BGE-105 (1.1) | | |
| DIO + VEH vs. DIO + BGE-105 (1.1) | ns | 0.3575 |
| DIO + VEH vs. DIO + SMG (30) | *** | 0.0001 |
| DIO + VEH vs. DIO + SMG | **** | <0.0001 |
| (30) + BGE-105 (1.1) | | |
| DIO + BGE-105 (1.1) vs. | ** | 0.0025 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

Fat Mass Percentage: Addition of BGE-105 to semaglutide treatment showed reduction in fat mass percentage and was able to restore the fat mass percentage to the similar level of lean control mice. The results are shown in FIG. 41B and Table 30 below.

TABLE 30
Fat mass percentage in Semaglutide treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | ns | 0.7449 |
| (30) + BGE-105 (1.1) | | |
| DIO + VEH vs. DIO + BGE-105 (1.1) | ns | 0.3837 |
| DIO + VEH vs. DIO + SMG (30) | *** | 0.0003 |
| DIO + VEH vs. DIO + SMG | **** | <0.0001 |
| (30) + BGE-105 (1.1) | | |
| DIO + BGE-105 (1.1) vs. | ** | 0.0058 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

Lean/Fat Ratio: Addition of BGE-105 to Semaglutide treatment showed significant increase in lean/fat ratio and restored it to the similar level as lean control mice (Lean Ctrl vs. DIO+SMG (30)+BGE-105 (1.1); p=0.7749). The results are shown in FIG. 41C and Table 31 below.

TABLE 31
Lean/fat ratio in treated mice

| | | Individual |
|---|---|---|
| Uncorrected Fisher's LSD Day 18 | Summary | P Value |
| Lean Ctrl vs. DIO + VEH | **** | <0.0001 |
| Lean Ctrl vs. DIO + BGE-105 (1.1) | **** | <0.0001 |
| Lean Ctrl vs. DIO + SMG (30) | **** | <0.0001 |
| Lean Ctrl vs. DIO + SMG | ns | 0.7749 |
| (30) + BGE-105 (1.1) | | |
| DIO + VEH vs. DIO + BGE-105 (1.1) | ns | 0.7048 |
| DIO + VEH vs. DIO + SMG (30) | ns | 0.0518 |
| DIO + VEH vs. DIO + SMG | **** | <0.0001 |
| (30) + BGE-105 (1.1) | | |
| DIO + BGE-105 (1.1) vs. | ns | 0.1160 |
| DIO + SMG (30) | | |
| DIO + BGE-105 (1.1) vs. DIO + SMG | **** | <0.0001 |
| (30) + BGE-105 (1.1) | | |
| DIO + SMG (30) vs. DIO + SMG | **** | <0.0001 |
| (30) + BGE-105 (1.1) | | |

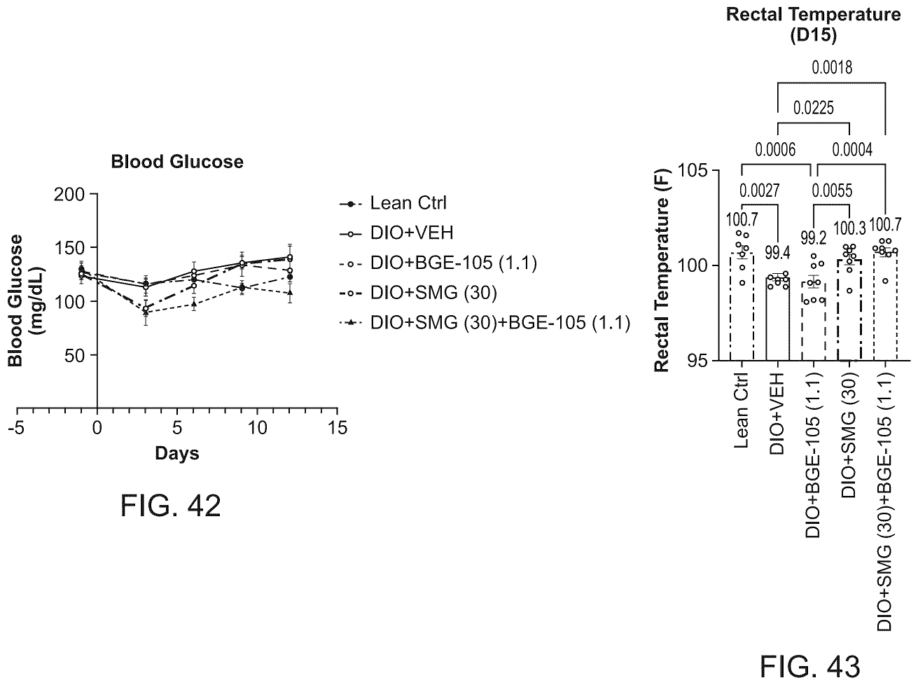

Morning non-fasted blood glucose: the morning non-fasted blood glucose in the Semaglutide+BGE-105 combination group was significantly lower than that in the Semaglutide monotherapy group (DIO+SMG (30) vs. DIO+SMG (30)+BGE-105 (1.1); p<0.0001). FIG. 42 and Table 32 below show the results of blood glucose.

TABLE 32
Blood glucose in Semaglutide treated mice

| | | Individual |
|---|---|---|
| Uncorrected Fisher's LSD Day 18 | Summary | P Value |
| DIO + VEH vs. DIO + BGE-105 (1.1) | ns | 0.6833 |
| DIO + VEH vs. DIO + SMG (30) | ns | 0.1997 |
| DIO + VEH vs. DIO + SMG | *** | 0.0002 |
| (30) + BGE-105 (1.1) | | |
| DIO + BGE-105 (1.1) vs. DIO + SMG (30) | ns | 0.3806 |
| DIO + BGE-105 (1.1) vs. DIO + SMG | *** | 0.0009 |
| (30) + BGE-105 (1.1) | | |
| DIO + SMG (30) vs. DIO + SMG | * | 0.0127 |
| (30) + BGE-105 (1.1) | | |

Rectal Temperature: the rectal temperature was measured in the afternoon on day 15. Semaglutide monotherapy significantly increased the body temperature than the VEH group (p=0.0225). However, the addition of BGE-105 to semaglutide had a more significant increase in body temperature in comparison to the VEH group (p=0.0018). The results are shown in FIG. 43.

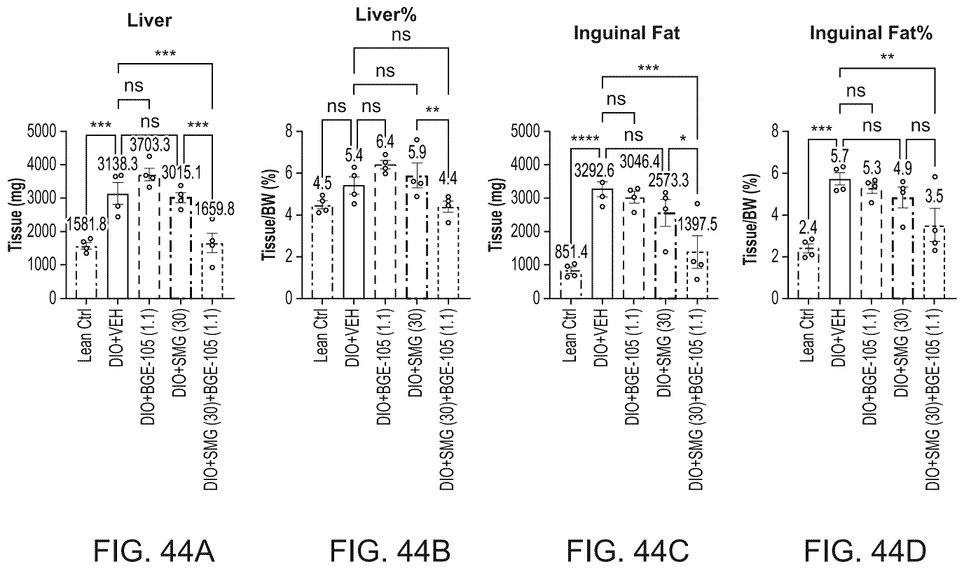

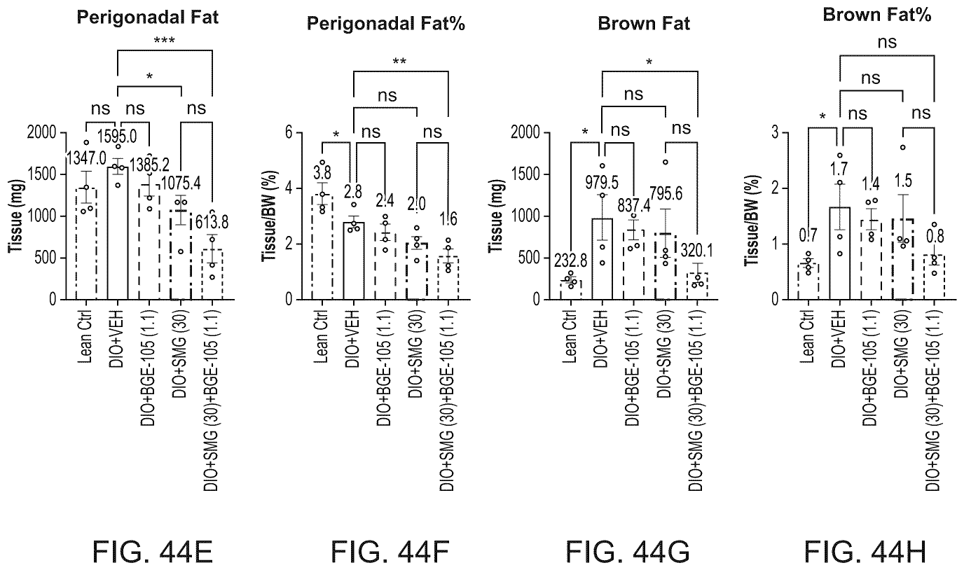

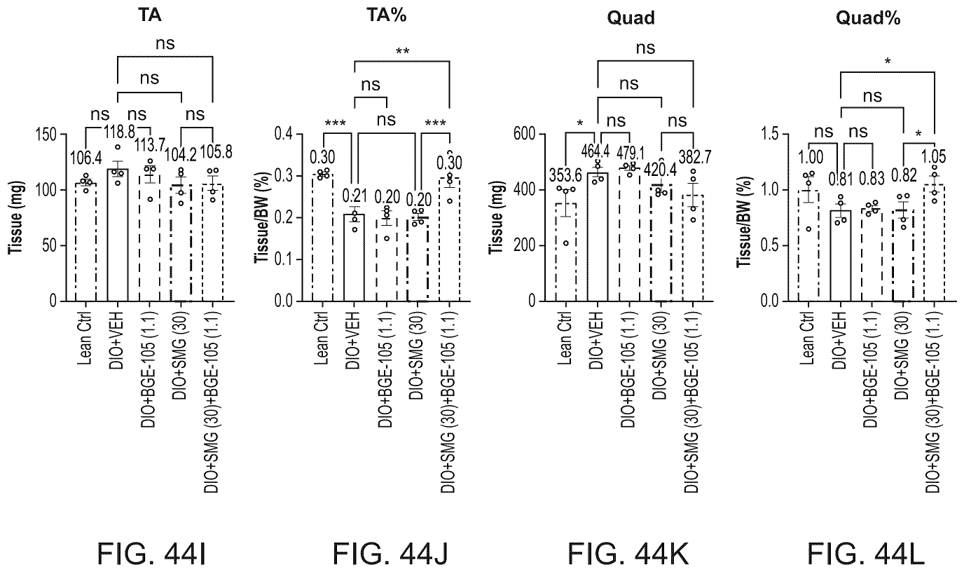

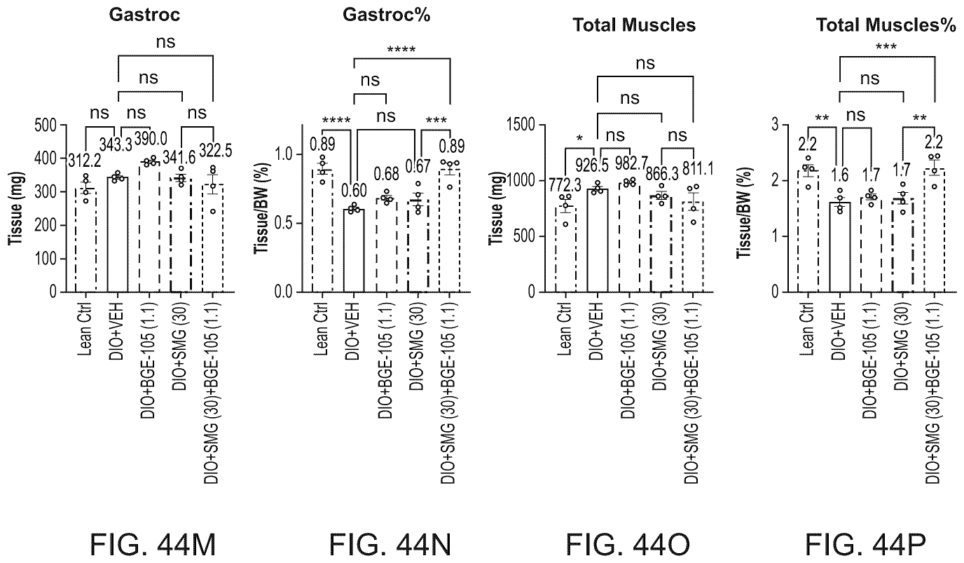

Terminal liver, fat and muscle harvest: Half of the mice (n=4/group) were taken-down four days after last dose of semaglutide to harvest the liver, fat and muscle for further test. Addition of BGE-105 to Semaglutide significantly reduced the fatty liver weight and inguinal fat in comparison to Semaglutide monotherapy and dramatically increased the muscle to body weight percentages to the similar level as lean control mice. The results are shown in FIGS. 44A-44P. Quad: quadricep; TA: tibialis anterior; Gastroc: gastrocnemius. Total muscle is the sum of TA, Quad and Gastroc.

### 6.8. Example 8: BGE-105 Significantly Reduced Muscle Atrophy in Healthy Adult Patients on Enforced Bedrest (Phase 1b Clinical Study)

The examples above demonstrate significant desirable effects when an apelin receptor (APJ) agonist (e.g., BGE-105 or BAL-1480) is combined with a representative GLP-1 receptor agonist (e.g., semaglutide or tirzepatide), in diet-induced obese (DIO) mice, a well-established model of human obesity. The data from the Phase 1b clinical study described in this example confirmed that BGE-105's positive effects on muscle retention and retention of lean body mass that were observed in the DIO mice translate to humans.

### 6.8.1. Topline Results

This double-blind, placebo-controlled, trial evaluated the safety and pharmacodynamics of BGE-105. Twenty-one volunteers underwent 10 days of bed rest while receiving infusions of BGE-105 or placebo.

After 10 days of bed rest, volunteers on placebo (n=10) exhibited muscle atrophy, reflected by statistically significant reductions in thigh circumference and ultrasound measurement of vastus lateralis muscle dimensions (cross sectional area and thickness) and muscle quality (fatty degeneration).

Treatment with BGE-105 (n=11) significantly ameliorated muscle atrophy caused by bedrest relative to placebo:
Muscle dimensions: Volunteers receiving BGE-105 showed a 100% improvement in thigh circumference (p<0.001) relative to placebo-treated volunteers, and ultrasound measurements showed a 58% improvement in vastus lateralis cross-sectional area (p<0.05) and a 73% improvement in vastus lateralis thickness (p<0.01).
Muscle quality: Ultrasound echo density measurements revealed that the Goutallier grade, an index that quantifies fatty degeneration in muscle, worsened in 8 of 10 volunteers on placebo vs. only 1 of 11 volunteers receiving BGE-105 (p<0.005).
Muscle protein synthesis: Proteomic analysis of muscle microbiopsy samples revealed that bed rest decreased production of muscle proteins, and this effect was significantly ameliorated by BGE-105 (p<0.005). The higher rate of muscle protein synthesis in the drug vs. placebo group provides a potential mechanistic basis for BGE-105's protective effect on muscle dimensions.

### 6.8.2. Protocol

The human clinical study assesses apelin effects of BGE-105 with both single and multiple doses. Two groups (Group A “Part A”, single-ascending dose (SAD), and Group B “Part B”, multiple dose (MD)) of healthy older adults participate in the study for approximately 42 days including a Screening/Pre-Treatment Period of up to 14 days, a Treatment Period of 5 days for Part A and 7 days for Part B, and a Follow-up Visit 27 days after the first administration of study drug (BGE-105 or placebo).

In Part A, of 24 subjects enrolled (3 SAD cohorts, 8 subjects each), a total of at least 12 subjects are ≥65 years of age (≥4 subjects in each cohort). The remaining subjects are ≥18 years old. In each cohort, 6 subjects receive BGE-105 and 2 subjects receive placebo for a total of 18 BGE-105-treated subjects and 6 placebo treated subjects, for a total of 24 subjects. In addition to characterizing the PD effects associated with acute BGE-105 exposure, the use of a 48-hour “drug holiday” between first and second doses in the SAD cohorts, infusions permit gathering information related to tachyphylaxis and durability of apelin-like effects.

In Part B, of the 30 subjects enrolled, all of which are ≥65 years of age. The 30 subjects who qualify are enrolled to receive treatment in either Cohort 1A (placebo), Cohort 1B (240 mg BGE-105 daily), or Cohort 1C (240 mg/1440 mg). Cohort 1A include 10 subjects receiving placebo normal saline (NS), Cohort 1B include 10 subjects each who receive BGE-105, and Cohort 1C include of up to 10 subjects each who receive BGE-105. Subjects participating in this study for approximately 81 days including a screening period of up to 16 days, an Outpatient Pre-Treatment Period of 5 days (Day −5 to Day −1) with heavy water and D3-creatine (D3-Cr), and a Treatment period of 10 days on bed rest with heavy water/D3-Cr and BGE-105 or Placebo, and a post-dose Follow-Up Period (Days 11 to Day 60) that includes 2 follow-up visits on Days 11, 12, 13, 14, 21, 30, and 60 days after the first administration of study drug (BGE-105 or Placebo). In the multi-dose cohorts (1A, 1B, 1C), PD parameters for effects on changes in insulin sensitivity and muscle indices during a period of bed rest are measured and evaluated to better inform decisions regarding choice of dose and direction of further development.

### Objects and Endpoints

### Primary Objective

To evaluate the safety and tolerability of single ascending doses and multiple ascending doses of BGE-105 in healthy adult subjects (>18 years of age in Part A, >65 years of age in Part B) with an emphasis on older subjects (>65 years of age in Part A) after administration of BGE-105 by intravenous (IV) infusion.

### Secondary Objectives

To characterize the pharmacodynamic (PD) effects of BGE-105 after IV infusion in healthy adult subjects;

To characterize the pharmacokinetics (PK) of BGE-105 after IV infusion in healthy adult subjects;

To characterize the PK/PD relationships of BGE-105 on predefined biomarkers (including, but not limited to, glucose, insulin, and insulin sensitivity) and PD variables (such as changes in systolic and diastolic blood pressure, heart rate), and in the multiple dose cohorts (Part B), measurement of muscle protein synthesis rate from administration of heavy water and a micro-(small needle) biopsy of the vastus lateralis, D3-creatine (D3-Cr) total skeletal muscle mass from urine samples, and muscle circumference, cross-sectional area, color flow analysis, anterior-posterior (AP) diameter, and echo density by ultrasound of the vastus lateralis and the gastrocnemius muscles.

### Secondary Endpoints

PK parameters of BGE-105 including, but not limited to:
Area under the serum concentration time curve (AUC) over the 24-hour dosing interval and from time 0 to the time of the last observed serum concentration following the final dose (AUC₀₋ₗₐₛₜ)
AUC from time 0 to infinity (AUC_{0-inf})
Maximum observed serum concentration (Cₘₐₓ)
Time to reach Cₘₐₓ (Tₘₐₓ)
Terminal elimination half-life (T_{1/2})
Evaluation of steady-state and time to reach steady-state by evaluation of trough BGE-105 concentrations
Total body clearance (CL) and volume of distribution (Vz)
Assessment of attainment of steady-state by comparison of:
   up through 96-hour concentrations for Part A and
   up through 336-hour concentrations for Part B.

PD parameters including, but not limited to changes in:
Systolic and diastolic BP
Pulse rate and heart rate
Physical Function Tests Day 1 and Day 11 (48 hours after the start of the last infusion), and at a post-dose follow-up visits through Day 60. Physical Function Tests include assessment/rehabilitation of 1) sit to stand test, 2) short physical performance battery (SPPB), and 3) Tinetti Performance Oriented Mobility Assessment (POMA).

Biomarkers including, but not limited to changes in:
Plasma glucose
Plasma insulin
Insulin resistance (HOMA-IR)

Multi-dose (Part B, MD cohorts) only PD parameters assessed before and after bedrest:
Skeletal muscle circumference, cross-sectional area, color flow analysis, AP diameter, and echo density
D3-creatine total skeletal muscle mass
Muscle protein synthesis rate (contractile and sarcoplasmic) after Heavy Water administration in blood, urine and saliva samples
Micro (fine needle) skeletal muscle biopsy (of the vastus lateralis) to determine “flux” proteomics, the rate of synthesis of hundreds of muscle proteins including contractile, mitochondrial, sarcoplasmic, membrane-bound, and matrix.

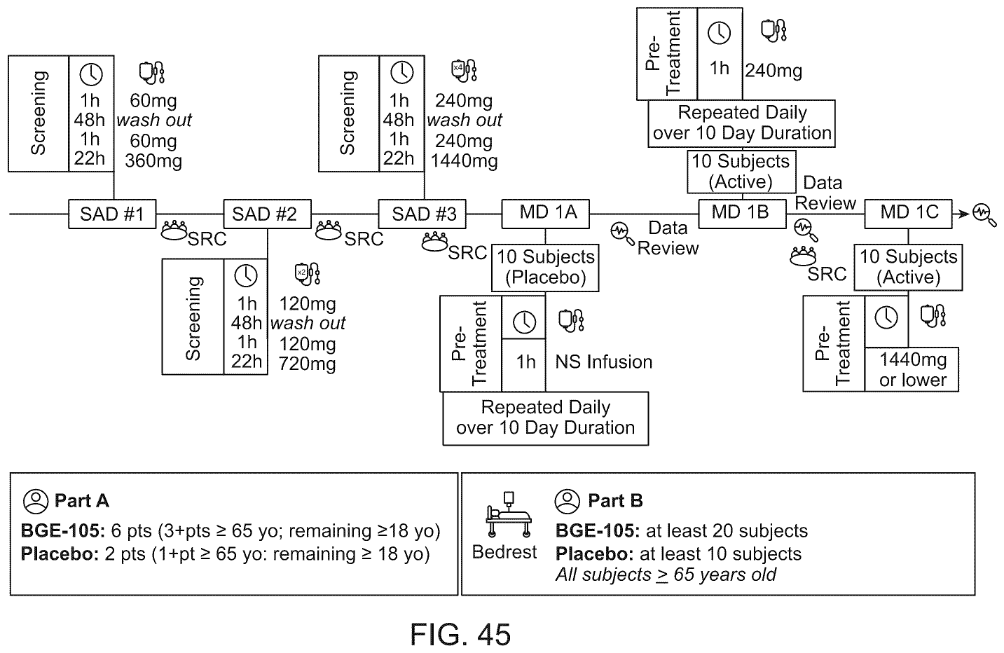

This study is a randomized, placebo controlled, double blind, single ascending dose (SAD) and a single-blind, multiple dose (MD), study, in up to 72 healthy adult subjects. There are 6 cohorts: 3 cohorts (8 subjects each) in Part A and 3 cohorts (10 subjects each) in Part B. Cohorts can be split and dosing staggered by 1 day to facilitate collection of data on heavy procedure days (e.g., the 10 subjects in the MD cohort can be split into groups of 5, staggered 1 day apart). The study design is provided as FIG. 45.

In each of the 3 cohorts in Part A (SAD cohorts), there are a total of 6 healthy male or female receiving BGE-105 (at least 3 subjects≥65 years old, remaining subjects≥18 years old) and 2 Healthy male or female receiving placebo (at least 1 subject≥65 years old, remaining subject≥18 years old) via IV infusion.

After a baseline period of 24 hours in the clinic, all subjects receive a LD, 1-hour infusion on Day 1. After a 48-hour wash-out period, in which PK and PK/PD data iscollected, subjects receive a 23-hour infusion (1-hour LD followed by a 22-hour maintenance dose). See FIG. 45 for the different dose levels in each SAD cohort. See e.g., Table 33 below. Part A is double-blinded. The cohorts of Part A is conducted sequentially starting with SAD cohort 1.

In each of the 3 cohorts in Part B, 30 total subjects are enrolled to receive treatment in either Cohort 1A, Cohort 1B, or Cohort 1C. Cohort 1A includes 10 subjects who all receive placebo NS over a 1 hour infusion for 10 days on Days 1 through Day 10. Cohort 1B includes 10 subjects each who receive BGE-105, 240 mg over 1 hour infusion, up to 10 days on Days 1 through 10. Cohort 1C includes up to 10 subjects each who receive BGE-105 at a dose not to exceed 1400 mg over 24 hours up to 10 days. Patients are at bed rest for the entire 10-day duration of treatment.

The dose of BGE-105 for Cohort 1C is a dose not to exceed 1440 mg over 24 hours which was the highest dose given in the SAD and was well tolerated. The dosing regimen over the 10 days is guided by HOMA-IR data as required from Cohort 1B and the dose, dose regimen, or both may be changed.

The Pre-Treatment Period for all 3 cohorts starts on Day −5 and continue through Day −1. All subjects in each cohort are admitted to the unit on Day −2. All subjects start on bed rest on Day 1 and continue to Day 10.

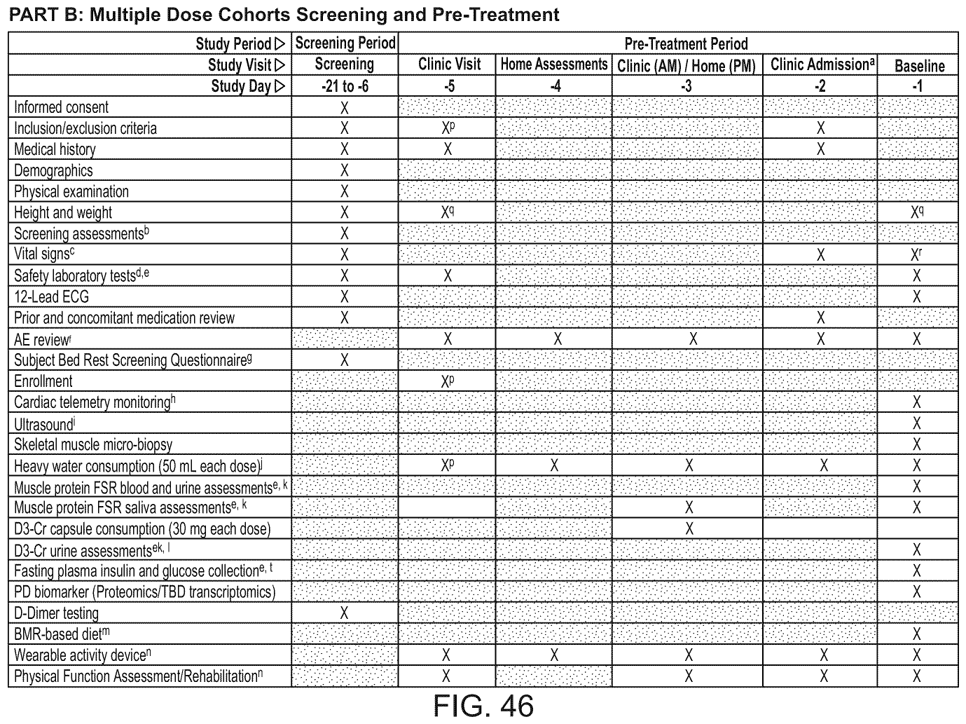

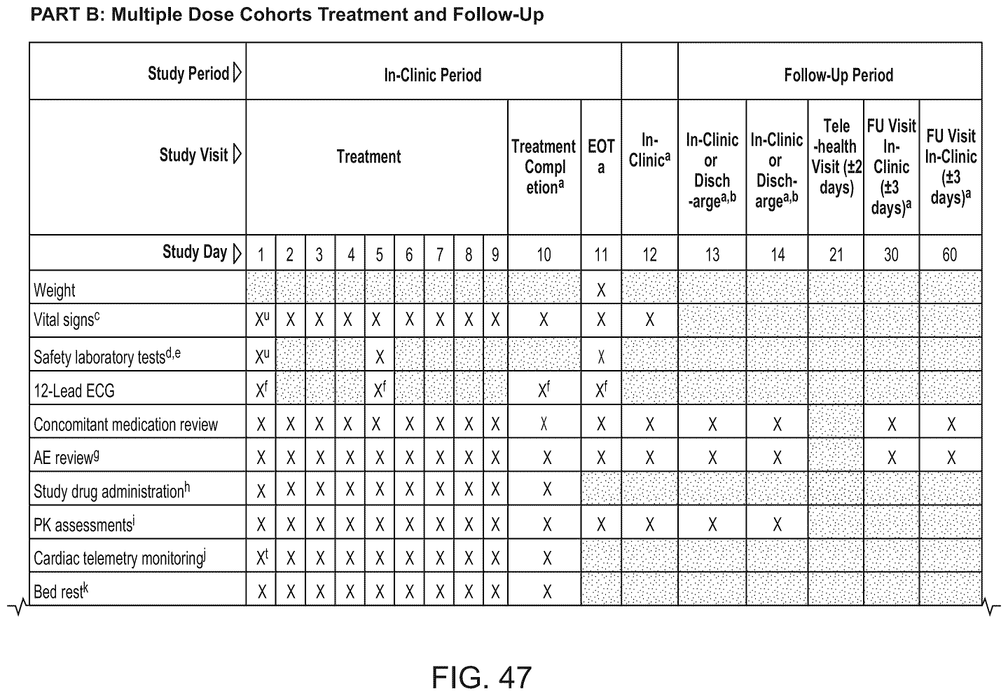

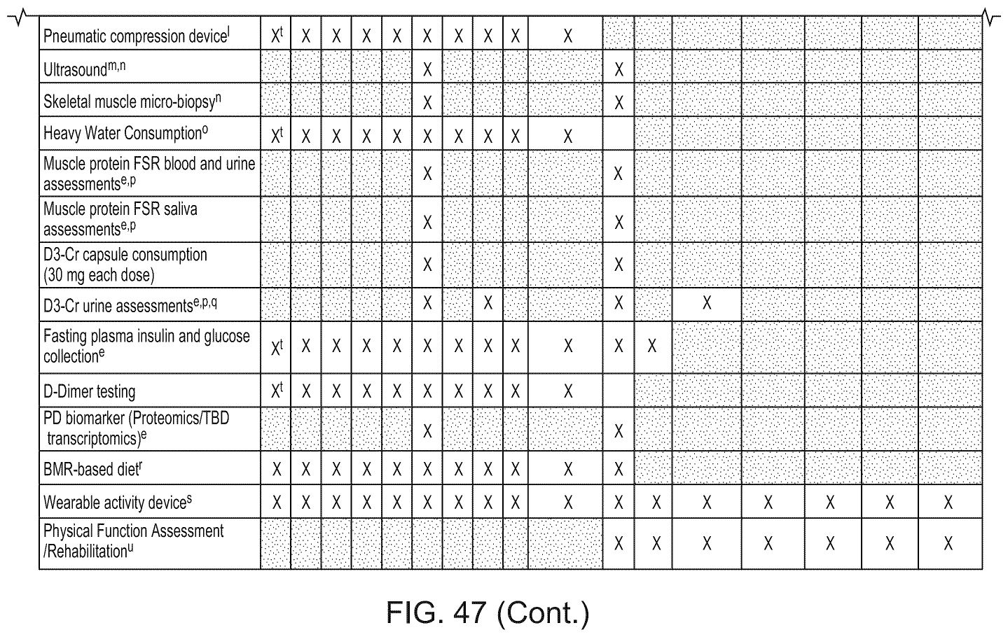

All multiple dose (Part B) cohort subjects receive heavy water and D3-Cr starting on Day −5 and continue through Day 10 according to FIGS. 46-47.

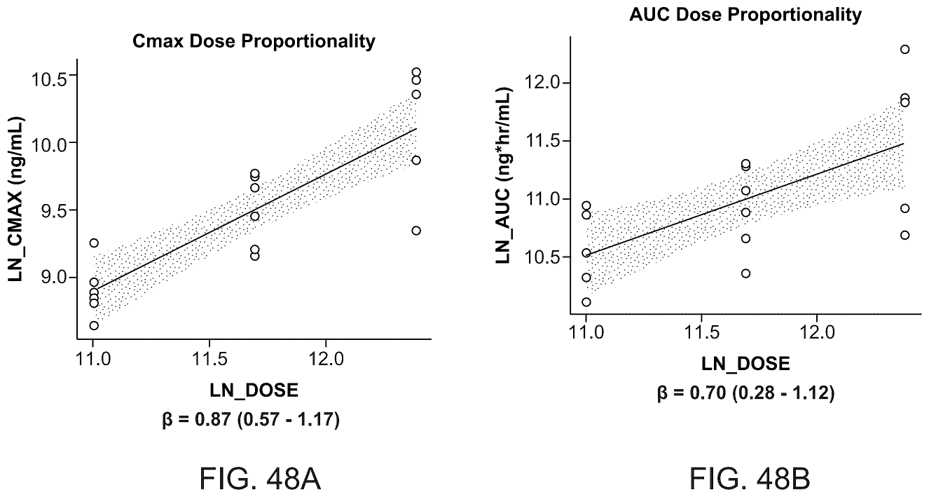

Subjects receive heavy water starting on Day −5 and D3-Cr starting on Day −3 and both continue through Day 10 according to FIGS. 47-48.

After the completion of multiple dose Cohort 1A, a pharmacodynamic evaluation is performed by the Sponsor of the results from samples collected from the skeletal muscle biopsy, blood, urine, and saliva to measure the effects on skeletal muscle. HOMA-IR is evaluated to assess insulin resistance. The data is unblinded to the Sponsor and is used to confirm the number of days of bed rest for Cohort 1B and 1C (e.g., 10 days or less). In addition, between Cohort 1B and Cohort 1C, unblinded HOMA-IR is evaluated by the Sponsor to confirm the dose and dosing regimen for Cohort 1C. Data from other measures such as ultrasound measurements, pharmacokinetic data and the data for muscle mass and muscle protein synthetic rates was reviewed by the Sponsor in an unblinded manner after each cohort.

Subjects have additional in-clinic and home assessments during the Pre-Treatment Period and an extended Follow-Up Period through Day 60.

For the multiple dose cohorts (Part B), Cohort 1A is conducted to confirm the effects on skeletal muscle induced by bed rest as well as the effect on HOMA-IR. The data from Cohort 1A is used to confirm 10 days of bed rest is sufficient to characterize the effect on muscle protein synthesis rate after administration of heavy water and via micro-(fine needle) skeletal muscle biopsy of the vastus lateralis, D3-Cr total skeletal muscle mass determination from a fasting urine sample, and muscle circumference, cross-sectional area, color flow analysis, anterior-posterior (AP) diameter, and echo density by ultrasound of the vastus lateralis and the gastrocnemius. Cohorts 1B and 1C proceeded with 10 days of bed rest or less if evidence a shorter period can elicit measurable effects on skeletal muscle.

The cohorts of Part A and the cohorts of Part B was conducted sequentially starting with SAD Cohort 1. This process was repeated for each cohort in Parts A and B of the study.

### Dosage Rationale:

Based on preliminary results from SAD cohorts (BGE-105-101), an IV dose of BGE-105 up to 4-fold higher than the maximum dose studied in the previous, completed Phase 1 trials (BGE-105-101) was proposed.

Given the good tolerability, lack of human safety findings and lack of dose limiting toxicities in both tox species (NOAEL is maximum dose tested in both rat and dog), this proposed dose increase is justified as long as predicted human exposures are not expected to exceed tox species exposures. The updated exposure margins considering the highest maximum exposures in rats and dogs are calculated as follows:
Maximum exposure in the rat (male) was in the 14-day IV study and at the 300 mg/kg dose, the AUCₗₐₛₜ is 1070 μg*hr/mL.
Maximum exposure in dogs occurred at the 300 mg/kg dose in the 9-month oral toxicity study and the AUCₗₐₛₜ is 1310*μg·hr/mL.

Therefore, based on the PK of the single dose of 240 mg loading dose followed by 1440 mg IV for 22 hours, multiple dosing with this dose (the highest proposed dose for use in this study) the predicted human exposure margins would be:
Human:Rat=1036/1070=0.97
Human:Dog=1036/1310=0.79

Thus, all available safety data continues to support use of doses up to 240/1440 mg and human exposures do not exceed toxicology coverage, even at the highest proposed dose.

TABLE 33
Cohorts, Doses, and Dosing Regimens

| | | | |
|---|---|---|---|
| | Planned | Planned IV infusion | Planned number of |
| Cohort | Doses | Dosing Regimen | subjects |

Part A SAD (24 subjects)

| | | | |
|---|---|---|---|
| Cohort | 60 mg/ | LD 60 mg/1 h | Total: 8 subjects |
| 1: | 360 mg | 48-hour washout period | BGE-105: 6 (at |
| | | LD 60 mg/1 h + MD | least 3 ≥ 65 yo, |
| | | 360 mg/22 h | remaining ≥ 18 yo) |
| | | | Placebo: 2 (at |
| | | | least 1 ≥ 65 yo, |
| | | | remaining ≥ 18 yo) |
| Cohort | 120 mg/ | LD 120 mg/1 h | Total: 8 subjects |
| 2: | 720 mg | 48-hour washout period | BGE-105: 6 (at |
| | | LD 120 mg/1 h + MD | least 3 ≥ 65 yo, |
| | | 720 mg/22 h | remaining ≥ 18 yo) |
| | | | Placebo: 2 (at |
| | | | least 1 ≥ 65 yo, |
| | | | remaining ≥ 18 yo) |
| Cohort | 240 mg/ | LD 240 mg/1 h | Total: 8 subjects |
| 3: | 1440 mg | 48-hour washout period | BGE-105: 6 (at |
| | | 240 mg/1 h + MD | least 3 ≥ 65 yo, |
| | | 1440 mg/22 h | remaining ≥ 18 yo) |
| | | | Placebo: 2 (at |
| | | | least 1 ≥ 65 yo, |
| | | | remaining ≥ 18 yo) |

Part B: MD (30 subjects ≥ age 65 years)**

| | | | |
|---|---|---|---|
| Cohort | Placebo | Placebo Normal Saline | N = up to 10 |
| 1A: | NS | from Days 1 to Day 10 | |
| Cohort | 240 mg | BGE-105 240 mg for | N = up to 10 |
| 1B: | BGE-105 | 1 h every day up to | |
| | | 10 days from Day 1 | |
| | | to Day 10 | |
| Cohort | 240 mg/ | BGE-105 (dose and | N = up to 10 |
| 1C: | 1440 mg | regimen to be confirmed | |
| | | from 1B cohort) TBD | |
| | | mg over TBD hrs | |
| | | every day us to 10 days | |
| | | from Day 1 to Day 10 | |

Abbreviations: h = hour; LD = loading dose; MD = maintenance dose; yo = years old; TBD = to be determined
* The total number of subjects in Part B may be decreased or increased up to 48 subjects total.
**Heavy water 3 times daily starting on Day −5 through Day 10. D3-creatine 1 capsule on Day −3, Day 6, and Day 11.

A matching BGE-105, Placebo for Infusion product is created by using saline in corresponding IV bag sizes. If a label is added to the saline bags used to create active BGE-105 for infusion, the label for the placebo saline bags match to maintain the blind.

### Study Duration

Subjects in Part A (SAD) participate in this study for approximately 42 days including a Screening/Pre-Treatment Period of up to 14 days, a Treatment Period of 5 days, and a Follow-up Visit 27 days after the first administration of study drug (BGE-105 or placebo).

Subjects in the Part B (MD) participate in this study for approximately 81 days including a Screening Period of up to 16 days, an Outpatient Pre-Treatment Period of 5 days (Day −5 to Day −1) with heavy water and D3-creatine (D3-Cr), and a Treatment Period of 10 days on bed rest with heavy water/D3-Cr and BGE-105 or Placebo, and a post-dose Follow-Up Period (Days 11 to Day 60) that includes follow-up visits on Days 11, 12, 13, 14, 21, 30, and 60 days after the first administration of study drug (BGE-105 or placebo).

### Part A: Cohorts Screening and Pre-Treatment

Subjects in Part A are admitted to the clinic on Day −2. On Day −1, baseline assessments is performed, and the subject is randomized to blinded treatment with study drug (BGE-105 or placebo). An outline of the protocol for Part A cohorts is described below:
a. Subjects will be admitted to the clinic on the evening of Day −2 and will fast overnight for Baseline procedures on Day −1.
b. Screening-specific assessments include a pregnancy test (for subjects that are pre-menopausal), urine drug screen (dipstick test), alcohol breath test, and screening for HIV antibodies, HbsAg, and HCV antibodies.
c. Vital signs include diastolic and systolic blood pressure and pulse. In addition, temperature and respiratory rate should be collected on Day −1 at the timepoint that mimics Day 1 predose.
d. On Day −1, blood pressure was collected at timepoints that mimic Day 1 and Day 3 as closely as possible, based on the anticipated dose start time (2 h, 3 h, 4 h, 6 h, 8 h, and 12 h).
e. Safety laboratory tests include complete blood count, clinical chemistries, and urinalysis. Subjects are to be fasting for a minimum of 8 hours.
f. Continuous cardiac ECG telemetry monitoring was used, starting 24 hours before the administration of study drug.
g. Blood samples for plasma insulin and glucose monitoring should be collected in the morning, before breakfast and after fasting for 8 hours overnight.

Abbreviations: AE=adverse event; ECG=electrocardiogram; HbsAg=hepatitis B surface antigen; HCV=hepatitis C virus; HIV=human immunodeficiency virus; PD=pharmacodynamic; SAD=single ascending dose TBD=to be determined.

### Part A: SAD Cohorts Treatment and Follow-Up

On Day 1 for Part A, subjects receive LD, 1-hour infusion. On Day 3, after a 48-hour washout period, subjects then receive a 23-hour infusion (1-hour LD followed by a 22-hour MD). No earlier than 24 hours after the end of infusion, subjects is discharged from the clinic on Day 5 (End of Treatment Period [EOTP]). A treatment protocol for the SAD cohorts of Part A is described below:
a. Predose assessments (vital signs and PK assessments) should be done within 30 min of the start of infusion.
b. Vital signs include diastolic and systolic blood pressure and pulse. In addition, temperature and respiratory rate should be collected on predose Day 1 and EOT Day 5.
c. Safety laboratory tests include complete blood count, clinical chemistries, and urinalysis.
d. Assessments should be done within 1 hour after the end of the LD infusions on Day 1 and Day 3.
e. AEs was recorded from the start time of the first BGE-105 or placebo study drug infusion until the end of the subject's participation in the study. At discharge from the unit, any ongoing AEs was documented in source and in EDC be followed until the event is resolved or stabilized. Any new AEs occurring after discharge will clearly note the start date and end date.
f. Study drug was administered via IV infusion as a LD, 1-hour infusion on Day 1. After a 48-hour wash-out, study drug will then be administered as a 23-hour infusion (1-hour LD followed by a 22-hour MD).
g. Continuous cardiac ECG telemetry monitoring was used, continuing through the end of dosing on Day 4.
h. Blood samples for plasma insulin and glucose monitoring should be collected in the morning, before breakfast and after fasting overnight.
i. Assessments can be done any time after 24 hours after the end of infusion and before discharge.

Abbreviations: AE=adverse event; ECG=electrocardiogram; EOS=end of study; EOT=end of treatment; FU=follow-up; IP=investigational product; IV=intravenous; LD=loading dose; MD=maintenance dose; PD=pharmacodynamic; PK=pharmacokinetic; SAD=single ascending dose; TBD=to be determined.

### Part B: MD Cohorts Screening and Pre-Treatment

Subjects in Part B are admitted to the clinic on the evening of Day −2 and fast overnight from 10:00 PM for Baseline procedures on Day −1. A pre-treatment protocol for the MD cohorts of Part B is described below:
A. Subjects was admitted to the clinic on the evening of Day −2 and will fast overnight from 10:00 PM for Baseline procedures on Day −1. Subjects are to be fasting from 10:00 PM onward (min 8 hours fast) each night but may drink as much water as requested.
B. Screening-specific assessments include a pregnancy test (for subjects that are pre-menopausal), urine drug screen (dipstick test), alcohol breath test, and screening for HIV antibodies, HbsAg, and HCV antibodies.
C. Vital signs include diastolic and systolic blood pressure and pulse.
D. Safety laboratory tests include complete blood count, clinical chemistries, and urinalysis.
E. Samples are to be collected in the morning while subject is fasting from 10:00 PM the night before.
F. AEs was recorded from the start time of the first consumption of heavy water until the end of the subject's participation in the study. At discharge from the unit, any ongoing AEs was documented in source and in EDC was followed until the event is resolved or stabilized. Any new AEs occurring after discharge will clearly note the start date and end date.
G. Subject Bed Rest Screening Questionnaire.
H. Continuous cardiac ECG telemetry monitoring will start 24 hours before the start of the first BGE-105 or placebo infusion.
i. Muscle circumference, cross-sectional area, color flow analysis, AP diameter, and echo density of the vastus lateralis and gastrocnemius was measured by ultrasound. Ultrasound imaging must be conducted by the same technician throughout the study for all subjects. Ultrasound readings must be done by the same technician throughout the study for ALL subjects.
J. On Day −5 through Day −1, subjects will receive 3 doses of heavy water spaced approximately four hours apart, in the morning, afternoon, and evening. On Day −5, subjects will check into the clinic and be discharged to home approximately 1 hour after the last evening dose. Subject was dispensed adequate amounts of heavy water for home consumption until the next clinic visit. The site will telephone the subjects daily and remind them to consume heavy water as instructed.
K. Samples are to be collected before consumption of heavy water and the D3-Cr capsule, as applicable.
L. The samples for the D3-Cr urine assessments are to be collected from the second urination after waking up and while fasting from 10:00 PM the night before. The D3-Cr urine assessment must be done 48 h (+60 m) after the consumption of the D3-Cr capsule.
M. Subjects began a BMR-based diet starting with breakfast on Day −1 and site staff will encourage subjects to consume liquids. The total amount of meals in percentage and liquids in mL was recorded.
N. A wearable activity device such as a FitBit was dispensed to the subjects to wear from Day −5 through the last FU Visit on Day 60 to explore recording physical activity parameters. The site will adequately train subjects on how to operate the wearable at home and was instructed on charging the device and home-use.
O. Physical Function Assessment/Rehabilitation includes 1) the Sit to Stand Test, 2) Short Physical Performance Battery (SPPB), and 3) the Tinetti Performance Oriented Mobility Assessment (POMA). The baseline assessment for these tests was conducted by the occupational therapist while the subject is in-clinic anytime during the Pre-Treatment Period (Day −5, Day −3, Day −2 or Day −1). There was one occupational therapist assigned to each subject throughout the study and they will conduct all of these assessments for the subject to minimize bias and learning effects. These assessments will only be conducted once during the Pre-Treatment Period.
P. Study eligibility was confirmed with review of inclusion/exclusion criteria before assigning the subject number and the first consumption of heavy water.
Q. Weight only.
R. On Day −1, vital signs was collected at timepoints that mimic Day 1 as closely as possible, based on the anticipated start time of the BGE-105/placebo infusion (predose [additionally temperature and respiratory rate], 0.25 h, 0.5 h, 0.75 h, lh, 2 h, 3 h, 4 h, 6 h, 8 h, and 12 h).
S. Fasting plasma insulin and glucose collection was conducted at the timepoint that mimics Day 1 as closely as possible, based on the anticipated start time of the BGE-105/placebo infusion

D3-creatine capsules in Part B, is provided to subjects during the pre-treatment and treatment periods before breakfast and after fasting overnight at timepoints described previously. D3-creatine capsules are filled using creatine (methyl-D3) powder. Each capsule contains 30 mg of D3-creatine and cellulose filler.

Abbreviations: AP=anterior-posterior; BMR=basal metabolic rate; D3-Cr=deuterated creatine; ECG=electrocardiogram; FSR=fractional synthetic rate; HbsAg=hepatitis B surface antigen; HCV=hepatitis C virus; HIV=human immunodeficiency virus; PD=pharmacodynamic; TBD=to be determined.

### Part B: MD Cohorts Treatment and Follow-Up

A treatment protocol for the MD cohorts of Part B is described below:
A. For bed rest duration of >10 days, the Treatment Completion procedures was conducted on the day of the last BGE-105 infusion. EOT procedures was conducted 1 day after the last BGE-105 infusion. Day 12, 13, 14, 30 and 60 procedures was conducted 2, 3, 4, 20, and 50 days after the last BGE-105 infusion, respectively.
B. Subjects will remain in the clinic through Day 13, with the option to stay an extra day if returning to the clinic for protocol assessments on Day 14 is not logistically feasible.
C. Vital signs was collected daily and include diastolic and systolic blood pressure and pulse. On Days −1, 1, 5, and 10, vital signs was collected at the following timepoints: predose (additionally temperature and respiratory rate), 0.25 h, 0.5 h, 0.75 h, 1 h, 2 h, 3 h, 4 h, 6 h, 8 h, and 12 h after the start of study drug infusion. Vital signs will also be collected on Day 10 (23 h) and before discharge on Day 12.
D. Safety laboratory tests include complete blood count, clinical chemistries, and urinalysis.
E. Samples are to be collected in the morning. Subjects are to be fasting from 10:00 PM onward (min 8 hours fast) each night but may drink as much water as requested.
F. ECGs are to be taken predose and immediately following the completion of the 1-hour infusion. On Day 11, an ECG will also be taken again 23 h after the start of the Day 10 study drug infusion.
G. AEs was recorded from the start time of the first consumption of heavy water until the end of the subject's participation in the study. At discharge from the unit, any ongoing AEs was documented in source and in EDC was followed until the event is resolved or stabilized. Any new AEs occurring after discharge will clearly note the start date and end date.
H. BGE-105 study drug was administered via IV infusion as a 1-hour infusion.
I. PK samples were collected daily predose. On Days 1, 5, and 10, PK samples were collected at the following timepoints: predose, 0.25 h, 0.5 h, 0.75 h, 1 h, 2 h, 3 h, 4 h, 6 h, 8 h, and 12 h after the start of study drug infusion. Samples will also be collected 24 h (Day 11), 48 h (Day 12), 72 h (Day 13), and 96 h (Day 14) after the start of the last infusion. PK samples were taken from the opposite arm used for the IV infusion.
J. Continuous cardiac ECG telemetry monitoring was used starting on Day 1 pre-dose and continuing through the end of dosing on Day 10.
K. Subjects were asked to remain in their beds, apart from using a wheelchair to be taken to the shower and they will shower seated. They will also use a wheelchair to get to and from the bathroom. When not in bed, subjects were in a wheelchair.
L. Subjects will wear the pneumatic compression cuffs the entire duration of bed rest, except for when in a wheelchair to use the bathroom and shower.
M. Muscle circumference, cross-sectional area, color flow analysis, AP diameter, and echo density of the vastus lateralis and gastrocnemius were measured by ultrasound. Ultrasound imaging must be conducted by the same technician throughout the study for all subjects. Ultrasound readings must be done by the same technician throughout the study for all subjects.
N. Ultrasound and skeletal muscle micro-biopsy were conducted on Day 6 predose and on Day 11 at the 24 h timepoint.
O. On Day 1 through Day 10, subjects will receive 3 doses of heavy water spaced approximately four hours apart, in the morning, afternoon, and evening.
P. Samples are to be collected before consumption of heavy water and the D3-Cr capsule, as applicable.
Q. The samples for the D3-Cr urine assessments are to be collected after an overnight fast starting at 10 pm the night before AND from the second urination after waking up while still fasted On Days −1, 8, and 13, the D3-Cr urine assessments must be done 48 h (+60 m) after the consumption of the D3-Cr capsules.
R. Subjects were on a BMR-based diet ending at 24 h on Day 11 (after the ultrasound, muscle microbiopsy, and laboratory samples are collected) and site staff will encourage subjects to consume liquids. The total amount of meals in percentage and liquids in mL was recorded.
S. A wearable activity device such as a FitBit and corresponding electronic tablet was dispensed to the subjects to use from Day −5 through the last follow up (FU) Visit on Day 60 to explore recording of physical activity parameters. The site will adequately train subjects on how to operate the wearable at home and was instructed on charging the device and home-use. Subjects were able to keep the device and tablet for personal use at the Day 60 Follow-Up Visit.
T. Procedures are to be conducted before starting the period of bed rest.
U. Physical Function Assessment/Rehabilitation includes 1) the Sit to Stand Test, 2) Short Physical Performance Battery (SPPB), and 3) the Tinetti Performance Oriented Mobility Assessment (POMA). The Sit to Stand Test was conducted on Day 12, 13, 14, 30, and 60. The SPPB was conducted on Day 12 and 30. The POMA was conducted on Day 13, 14, 21, 30, and 60 (only if the Day 30 score was not within +10% of the baseline score). There was an occupational therapist assigned to each subject throughout the study and they will conduct all of these assessments for the subject on each study day to minimize bias and learning effects. These assessments will only be conducted once on each study day. If the Day 30 POMA score is not within +/−10% of the baseline score, subjects may have additional telehealth appointments between the Day 30 and Day 60 if needed. The POMA was conducted and documented in the source.

Abbreviations: AP=anterior-posterior; BMR=basal metabolic rate; D3-Cr=deuterated creatine; ECG=electrocardiogram; FSR=fractional synthetic rate; HbsAg=hepatitis B surface antigen; HCV=hepatitis C virus; HIV=human immunodeficiency virus; PD=pharmacodynamic; TBD=to be determined.

### Follow-Up Period

For Part A, on Day 28 (approximately 27 days after administration of the first dose of study drug), subjects are contacted by telephone for safety follow-up assessments. The date on which the subject completes the Follow-up Visit is the subject's end of study date (or EOS). For Part B, subjects remain in-clinic through Day 13 for safety follow-up assessments, laboratory sample collection, and physical function rehabilitation following 10 days of bed rest. Subjects have the option to remain in-clinic for an additional day if returning to the clinic for protocol assessments on Day 14 is not logistically feasible. Subjects have a telehealth visit on Day 21, and in-clinic visits on Days 30 and 60 for safety follow-up assessments.

### Pharmacokinetic and Pharmacodynamic Assessments

### Ultrasound

For subjects in Part B only, an ultrasound is performed at timepoints described previously to measure muscle circumference, cross-sectional area, color flow analysis, AP diameter, and echo density of the vastus lateralis and gastrocnemius on one of the legs. The leg measured has to remain consistent.

Ultrasound images were collected during the following study visits: Day −1 (baseline), Day 6 (pre-dose), and day 11 (end of treatment). Ultrasounds were performed of both the vastus lateralis and gastrocnemius to measure cross-sectional area, color flow doppler, antero-posterior (AP) diameter, and echo density. Muscle circumference was also measured. Ultrasound imaging was conducted by the same operator throughout the study for all subjects. Ultrasound readings were done by the same reader throughout the study for all subjects. The ultrasound operator and reader were blinded to the study treatment, whether subjects received active study drug BGE-105 or placebo. The leg measured (right vs. left) and leg location (medial vs. lateral) remained consistent for all ultrasounds.

Muscle circumference was measured in centimeters (cm) using a measuring tape and recorded on the ultrasound worksheet at the time of imaging. The targeted measurements for circumference that were acquired at the below markers: 15 cm superior of the mid patella for the Vastus Lateralis, and 3-inch inferior to the popliteal vessels for the gastrocnemius.

### Ultrasound Procedure

The ultrasound images were collected before conducting the skeletal muscle micro-biopsy.

Prior to testing, subjects were instructed to wear shorts on testing day to avoid compression of the upper leg musculature and to expose the upper portion of the thigh.

Subjects were required to lay supine on an examination table with both legs fully extended for a minimum of 5 minutes to allow for fluid shifts to occur.

Each subject was instructed to lay on their non-dominant side to obtain skeletal muscle ultrasound images of the vastus lateralis and gastrocnemius in the dominant leg.

Subjects were positioned with their legs on top of one another and slightly bent at the knee. Ultrasound images of the vastus lateralis will be captured at 50% of the straight-line distance from the greater trochanter and the lateral epicondyle of the femur.

To ensure proper probe placement and consistent image capture location, a dotted line was drawn transversely and longitudinally along the surface of the skin from the aforementioned location

All measures of muscle morphology were obtained using a B-mode, 12-MHz linear probe US (General Electric vivid E9) to provide acoustic contact without depressing the dermal layer of the skin.

Longitudinal B-mode and transverse field of view (FOV) images were acquired during each exam and analyzed.

Ultrasound settings remained fixed for examination of each subject:

image gain was set at 50 decibels (dB), dynamic range was set at 72, and image depth was set at 5 cm

Three panoramic transverse images (PTI) were captured in the transverse plane, perpendicular to the long axis of the muscle.

These images utilized the extended-field-of-view ultrasonography in order to include entire area of the vastus lateralis in a single panoramic image.

Additionally, three single longitudinal images (SLI) were captured in the longitudinal plane, parallel to the long axis of the muscle.

### Single Still Longitudinal Image (SLI) Selection:

i. Consistent pressure was applied by probe for Minimal muscle compression
ii. To avoid affecting the measurement value, the probe was carefully set on the thigh using plenty of ultrasound gel so that the probe does not directly touch the skin to push the soft tissue
iii. Superficial aponeurosis of vastus lateralis (adipose tissue/muscle interface) was as close to horizontal as possible
iv. Entire length of image consisted of muscle fibers (no aponeuroses or inconsistencies in probe pressure or placement)

This same technique was applied to ultrasound of the gastrocnemius

Either the medial head or lateral head of the gastrocnemius was used for ultrasound evaluation—not both.

The same head was used for all gastrocnemius ultrasounds throughout the study.

### Heavy Water Consumption and Assessments

For subjects in Part B only, heavy water, deuterated H2O (D2O), is provided to subjects to drink during the pre-treatment and treatment periods before breakfast, in the afternoon, and after fasting overnight, at timepoints described previously.

Blood samples (10 mL) and urine samples are collected at timepoints described previously to assess skeletal muscle protein fractional synthetic rate (FSR).

### Plasma Insulin and Glucose

Blood samples (4 mL) for plasma insulin and glucose monitoring are collected in the morning, before breakfast and after fasting overnight at timepoints described previously.

### Proteomics

For subjects in Part B only, blood samples (4 mL) for proteomic analysis are collected at timepoints described previously.

### Bioenergetics

For subjects in Part B only, blood samples (8 mL) for bioenergetic assessments are collected at timepoints described previously.

### Sample Size:

No formal sample size calculation has been done. Cohort and overall study size are based on practical considerations. The study plan is to enroll up to 72 volunteer subjects to receive at least one dose of BGE-105 or placebo.

In the SAD (Part A), 18 subjects received BGE-105 and 6 subjects received placebo.

For the MD (Part B), 30 subjects receive BGE-105 or placebo. All Part B subjects are ≥65 years of age. A subject may be replaced on a case-by-case basis at the discretion of the sponsor. The replacement subject is assigned the same treatment as the subject being replaced.

### Analysis Populations:

The Safety Analysis Set include all subjects who had received≥1 administration of study drug (either BGE-105 or placebo). The Safety Analysis Set is used for safety analysis. Subjects is analyzed based on the actual treatment received.

The Pharmacokinetic (PK) Set include enrolled subjects who had received≥1 administration of study drug without any event and/or major protocol deviation affecting the PK evaluation and with completed PK profile(s). The inclusion/exclusion of subjects with incomplete PK profile(s) in this set is agreed upon between the Sponsor and the CRO before the PK concentration dataset is locked.

The Pharmacodynamic (PD) Set include all enrolled subjects who have completed the study without any protocol deviation affecting the PD evaluation with a baseline sample and ≥1 postbaseline sample for PD evaluation. The inclusion/exclusion of subjects with incomplete PD profile(s) in this set is agreed upon between the Sponsor and the CRO before the PD concentration dataset is locked.

The Pharmacokinetic/Pharmacodynamics (PK/PD) Set include all subjects who are in both the PD Set and the PK Set.

### Pharmacokinetic Analysis:

Individual BGE-105 plasma concentrations is listed and descriptive statistics including means, geometric means, medians, ranges, standard deviations, and coefficients of variation is provided. Corresponding concentration-time profiles (individual and means) is displayed in plots.

Relevant plasma PK parameters are derived for BGE-105 by standard noncompartmental methods and tabulated along with descriptive statistics and graphs.

The linear dose-proportionality of Cmax, AUC0-t, and AUC0-inf (if applicable) are investigated for BGE-105 using an exponential regression model (“power model”).

### Pharmacodynamic Analysis:

Data is analyzed as change from baseline within each dose cohort and treatment group. Treatment comparisons are made as a linear contrast of all treated compared to all placebo and for each individual dose cohort versus all placebo based on time-matched samples acquired during the 24-hour period on Day −1. P-values are reported uncorrected for multiplicity.

For Part B, changes in leg circumference, cross-sectional area, color flow analysis, AP diameter, and echo density of the vastus lateralis and gastrocnemius, as measured by ultrasound, are analyzed first as a linear contrast comparing all treated to placebo and then as individual dose comparisons to placebo. P-values are reported uncorrected for multiplicity.

Skeletal muscle protein synthesis rate (%/time) for individual muscle proteins are compared first as a linear contrast including all doses versus placebo and then as individual dose comparisons to placebo.

The Benjamini-Hochberg procedure are used to control the false positive discovery rate for the multiplicity of proteins assessed.

D3-creatine total muscle mass is analyzed as change from baseline within each cohort and treatment group. Treatment comparisons is made as a linear contrast of all treated compared to all placebo and for each individual dose cohort versus all placebo based on time-matched samples acquired.

The micro needle biopsy is analyzed by three different measures:

Targeted Biomarkers: The means of targeted biomarkers (creatine kinase-muscle [CK-M], etc.) for each MD cohort is compared using analysis of variance (ANOVA).

Proteomics: The proportion of proteins with high vs. low FSR is compared against baseline values.

Changes in Protein (Exploratory): the detection of individual proteins with significant changes over time, after correction for multiple comparisons.

The Sit to Stand Test and the SPPB are analyzed as a change from within subject and as applicable by each dose cohort and treatment group. The POMA assessment is an exploratory measure. Any changes from baseline are summarized within subject and as applicable by each dose cohort and treatment group.

### Pharmacokinetic Pharmacodynamic Analysis:

Individual biomarker plasma concentrations were listed and descriptive statistics including means, geometric means, medians, ranges, standard deviations, and coefficients of variation are provided. Corresponding concentration-time profiles (individual and means) are displayed in plots.

Relevant PD parameters are listed and tabulated along with descriptive statistics and graphs.

The relationship between BGE-105 and plasma and other PD parameters were investigated using a graphical exploratory and simple modelling approach.

### Unblinded Data Review:

There are unblinded data reviews conducted by the Sponsor after the completion of each cohort. An unblinded data review takes place after the completion of Part A to confirm the study design and dose for Part B. A Sponsor unblinded data review is also conducted after the completion of each of the MD cohorts.

### 6.8.3. Results for 3 SAD Cohorts in Part A—BGE-105-101 Study

PK data from the 3 SAD cohorts demonstrated dose proportionality displayed in FIGS. 48A-B. The Cmax remained within the expected range and for the highest dose (240 mg/1440 mg) the AUCₗₐₛₜ was 1062 μg*hr/mL (Table 34 below).

For the SAD cohorts in Part A, all doses were well tolerated including the highest dose of 240 mg/1440 mg. There were no emerging safety concerns or trends and there were no serious adverse events. A maximum tolerated dose from the 3 SAD cohorts was not determined.

TABLE 34
Summary of Pharmacokinetic Parameters of Part A (Single Ascending Dose) of Ongoing Phase 1 Study BGE-105-101
Mean (% CV)

| | SAD1 (60 mg/360 mg) | | | | SAD2 (120 mg/720 mg) | | | | SAD3 (240 mg/1440 mg) | | | |
|---|---|---|---|---|---|---|---|---|---|---|---|---|
| | Day 1 | | Day 3 | | Day 1 | | Day 3 | | Day 1 | | Day 3 | |
| Cmax (μg/mL) | 7.430 | (22%) | 10.147 | (16%) | 13.662 | (25%) | 14.808 | (32%) | 26.720 | (41%) | 35.620 | (44%) |
| Dose-normalized | 0.12 | (22%) | — | | 0.11 | (25%) | — | | 0.11 | (41%) | — | |
| Cmax | | | | | | | | | | | | |
| AUC0-24 (μg · hr/mL) | 34.701 | (28%) | 173.033 | (19%) | 53.736 | (32%) | 266.900 | (31%) | 107.216 | (57%) | 612.045 | (44%) |
| AUClast (μg · hr/mL) | 37.384 | (29%) | 306.409 | (19%) | 56.810 | (33%) | 449.858 | (32%) | 113.960 | (58%) | 1062.000 | (45%) |

| | | | | | | | | | |
|---|---|---|---|---|---|---|---|---|---|
| AUCinf (μg · hr/mL) | 39.582 | (31%) | — | 58.814 | (34%) | — | 119.315 | (60%) | — |
| Dose-normalized | 0.66 | (31%) | — | 0.49 | (34%) | — | 0.50 | (60%) | — |
| AUCinf | | | | | | | | | |
| Half-life (hr) | 9.1 | (20%) | — | 8.6 | (12%) | — | 8.9 | (17%) | — |

FIG. 48A-48B shows the dose proportionality for the 3 SAD cohorts. The 95% confidence interval for the slope of dose vs Cmax (A) and AUC(B) includes 1, suggesting that BGE-105 PK is linear across the dose range. Low point estimates and wide confidence interval were likely due to between-subject variability.

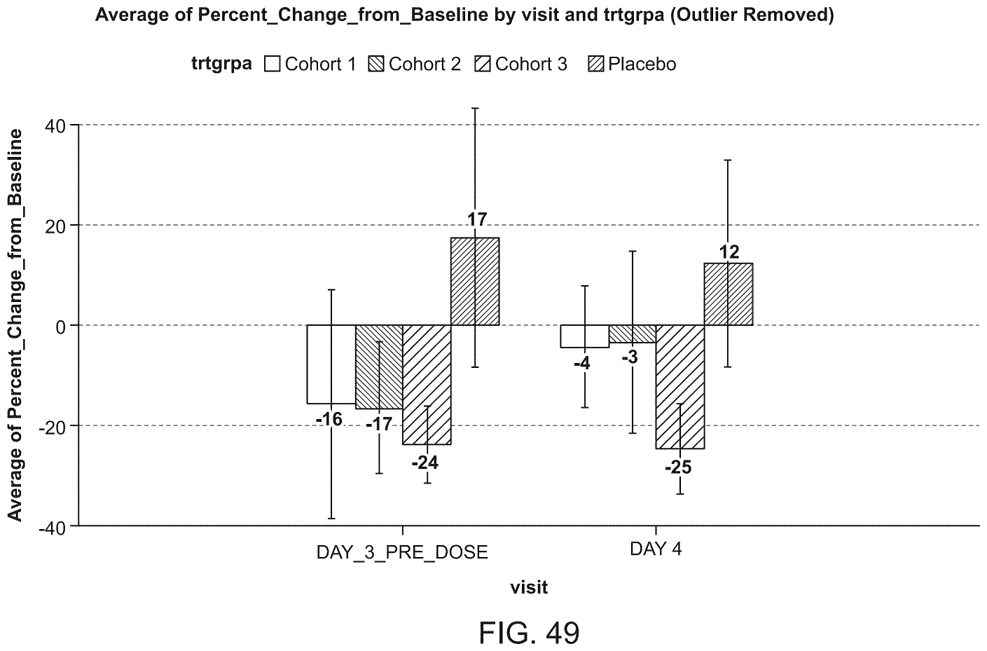

FIG. 49 shows that there is there is a 17% increase from baseline in HOMA-IR on the Day 3 Predose visit and 12% increase from baseline in HOMA-IR on the Day 4 visit for the Placebo group of the SAD study (Part A). In the BGE-105 treated patients (cohorts 1,2, and 3) of the SAD study (Part A), there is a decrease in the percent change from baseline in HOMA-IR for both visits with the largest decrease in the highest dosing group (cohort 3).

### 6.8.4. Results for 3 MD Cohorts in Part B—BGE-105 Study

In the double-blind, placebo-controlled study, 21 healthy volunteers aged≥65 years underwent strict bed rest for 10 days while receiving daily IV infusions of placebo (n=10) or a fixed dosage of BGE-105 (n=11). One day before (baseline) and 5 and 10 days after initiation of bed rest, key muscle atrophy endpoints were measured: thigh circumference; cross-sectional area (CSA) and A-P diameter of vastus lateralis (ultrasound); ultrasound muscle quality grade, an index that quantifies fatty degeneration in muscle (ultrasound echo density); and muscle protein synthesis rate (biopsy). Parameters measured during the 10-day bed rest period include thigh circumference, muscle size, muscle quality (e.g., fatty degeneration), and muscle protein synthetic rate. Table 35 summarizes the results.

TABLE 35
Key aspects of muscle atrophy outcomes after 10-day bed resting in older adults

| | | | Cross- | AP | | Cumulative |
|---|---|---|---|---|---|---|
| | | Thigh | sectional | diameter | Muscle grade | protein |
| | | circumference | area (CSA) | (thickness) | (progression) | synthesis |
| % change: | Placebo | −6.4% | −21.2% | −19.5% | −80% | 15.9% |
| baseline to D10 | | | | | (8 out of 10) | |
| | BGE-105 Single | +0.8% | −5.7% | −8.0% | −9% | 22% |
| | dosage of 240 mg | | | | (1 out of 11) | |
| | daily (cohort MD 1B) | | | | | |
| p value | | <0.0001 | <0.01 | <0.05 | <0.005 | <0.005 |

*Effect size based on Cramer's V. All percentages are relative to baseline values measured 1 day before initiation of dosing.

Measurements of thigh circumference and the vastus lateralis are among the gold standard markers of skeletal muscle atrophy. The vastus lateralis, the largest muscle in the quads, is among the commonly studied muscles given (a) its functional importance in mobility and disability, (b) location and architecture of the muscle, which results in ease of ultrasound measurement and biopsy, and (c) atrophy is more profound in older patients than the muscles in the lower leg. Endpoint metrics were measured at baseline, after 5 days of bedrest, and after 10 days of bedrest, including endpoints such as limb circumference, muscle area via ultrasound, and measurement of muscle quality via ultrasound (measured normal muscle vs fat), fractional synthetic rates. A number of endpoint metrics correlated with muscle size and muscle function biochemically, such as the thigh circumference, size of the muscle via ultrasound, to calculate diameter, thickness, and cross-sectional area of the muscle (calculated after ultrasound).

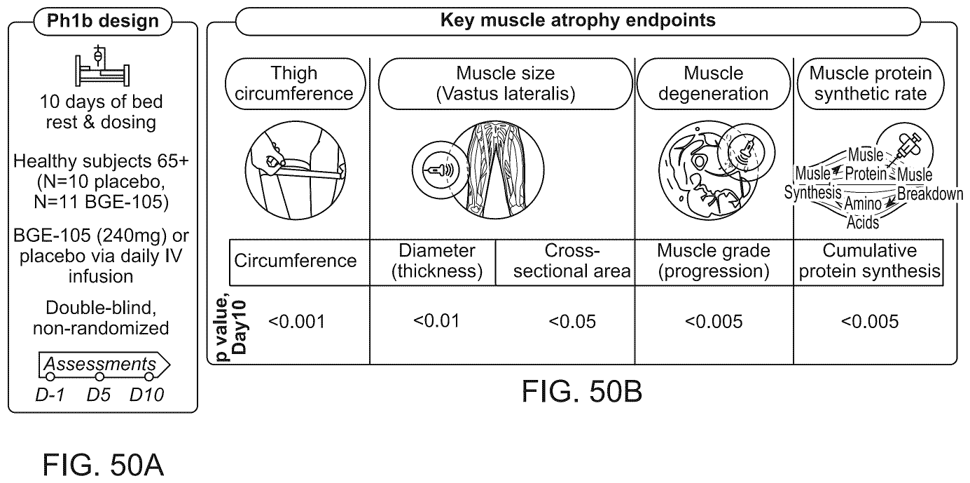

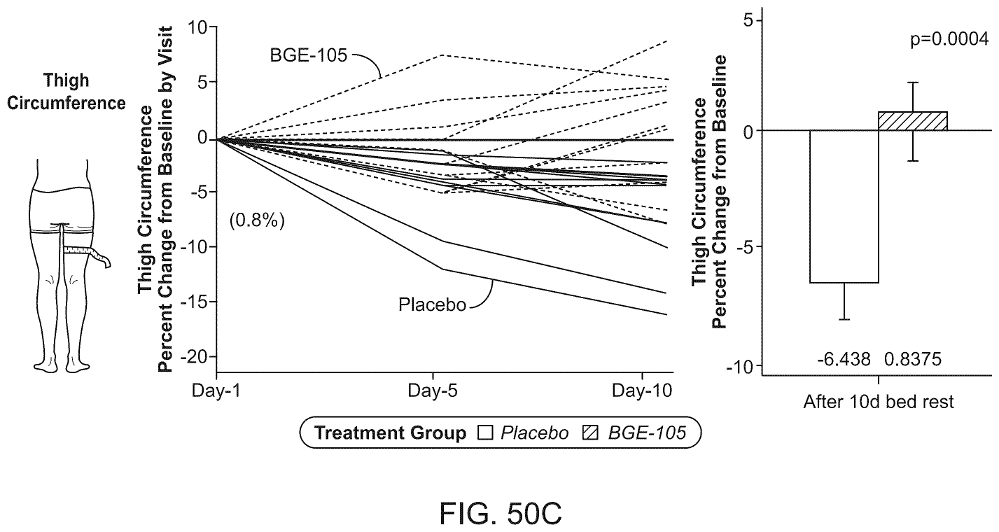

FIGS. 50A-50B shows that patients treated with BGE-105 (Cohort 1B) had an increase in thigh circumference, muscle size, muscle quality, and muscle protein synthesis rate as compared to the placebo group (Cohort 1A).

FIG. 50C shows that BGE-105 significantly prevented bed rest-induced reduction in thigh circumference (middle panel). Older patients experienced about 3.2-4.7% loss of total lean body mass, and about 6-7% loss of lean leg mass (cohort 1A, MD1A). After the 10-day bed rest period, older patients in the placebo group showed a 6.438% reduction of thigh circumference while older patients in the BGE-105 treated group show a 0.8% increase in thigh circumference, <0.0001. Measurements were made 15 cm superior of the mid patella (left panel). Dashed lines represent the treated patients, versus the solid lines showing the placebo subjects. FIG. 50C shows that treated patients with BGE-105 show higher thigh circumference as compared to the placebo (cohort 1A). All percentages are relative to baseline values measured 1 day before initiation of dosing.

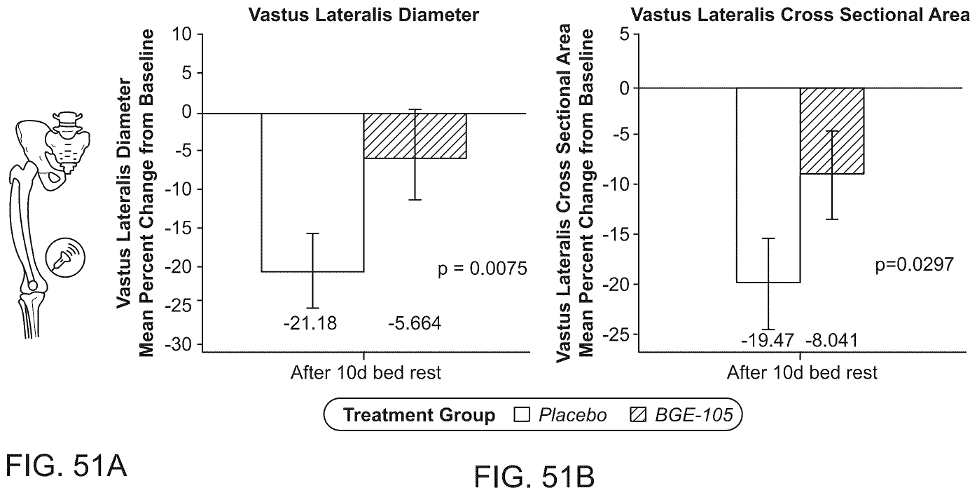

Muscle size was measured as a function of ultrasound, and the results showed that there was a 21% decrease in the placebo group (Cohort 1A) as compared 5.664% decrease in the treatment group (Cohort 1B), showing about a 75% improvement in muscle dimension. FIG. 51B shows that BGE-105 significantly prevented bed rest-induced reduction in thickness and cross-sectional area of the vastus lateralis via ultrasound (left panel). After 10-day bed rest, subjects with BGE-105 treatment had only a 5.664% reduction of vastus lateralis diameter—as compared to patients in the placebo group showing a 21.18% reduction of the vastus lateralis diameter (p=0.0075) (middle panel). Similar results are seen in the measurements of vastus lateralis cross sectional area (right panel). BGE-105 treated subjects showed a 8.041% reduction of the cross-sectional area of the Vastus Lateralis, which is a significantly reduction as compared to the 19.47% reduction of the cross-sectional area of the Vastus Lateralis observed in the placebo group (p=0.0297). Percentages are shown as the mean percent change from baseline.

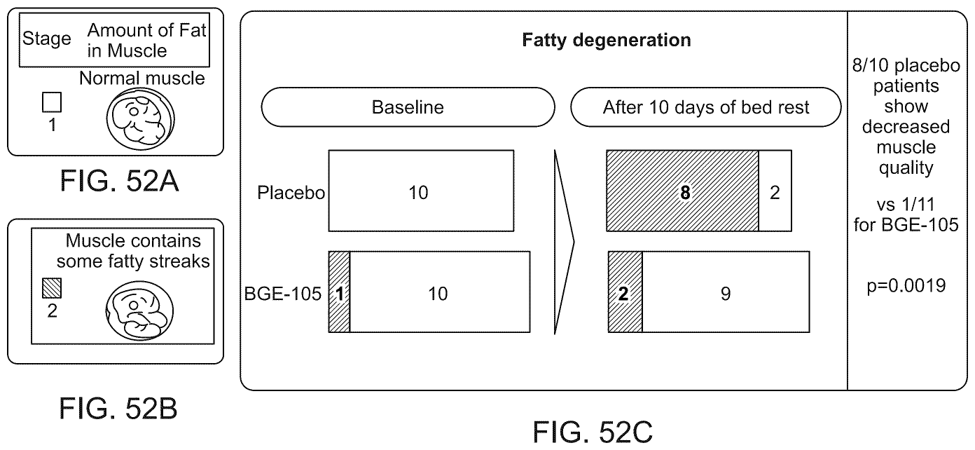

FIG. 52A-52C, show that BGE-105 significantly prevented bed rest-induced fatty degeneration of the vastus lateralis as determined via echo density; an echo density measurement determines the muscle quality. Muscle grading was performed according to panels A-B. The echo density measurements showed a 6.4% decrease in the placebo group (Cohort 1A), as compared to a 0.8% increase in the treatment group (Cohort 1B, 240 mg daily).

FIG. 52A illustrates amount of fat in normal muscle (1), FIG. 52B illustrates muscle containing some fatty streaks (2). Normal muscle (grade 1), and muscle that contains some fatty streaks (grade 2). After 10 days of bed rest, eight (8) out of ten (10) patients in the placebo group advanced to a grade 2, whereas only 1 out of 11 BGE-105 treated patients moved to a grade 2 after 10 days of bed rest. (p=0.0019). The echo density measurements showed that 80% of patients went from an ultrasound muscle quality grade of 1 (normal muscle tissue) to a 2 (muscle tissue with fatty streaks), whereas in the BGE-105 treatment group (Cohort 1B), only 1 patient went from a grade of 1 to a 2, showing statistical significance.

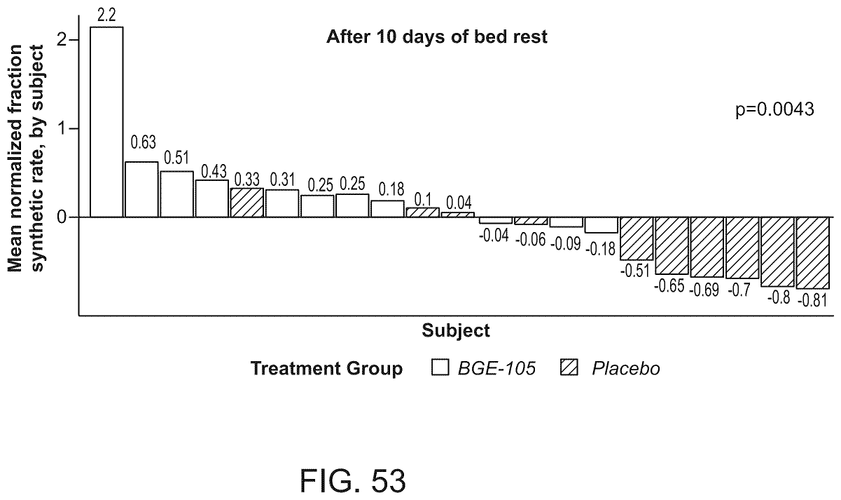

FIG. 53 shows that BGE-105 resulted in an elevated rate of muscle protein synthesis in the vastus lateralis, measured via microbiopsy. The graph represents a ratio of the BGE-105-treated group versus the placebo group. The bars above the x-axis represent a higher level of muscle protein synthesis versus the placebo group after 10 days of bedrest. The muscle protein synthetic rate showed the cumulative muscle synthesis rate over the course of 10 days of bed rest. The muscle cumulative protein synthesis rate significantly increased in the BGE-105 treatment group (Cohort 1A) as compared to the placebo group (Cohort 1A). After 10 days of bed rest, 8 out of 11 (about 72.7%) of BGE-105 treated patients experience elevated rate of muscle protein synthesis, and have a mean normalized fraction synthetic rate ranging from 0.18-2.2. In comparison, only 3 out of 10 (30%) of placebo patients experienced elevated rate of muscle protein synthesis. The mean normalized fraction of synthetic rate ranges from 0.04-0.33. The results demonstrate that BGE-105 is efficient in inducing or maintaining muscle protein synthesis in the vastus lateralis (p=0.0043).

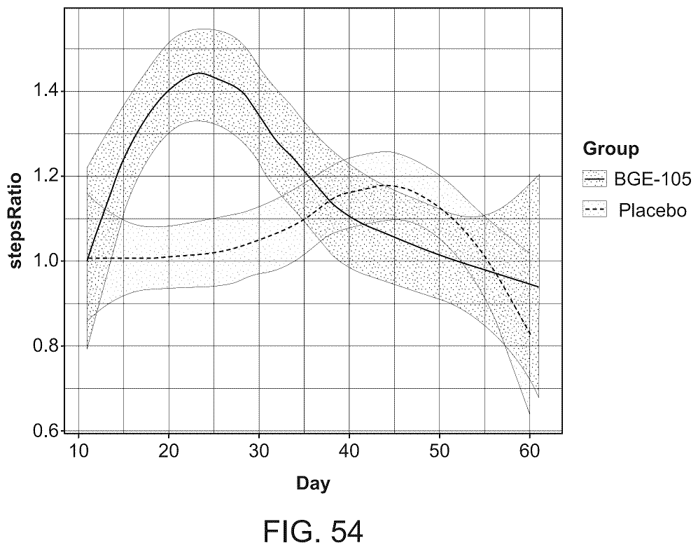

FIG. 54 shows the step ratio of patients who wore a wearable activity device during Day 10 through Day 60 (post-bedrest time period) of the Phase 1b, MD, Part B clinical trial of Example 8. The ratio is calculated by dividing eat subjects later step counts by their baseline step count, which is the mean daily steps seen over the pre-bedrest period. Patients treated with BGE-105 showed an increase in physical activity by determining the number of steps taken by the patient using the wearable activity device as compared to the patients who received a placebo. The activity levels start off near the baseline and then increase for the BGE-105 treated group for several weeks before the curve converges again.

Muscle atrophy, loss of muscle mass and strength, is a universal feature of human aging that increases the risk of multiple morbidities, shortens lifespan, and diminishes quality of life. Hospitalization and periods of forced inactivity greatly accelerate this loss in older people.

The analysis of the inventors' unique human aging cohorts revealed that the apelin pathway is a strong predictor of healthy longevity and muscle function, and translated directly into the clinical finding of this study that apelin pathway activation with BGE-105 improved muscle physiology in older adults.

Analysis of proprietary human biobanks showed that apelin pathway activity, which declines with age, was positively associated with longevity, mobility, and cognitive function. Apelin, the natural ligand of APJ, is secreted by skeletal muscle in response to exercise and regulates multiple aspects of muscle metabolism, growth, and repair.

The double-blind, placebo-controlled clinical trial evaluated the safety and pharmacodynamics of BGE-105. Twenty-one volunteers underwent 10 days of bed rest while receiving infusions of BGE-105 or placebo.

Volunteers on placebo (n=10) exhibited muscle atrophy, reflected by statistically significant reductions in thigh circumference and ultrasound measurement of vastus lateralis muscle dimensions (cross sectional area and thickness) and muscle quality (fatty degeneration).

Treatment with BGE-105 (n=11) significantly ameliorated muscle atrophy relative to placebo:

Muscle dimensions: Volunteers receiving BGE-105 showed a 100% improvement in thigh circumference (p<0.001) relative to placebo-treated volunteers, and ultrasound measurements showed a 58% improvement in vastus lateralis cross-sectional area (p<0.05) and a 73% improvement in vastus lateralis thickness (p<0.01).

Muscle quality: Ultrasound echo density measurements revealed that the muscle quality grading scale, an index that quantifies degeneration in muscle, worsened in 8 of 10 volunteers on placebo vs. only 1 of 11 volunteers receiving BGE-105 (p<0.005).

Muscle protein synthesis: Proteomic analysis of muscle microbiopsy samples revealed that bed rest decreased production of muscle proteins, and this effect was significantly ameliorated by BGE-105 (p<0.005). The higher rate of muscle protein synthesis in the drug vs. placebo group provides a potential mechanistic basis for BGE-105's protective effect on muscle dimensions.

Apelin agonist BGE-105 resulted in statistically significant improvement vs. placebo in muscle size, quality, and protein synthesis in volunteers≥65 years old during 10 days of bed rest, with no serious adverse effects. BGE-105 treatment resulted in statistically significant prevention of muscle atrophy relative to placebo in healthy volunteers aged 65 or older after 10 days of strict bed rest.

BGE-105 was well tolerated in the study in terms of safety.

BGE-105 significantly reduced muscle atrophy across multiple key endpoints, in healthy volunteers aged≥65 years. The higher rate of muscle protein synthesis in BGE-105 treated vs. placebo group provided a mechanistic basis for BGE-105's protective effect on muscle dimensions. The findings of the Phase 1b clinical trial support investigation of BGE-105 as a treatment of a wide range of age-related syndromes driven by loss of muscle. These conditions include acute myopathies in hospitalized patients on mechanical ventilation, as well as chronic medical conditions that are common among millions of older people but lack approved therapeutics for prevention or treatment, representing an enormous unmet clinical need.

### Proteomic Signatures of Physical Function and Mortality

Proteomic profiling and analysis were performed on serum collected from the phase 1B clinical trial subjects (treated vs placebo) of Example 9. 11 treated and 11 placebo subject's serum levels were profiled for their proteomics collected at day −1 (baseline), day 5 and day 11. A linear regression model was implemented with an interaction term between treatment group and day to identify proteins whose differential abundance between the treatment groups influenced the average rate of change per day of a given protein. This model was fit separately for all proteins measured, and the resulting coefficient (on the interaction term) for each protein was used to rank order all proteins from most positive to most negative coefficient.

To test if the effect of BGE-105 on the plasma proteome significantly affected proteomic signatures of physical function and mortality, an enrichment analysis was performed using GSEA method, using (1) the aforementioned ranked list of proteins affected by BGE-105 and (2) various protein sets. For each human aging cohort phenotype (various physical function phenotypes and mortality), two protein sets consisting respectively (to preserve directionality) of the proteins positively and negatively associated (p<0.05) with that phenotype. Significant phenotypes with their GSEA plots and p-values (significant phenotypes were not shown when their protein sets overlapped substantially with those of already shown phenotypes).

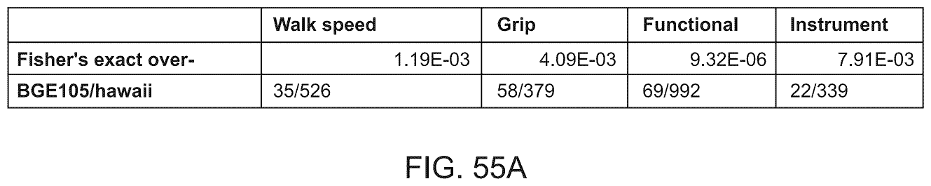

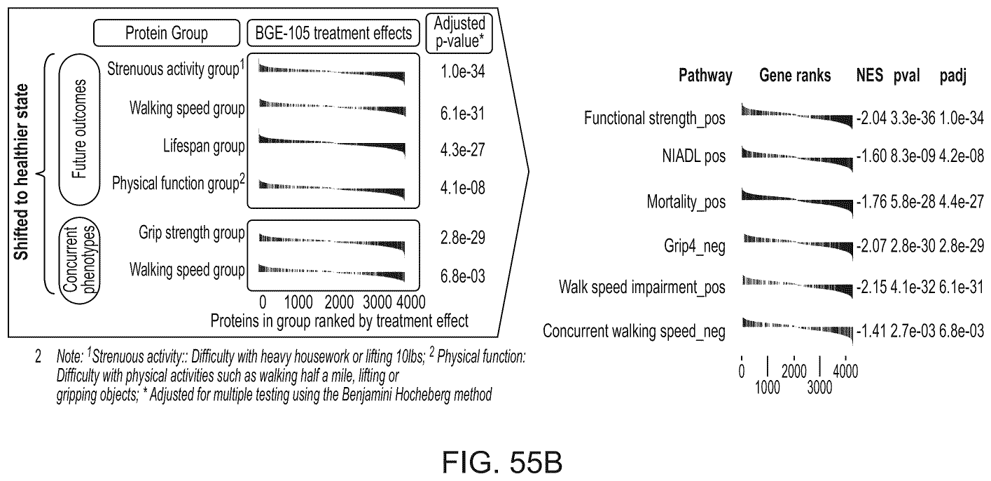

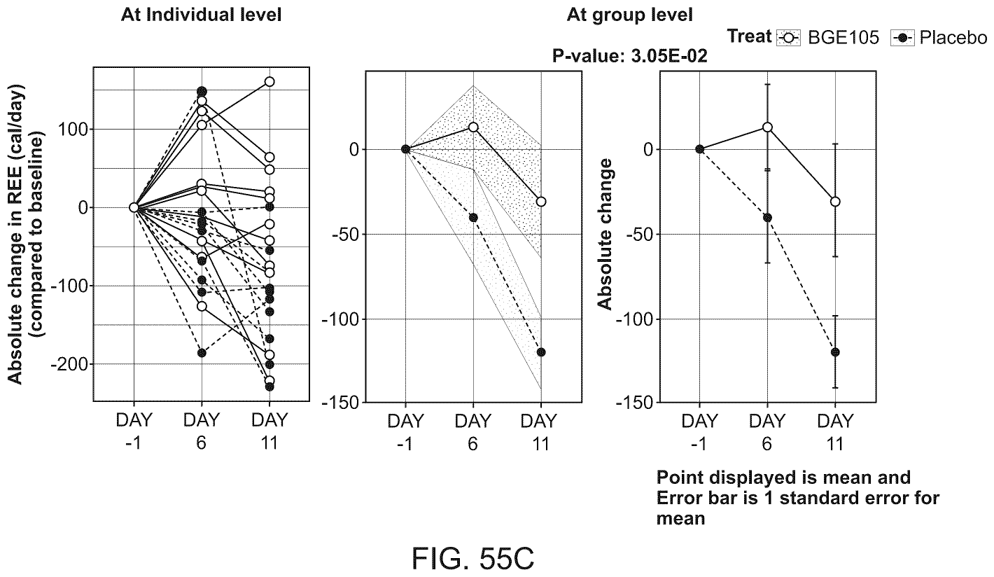

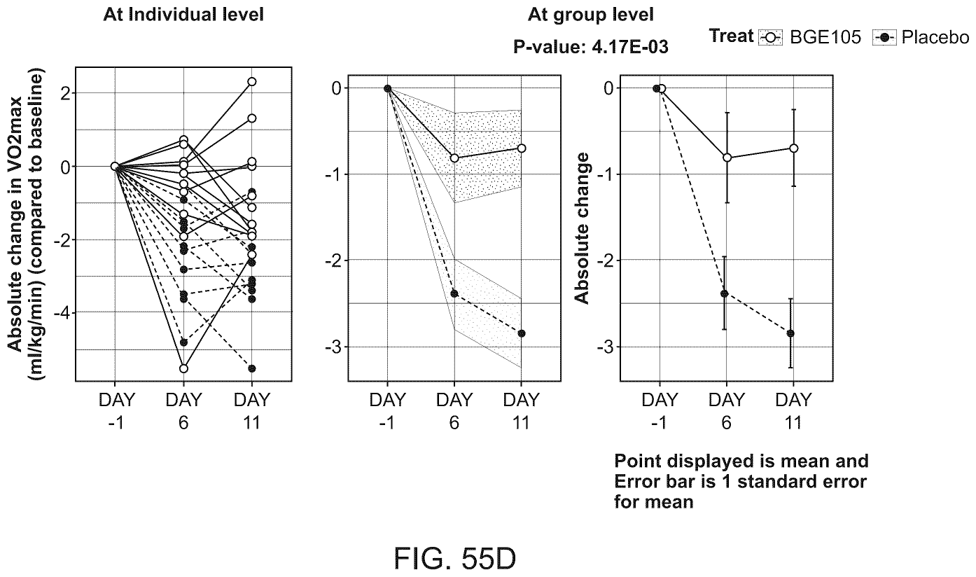

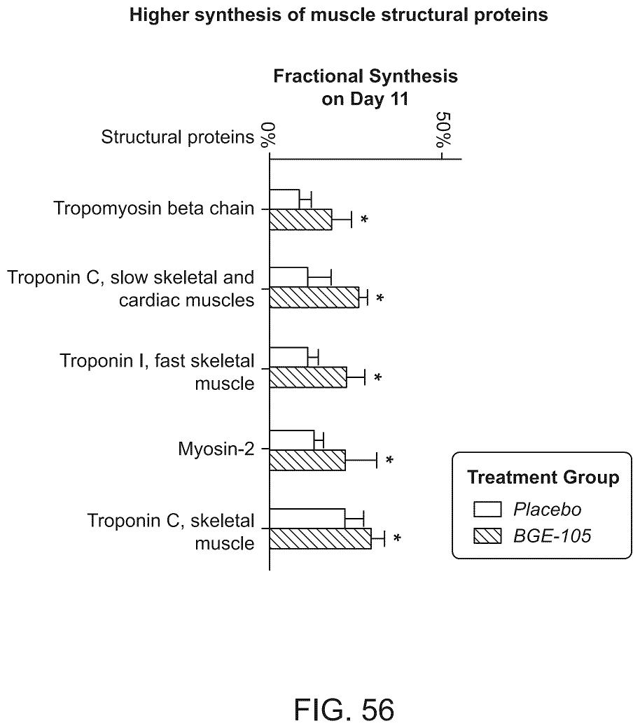

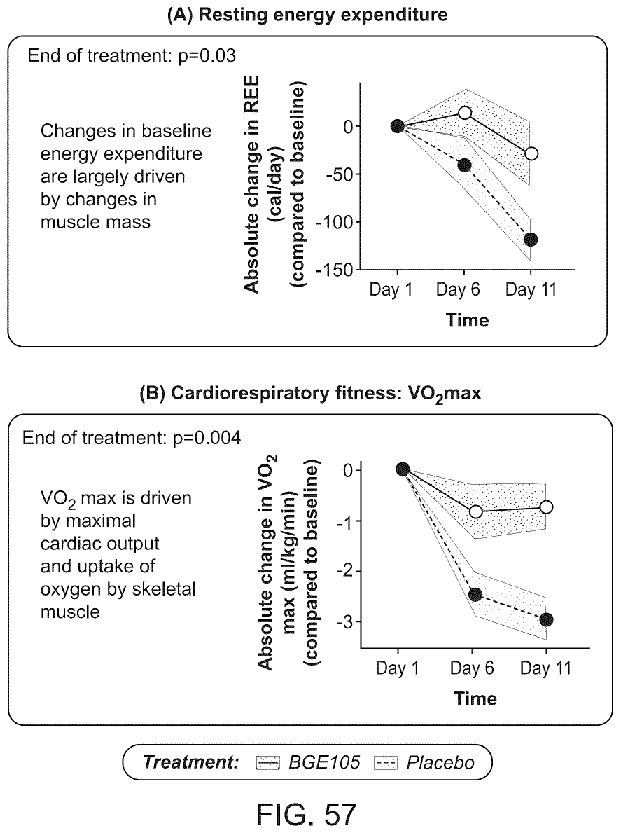

The proteomics data showed significant enrichment of proteins that were associated with concurrent and future decline in muscle function; i.e. the clinical trial data analysis showed a trend in the proteome shifted towards a more healthy functional outcomes for individuals that were treated with BGE-105 under bedrest condition. This observation reinforced the initial observations of the importance of apelin in preservation of grip strength with aging suggesting potential long-term benefits of treatment with BGE-105. FIG. 55A shows proteomics data from patients in the phase 1B clinical trial, providing the number of proteins associated with frailty (functionality), walk speed, instrumental activities of daily living (IADL) (functional instrument), and grip strength that are changed in patients treated with BGE-105 in the phase 1b clinical trial of Example 9. IADL is a specific class of functional activities being measured. For frailty (functionality), 69 out of 992 proteins associated with frailty in patients of the phase 1b clinical trial were changed by treatment with BGE-105 (p<0.05). For walk speed, 35 out of 526 proteins associated with walk speed in patients of the phase 1b clinical trial were changed by treatment with BGE-105. For grip strength, 58 out of 379 proteins associated with grip strength were changed by treatment with BGE-105. FIG. 55B shows that BGE-105 shifted the serum proteome towards a healthier state, recapitulating the benefits of naturally high apelin levels in subjects treated with BGE-105. Apelin is an exerkine released from skeletal muscle in response to exercise stimulation to increase muscle regeneration and stem cell activation, and/or reduce muscle atrophy and inflammation. Exercise stimulates release of apelin from skeletal muscle into systemic circulation. Target tissues include skeletal muscle, heart, and the CNS. Results in muscle regeneration, stem cell activation, reduced muscle atrophy and reduced inflammation.

Protein group definitions for the proteomics data shown in FIG. 55A-C is provided in Table 36 below:

### Protein Group Definitions

TABLE 36
Protein Group Definitions

| Technical | Presentation | Association | |
|---|---|---|---|
| name | name | Directional | Definition |
| Concurrent | Walking speed | negative | Proteins negatively associated with walk speed (m/s) |
| walking | (concurrent | | measured at exam 4 in the hawaii cohort |
| speed | outcomes) | | (p-value < 0.05). The model implemented was a linear |
| | | | regression with walk speed variable (m/s), corrected |
| | | | for confounders: Age, pack years smoked, and |
| | | | monthly alcohol consumption. |
| NIADL | Physical | positive | Proteins positively associated with difficulty with |
| | function | | aspects of basic function or general aspects of physical |
| | (future | | strength and mobility, such as walking half a mile, |
| | outcomes) | | lifting 10 lbs, or gripping small objects (N = 896 with |
| | | | 463 events; p-value < 0.05). The model implemented |
| | | | was a Cox regression and corrected for confounders: |
| | | | age, pack years smoked, and monthly alcohol |
| | | | consumption. |
| Mortality | Mortality | positive | Proteins positively associated with all-cause mortality |
| | (future | | in Hawaii cohort (N = 933 with 897 events; |
| | outcomes) | | p-value < 0.05). The model implemented was a Cox |
| | | | regression and corrected for confounders: age, pack |
| | | | years smoked, and monthly alcohol consumption |
| Grip4 | Grip strength | negative | Proteins negatively associated with grip strength |
| | (concurrent | | measured at exam 4. The model implemented was a |
| | outcomes) | | linear regression with grip strength (kg), corrected for |
| | | | confounders: Age, pack years smoked, and monthly |
| | | | alcohol consumption. |
| Walk speed | Walking speed | positive | Proteins positively associated with walk speed |
| impairment | (future | | declining below 0.8 m/s (N = 884 with 243 events; |
| | outcomes) | | p-value < 0.05). The model implemented was a Cox |
| | | | regression and corrected for confounders: age, pack |
| | | | years smoked, and monthly alcohol consumption. |
| Functional | Strenuous | positive | Proteins positively associated with increased difficulty |
| strength | activity | | with heavy housework or lifting 10 lbs (N = 933 with |
| | (future | | 379 events; p-value < 0.05). he model implemented |
| | outcomes) | | was a Cox regression and corrected for confounders: |
| | | | age, pack years smoked, and monthly alcohol |
| | | | consumption. |

### SomaSignal Test Using Proteomics Data

Next, a linear mixed effect model was implemented in the proteomics data to capture difference in a SomaSignal test between the two groups (treated and placebo) in the average rate of change per day.

When assessing resting energy expenditure, the Somasignal test was used to predict an individual's resting energy expenditure in calories per day (cal/day) using 122 aptamers. Population used: UK-based study of 9,022 individuals (aged 29-64 yrs), Model performance: CCC═0.66, R²=0.46 (CI: 0.42-0.49). FIG. 55C shows the absolute change in REE (cal/day) compared to baseline (end of treatment, p=0.03). As shown, changes in baseline energy expenditure were largely driven by changes in muscle mass. As shown in FIG. 55C, the proteomics data predicted that the resting energy rate was “rescued” by BGE-105 treatment.

When assessing cardiorespiratory fitness (VO₂) max, a SomaSignal test was used to predict estimated peak exercise capacity using 52 aptamers. North-american-based study of 743 individuals (aged 15-65 yrs), Model performance: CCC=0.85, R2=0.75 (CI: 0.68-0.81). As shown in FIG. 55D, VO₂ max was driven by maximal cardiac output and uptake of oxygen by skeletal muscle. BGE-105 treatment shifted the proteome towards predicted higher basal metablic rate and VO₂ max by the SomaSignal test (end of treatment, p=0.004).

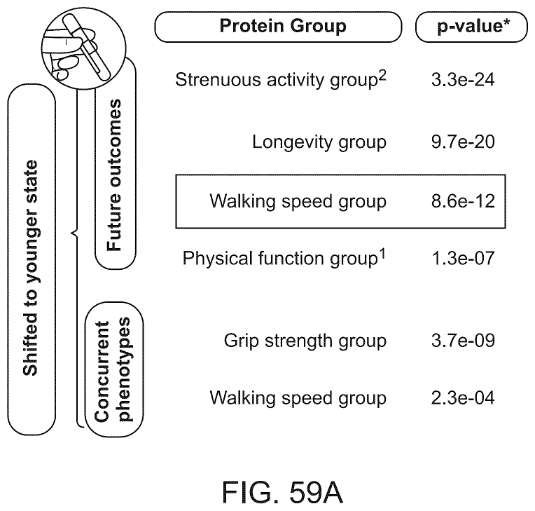

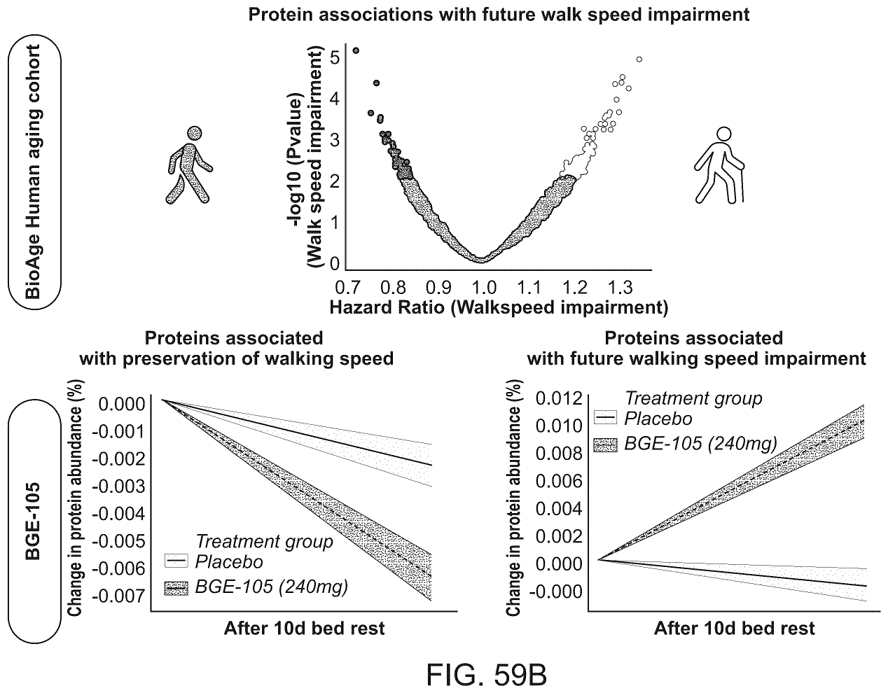

FIGS. 59A-59D show BGE-105 shifted the serum proteome towards a healthier state, recapitulating benefits of naturally high apelin levels. FIG. 59A summarizes shifting of serum protein groups showing concurrent phenotype (grip strength group, walking speed) and future outcomes (strenuous activity, longevity, walking speed, physical function). The changes represented an important biological validation, demonstrating the potential of these proteins as PD markers in future trials. FIG. 59A summarizes shifting of serum protein groups showing concurrent phenotype (grip strength group, walking speed) and future outcomes (strenuous activity, longevity, walking speed, physical function). FIG. 59B shows protein associations with future walk speed impairment in the human aging cohort. FIG. 59B shows proteins associated with preservation of walking speed. BGE-105 (240 mg) reduced reduction of protein abundance after 10 days bed rest. FIG. 59B shows protein associated with future walking speed impairment. The data demonstrate BGE-105 prevented increase of protein abundance after 10 days bed rest.

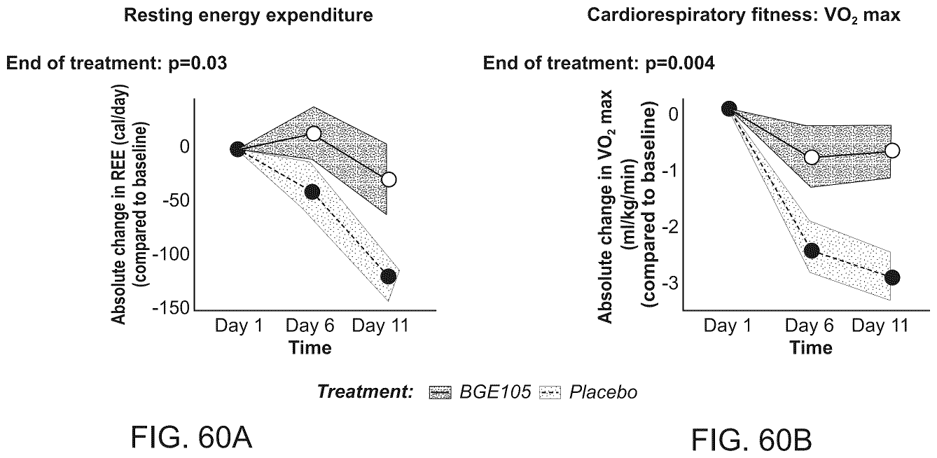

BGE-105 treatment shifted the proteome towards higher basal metabolic rate and VO₂ max assessed by SomaSignal Tests. FIG. 60A-60B illustrates resting energy expenditure (REE) and cardiorespiratory fitness in subjects treated with BGE-105. As shown, BGE-105 prevented reduction of absolute change in resting energy expenditure compared to baseline (FIG. 60A; p=0.03). The data show that changes in baseline energy expenditure were largely driven by changes in muscle mass. BGE-105 prevented reduction of absolute change in VO₂ max compared to baseline (FIG. 60B; p=0.004). V02 max is driven by maximal cardiac output and uptake of oxygen by skeletal muscle.

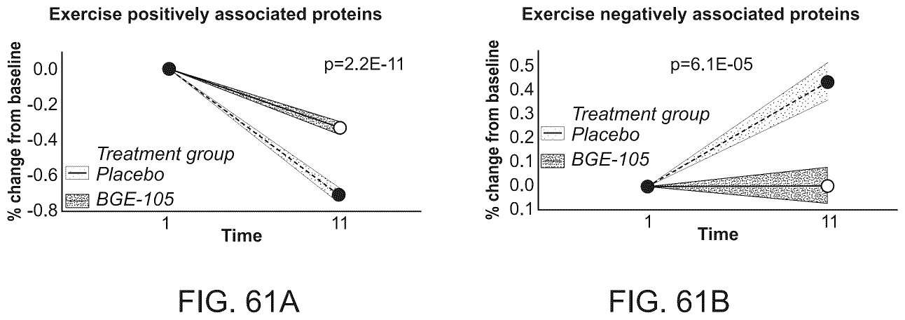

FIG. 61A-61B illustrates correlation of exercise and proteomes in BGE-105 treated subjects. BGE-105 prevented increase of exercise negatively associated protein change from baseline (p=6.1E-05). The result showed that BGE-105 treatment changes plasma proteomic levels, recapitulating the effects of exercise on the proteomic level.

BGE-105 significantly changed the serum levels of some of the same proteins that are associated in BioAge longitudinal aging cohort data with future decline in physical function as assessed by walk speed, activities of daily living (functional instrument) and grip strength. These proteins can then be used to try to identify patients who are responding to BGE-105 treatment and are likely to have reduced muscle atrophy.

### Summary

These results indicate efficacy of BGE-105 in humans. BioAge completed a Phlb trial in healthy older volunteers (≥65 years old) who underwent 10 days of continuous bedrest. BGE-105 (240 mg) or placebo was administered once daily intravenously for 10 days, and normal activities were resumed on Day 11. The study demonstrated that compared to placebo, BGE-105 significantly reduced muscle loss as measured by vastus lateralis muscle thickness (75% reduction in loss) and cross-sectional area (>50% reduction in loss) (FIG. 48). This preservation of muscle mass was supported by the maintenance of skeletal muscle fractional protein synthetic rates (as measured by D20 method and muscle biopsy). Crucially, the synthesis of myofibrillar structural proteins such as myosin and troponin was preserved compared to placebo (FIG. 51).

Transcriptomics (by single-nucleus RNAseq) of muscle tissue from study subjects demonstrated that BGE-105 prevented bed rest-induced downregulation of mitochondrial biogenic regulator PGC-lu and all respiratory complexes (FIG. 30). At the proteomic level, BGE-105 treatment shifted the plasma proteome towards an estimated higher basal metabolic rate and VO2 max by SomaSignal test (FIG. 52). Previous studies in hospitalised patients documented the challenge of overcoming the anabolic resistance in this patient population, wherein nutritional supplement fails to maintain muscle protein synthesis and exercise training is a barrier for older, frail patients.

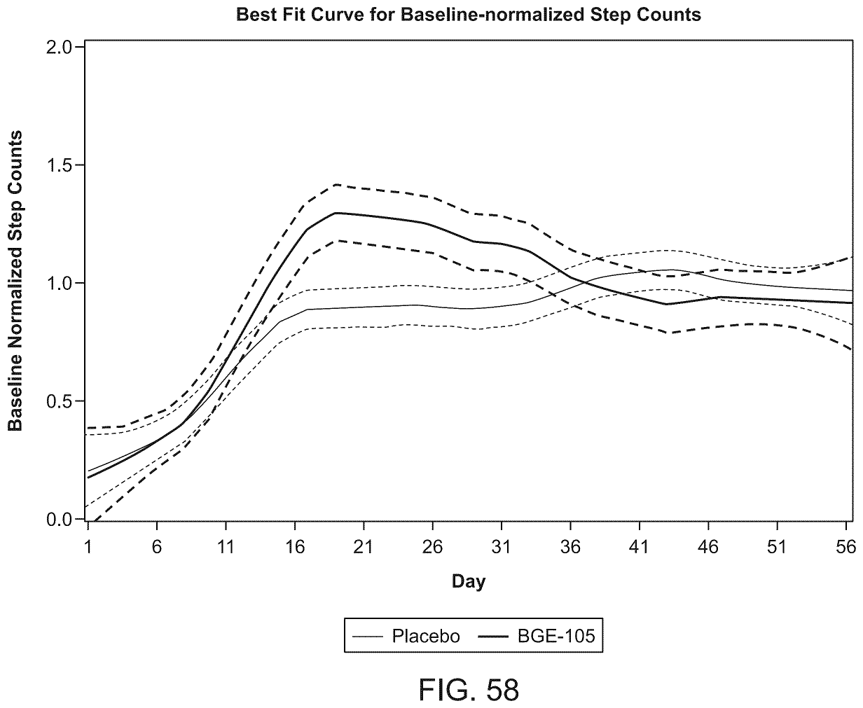

FIG. 58 shows BGE-105 improves post-bedrest recovery by wearable accelerometer.

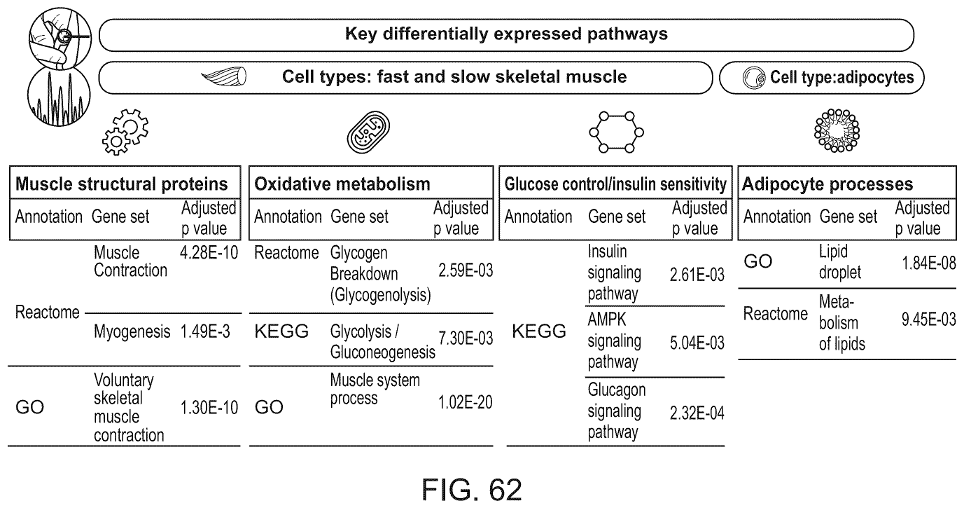

FIG. 62 illustrates differentially regulated pathways showing BGE-105 beneficial effects on muscle and adipocyte cells.

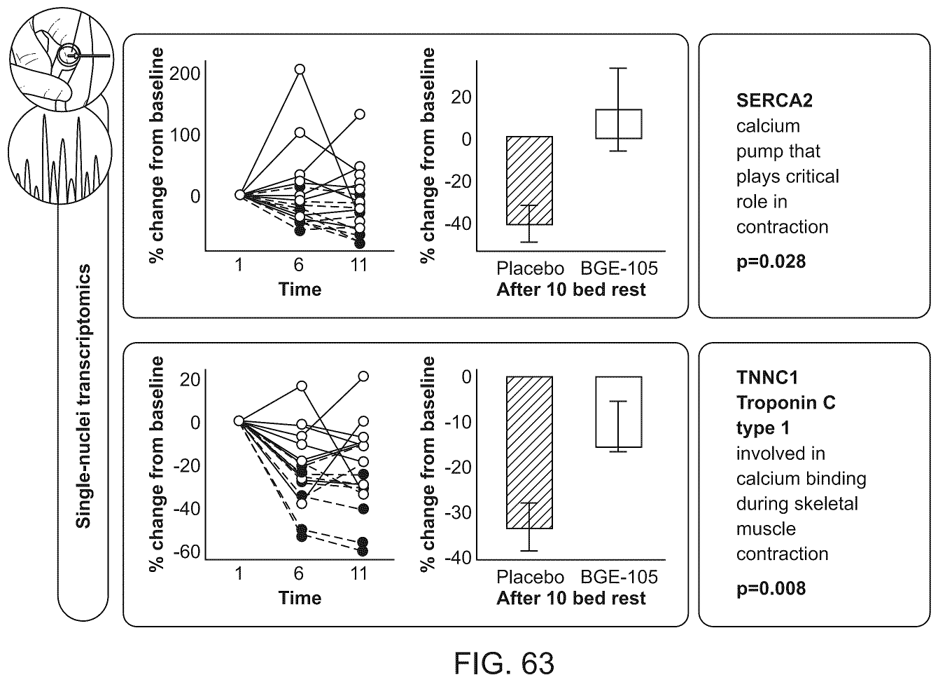

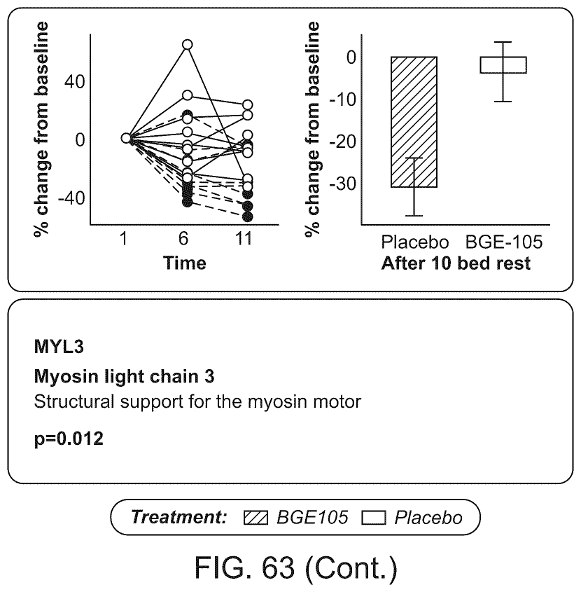

FIG. 63 shows BGE-105 prevented bed rest-induced downregulation of muscle contractile proteins in fast and slow skeletal muscles. p values reflect change after 10 days of bedrest, compared to baseline. BGE-105 prevented reduction of troponin C (TNNC1), which is involved in calcium binding during skeletal muscle contraction (p=0.008); myosin heavy chain beta (MYH7), which provides structural support for the myosin motor (p=0.012); and calcium-ATPase type 2 in the sarco-/endoplasmic reticulum (SERCA2), which plays a critical role in contraction (p=0.028). p values reflect change after 10 days of bedrest, compared to baseline.

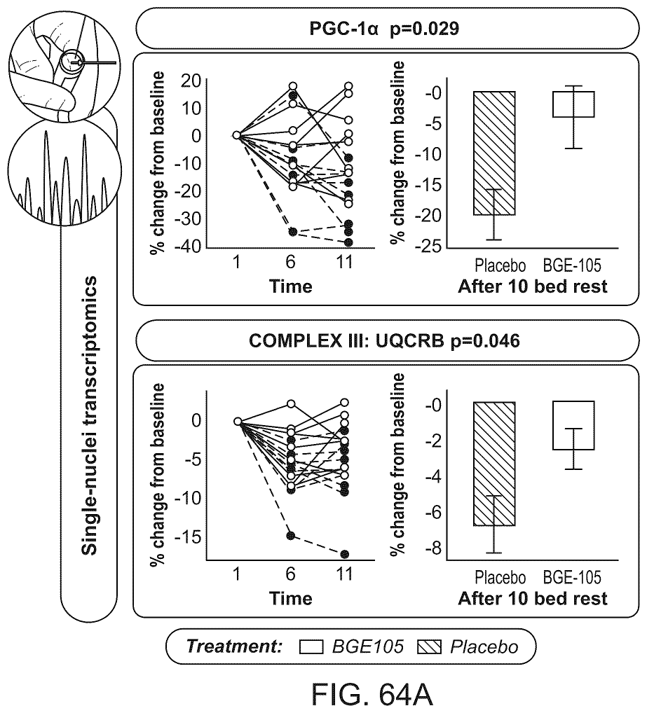

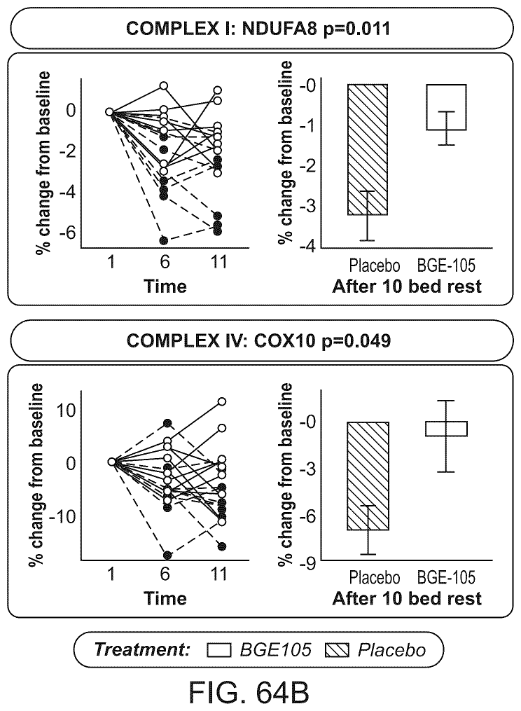

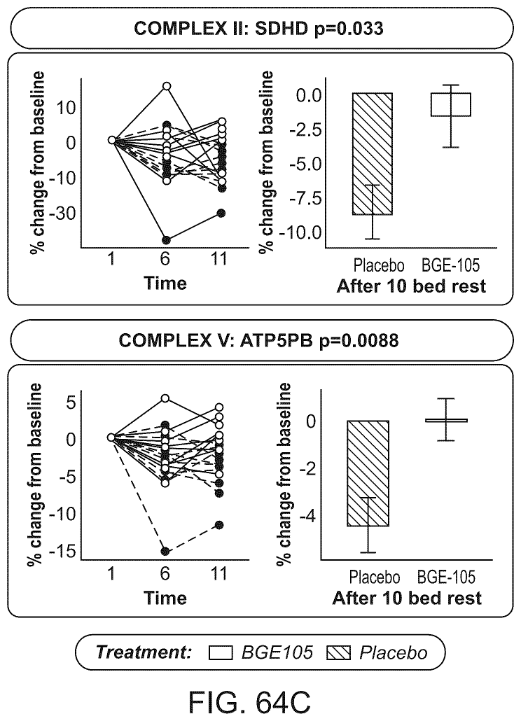

FIGS. 64A-64C show BGE-105 prevented bed rest-induced downregulation of mitochondrial biogenic regulator PGC-1α and all respiratory complexes. Shown are representative genes including PGC-1α (p=0.029), COMPLEX 1: NDUFA8 (p=0.011), COMPLEX II: SDHD (p=0.033), COMPLEX III: UQCRB (p=0.046), COMPLEXT IV: COX10 (p=0.049), COMPLEX V: ATP5PB (p=0.0088).

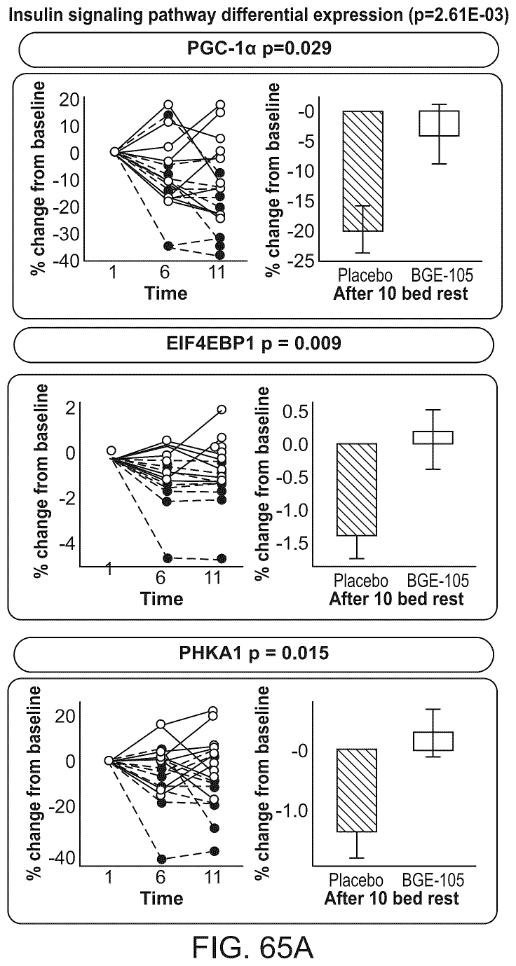

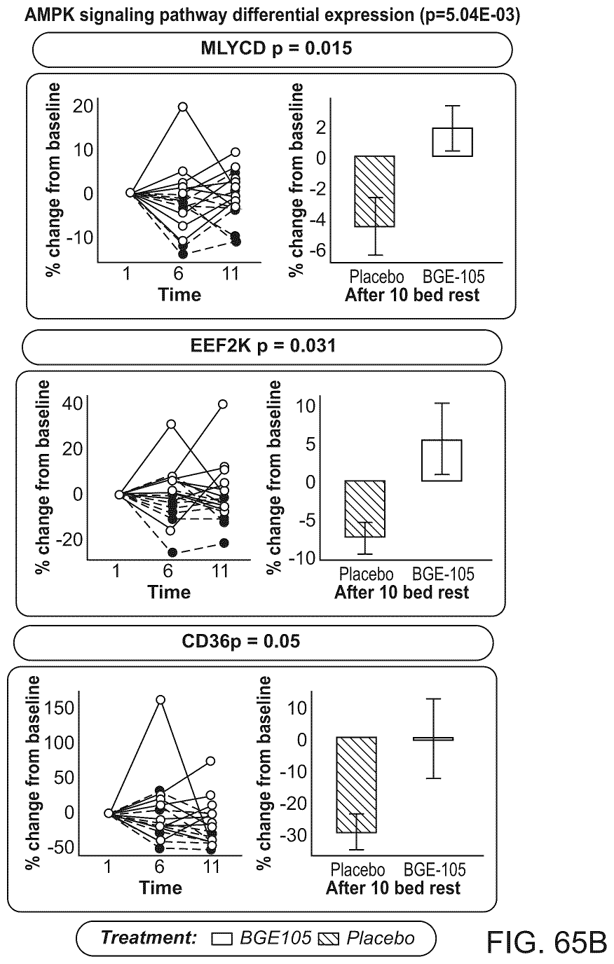

FIGS. 65A-65B show single-nuclei transcriptomics of BGE-105 preserved gene expression involved in glucose metabolism. FIG. 65A shows differential expression of representative genes (PGC-1α, p=0.029; EIF 4EBP1, p=0.009; PHKA1, p=0.015) in insulin signaling pathway (p=2.61E-03). FIG. 65B shows differential expression of representative genes (MLYCD, p=0.015; EEF2K, p=0.031; CD36, p=0.05) in AMPK signaling pathway (p=5.04E-03). p values reflect change after 10 days of bedrest, compared to baseline.

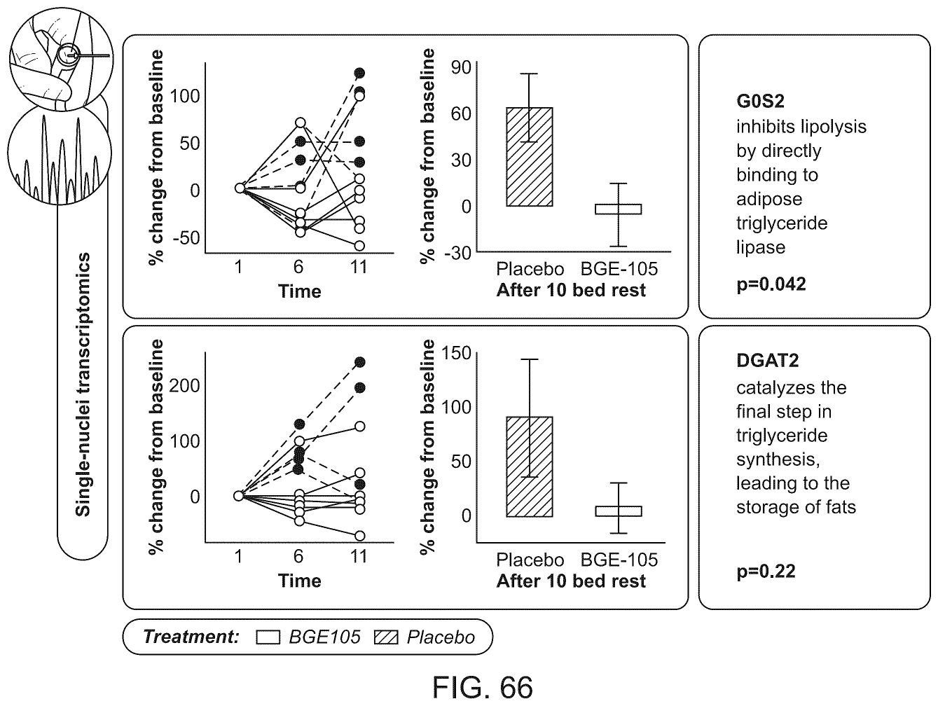

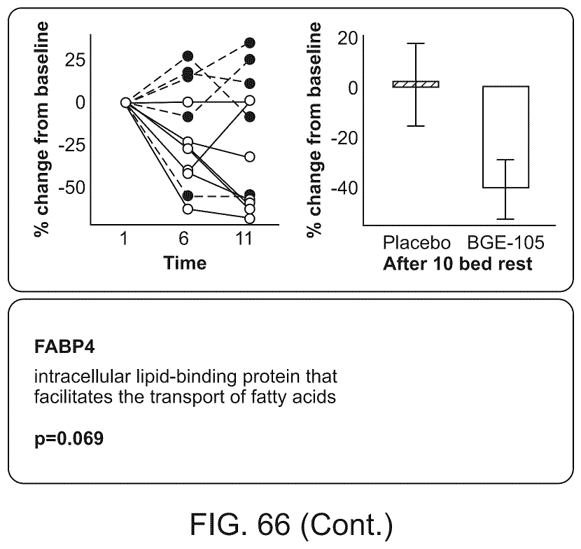

FIG. 66 shows BGE-105 decreased expression of genes involved in fat storage in muscle interstitial adipocytes. p values reflect change after 10 days of bedrest, compared to baseline, based on 727 adipocytes captured. BGE-105 prevented reduction of G0/G1 switch gene (GOS2), which inhibits lipolysis by directly binding to adipose triglyceride lipase (p=0.042); cyl-CoA:diacylglycerol acyltransferases 2 (DGAT2), which catalyzes the final step in triglyceride synthesis, leading to the storage of fats (p=0.22); and fatty acid binding protein 4 (FABP4), which is an intracellular lipid-binding protein that facilitates the transport of fatty acid (p=0.069).

This study piloted wearable activity monitoring; encouraging accelerometry results demonstrated that post-bedrest recovery of activity (starting on Day 11; bedrest and BGE-105/placebo treatment occurred from Day 1 to 10) was more pronounced in the BGE-105 group (FIG. 58).

This provides a human model, and serves as proof-of-concept that acute muscle loss can be ameliorated by BGE-105 regardless of nutritional and exercise capacity.

In summary, preclinical and clinical studies demonstrate target engagement, efficacy, including improvements in both overall activity and muscle atrophy, favorable pharmacokinetics, and human safety in over 200 subjects shown in a series of Phase 1a/1b trials. BGE-105 is a highly selective and potent agonist of the apelin receptor. BGE-105 has been shown to preserve muscle size and quality in older volunteers (≥65 years old) compared to placebo in a 10-day bed rest study.

### 6.9. Example 9: snRNAseq Analyses of Human Muscle Tissue Samples from Healthy Adult Patients (Phase 1b Clinical Study)

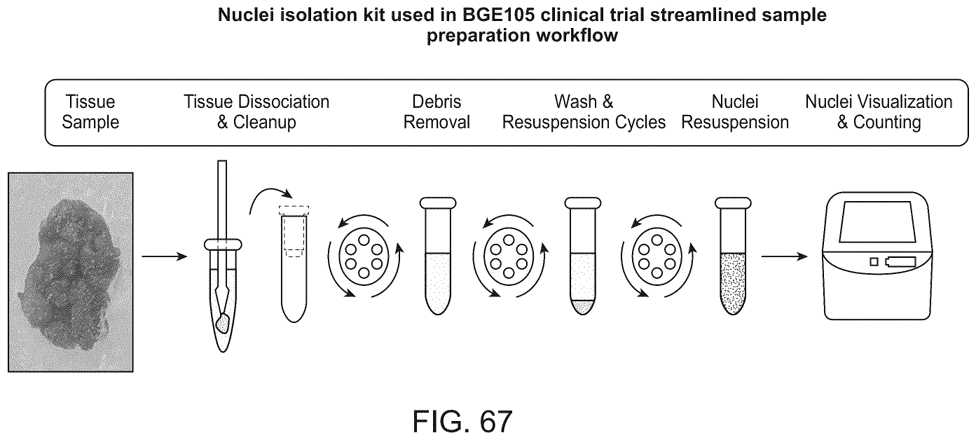

FIGS. 67-89 show details and results of snRNAseq analyses of human muscle tissue samples from the above Phase 1b clinical study of BGE-105 for treating muscle atrophy, including loss of muscle mass and function. These proteomics results are consistent with the human clinical data of efficacy (see Example 8).

FIG. 67 illustrates nuclei isolation for 10× Genomics single cell transcriptome gene expression technology used to assess tissue samples. FIG. 67 illustrates workflow of a nucleic isolation kit used in BGE-105 clinical trial streamlined sample preparation. Tissue samples were collected from patients and processed in a laboratory. Tissues were dissociated and cleaned up. The debris were removed and the sample (e.g., nuclei) was washed, resuspended for multiple cycles according to routine tissue processing procedures. Cell nuclei were visualized and counted using standard transcriptome gene expression technologies, for example, 10× Genomics single cell library technology is used for assessing tissue samples.

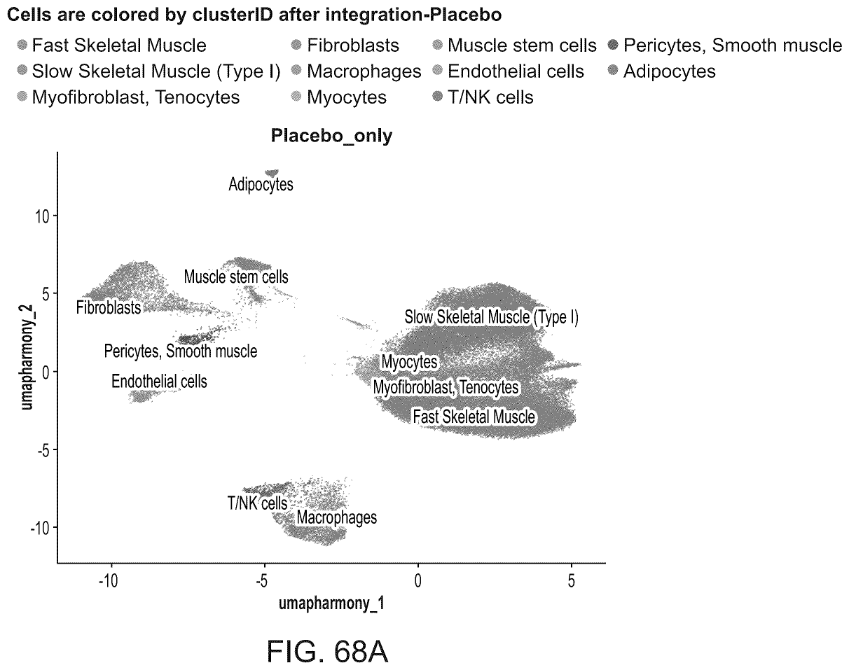

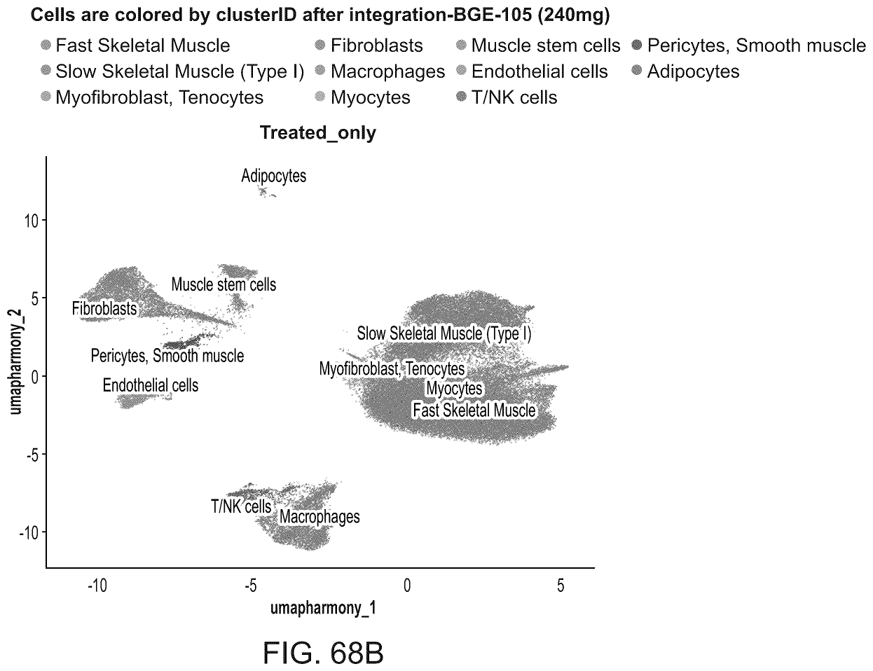

FIGS. 68A-68B show 11 cell types were identified, consistent with published muscle atlas (see e.g., Perez et al., 2022, Single nuclei profiling identifies cell specific markers of skeletal muscle aging, frailty, and senescence. Aging (Albany NY). 2022 Dec. 13; 14(23):9393-9422. doi: 10.18632/aging.204435. Epub 2022 Dec. 13. PMID: 36516485; PMCID: PMC9792217). Cell type clustered include fast skeletal muscle, slow skeletal muscle (type 1), myofibroblast, tenocytes, fibroblasts, muscle stem cells, pericytes, smooth muscle, endothelial cells, T/NK cells, and adipocytes. Two methods and two annotations were used. Top 20 variable genes within clusters were used as markers for cell type annotation in placebo and BGE-105 treated groups.

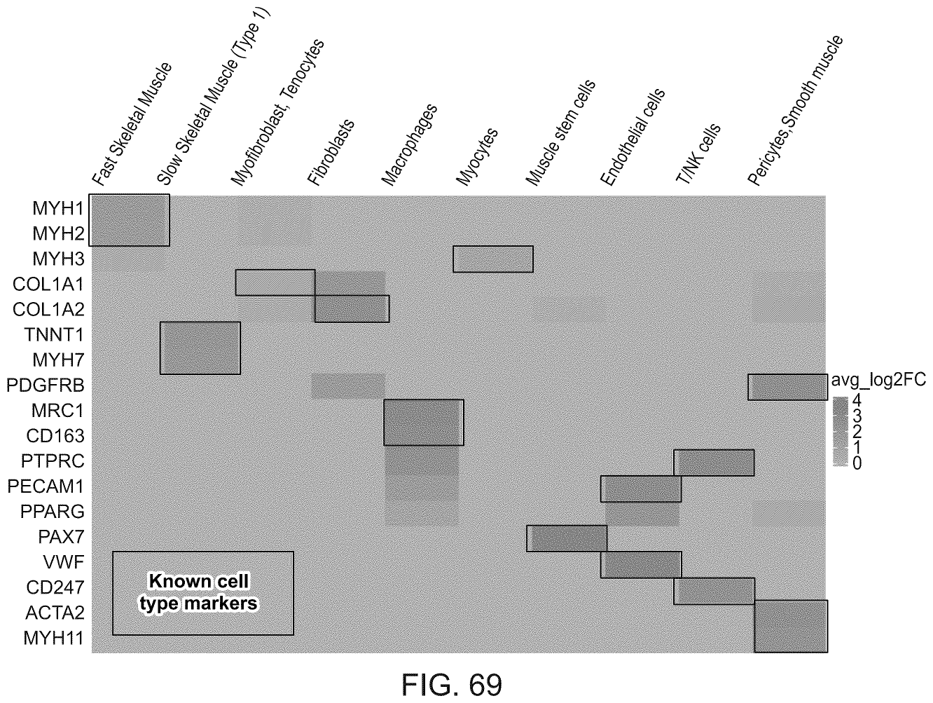

FIG. 69 shows that there is consistency between the top 20 cell-type specific expressed genes and known cell type markers. Plot shows log 2(fold change) in expression in a specific cell-type compared to rest for the top 20 cell-type specific expressed genes. Analysis was performed using all samples. Biomarkers tested include MYH1, MYH2, MYH3, COL1A1, COL1A2, TNT1, MYH7, PDGFRB, MRC1, CD163, PTPRC, PECAM1, PPARG, PAX7, VWF, CD247, ACTA2, and MYH11. For example, MYH1 and MYH2 gene expression were predominantly detected in fast skeletal muscles, which TNT1 and MYH7 gene expression were predominantly detected in slow skeletal muscles.

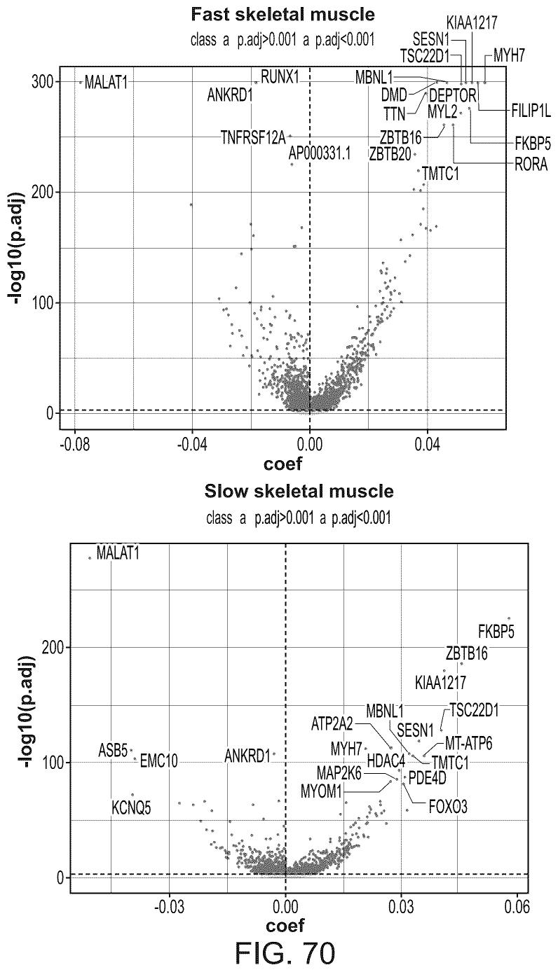

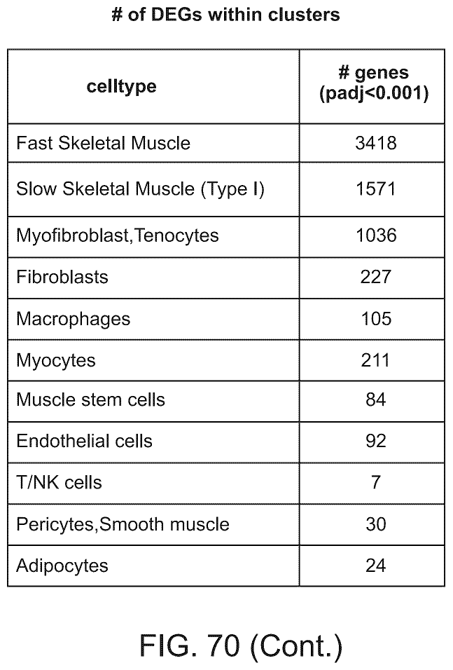

FIG. 70 shows that differentially expressed genes (DEG) associated with BGE105 were identified for each cell types including fast skeletal muscle and slow skeletal muscle.

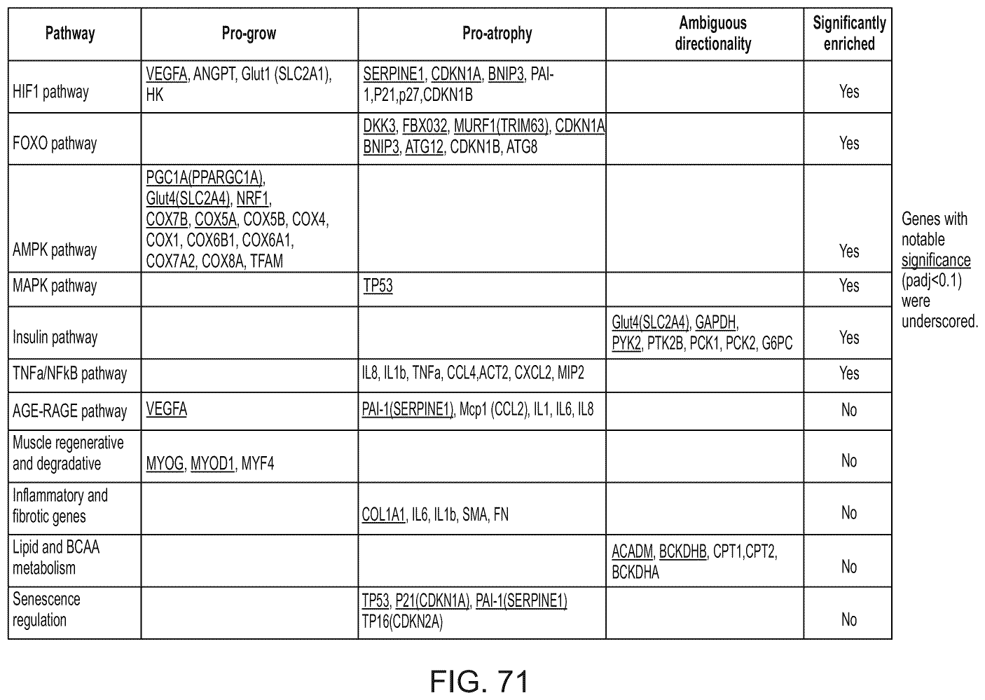

FIG. 71 shows that signaling pathways that control muscle loss and promote muscle growth were enriched in BGE105 treatment associated genes in fast skeletal muscle. Genes with notable significance (padj<0.1) were underscored.

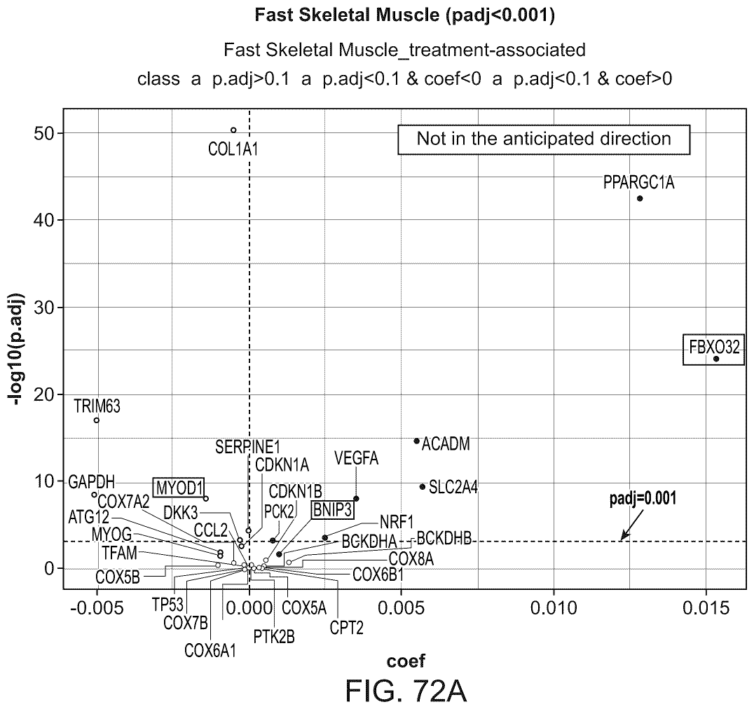

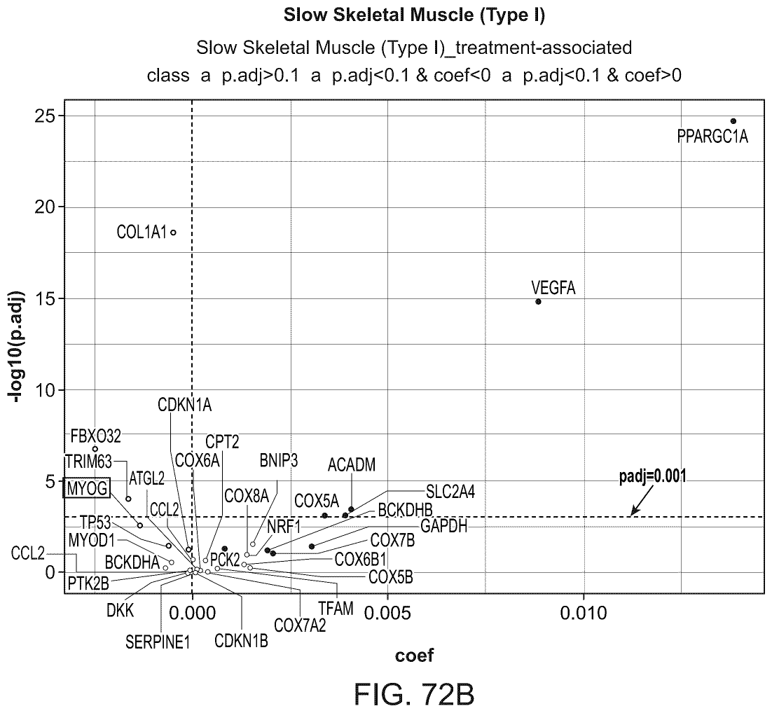

FIGS. 72A-72B show that treatment association of most of significant genes (padj<0.1) in muscle growth/loss related signaling pathways were in the anticipated direction. Shown are detection of genes associated with fast skeletal muscle treatment (padj<0.001) (FIG. 72A) and slow skeletal muscle (Type I) treatment (FIG. 72B).

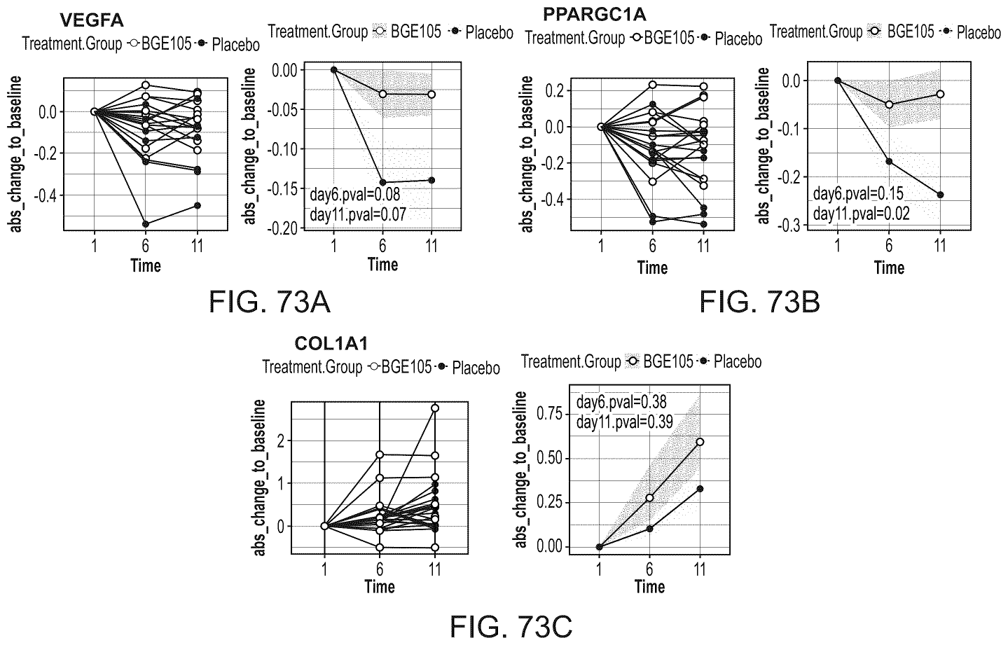

FIGS. 73A-73C show that bulk expression level of VEGFA (FIG. 73A), PPRGC1A (FIG. 73B) and COL1A1 (FIG. 73C) were higher in treated group than that in placebo group on day 11.

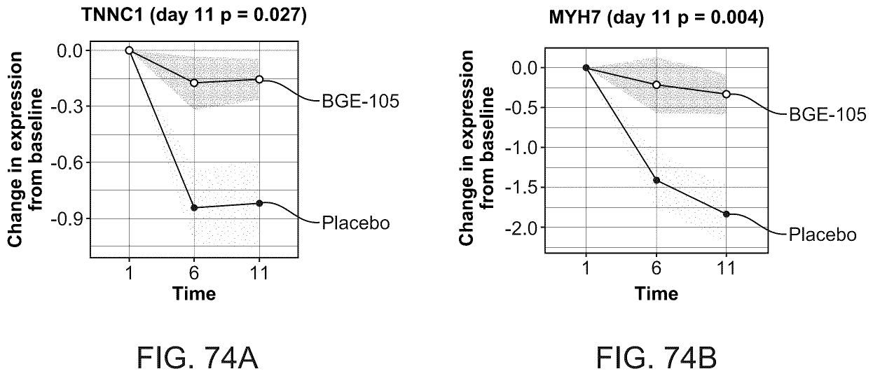

FIGS. 74A-74B show that bulk expression level of TNNC1 and MYH7 were higher in treated compared to placebo on day 11. Shown is bulk expression level: mean of gene across all cells for each patient. Cell types were ignored. TNNC1 (FIG. 74A), and MYH7 (FIG. 74B) are both slow skeletal muscle cell markers.

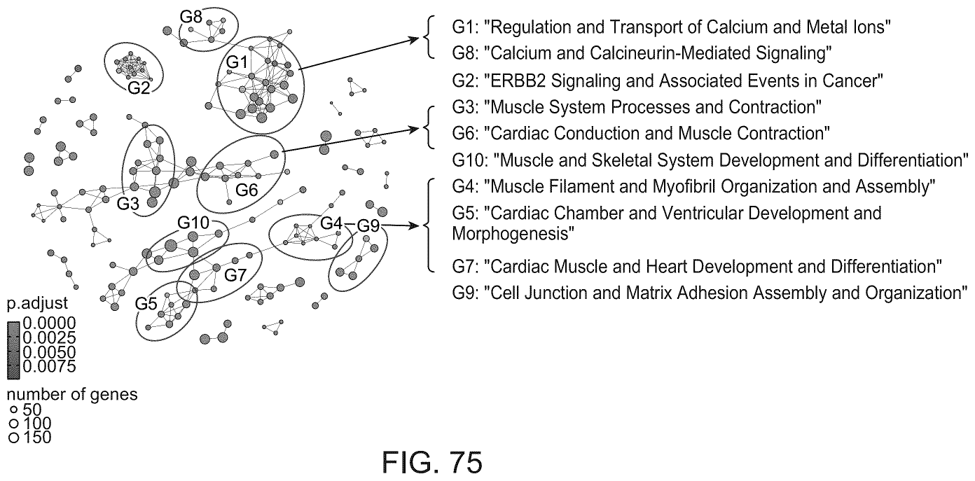

FIG. 75 shows that for fast skeletal muscle: 10 groups of 5+ enriched pathways were identified. These include pathways involve in: regulation and transport of calcium and metal ions, calcium and calcinenurin-mediated signaling, ERBB2 signaling and associated events in cancer, muscle system processes and contraction, cardiac conduction and muscle contraction, muscle and skeletal system development and differentiation, muscle filament and myofibril organization and assembly, cardiac chamber and ventricular development and morphogenesis, cardiac muscle and heart development and differentiation, and/or cell junction and matric adhesion assembly and organization.

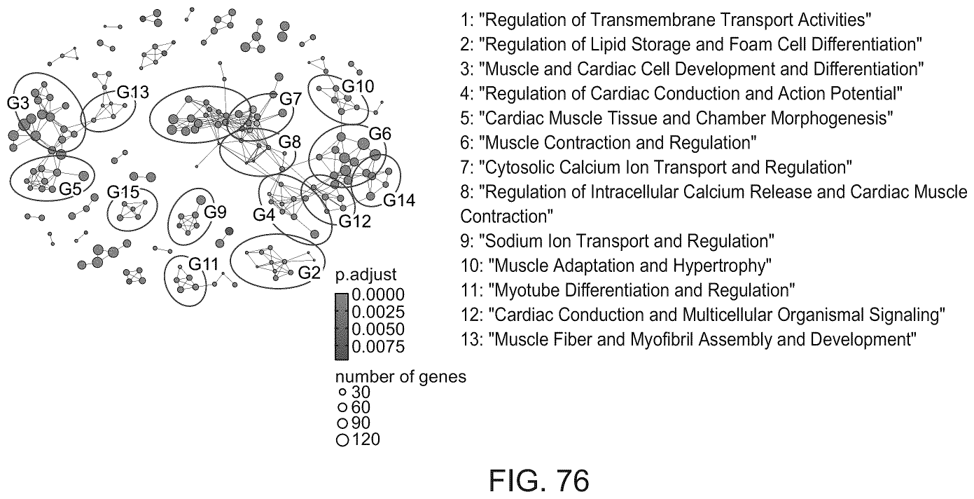

FIG. 76 shows that for slow skeletal muscle: 13 groups of 5+ enriched pathways were identified. These include pathways involve in: regulation of transmembrane transport activities, regulation of lipid storage and foam cell differentiation, muscle and cardiac cell development and differentiation, regulation of cardiac conduction and action potential, cardiac muscle tissue and chamber morphogenesis, muscle contraction and regulation, cytosolic calcium ion transport and regulation, regulation of intracellular calcium release and cardiac muscle contraction, sodium ion transport and regulation, muscle adaption and hypertrophy, myotube adaptation and hypertrophy, cardiac conduction and multicellular organismal signaling, and/or muscle fiber and myofibril assembly and development.

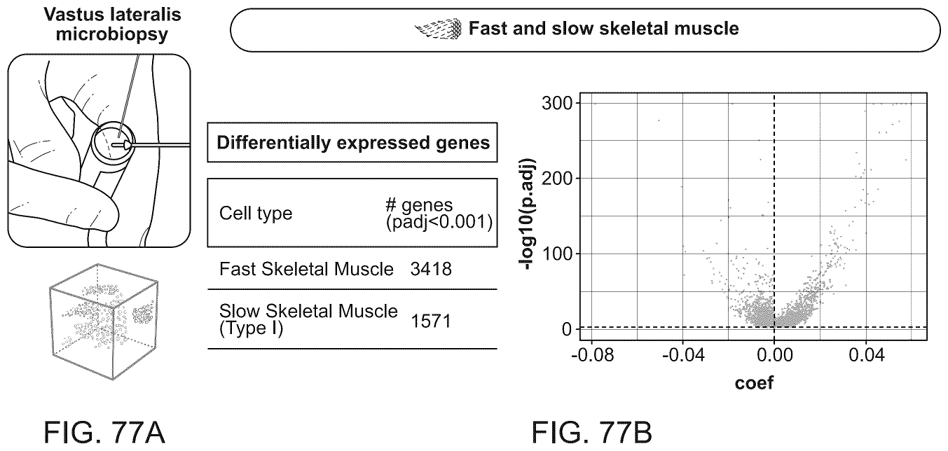

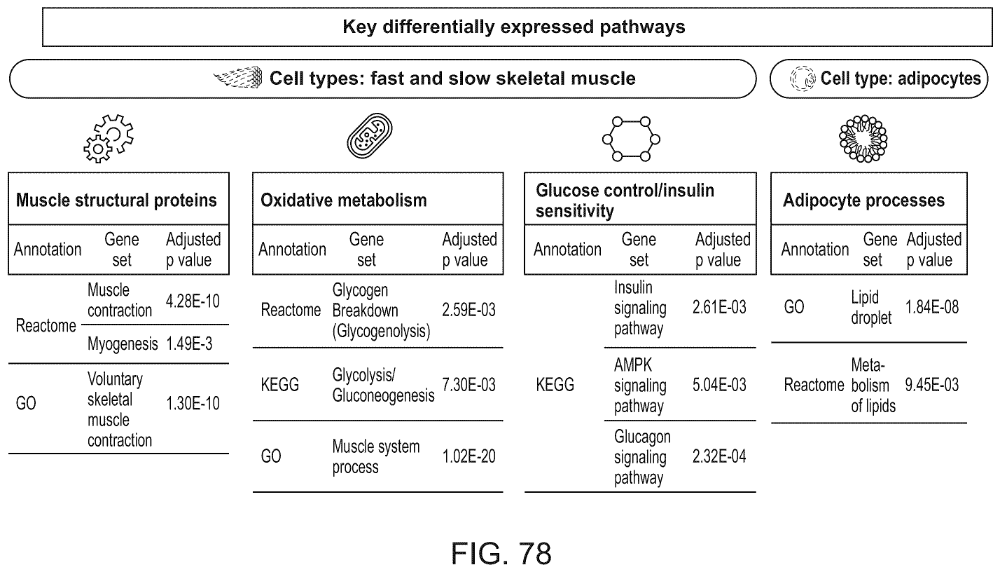

FIGS. 77A-77B show cell type-specific patterns of differential gene expression associated with BGE-105 treatment identified in muscle biopsies collected from vastus lateralis. Shown are differentially expressed genes detected in fast skeletal muscle (3418) and slow skeletal muscle (Type 1) (1571).

FIG. 78 shows that differentially regulated pathways suggest BGE-105 beneficial effects on key muscle and adipocyte processes.

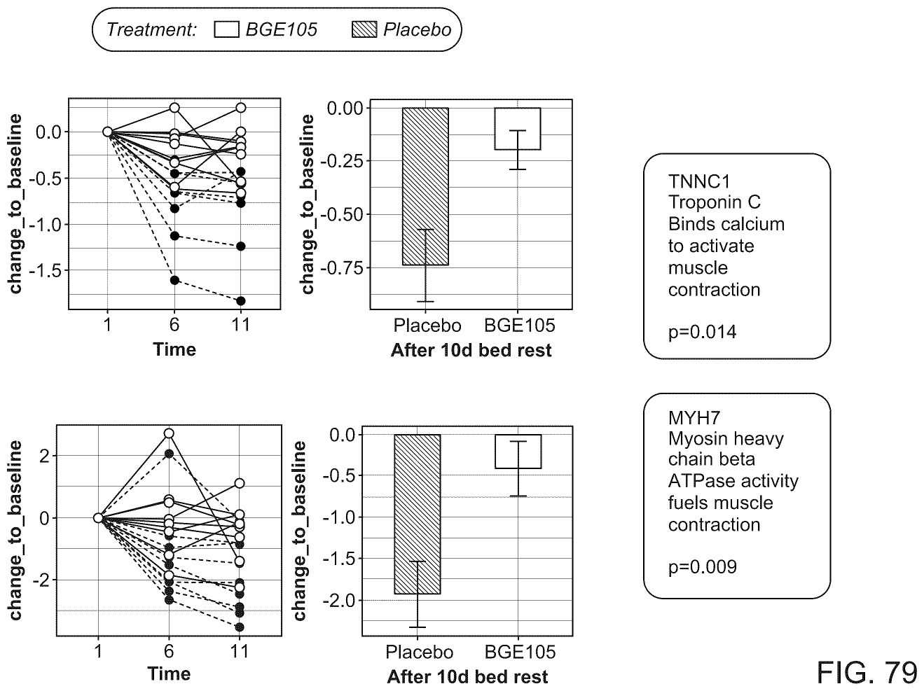

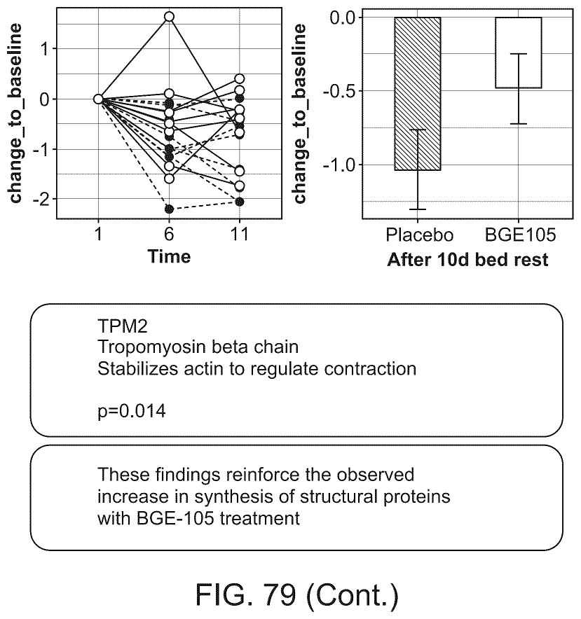

FIG. 79 shows BGE-105 prevents bed rest-induced downregulation of muscle contractile proteins in fast and slow skeletal muscles. p values reflect change after 10 days of bedrest, compared to baseline. BGE-105 prevents reduction of troponin C (TNNC1), which binds calcium to activate muscle contraction (p=0.014); myosin heavy chain beta (MYH7), which is involved in ATPase activity fuels muscle contraction (p=0.009); and tropomyosin beta chain (TPM2), which stabilizes actin to regulate contraction (p=0.014). These findings reinforce the observed increase in synthesis of structural proteins with BG5-105 treatment.

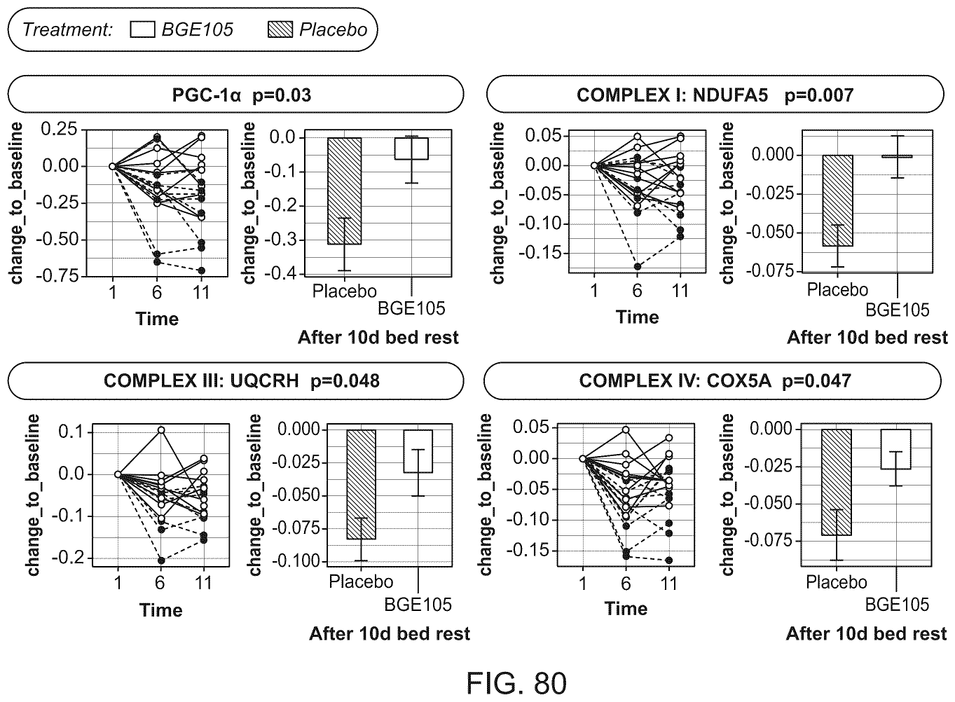

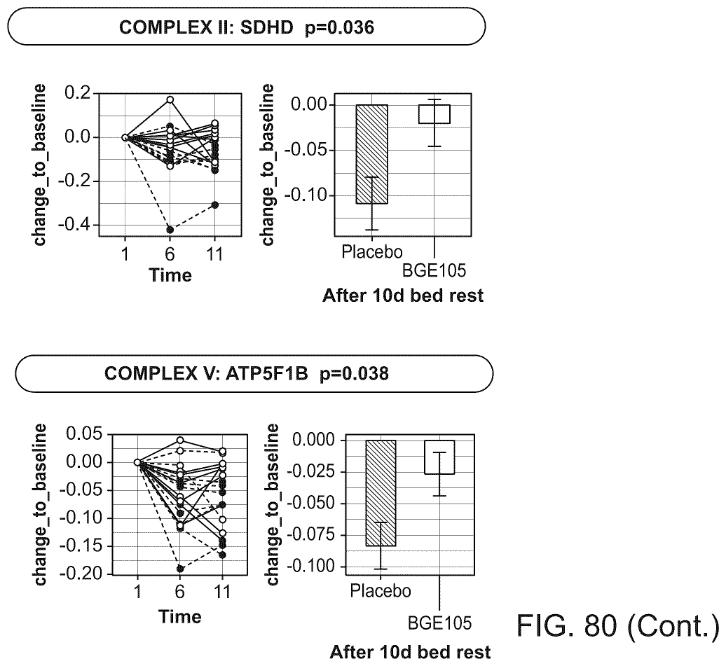

FIG. 80 shows BGE-105 prevents bed rest-induced downregulation of mitochondrial biogenic regulator PGC-1α and all respiratory complexes. Shown are representative genes including PGC-1α (p=0.03), COMPLEX 1: NDUFA (p=0.007), COMPLEX II: SDHD (p=0.036), COMPLEX III: UQCRH (p=0.048), COMPLEXT IV: COX5A (p=0.047), COMPLEX V: ATP5F1B (p=0.038).

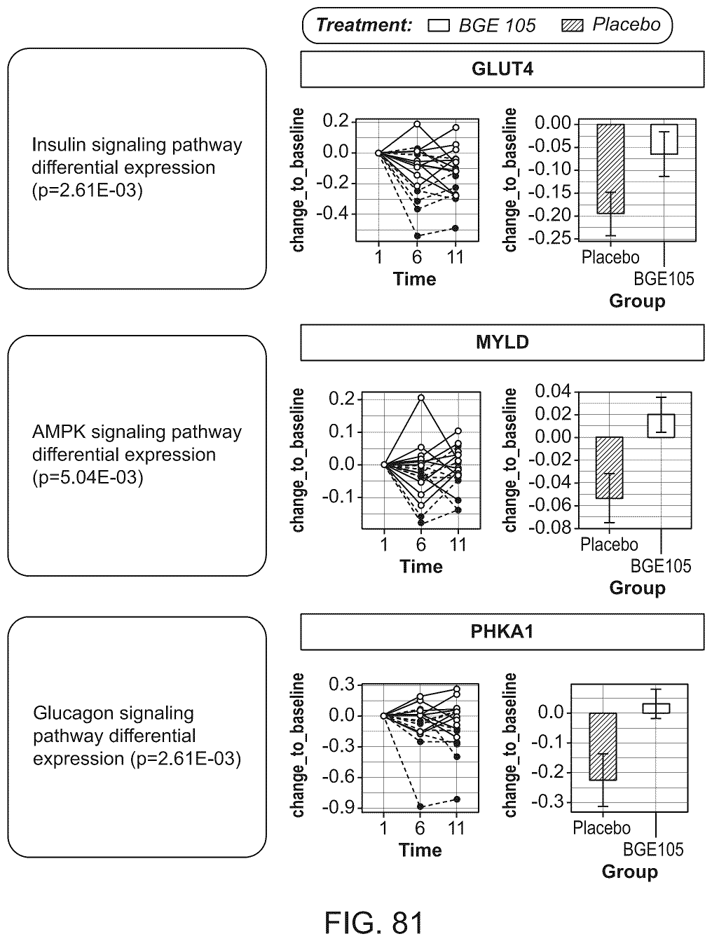

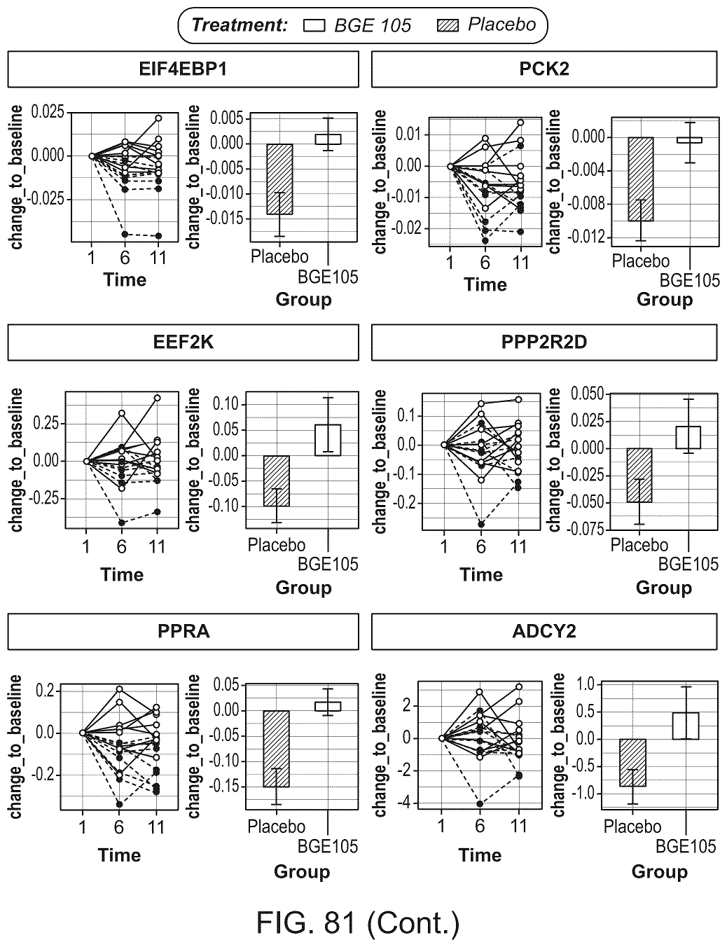

FIG. 81 shows BGE-105 prevented detrimental expression level of genes involved in muscle metabolic processes. Shown are differential gene expression of insulin signaling pathway (p=2.61E-03), AMPK signaling pathway (p=5.04E-03), and glucagon signaling (p=2.61E-03). Exemplary genes are GLUT4, EIF4EBP1, PCK2 (insulin signaling pathway); MYLD, EEF2K, PPP2R2D (AMPK signaling pathway); and PHKAA1, PPRA, ADCY2 (glucagon signaling pathway).

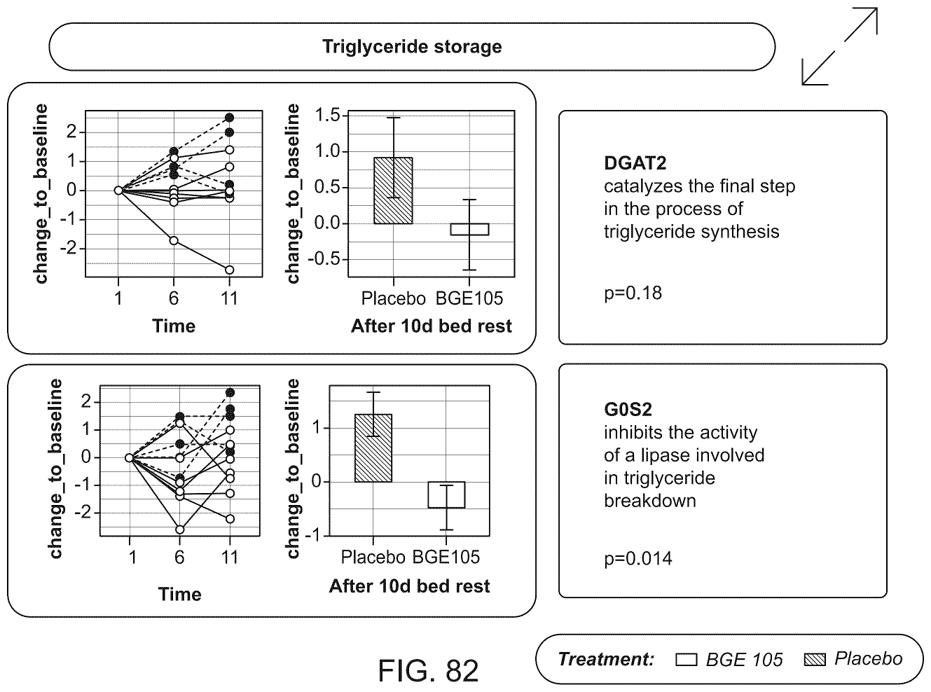

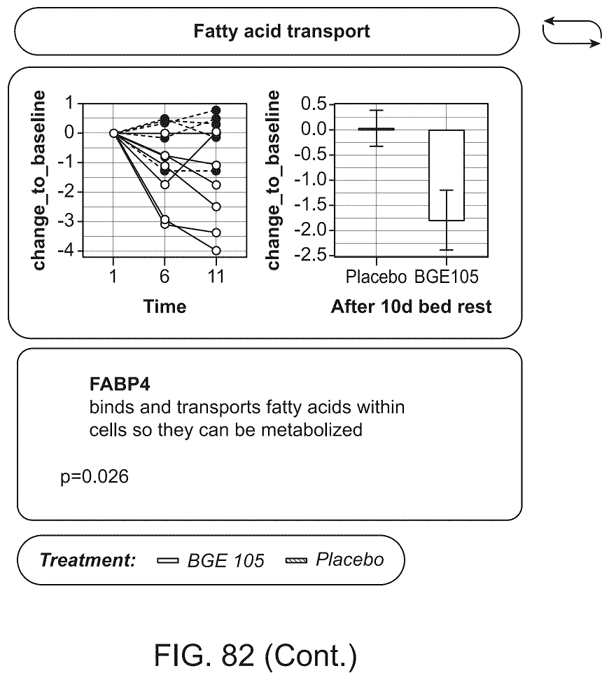

FIG. 82 shows BGE-105 prevents bed rest-induced upregulation of genes involved in triglyceride storage and fatty acid metabolism, potential mechanism for promoting fat loss. Exemplary genes include DGAT2, which catalyzes the final step in the process of triglyceride synthesis (p=0.18); GOS2, which inhibits the activity of a lipase involved in triglyceride breakdown (p=0.014); and/or FABP4, which binds and transports fatty acids within cells so they can be metabolized (p=0.026).

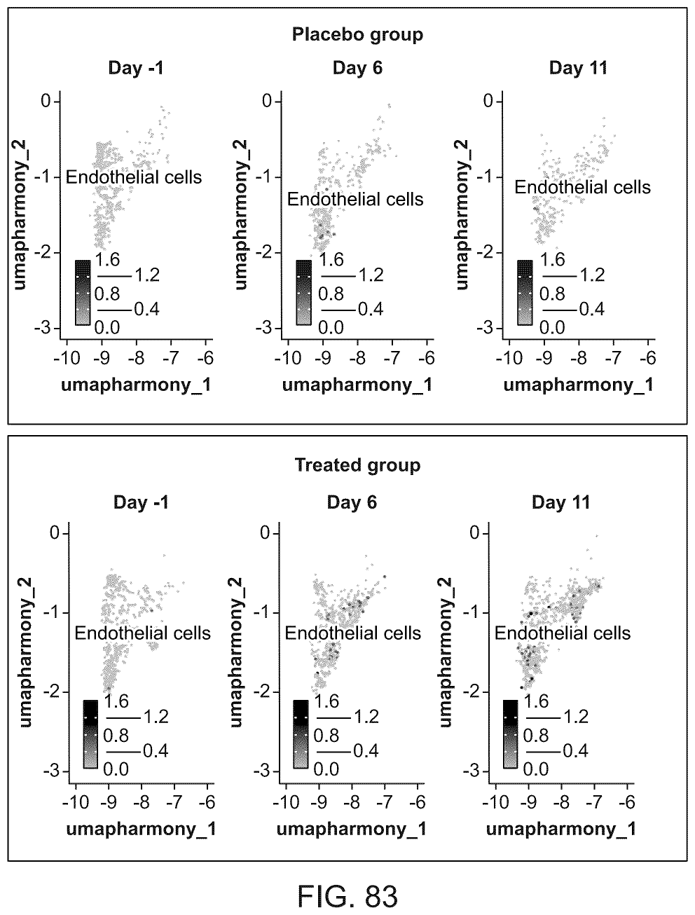

FIG. 83 shows APLNR was expressed in more Endothelial cells in the treated group. A small proportion of cells expressed APLNR (272 cells). 46% of these cells were within the endothelial cells. T-test of APLNR expression level in detected cells between treated and placebo groups: Day6_t.stat=2.02, Day6_p.val=0.04, Day11_t.stat=2.33, Day11_p.val=0.02.

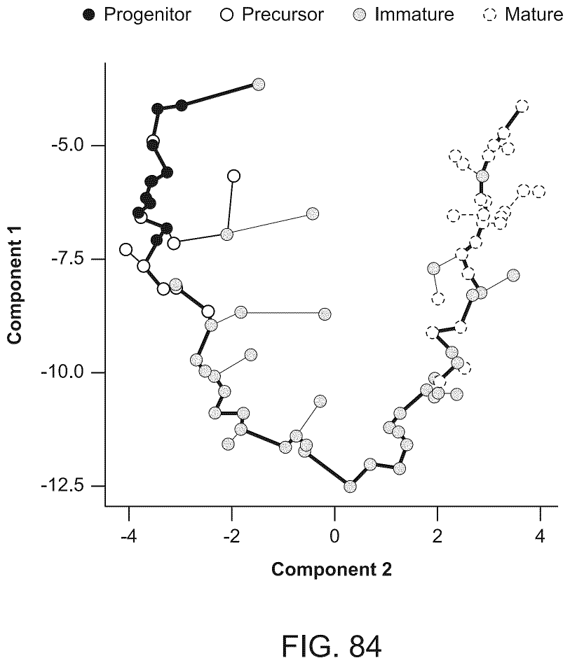

FIG. 84 Cell differentiation trajectory and pseudotime inference. Pseudotime inference for cell differentiation is a computational approach to model dynamic changes and transitions between different cellular states such as transition between progenitor, precursor, immature, and mature cell states. It allows for the distinction between early and late stages of a biological process. Pseudotime analysis can help uncover the chronological sequence of gene expression changes during a biological process, thereby allowing us to understand the underlying molecular mechanisms.

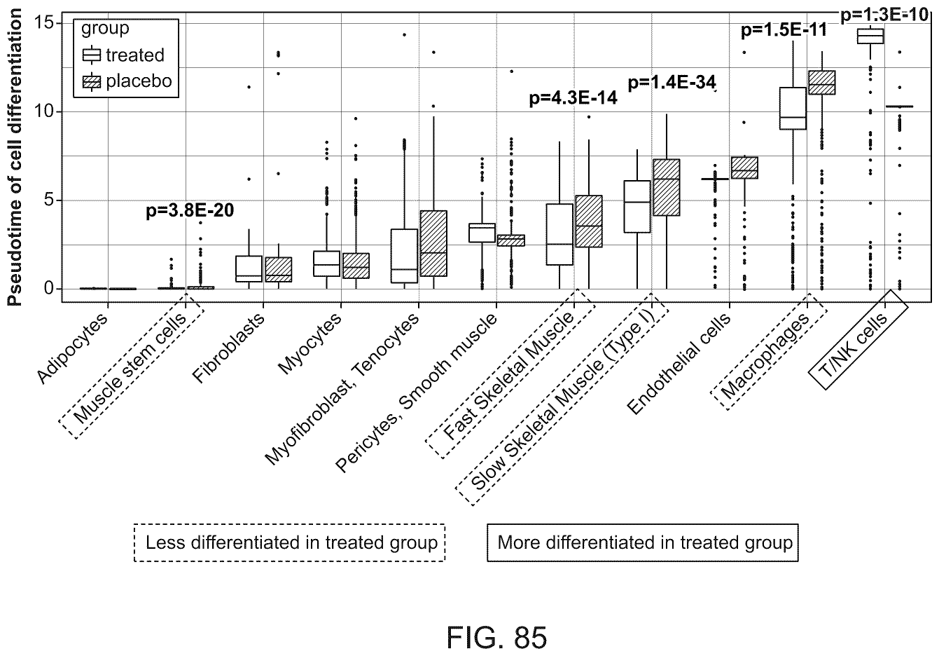

FIG. 85 shows a plot indicating cells were less differentiated with treatment for Fast/Slow skeletal muscle, macrophages, T/NK cells and muscle stem cell. Baseline muscle stem cells (treated and placebo) were set as the root. As shown, muscle stem cells, fast skeletal muscle, slow skeletal muscle (Type I), and macrophages are less differentiated in treated group, whereas T/NK cells are more differentiated in treated group.

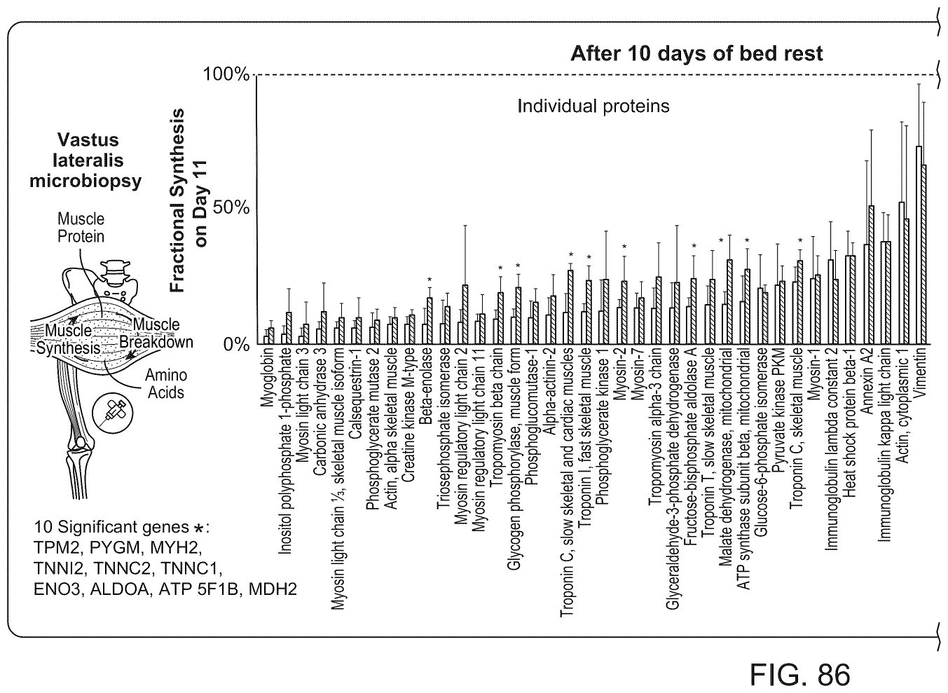

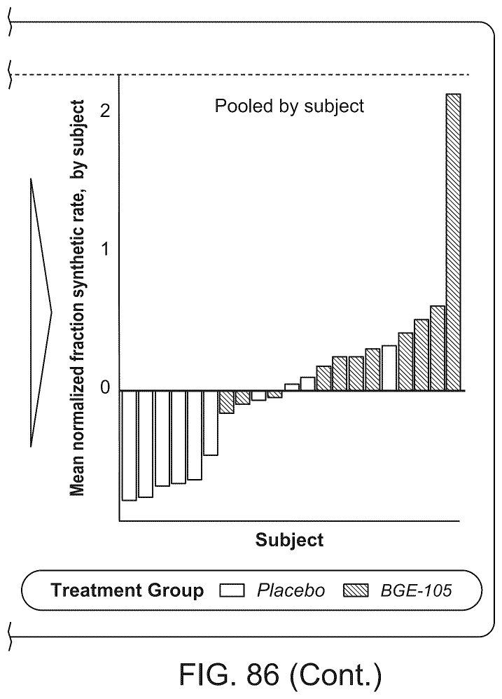

FIGS. 86-88 illustrate the results of secondary analyses. Evaluation of signal from protein synthesis rate analysis. Investigation of aging and muscle signatures from published research.

FIG. 86 shows BGE-105 resulted in relatively higher muscle protein synthesis in the vastus lateralis, measured via microbiopsy (p<0.005). Shown is muscle protein synthetic rate after 10 days of bed rest. Asterisks indicate 10 significant genes: TPM2, PYGM, MYH2, TNNI2, TNNC2, TNNC1, ENO3, ALDOA, ATP 5FIB, MDH2.

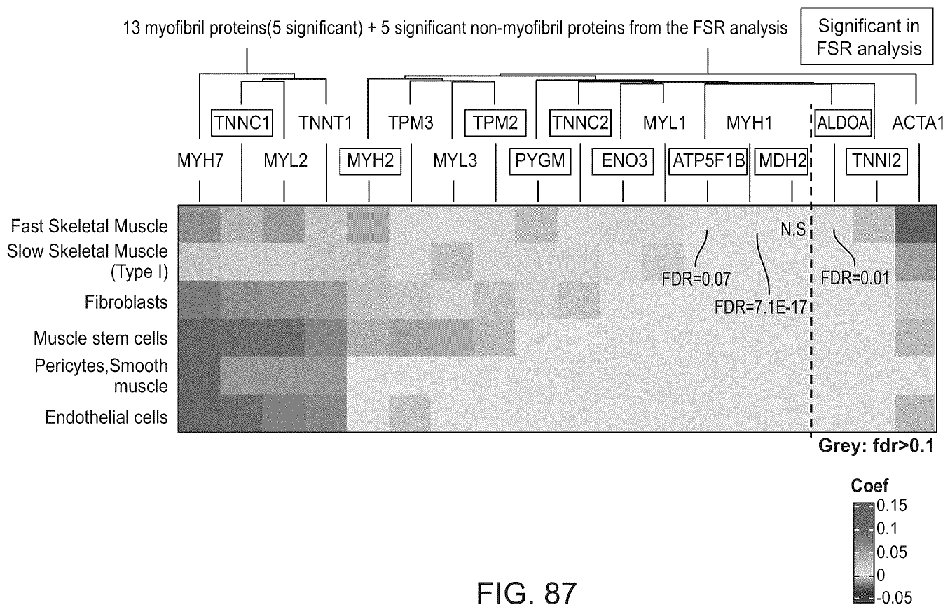

FIG. 87 shows the validation of muscle protein synthesis assay results in snRNA-seq analysis. 15 out of 18 muscle proteins show statistically highly expressed in fast skeletal muscle in treated compared to the placebo group. 13 myofibril proteins (5 proteins were significant in FSR analysis: TPM2, TNNC1, TNNC2, TNN12, MYH2) and 5 significant non-myofibril proteins in FSR analysis (5 proteins: ENO3, PYGM, ALDOA, MDH2 ATP5F1B). All 38 proteins in the heavy-water were also measured in snRNA-seq data.

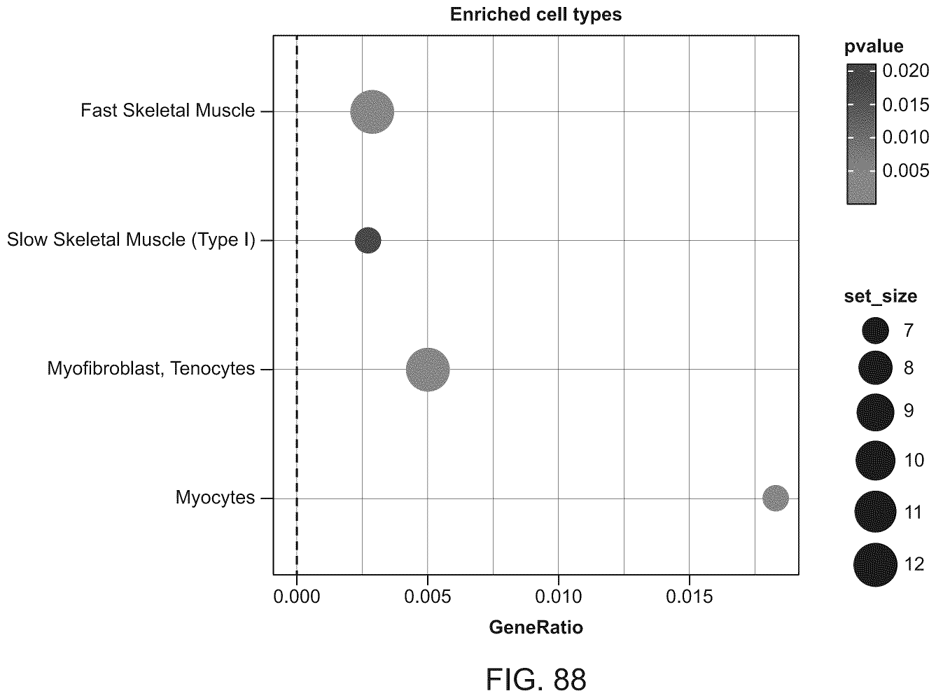

FIG. 88 shows that BGE-105 treatment shifts the transcriptome of fast/slow skeletal muscles towards a state linked to younger muscle. Transcripts negatively associated with BGE-105 treatment showed enrichment for the muscle aging signature. The result correlates with report on gene expression changes in old vs young muscle (see e.g., Perez et al., 2022).

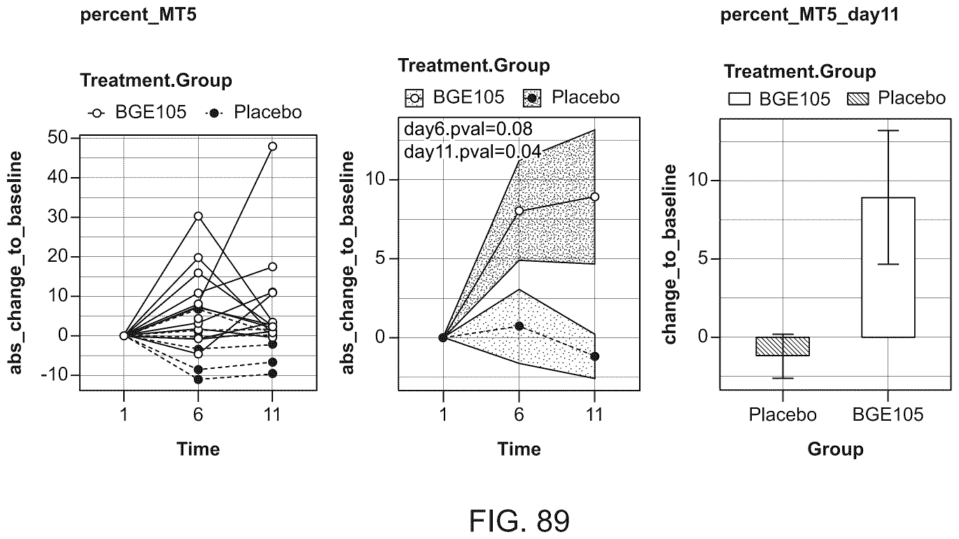

FIG. 89 shows graph that indicate percentage of cells that have Mitochondria reads more than 5% within a sample. Y-axis is the percentage of cells that MT reads>5% within a sample (62 samples=21 patients*3 time points) compared to that in baseline. P values of T-test are day 6, p=0.08; day 11, p=0.04. Cells that have low qualities have been removed before calculating the proportion of cells that have more than 5% Mitochondrial reads for each patient (cells with less than 200 genes or less than 500 reads were removed, total remaining MT cells(>5%).

### 6.10. Example 10: The Effect of BGE-105 on Weight Loss or Muscle Loss in DIO Mice Treated with GLP-1 Analog

In this study, diet-induced obese mice (middle aged or aged) are treated with BGE-105 and a GLP-1 analog in order to determine the effect of BGE-105 on weight loss and muscle loss.

### Methods

Diet-induced obese (DIO) mice are generated by placing middle aged or aged C57BL/6 mice on a diet (60 kcal % fat) for several weeks. Age matched lean control mice are maintained on diet (10 kcal % fat) (Research Diets, Inc.). Initial body weight is recorded before high fat diet feeding and then body weight is monitored weekly or biweekly (the typical DIO mouse body weight is between 40-50 grams. Leans are typically 25-35 grams).

When the 60% high fat diet (HFD) feeding mice are ready for drug treatment (˜12 weeks of feeding), mice are switched to (40% kcal fat) for 2 weeks prior to drug treatment. Control mice are given diet (13% kcal fat).

Body weight, body composition, and fed glucose and grid hang test are measured for randomization.

After randomization, mice are given treatment as the following groups (n=8-10):
Group 1. Age matched lean mice;
Group 2. Vehicle control in DIO mice;
Group 3. GLP-1 receptor modulator-1: semaglutide in DIO mice;
Group 4. GLP-1 receptor modulator-2: tirzepatide in DIO mice;
Group 5. BGE-105 (50 milligrams per kilo (mpk) in drinking water, pH 8.5) in DIO mice;
Group 6. GLP-1 receptor modulator-1+BGE-105 combination in DIO mice; and
Group 7. GLP-1 receptor modulator-2+BGE-105 combination in DIO mice.

Primary and Secondary Endpoints include:
weekly body weight, clinical evaluation & body condition score;
weekly fed glucose and food intake;
endpoint grid hang test and oral glucose tolerance test (OGTT); and
endpoint body composition by tissue harvest and muscle/fat weight ratio.

At the end of treatment, an endpoint grid hang test is applied to assess the muscle endurance and strength. The mouse is placed on the grid and carefully lowered so that the mouse begins to hang. Once the grid is completely parallel with the horizontal plane, the timer is started. The timer is stopped when the mouse falls onto the padded floor and the time to fall is recorded and graphed.

At the end of treatment, an oral glucose tolerance test (OGTT) is applied. One day before the OGTT test, mice are placed in clean cages with paper bedding, no food, no running wheel and free access to water only from 5 PM onward. The next day, at 9 AM, an oral glucose challenge (2.5 g/kg, bw) is given to fasted mice after baseline blood collection (indicated as Time 0). Blood glucose analysis is performed at (0-predose), 15, 30, 60, and 120 minutes post-glucose challenge.

After OGTT, mice are refed with diets and treated with drugs for 3 more days.

On the day of necropsy, blood plasma and organ/tissue are collected. Tissue is weighed and processed for further tests.

A terminal cardiac blood sample (EDTA plasma) is obtained for clinical chemistry analysis, using parameters such as: ALT, AST, Total Triglycerides, Total Cholesterol, Total Protein, Albumin, Creatinine, and BUN.

Tissue: Half of the tissue samples are frozen down for molecular biology analysis and the other half are fixed in 10% neutral buffered formalin (or gembedding in OTC) for histological analysis to measure and quantify fat: Inguinal fat, perigonadal fat; muscle: quadricep, tibialis anterior, gastric, soleus; liver; pancreas.

Body composition is measured by Echo-MRI (baseline, 2nd week and 4th week, optional) or Muscle weight/Adipose tissue weight ratio is used to compare mice lean/fat ratio at endpoint.

### Results

Treatment of mice with semaglutide and BGE-105 (treatment Group 5) results in weight loss while preserving muscle function and reducing muscle loss.

### 6.11. Example 11: BGE-105 Prevents Atrophy in Immobilized Human Muscles

Two groups of healthy older adult humans (e.g., n=10 per group) who are moderately active remain in bed continuously for 10 days, except for toileting, and they consume a eucaloric diet providing the recommended dietary allowance for protein (0.8 g/kg of protein per day). One group is given 200 mg of BGE-105 a day, while the other receives a placebo. Measurements before and after bed rest include muscle function and protein synthesis.

BGE-105 is shown to prevent or attenuate muscle atrophy in immobilized human muscles during periods of disuse.

### 7. EQUIVALENTS AND INCORPORATION BY REFERENCE

While the various embodiments of the present disclosure have been particularly shown and described with reference to a preferred embodiment and various alternate embodiments, it will be understood by persons skilled in the relevant art that various changes in form and details can be made herein without departing from the spirit and scope of the disclosure.

All references to issued patents and patent applications as well as non-patent documents cited within the body of the instant specification are hereby incorporated by reference in their entirety for all purposes.

1.-41. (canceled)

42. A method of increasing total weight loss caused by administration of a pre-determined dose of tirzepatide or pharmaceutically acceptable salt thereof to a human subject in need thereof, the method comprising:
co-administering to the subject being treated with the pre-determined dose of tirzepatide or pharmaceutically acceptable salt thereof a dose of an apelin receptor agonist of the structure: 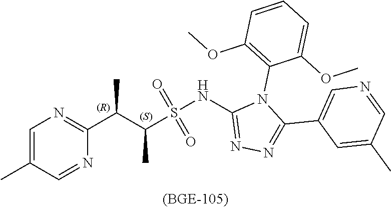
or pharmaceutically acceptable salt thereof that is effective to increase total weight loss.

43. The method of claim 42, wherein the subject is overweight.

44. The method of claim 42, wherein the subject is obese.

45. The method of claim 42, wherein the subject has a disease or condition selected from obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, and nighttime hypoglycemia.

46. The method of claim 42, wherein the subject has diabetic obesity.

47. The method of claim 42, wherein the subject has type 1 diabetes, type 2 diabetes, or gestational diabetes.

48. The method of claim 42, wherein the subject has one or more of hypertension, dyslipidemia, obstructive sleep apnea, and cardiovascular disease.

49. The method of claim 42, wherein the apelin receptor agonist is administered orally.

50. The method of claim 49, wherein the apelin receptor agonist is administered daily.

51. The method of claim 42, wherein the pre-determined dose of tirzepatide or pharmaceutically acceptable salt thereof is administered subcutaneously.

52. The method of claim 51, wherein the pre-determined dose of tirzepatide or pharmaceutically acceptable salt thereof is at least 2.5 mg administered subcutaneously once weekly.

53. The method of claim 42, wherein the co-administering stimulates in the subject:
muscle mass preservation or an increase in muscle mass relative to a baseline level;
loss of fat but not lean muscle relative to a baseline level;
increased lean mass percentage relative to a baseline level; and/or
increased lean/fat mass ratio relative to a baseline level.

54. The method of claim 42, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.

55. The method of claim 42, wherein the apelin receptor agonist is a pharmaceutically acceptable salt of (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.

56. A method of increasing total weight loss caused by administration of a pre-determined dose of semaglutide or pharmaceutically acceptable salt thereof to a human subject in need thereof, the method comprising:
co-administering to the subject being treated with the pre-determined dose of semaglutide or pharmaceutically acceptable salt thereof a dose of an apelin receptor agonist of the structure: 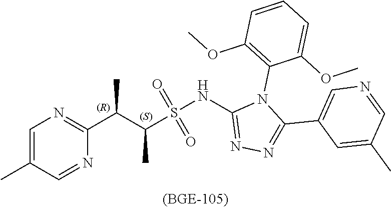
or pharmaceutically acceptable salt thereof that is effective to increase total weight loss.

57. The method of claim 56, wherein the subject is overweight.

58. The method of claim 56, wherein the subject is obese.

59. The method of claim 56, wherein the subject has a disease or condition selected from obesity, obesity-linked gallbladder disease, obesity-induced sleep apnea, diabetes, excessive appetite, fatty liver disease, non-alcoholic fatty liver disease (NASH), dyslipidemia, metabolic syndrome, insufficient satiety, hyperinsulinemia, and nighttime hypoglycemia.

60. The method of claim 56, wherein the subject has diabetic obesity.

61. The method of claim 56, wherein the subject has type 1 diabetes, type 2 diabetes, or gestational diabetes.

62. The method of claim 56, wherein the subject has one or more of hypertension, dyslipidemia, obstructive sleep apnea, and cardiovascular disease.

63. The method of claim 56, wherein the apelin receptor agonist is administered orally.

64. The method of claim 63, wherein the apelin receptor agonist is administered daily.

65. The method of claim 56, wherein the pre-determined dose of semaglutide or pharmaceutically acceptable salt thereof is at least 3 mg that is administered orally once daily.

66. The method of claim 56, wherein the pre-determined dose of semaglutide or pharmaceutically acceptable salt thereof is at least 0.25 mg administered subcutaneously once weekly.

67. The method of claim 56, wherein the co-administering stimulates in the subject:
muscle mass preservation or an increase in muscle mass relative to a baseline level;
loss of fat but not lean muscle relative to a baseline level;
increased lean mass percentage relative to a baseline level; and/or
increased lean/fat mass ratio relative to a baseline level.

68. The method of claim 56, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.

69. The method of claim 56, wherein the apelin receptor agonist is a pharmaceutically acceptable salt of (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide.
